SEQUENCE LISTING (251 sequences)

SEQ ID NO: 1 - DNA, 647 bp, Homo sapiens
ttgaacctccgactgcaacacagattaggatacagcagtggctacaattacacacttgcttgggaagttgggttgggagggggggtgggtgctgcatgtgacaggccatgaaaggtccctgcctataaaacagactgaaaaacaaacccttaatttaaatgttaatgaaagtgctgtggagacatcagaggcttcccggctgctcagagaacagctacaccagccacagaatttatggctccccttcaccctcccgcctccctctctgcctcatcacccactgaaatttaaagacagaaaaaaaagaaaactaattttaacattaactccatgcttccactttgggatgtgattgctaaattatctgttggcagaacggtgatcaaaggtggaaaaacacaggcacgttgcacacacaacccagaaaagtcagcagcagctaaagcaggaaaattcccatttcccttcctgatgagcagagtggaagaaagagtggtgcaggtagggactggagccaggaggtgtaggcagtgggaggaagagaaagatgactgatccgtccagcgtgctcccaaggccctgtctcaacctcatctgagaacttaccacactctactacctattttacttgtcggtctctcccatca

SEQ ID NO: 2 - DNA, 361 bp, Homo sapiens
cttatcacttcctaattattttctaccttttactaacctatgctctggagatttataactattatatctgtgtggtgcaaacatcatataatggtgtgctactgcacattgcttcccaactctgcatgactctgatgtcacattgatagcttgaaattggccatggtgggtgtagctctacaatgaaaatcagcaaatgctacgaatcaagactttttttccctctaatgttgaacatttaagaacacattgtataatatcctaagaagatagtcaaaatgcacaatggcctccttctatcccaaataaccagccagataagcaagcatctgtaatgtccctcagaaaccaagtgcagcca

SEQ ID NO: 3 - DNA, 406 bp, Homo sapiens
tgctgaaccaattaaggccagtggtaccacctgagattttgaaaataaatcagtgggtttttatttcagctgtttggaaatgactaatagagtaatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaaatgaatacttatactcctttaaatactaattaagaggccctggtaaccatttatcagtaatattaatggcatgaaaattgaaaaagtatgactcaaaggtgaattcagggca

SEQ ID NO: 4 - DNA, 607 bp, Homo sapiens
ttcttccactcctttccctgctgcaggaatcagtcccctggactctgatgtgtggctgggttggctgggttgccctgggcagacttattagcttggactctttcgcatgcaaaagatagcacctgtgtgactgtacatattgaaaatgcaattgcaggtgtatttgcaaattggattttgagggggaaatttaagcaaatccttttgaaagcaaatccctccaaggtgtgagtaataaagaaacaagaagaaaggtgtcaatcaaaaacctattaaaatctagtttccagagaagaagctgactgtctaatgacatttatgctttcatagctaggcatttaacagtcattttgtgtcagttgcttcctttatacatgtcacgtggcacatttttcagttttctgtatttctgagaccttaatcccaatcactcttaaatgcttgaaatgagcccatgttcctaaaaggagggggtttttgtccacctgaatttctctgcaggaatctacaatgatgcctcaagcatcagtatatgaagtcaaccactgtcattcatccaattcattcattccattcattcttttagttagccctaaaactccatggt

SEQ ID NO: 5 - DNA, 622 bp, Homo sapiens
caactcgcttagagtcacctgctagaggtttctggttatttggagatttgctggctctgccatggacatgatggcatattcgtggatgatatggagagtgacaaaatggctgagtgtctaattattaatcaaatcacagattcatatctctctccccctcctccactagtttccttggagacacagttttttgtcaattttttgaattgcattaatattaactatgactttgaatcaggaagaatgcaaaatatctgaagagtagtatttatatcttccacgtgacatttgcatcacatggataaactttaaaaatgttcttgagaacacgggaaaataaggaaatagagttcaaaaataaagtatagaacttgttagactagtttctgtttaaaactcggtggattatacctctttgttttaaaccattagaacagcaatgattaaatatcagattccttaaagagtcaccagttccttttggaaagttttgattaaagcattgtatttattaaactgttctacccacacatctttccttcaagatgttgtttttcccacgtctactgttttattacatgacagttattgaaaggttagatgtggtttaatttctcaaggt

SEQ ID NO: 6 - DNA, 392 bp, Homo sapiens
tggagaatacattcttgactcaatgaatactatcacttttttggttaaacagtccaaatttgacatccagaacatttctttaaagtcaccattttcctggaaacactctaattttgagatgtctagggctatatttagcttttgaacagtagtctttgaaaaaatatggttatgaaacagattgatttagaggagcattataattagctgccgaatacgttagaaggatagattattctacatgaatcacagagctcactttaagtgtcattccccattctaaactgccagcttaatttcttctcatttttcaccacgagagccttttttctattatgccaataataaagggttagttgtcatgaaataggcatacctttgtgaaataatga

SEQ ID NO: 7 - DNA, 634 bp, Homo sapiens
actaaggaatagacaatgggttctttatgaatatgtgagcatgcctaaaagcaacaaaatcacacttttgttaaagcatttgtgtgtattttgcacaatttttaaaagtttaacctctgtagatcaatgtgcttccttaaaagactttaataaattaaagtgcagagagctttttttcaccaggcagaaaaagctgatgagacaggtttagtttaatctcaggcaatagaagtcattaaccattctctcagggattaaaatatagcttgcccttttcttctctcacttatcattagcatcactttaatctcctagcgtttcacactgctattttaatcattacttactgccttttgacaagtgcagaacatgctttttctcattgaggcaatatcctggttttagctgacaaatgtaggacataatgtaaaaatatcaaagtgacatctcttaaacacaccttcatcataaaaactctaaagttacaatattcctttgtctagtatttccatgaaaattatcatttgaaaggtttatatcttaaataaagaaaaaagtttcttccaaatacaacaaaaactagtttagagaaatagtatgctgaataaatgagatctaaaaatatgcacataca

SEQ ID NO: 8 - DNA, 427 bp, Homo sapiens
agactgaggagacgcctcagaggaatttgtacggggaaaggtctgaggaaaaaggatcggatcccaatgggccgaactccttcaaaatgttcccgaggtccacgtgacgttccctcctcccacccagcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagagaaactcgggtttctaacttttaaagtgggattatatgtcttctccaatatatgggctccctgcttttataacatgctgctgttacatctaataaaaaccattttgaatgaaggcagcctttcatc

SEQ ID NO: 9 - DNA, 405 bp, Homo sapiens
tagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaa

SEQ ID NO: 10 - DNA, 450 bp, Homo sapiens
cccctccccactccttgcccctgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccac

SEQ ID NO: 11 - DNA, 279 bp, Homo sapiens
ttcccagccttaccttgttgcaggcctccagggtctgcagcggggtgaggcagggtctgggagatgcagtgaatgcaggaaggggtgtctgcctgacagccccatccctgcagattttgatggctccgtaaaaatgtctcctcataaactgtcaaagtgtcacaaattaattaactgttagctagagtgactcagtgtgggggggggtgggggagaatggctcagtgatgctgagcggggtggtggcggtgctgttctctgcgtgtgctgccttttgat

SEQ ID NO: 12 - DNA, 568 bp, Homo sapiens
acttggaggaaaggcagaggaaaatggtcactccagcagtgctgccaggacaccaggaccctgctccatacacctaaacctcagcttgcccttcccgtggaaataaggagatgaagcactcagaaaacacaatgcagtttcattttcagggtgggattttttccttctatgtatctcatgagtcactagattagcacctttccaattactgggaggcaaaagccctattatcatagaaaccctcctgcagagtgacggctctattctttctgacggcagcagccagtcccacagagagctgcagagctcagcaagctgggagcctgaagttccaggtaacttcatgtcctcgtttcaaaagtgaagtttaaagaactcacatcacagccttagcagacttctcacactccacagaacacgccaggacagcccaggaaggaaggctgaggaaggttcaggagcacatcccagaggctgcacacagggcccggggctgcctcgaacacactctgcaagggtctgcagcactgaacgttctgtctgagctgagggagaactgcatgacaca

SEQ ID NO: 13 - DNA, 621 bp, Homo sapiens
tgccacttgaacaatgactgtcattcattataacttttgcaaacccaccaaaagctcctgatggcatgcatacccatgcatcacacgtgcatacgtgcacacatgccttttaaagaccccacatgggtgattgcaagggctgtgtttgacagttgtcatctcttaaaatgatgtaggttcctgaagttcctcatgcaggactgtcagcctgacagccttagcaagtcagtcctcgttctcaaatgggaaaatgacatgggagggccttttcctccctttttggtagatctgccattcatcagcatcattctcttctcagccacttgctcataaagccttccggaggcaattactcaggtgtacatgacaggcctacattcctgtcaggggtgtgggctgggttagagaggatgcgggtgacactctgttccagcagggaccatgcctgcccaccaggaagcacatgccgttctcagtagaattctcttttcagagtttaggaagccactagcgctataataactgcttgctgtggtttgtggtcaagagcaatcatagcactgattttatttttctaatttcattctgaaatctagacaggaggtgagactgcatcacgtt

SEQ ID NO: 14 - DNA, 737 bp, Homo sapiens
aaacaagaacccgggggaatccaactgctcagcatgtgggaagggctctgcgcagcccgcccctgctcctggggccgcagggaaaagtgggtgaggccagcactctgtgcctgggatgccactgctgtcctgcgatgttgtgctgctcccccgcatggctgctgctacgggctcttgccacagctggacaccctcccctgagctccagtgccggacaactgttttctgagcatgctaagcaatgaccctctgctgacgggagaagttactttagaggggacagaacttaggaagacccaggtgtgctgtgaggcaagtggctccctccctgacccttactgctcagtgtgcaaggttaccaccttctcctcctccccacacccatcccccctctctcccaaggtgcccctcattgctgctgtcactcatcattagcagtcactgtcattgttaggacagcccctgtcaggctgcagagccaagggagggggcaggagaatgtcagatgactcacacaacctcaaaagagggggatggacacacaacacagaacctagctacaagaagcacctcgtgagactctggttcaaccctttctttttccagagaagaaagagcagcttagcagagtttcctgacaagtccctctgcttcccactagtcaggcttggagggaggctggcctaggagaaaggccaggccaggaagtcagcatgaggctgggcaggtgacaggtg

SEQ ID NO: 15 - DNA, 673 bp, Homo sapiens
aggttgagtggattgaggagaaaatgggaattttcatgtttttcaataaatttagctttaaatggaaggagagagatagtatctagatggaaatacaaaatcacaagataatctttctcacttttagacagaggagaaatcaccacataactggaaacactgaaaataggagaggggaaagatggctgacagagtcaggcttgacaggaggaggatggggtccaggacacagtgtcagaggaaagaggagtatagacaggaggaggtggggacacagttcccctactctgacaggaggcaaggtgggaataattggtactgatagatctaagtttatgggaggtgggaggaagttcatagagttcctacctgacaacctagattcatctgaaggaggagtgccagttaagagactgttacaagaagtatcgagaacctgagttgatgacattgggaacaaagctgcatacttgagacactgtaatcatttaacaaaaggtgaagttcacaccatacaagcactgcaagaatgtagagcaattagaattctcatagactgatggtggaaaagtaaaatggtacaaacactttggaaaatctttaggtggtttctcaaaaagttaaacaaagtcctaacatgtgactcagccacccactagtaagtatttaccca

SEQ ID NO: 16 - DNA, 1125 bp, Homo sapiens
attgatggagttggggctctgtgagtgtggggccctgtgattccttgcactaattccaggagatgggtctcctaacagcatgatcatgtggatgtcctgtgagccctggcggggctgtacagagagaacaaactgccgagcacagtgcagctgccttatcagatggggcacacaggcttgcacagtgagcatctcggcagcagccttggtagactgcagaccagagactcgtcccccactttccagagttgctaaattccaatgttttgagaaacacaagagaaagggttggtgggggtcctggaaaaatgatttagtgagtaggggttaagccgtatgttgtttcagttagaaaaatgaaaagttaaaggcgaaagatttgcacgatctgaagagaaaccagaatgacgagttaatggcacatgtttacatacgtaacaaacctgcatgctgtgcacatgtaccctaaaacttaaagtataataataataataagaaaaatgaaaagttacatttcttatatctgagcattgtctcattaatatgaagtagtcataagcaccttgtgttttggggatttaccttacaaaacactcacaatatctttcctcattatctggtcaccaaattaatttattcatcattcatatatccattcatccgatattggagaatctgctacagtcatagtgtagaggtgtacaatggtgactaagatgtagttccacgttcttacaattcttactagtgacgtggataggagataaggaaccgcagtgaaaaggatattgactgggcacaaaaagtttggggttttaatcctatttttgtccttcaactgctgggcaactttgggccaactacacaacctttcttggcttccctttcttcatttgtaaaattggactaaattatctttaatttcttactaacttctaaaatactttactgaagaaaagagacatacacatctgatgtaaatgcaagtatgtgtgtgtgtgtgtgtgtgtgtgtgtttaatgggggcatagctggctcctttattaggctttggtttttcttcatgtatctgatttccagaaatgagagtgaaaatacattttattttgctcccacgtgtttact

SEQ ID NO: 17 - DNA, 742 bp, Homo sapiens
actggggtcacaaagaagaaccagggaacgctgttggggaacccatgcaatgaaatgtgaagggaaagagggcgggcagacgtccccaaacccgctcagcaaagacaagcttcgggcaaattcactcacgttgacgctgttcggatggagcaggccaattggaaaagccgggttcagacacgactctagagggaagagaagaaggcaagtgtgaggagggcgggcgtcggggagaagcgggcccgggtgtgctggcggacgtagggcctcaccgtcgcggtcctcagcgtctctgcggggtcacgggccaggctgcggggctctgagcgcctgcctcgccgtcccggggcttaacggctgctggagccactttataattagccccaaaccgaaggaggcgcgcgcgccccaatcgccggcgggctgcagctgacgcaggccctacgccagccccgcgccgacgccttcgcgggaggggtcggtcccccagcacccgcgcgccgctccccccgccgagctcgggtgggggccacagggggcgctcggtggctccccacacggccaggtgacaaacccaaggcagttaggaagtttggggagagtgagataggatcaagtttctccgcataaagtcactgaaattgcaccgtgggatgcgggctccagggagggaagcgaggtgggggcgtttggaaggatagatggactggcttagacttctggaatgtgctggagacccagc

SEQ ID NO: 18 - DNA, 402 bp, Homo sapiens
cccactctcacagcctagggagaagaggaggtgatggaggaggaggtgacaggccccctccacccccaccccacgcccacccccacgtggccccacagctgtctgtctgtgtcaaatgttctaagcagaggttgggggcttcactccccacacaaagagagttcagagctgtcagcacctttcccacggtacactaggtgctgaggaacaggtgataattacagccctgtcaccctcatcacctctgagctggatgagtttttttctaaaaacaaatgttgtgctgcctgaagatcaaagttggagcttaggttggggaaatacacacatgcaggctgctgctcttttccatgaagcacagggaattggcttggaaaacaccttggagatggctgtcttggc

SEQ ID NO: 19 - DNA, 604 bp, Homo sapiens
gactctacctgctgtaaaggcaaatgaatgatcctttgattaaaaggcactctacagagcggcacaggcacagacagaaacctagggaaaggcaaagaggagtgaaaactggaggcttcctttccatcctgtcgtcaagagcaagagaccatcgtcaggacctctctggagagagtcagagcctgggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaatgctcttctcactctgctctgggtctcccttccgtccctccctctctccctccctcccttccttcttctttcctctgcttctctttgtcatctcttccaatcctcacatctgataagcctgtataccctcataagattcctaattgtgggatatgcttttaagcgtccatccaggccaagatct

SEQ ID NO: 20 - DNA, 567 bp, Homo sapiens
taagtccgtttgggcagcctgagatttcatcaggtgttttttggcatctgtgttagactgtttgctcattttgtgtttgtaagtggctaaaaatccttaaatctttctcataggaaccagttataaaccaggtctcacaattctgttacttaagcagaatttccagctctaagtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggcgtccgataatcaataaactttggttaaaagtattcagcattctggggattcacattaagtggtggagccaaccacttaagagtatctgtctctgggtgggcaaggagctaggcttcctaggagggcatgacaaggcaccctttctgcttatttcacccggatgctgactcctt

SEQ ID NO: 21 - DNA, 637 bp, Homo sapiens
gagaccacaggctgagaggccaggcagggttcaccaggacactctgaaagagctgtctggctcccaggcccacaggccttcgagggaatagacacataagccttgctcctttttctcctggtatctgcccacacccatatcctggaaagctgcgtgatttatgtccaccagaggggatttgctctgaggctgtggcctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatca

SEQ ID NO: 22 - DNA, 586 bp, Homo sapiens
agggaggaactaaagtccattcatatgtccatattctcaatttgggaaaaaatgtggctttgtcttcatcctcttttcctgagtgtataaggccagaagccaaaggcaagagcaggacattccaggagcctttatgattgctctaagtcacatctgctctcgagctcccattccactgtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctgccattctcttcggaggtgagcacctcctgctcgggaggaactgacagcccctttcagaaatgtgaaggcagtgaagtcccaaaccagaaacagtgtgaccacctaggctcacccaaggaccgtcctgtgcttctgggcaaccaatggatgtagggggttaggaggcagtggggattcgtca

SEQ ID NO: 23 - DNA, 568 bp, Homo sapiens
catcccgctggccatctgcatcagcccgacccccacaggatagaagactggcacatccaagttgctccttccacaccctctccccactgaggcctcgtctcctccgccctgtggccaattgaaggtgaactcctaacgtgcattctgcctgggatcccagaggttgtggcagccagatgagtcacctcacaaatacagcacgtgttcctcgggggcggcggtgggaggaaacacgtgaagagctctctctgtgataatagaccatttagagcttcagatggcgaaaacaagctgatcaggtgcaggccctccaggaggctggcaggtgactcccagccagcacgccctccatcgcacgttggggaagctggaaagagcctggtggctgccgggcctccctctggcatcctctctccctccaaggctgtgtgctccccaccttgtctctccttttttcattccattgctctgttctgtttctgtctacccattcgtcttttcttcctcttctctttctgttgccattctctttctcttctcctctcaacatgtccttctctctacccgt

SEQ ID NO: 24 - DNA, 447 bp, Homo sapiens
tgactgtgaagtgtctgtctgaactcataatcagcttttagctggtcccaaacagtgagtgctaccagctccattaatcccactgacagggatttatctttctcagctgaatgaatgatgctcccagccttgccccagaaatgtgggctgggctattcaacacttgtcaccagcaagctcttttcctgaggctgcatactccagccacccaggtgactgagtactttgtcatcttcttctcagagcagtggcagaaccacaaagcaaactcccacccatgacccacccacccaccttctgacctagtttggatgctccaagccataatgataaagtaatgttgtgaagagggaaagaggtttgcagtcagacagcctgaacccaggttctgccatgtacaggctgaagcttggcaagcctctgcacctctctgaggtccatttgcac

SEQ ID NO: 25 - DNA, 654 bp, Homo sapiens
tcccacatctgtttctctggctctgcctcttctgcctccttccatctcttatagggagccttgtgatgacattgggccaaccaaatattccaggatcatgcctgcatctctcaagatccttaacttaatcatatcagcaaagtccctgcaccatgtgaggtaacatattcacagtagtaggtgctcaataaatagatactaaagatagcagcattttttattagaccttctcacagcttctggggattagaatgagacatctttggcagccattgttctccccatcatgggtaacatgcagtgggcgcttccagtgccaggatttcggtgggtttaactggagtgtgcagataagtagttggcgtcaagaactcctgctgcttcccttgctggaaagatggggtggctttgaatgacatcccagaatctgattcttccgtctgcggaatgtgcataggatgtctgaatcacactattgtcgtgagaaatgaggtagaatattctctgatgtggcaagcttaggccagcatctagcccaattccttgtatataatggaaattcttaagatgttagggttcccctctccctctctctcctttttcctcgcctacaaaggaagacatcttttgtgaggcctccacagcattcttgat

SEQ ID NO: 26 - DNA, 418 bp, Homo sapiens
tccattgcagcttctctctctctctctctctctctgagtcatctttacaagcccctgaaacaatggtatttaaggagggagctttctcaacacctgaaatccctacagaaaggttgagcttccttggtaaagcatttacctgctgggggagggtgcatggagtgacatggttttcacatttgagaaaggtaagtgggagggaatatcagacaactgcagagtgcagattatcatttaaataaaatccccatggggctcgctggcagcattaaaattgctcctgcctctgtggtgctgtcacagaacagcctctctcctttattctgctttaccatagaaagaggcagtgtggtgcagtggaaataccccagggctgaatcctaggattcaagtctgatccatcatctctgggctgtgt

SEQ ID NO: 27 - DNA, 368 bp, Homo sapiens
tgggactcagcaacttgactaacatcacagacacagttctcagatttatatatcctaattccaaacccaatattcttactactagaatatgctggaccgttttactcctaacttggttaagaagccaaatataggaggaaattaacaaatcaaggtagttttagttactcattggtctgtgggttcacagcattttaatggagccaactttgctcttaagatgagtaagcagttggtatgactcaggaatctctaagaatgtttctaccttaaaaggcaggtttttgtttcttaatcctttaacaattaatattttaattaagggaacattggatagacattgtaaggtgcaaaatacctctagaagg

SEQ ID NO: 28 - DNA, 283 bp, Homo sapiens
agttcttgctttctccaatttgtttatttttatgtgacctagctttatacattgcaatttaaaggaaatgaaaaacagttttgttattttcttgtgttatctgggagattatcttattcttttagggttcaagtgcatagagttggtgcaatcagccacagtttatgacacagtataatttatgcttcttgcattctgttttaaatttattttcaccttttattttccttccttttatccctttgtattatgtctaatatcacctcatcttcaccccaatcca

SEQ ID NO: 29 - DNA, 290 bp, Homo sapiens
tgctcatgcacaatcactacttctatctcttcatcaatggtaacacgattctgacttccaacagaatggacactttgcaagttttgaaatgtatgaaaaggaaaaatatgtctttttctgcttatggcttttttagtcaatgttatattatgcactttatttatgtaattttgtcttgctatagtttgtttgcctttattgacgaataataaatatactagtatgtgaacacaccactctttaattatttagtctacttttatttccaggtgttggatctttttcatcga

SEQ ID NO: 30 - DNA, 470 bp, Homo sapiens
ctaattttaaagccttcatgatctatgtcccacaggaccatatgtcccatagcagtcaaaatcaaagacaaattcttggacagaactggattgatgtaataaatttctagggcatatctgaggccaggtgcatgtgacatttgagtaggagtcaaggatgtaaaactgcctacatagtcgtgttaaatgatcagaaattaacgtaacatgtttttgttaagatggtttcatttgtctgggaagctgcattcttagaacaaaaagagtcaaggacagaaatgtattatgagataagttccaccttcagactcaacctacgcaagtcaaaaaaaagttagattcgccttgagttcatgaacaagtggtgcttgacatccaaagaaatggctggagtaagtgatgttgccctcttttttatttctttggaacagagaagcagaaatccatactacctcccatctggctccatg

SEQ ID NO: 31 - DNA, 566 bp, Homo sapiens
tatgctgtggcctggggatgggagggagaatccacaggctgtggggatatttcataggcttcatctaggcattaaatgaggaaatgaatgcaaaagtgcccagcacagtgcctgacacataaaagcaccaataagcttcagctgtcttcccccttcttcaaactacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaacttgagctactttgtgtggtggtatttgtggaaagagcaggaatattgctgtcagtcctgagaggtggcatttactggcagatagacctcagacaagtgattttactcctgtaagcctgaatttgtttatagtaccccaactcataagattggtgaga

SEQ ID NO: 32 - DNA, 407 bp, Homo sapiens
acctcaggtcccacatatactgtggtctatcacctggttactctaaaatgccccaactcaccttcttccttaaagcagctcccactcctaacatttgtatctcaggctgattattcaggtttagaaatttcaaggtcatctatgactcactgttttgatcatgtcagtcacaaagttctaaagcttctgctttggaatgtttcttgtactgaatgcttccttccaattctcattgccagcaccagcctccagatctttatcaagtcatagctgggttgttgtaacagcttcccagcagagatggcatgaaggaaggcaactaaacttttctgagcacctcatatgtatgtaatgttttcaatgcactatcatatttaactctcataacaataccacgaagcaggtat

SEQ ID NO: 33 - DNA, 324 bp, Homo sapiens
ggggttacctttgggaaaataaactcttattaaagtgtcacatcttggtggaaaggcttttaatttaatgattgctatctacagggaaaaatcacttataaaaatcttactatttctcccaaaagacaccaggttgagaacattaggacatcattctttcatcaatatttcctttgtatttcttagtattttacggttttccttaatacttcttattccttcaaaagctatacctccatactatatatttgaactgaaatgtctgattttagaacaagtcacatgtcatttttattttgtctttaaaaacttctgacactgaga

SEQ ID NO: 34 - DNA, 349 bp, Homo sapiens
ctcagtatcttatttcttagtcacactttaaaactgaagagattagtaattttttctatgtcaatatgtgaaatatgcaatacaaaattgtacaaacccatttaattccattctctgtcttttgtcaatcaaacatcctccctgataagagctcaattatttgattaattataaatcgctattttttgatatgatattcacagtagaataatttataatagacaaaattacaaacaataagatcttggtgttcagtgggatttatggtgttttcatgcaatggaataccgaactgacacaaaagtcatgttagagaagaatatttatagatgtggggaaaagttcatga

SEQ ID NO: 35 - DNA, 644 bp, Homo sapiens
gtggtgatgctggacaaggcaaggagtgggcagaggcgaggactgtggtccagctgaattttcagaggccctctcatgtgcgcccactctttggaatcattctcatagataccctatgggaagcccaccccccagcctggccccgcgtcacactgagtcatatctaatcataggctcccagggtgatttccttacctgcttttttctgtgtttttaagctcttcattacttcctaattgtcctgattttagttctttctcgttcttgtcaaacacccatttagaaaatgagtcatcattaactgcacgccactcttcacttagaattagacccatttcaccctttctctctctctttttttcctcttcaaaaaaactgggggattaaaaaaattacacttttctgacatcctttttttaatctcttttgctagcgcaaatgctattggctgctcaactgccttaagggtttcttctgcagcccaaatgaacagctttgtgcttccagcctttgatagctcctgggggcgttgatcactgagtcaaacctccaggtctcagcctcccaagtggcctgctcaggcatgagtcaaatcaagactagaccatcaggggacaagaagctactgctcaccttgcaagtga

SEQ ID NO: 36 - DNA, 304 bp, Homo sapiens
gtgtcagctccaggacgagccccgccagcctccaccctgtccaaccaggccctactcctcccctgcagatctgtggtatcttctgagccctaaaaacacgcctccagccagcttcctgtcctccacccggcctctcggggctcatcccatctattattcacagcacaaaatggatttttaatttgagaaatgaaatgactctcccaagtggccggggtggcagggagggggtggaggaaggccggagccgccgtggccgccacagccgccacggtgctgactcaggttcatcttggaaagctcg

SEQ ID NO: 37 - DNA, 445 bp, Homo sapiens
agagggatgcaaatgaggctgtgggggctggggacaccccttcctgtcagggtctccatggcacattgcctgggactgcaaggaagggcgagcctgggacaggcagagaagcctgaatggcctggagggccacttccttggctgccagggtcctgggcctgcagtcccctcccccagcactccatcatcatttcaagtgattggagctgaaggggatcgttagctaattaaagctgaggccactaattgtcccttttgaagagagagcagggctgtgcaggggaggagacagagggtctggggagggggatattggcaggcggggggctgggaacagggccatggccccttgtggggccttcttccagactgtgtgtttgaggggtcaggcattgtcagaagctccttaaagtgggtaaaggactagagaagcagatttggcgct

SEQ ID NO: 38 - skipped

SEQ ID NO: 39 - DNA, 744 bp, Mus musculus
cacttgggtgccttgactaaataaagatggattccatttttaatggtcagttccagtgccttccaattcctttgagggcgtcgtttggcaggctcaagtacagtagttgaggagaaaccatgaccagggcaattacttagggttcttttaagcactggtttagtggagattttttttttttttttttttgccgagccattataattcgtgaataataaagaacaagagtgttcagattgcttagggcatggcacgttcagaaaagatcgggcatccttccaaagacagcggggcttcgggaaacgataaagcgctatgatttttaaaagtcatacgcagaattcagaagcagatactacagaggagtgcctgcaagtaggtctgctcgctctgatgggtctgaagcgggagagtgggggtggggcgggggagtaaatgttccctgctttgttgctattaacaccttggcccttgtcaccaggtcctttgtaaatgttggcagctgttattagatgcgttatcaagcaccggtgtacacagcttactttacaaatcacccacccgcagatgccttacaaacctccacagccatcaaaatgcaccccaggacttcctcgaaataccatttcctccccaagaagaaccagcagcttctggctccactgagttctgtacctaagggctacttgcctctcaaggccagagaaccacgaatccagtagttcaccgagcttctctgttcct

SEQ ID NO: 40 - DNA, 1020 bp, Mus musculus
ggacttttggtatagcattggaaatgtaaatgagctaaatacctaataaaaatggaaaaaaaaataaaataaaagtagcattaaccaaaaaaaaacaaaaaaagaatatcacttactgttgcatttccttctttatgcagttctttcctacataaagtcttggataaaatgagaaaggcataatgtacctaagaactaagcaaagctaatgctagttagcaccacaggaatgctgacctctcattacagaacaattcaacaagaactcattcaccaacagtcagggttgagattctccccacctgcctgcagtatattatagttgaaatatggggccttggtaggttaagagactactgtaaagcttctagcactattaattgttcactctacccatgccacatctccccaggagatcagggaatattgtattgtggtgctccaaaagctcgttatgcaacgtacaaattatgttatgatgaaaggcaacaacaaaacctctgtaaggagtgttatcatttctctcttttcattaagtcacaatgcagaaactgagtaatctgaacaagtacctttaaagtaagagacttagaaagtggctggctacatgtaaagccgttcatgtgcttgcagttgtgtacaggagcaggctgctcacctgttacggagagccctttgttaagagcctttgtgagtactaaaagctttaggtttccttttcttttttaagctcttgctttaacagtgtatttctatgttcacctttcaggtaatgactgtctttggtctttttatcaaagagggaacaataacaccgcatggtattattttcacaagtattatagagtatatatttcacaagtttggtaccctttttatatcatatctttttattttagttatgtacctgattccaataaaaatcaagtaatgttgattgcaaaccccatctgtagtcatagaaatattacaatgatggacatgtagttagtttttcacttctcaaatactgtccatttc

SEQ ID NO: 41 - DNA, 942 bp, Mus musculus
aaacaaagagccgccttccattcttctgagatgtctttagacatcttgcttacccctgttacacagccccacagtcattaccaagttcaatcatctaatgtgggatcccaggaggtctgcattaaccatgaagcaatgaaaggtagagtgatcctgtatatggaccaactgtcttccccaccagactgaaagctccctggggtcagagacaactcttactcatcacagaggtaccagtatccagactatgctgtctcttcccaacctctgcttgctgtcaagcctgaagctagtcaggcatctacctctgcctggctgcagttctttctcttgaagaaaggattttttttttttttggtttcataatgattagtttttttcattatcaaaacctccctgagtcacaggaggtaacctaatatcaaaacctccctgagtcataaacaaccattgctactgctacattcaccattatggaaaaaaaaggctggtctgaaacttagcctcagtgttctttctgccacctaaggaggaggttgttgcatttatttatttggggggaggggtctagtcatagcatacatgttgaggtcagaaaacactttataggaactgatttattccttctacggtctgagtcctgggaacgaaactcgagtaatctgacttgagcataagcacctttagctatgagccatcttgctagcccacaatgtgtagggggcagtgctgatgtcaaggtactgttgcccaattggttcttgatctgtcagtaaagaaagccataggctaattgctgggtggaaggtacagtcgtgacttccaggttccgggagggaaacgggatgcaaggaaggggggagggagttcgacatgctttggagagaggagaacaaagcaaccatgtgaggtagtaatacactgtgacaactacaggcaggt

SEQ ID NO: 42 - DNA, 897 bp, Mus musculus
gaatgtattggattataacatgcactagtattaagtataaaagtatgttcaagaaaatttaaagttattttaaagactgatgtaaggaaagaaggaggaagttagtccattaaatcacactgagcaagagctaacttctacagagtagtcattaagaggtaattttacaatctaaattttttttttttaaaggatgctttcctttgttgtggtgtcaaggtgagaaactatgtagatttgattccgtaaatatctatggtaaggcaagtgacagggtgaactcagctgagctgatggaaatagttagaacatagaaacagtcaataactattcattttgtaaaaaaaaaaaataataataaaatagctccttcaagtcctgaagcgtaataaaatattagaaaacacaatggaaaaatgaggcactcacagaagaaatacagttggctctaaagcatagaaagtctcaattatttcaaaataatagaaaggtactttaagacagtgaacccggagtggtctctgggaacaggcagcttggtcctcaatctggcagtcagtgctaactacactgtgatcctggaaaagatatgctttacttcctcattcaaaaactcagaattatagtgctgcctgtgccctgatctacctgatgccccgccctacctgcctttgggggttgcaggtaaggtggtgtggaatcaggtgagaaatgtcacagcaagcacctctgaaagctgctgcaagctcctttaatacgctcacccgtgtcccattggtcccaagaaagcatctcttctaagcagcagttcctgattggctggcattgtctgtgtcagcaatccttggtctcttaggcagtcctcaattaggtcctcctgtagaaggtgactttgcc

SEQ ID NO: 43 - DNA, 547 bp, Mus musculus
agaatatgtcatatgcccagagtctggtacttatgagggtcatagtattatggtcaatccgacaaccactcactctgagtcaggcagagtgtgcaaagtgcactcatctgtgtcacttattgtcatatgtgcacaggtgagtgattaaaagcacaagtgtgcaccgcagctccaacatgtcatactgcggcctcagacaaagaaagcaacctctctaagcctagggtgttcttgtctatcaagtcgagggttataaaggaaaacaccatggggaagaattaaatgcaaacctccccccacaccacgcttatcaaatggctgctggcatgatgccttggtttaattcttacacagtcacataccaacgggtaaggctctcagagagtgttatataattaagacaagattcttgtgtttaagcagggtggaaaaagaaatacttactggaacacagacgtacaaaacaaacaagtttctagggaagaacgaagctgcagcactgtgtgggaacacacagaagaattagactgtatgcaggagaggggat

SEQ ID NO: 44 - DNA, 598 bp, Mus musculus
tgacaaatgttatggcgagcccacgtttctttcaggctgtatcttagtgctatgaaatccatattgcttcacgatcagcacttatcaagattaaagaaaaacaaactccatttatttttcctttattgacagattcatggcctacatccctgtgaataaatgagctggcaagaaggggaatgggacagccagtaagcttggtgacataaattacccttgatagggaaggctgatgtgggcatgtcatcaaattaatagctgaatgctaaggaccattacatttagcactgtttgccatatccttctatataataatgcctgagaataatgtgtaattaactctgtggctgactgacaaggccaaatgcagggcaatcaatcatattgtataaacacacgtccctgagtacacatgggaggcaaagggtgacttaaaatgtctcagcactttctgcctagattatggctctgagcttttgtgaggccagcagtcatagagactcttatctatagatctaagctgcctacagcatcttggtatatagtcttccttgctatttatcttgaccttttacttgtgttctttgagggggtgaca

SEQ ID NO: 45 - DNA, 686 bp, Mus musculus
atcttcaggtgtaccagcactgtgtaccagtctgtattctgtggggttggctgactccagcccacgctgtgtctgtaaatgaagttttattggaacacagtttagtgcttcctgttggtgatatagcacagagctgagtagctccaagagcaattatatagctctaaaagctgaggctgtttactttctgacctgctacagaataaggcttagacctcctgagtgagtgttctcaaatagcgatgctgagtggaattacctcaacagattcaacattgttcctgaagtacattcattatcatttactggtgttttaattggctttccttaaaaataaaacagaattaaagctgttacatgtagaattgtttatacagcagaacccgtatggtgttccttgaaggactgtgtttgagagaatcgcctttagagtgagaattacaaagacagtgggtcacgtagattaatggttgagatcctctgacctaagtaactgctacttttagctttattctattttcaactcggatctttgtttttgttttttactaagcaagtataagatggtaccaaaaaggatgttggctttccgaaggaaacagtttgccaccagttttttatattttcttctgtgccctgtgcgtgcctgccttttcactgctctagctagtaacgcccacgcttgg

SEQ ID NO: 46 - DNA, 600 bp, Mus musculus
tcatctgcagtcaatgaaggtctttattatcactaagtacgtcttatgagttttatatattgactttccttaagtaagccaagaacttataaaaagatcactcaaatagtatttgctataatcctcacacaattttaagagcaggaggcaggaggtctgtaatgtgaaaacaggaaagcagagagaggcaggaggtgaagcagatctgggtaattttagctgttggtagagaaatcatgaacacaccctggctttagtctgctgaagtggaaagctgtatacttattgttggagcacaaaagcagacagcatgaattgactatggtggtaaacaacttcagccctaacacaaatgaaactggccagtgactcattaggcatttagtgtttttactaagttttagttttatgaaaactaattccagtctagttattgaaatactccatgggttagttacttagagttcaatggaaaccattgcttttcttatttttttttttttttgtaaagcaattaaccattgctttaaaaagatttttgctggctaagaaaacagttgatgttctgggagaaaggaaaacctgactctcacttcatga

SEQ ID NO: 47 - DNA, 696 bp, Mus musculus
tccgtaaagagcacaaaacccaaatatttgggtgcatgggagatagatttcaactaaatacttttaatcatgcaattccaatagcacttaaattttttcaaggcatgtataaagggagaatagactaattcaatttcaaagtgcaagaaaaacatcaaaaactaaattcagaaagtttgcatgggaagaaaaaaaaacacaaattaatctcacttgtgattattctaaatacatttgaacaagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttattcataatgggggaacataaagtgaacttctgcacaccagaagtcaggttgtttgcttgctaataccattcccactcaatatcccatggaagttggatgactcgacctgcatcccaaggagtagttaccatcactaatgaggagaagcatttggaaggaagacagaattctgtagaccaaacgaagacctggaggctagtggaaa

SEQ ID NO: 48 - DNA, 518 bp, Mus musculus
aggcttcttctgcaatgtggagtttctagatccttcctatgatccaacccctcagacttcacatcctctccatcaccaggacagacgatcccattgagtaacatctggcttttgccttctgtgggcatatggctgtagctatcacagaagcatacagatctgctaggttcttgtgggcagcacacccatggtaccttttaaagggcctgctgtattcgctctttaacactcagcgtggcatcaacccagtggaaatgaactgggagctccctgcaggttctgtgctcccttggcaggcacagccttcccaccccacaccttcccctggagcattgagctctgcgggaacacaggcttgctgaaggaaccagcagggccatcgggagcactgggcctgggagggagatcaacagcacaaagtagagccagcacaaagtggagctgagagaagccttggagcatgaacaacatatctgcaccccaagaaaagcccttaaccgaagagcagagccatggca

SEQ ID NO: 49 - DNA, 622 bp, Mus musculus
agagagggagcaatttctttcagcaacggaaataggctttgttgtattttttaagtccccataaaaatgtaaagttctaacatctcaaattaccttatgttccacaggaatattaggtgttagcggatactctgcaggaaatgaaaaatgattacttgaaaccatttaattggctttcactcaacaaataccaaagtgcaatttgtggcctgatttgaatgctaatgcctgaattacaggcatggaatcaaagtgctagatgctttcagtgcagaatgccagtgcttaattaagttttccagtaactgtgtacagcaataggattcacagcgagtgcgcggatatttaaggttatttatttctgctatgtgaagtatttttcccttggaatctttgttgtacaaattgacatttaccaggtggtgaaaaagtttacaatcttgacaaattctttcattcaaattaatgttttccttatcaatcattccaaacttaaatgcctgtattattaggcacacagttggcatttaaaagtatggattaaggaactatgaatgcatctgatttgttactctgtataagcctataccaaatcaaattggttgtggttttccagaatttt

SEQ ID NO: 50 - DNA, 776 bp, Mus musculus
agtttcgtgtagttttaaaaaccctatttattcttttcttgtgaaatgtttcaaatgaattcaaagaaaatatgatgttttatttctttacaaaaggtgataaatattatttttatgaataatattcacattgggcccctatgtggacgctgaatagccctagtgtggagtaaggtacaaccttactggaggaaggatatcactatggggctgggctttgaggtctcagaagcccaagtcaattctctcactgctgcctgtggttcaagataaagaactctcagctccttctctagcatcaagtctgcctctacactgccttgtttcctgccataataataacagactaaccctctgaattgtaagccagccccaattaaatgttttctttataagagttgttgtggtaatggtgtactttcacagaaataaaatcctaactaagacattttccaaattgtatacccttgattctttttagttcttgtattgctctaggtagaacttcaaatactatattgaaaagacattggtagagtgggcagccttctcctattcctgatttgagttgcctctggtggagcaaggggtttatgcctggatagaatgacttattatagaatgacttagaatgaatggggttcttggtgctggtatggcctctggttgtgaaggctctggggaagtattgctcattcatgaggtgggggcttctctaaaaatcttcatgctagtatagcctctcggggatcagggtctgctggaagtgtgcataa

SEQ ID NO: 51 - DNA, 518 bp, Mus musculus
actcctctggtatactgcagaaaatccttccaaaaatcttcacaacagtgaggtttttaaacaggttggactcaaagcatggagaacggaaggagaaacacagtgggggcagaaattccagaggcaagctgagctggaaaacacatggccataatcagggggtggggaaacaaagcattcccacaggacagtgggaggcacagagcaacccaatgtcacttatacaccttatagaaatcctggcagagccacctacagactagcttattaatatcccaatgttgttttcaggagagactaaaattctaaaacgctttttaaatgatgtgttttgtatacacaagttttcacccacaccacttacagttttccaataaaagcctatctaaataaacagtttaaattcgttacacggggctgcatatttcagttctgaaatcaaagatgggaatcaatttgaattatgactgaaatcaccatttagaaaatggtctcatactggatgggttgttctcaga

SEQ ID NO: 52 - DNA, 696 bp, Mus musculus
tgatgtccaggttggctgttcaccaaggacacccagagagactgtatggtcttagagagtctgagtggtagcacatatttgaaatagaaagcaccaccctacccccagatggtctctgcctcctccacctactgaggtgacatctgcacttcctatgtaatctttactcacacctcttacaaagacagcatgtcgtcatcctgaccccagcagccagccgagctgggctttccagaagtcttcaagctggaggagtgggaggcaccatggcctgctcaaggggaacagagtgaattgatgccagacggtggcaggtggctattttcacttagcagttagtggctaatgaaagaggtgtgtgggtatctttctccccagaggccattcatttctctccagctgaaagagcaatggactgatctgaggcaggaccaaaagttcaaaggaattatgacagattggcccagcacccctgagtttatctgaataatgacgcataagcaccatccagtgttgacaaagctctgcaggctgtgaaggcttaaggggagccttggagctaagcacagagggcaatgatgttgcttaaatgtgcacaggtctggattaaagtggagtagttcttaggctgctcatgtcttcttaatcttcagctaggattgagactagcctccatgacacaggggatccatgaca

SEQ ID NO: 53 - DNA, 684 bp, Mus musculus
  misc_feature (8)..(8): n is a, c, g, or t
tcctgcgnccgcacgcgtgctcagttgccagcacacaaatgatgtcacatgagcatctataactccagttctaggagatctgacaccctcttctgaccttcattggcaccaggctctcagtgtttacacatacatgcaggcaaaaaacactcatacacacaaaataataaagaaatttaaaattaagaaagagaaaatcatagttttagtagcttttctcttccatttgtttgatttcttgccttatgacattgtttattgtgttcattggtgactgtctttacactgaggattctatgaatgggcagtttggaacacttctaacgctttgtactctgtcactggcatgctcacagtgtataccacctaccacagaggaacatccattgtttaaaaagcagttcaggttattttgaaacactaatacaaaataattttcagctgtaacgatgggaatataacaatttccttgactacaaagagaatataacaagtgcctatgattcaaatgcaaaagacaccgaatatccatcagtgcttttgaaaatggcaggacttactgtattgacacttaacaagcttttttttttttttgtctgaagcttgtttttaaatgttggaattttagttgaaaaatctatgtcactcttttactgcagttttctgctttcataacattttcaa

SEQ ID NO: 54 - DNA, 533 bp, Mus musculus
gcttatatgtgtgggcaatatgtatatattgttactttatatgtatgtgtataaagagagactgttaacatgttttcagaaactcttaaatacatataaaggcatacacaaatatctgcagaggtaattatattaaagtgcagagtttagctactatgttttgcatttgtaaaatgccatagaaacatcagctcattaattttgttcacctcgtaggactattattcagaatctttattcagccagggagggcattttgtatttagctattacttggctctctcccttttgtcataccatggcaggtagtctatacttaaacctgaaatttttagtgtcaaacttgtggcaaacaaagtccccttgttccttagctaatgagcacaaggttggggtttacatactatcactaggctttaaggtgaggaccgttgtccagcagaggggcctgtcatttttgagaggtatttgtgtattttataaaccagccaactgtctttcttttcttttccaccatcactcatttccttcca

SEQ ID NO: 55 - DNA, 506 bp, Mus musculus
tctcatcctatgcctttcttctaggtacttcagaaagaatctttcaaatcaataactgtgttctatccacagaggtttaatggaggagcaggaggctggaacgagatgaaggaaatgaattgttccagagatgaaaaagctcccaggcatatgtcataagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaatgagtttctcatttttttaatttaaattacttttgagctacttctagactgcagcagagttgataaaccacccagttcatatactcaaacctcccaactcctccacagctcagccaaaatttcccaaggacttgttttacccaaatcccagctccg

SEQ ID NO: 56 - DNA, 534 bp, Mus musculus
tgtttcacaaactacaggttcactggagcacagccatgccaacatggcatctaaggctggtatgacctcgagacccctccagcactccttcgttcagccatagaacaaggagacacagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattactggttaggatttctctgtttgctgagtgcattaacttttagcgcttcccatcactcattcttcttcacaccctcttgtcagttacaaatgtgcaggtggagagccacgaaaaaaccctgcagcaacggcaacagaaatcacagcaacaacaacaaatgaacagaaaggatagaaagcagatactcaaagatggagaaaaagatactcccgagaggct

SEQ ID NO: 57 - DNA, 621 bp, Mus musculus
aacctcagtggtttcagccaccatggaagagagagagacaggagtcagaatgtaaaggcatggggtttggtggaccagagagagagattctcagtgcagtgggaattgggctgctgggcagaatatacatcccatagactactcccccaccccctacggtgtaagaaaaaagacatctctatgacttggcttgcacaaactttcttgtgtgctacctttgcaacagcagatctcaggcaggtgggtggatgggtgtggaggaggagaaagcagaaagacaaaaggaagtgagtggggggaactcttgggaacctgttggcagagcgctctgagactctgaagctaagggaccatgttatgttaatctattctctaattgctcagcaacaaacacctcccacccgctgcaaggaagggcaggacaagactattccaagatctgcttagaaacaatgtcagtcacagcagagggcgcaaaagaaatggacctctttttagcataattgaaagacactacttacaactgaggggtctgagttgctgacagcaaccgccttagctaggaggaaaagctgaggggtaggctgtgctcaatgggagggtttgcaggctgactgttca

SEQ ID NO: 58 - DNA, 609 bp, Mus musculus
agcctgaattccacactacagctcttatattagaaaagaaactggatgaaaaaagatgggtctcagagaagatacttggagcaatagccacacaagagttgctcaagtatttccatcagcctaaatgactacacacctatttgccaccagctggcctcagctctgctctgaatgcccaccatcaactctctgagaaagtgcccctgactacactcagtgaagctgtgagttggtggctgccttttccccctgcaagataacaaggtaggccttacctcagacttaactcaaggtaaagtttaattgctctaacctgctctttgatgtgtgctgaggaggtgggcgccgtgtgggtacaagttgctatttatacagacctgacaagcgcatcccatcagcacccgctcctttgttctcttccaggcttttctgattctccaaacttaacatcctgcactcccccaagttgtttaatcgttccaaagtttgtaagacaagcaagggaatagtggccttcacatctgcacttgcagcccaaatatggtgcactgtgagagatccttaatttggagaggaaagcctgctgctgaccacctgcctcttagaact

SEQ ID NO: 59 - DNA, 679 bp, Mus musculus
ttcctggacactgaattctgagagacagaggtaggagaggggaaccatgggatgtgacgatttcaaagatttaccctgggcactgtataagaaaacgttttccaaagtgtctgacactgggtctgactgacacaaggcaccagcaaatgtcacctctcttccctgtgtgtcacacaacttcatttctcttgaaactaaaacttctcttctagttgcccttgacttccaatgtcaccgttctctagcccataccccattactaatcctccccgcccggttccgcctacgtcccttttgttcccactaaagatggctttgctttttcacctgtttaagtcttcctgcctcccacactgatgaattcagttagactgaaaaaaaaaaaaagaggcctcttcctccataaaaccccaattaatcaatcattcacgactgagcaatttggggtgatggtatttagtgggaagaactgctccagcactgtcaggcctgggatgtgacactaacagcagtgagacctaagaggagtaatttggcttttttcatatctcagtttgctcactcacagtaccttaactcttaagaccggtaagagtgaaaattacccccagaggaatcctagggcaaagacagccaggtgatcatcagtataaactaaggatgcctgttcataccagca

SEQ ID NO: 60 - DNA, 490 bp, Mus musculus
aggaactggacacacaaagcctttctcgttttttcttcctccctgtgtttcggtccaggggtcctaagtgtgtagctcccggggagatgtgggactgttctcacccagaaagaggaactagaaccacaagagcctaaccccccacggtgggaagacactctgtggcatcaccgggaggggaggccaggtgtgggcaactgcagctatttctaatccaccagaggaaagagagctttgactgcttgaaaagcaggctgattgtcaggggcggaacccagctcagagctgtcagggaagctcctggcagggggtggagacagggggcttggactgacaggcctgtgattttgaaagaaatgtctttaagagagtgacttaaagtaggagagaaatattattattaggtaatgcctgtcaggttatatttttttttgatgtgttcttggtatttcataatcattttgcctttagatggtaagcccttcctggt

SEQ ID NO: 61 - DNA, 625 bp, Mus musculus
taaaccttggactgagcccacaggatgctctggaaggaccttgggaaaccgacacacaaagcctgtctcggttttgttttgttttgttttgtcctccctgtgtcgcatccacaagtcccaagcgtgtagctcctgggaagctatgtggctgttttcaccaaggatgacttctcagagactgacacccataagagaggcccctccacccgagggtgggaggactctctgtggcatcaccgggaggggaggccaggtgtgggcactgcagctatttctaatccaccagaggaaagagagctttgactgcttgaaaagcaggctgattgtcaggggcggaacccagctcagagctgtcagggaagctcctggcagggggtggagacagggggcttggactgacaggcctgtgattttgaaagaaatgtctttaagagagtgacttaaagtaggagagaaatattattattaggtaatgcctgtcaggttatatatttttttttgatgtgttcttggtatttcataatcattttgcctttagatggtaagcccttcctggtcctgggccatgaactatgcccctcgggtctcaccccagccttggacatgcaatgggcaccaggaggta

SEQ ID NO: 62 - DNA, 2005 bp, Artificial Sequence
  CN2438
gcggccgcacgcgtttgaacctccgactgcaacacagattaggatacagcagtggctacaattacacacttgcttgggaagttgggttgggagggggggtgggtgctgcatgtgacaggccatgaaaggtccctgcctataaaacagactgaaaaacaaacccttaatttaaatgttaatgaaagtgctgtggagacatcagaggcttcccggctgctcagagaacagctacaccagccacagaatttatggctccccttcaccctcccgcctccctctctgcctcatcacccactgaaatttaaagacagaaaaaaaagaaaactaattttaacattaactccatgcttccactttgggatgtgattgctaaattatctgttggcagaacggtgatcaaaggtggaaaaacacaggcacgttgcacacacaacccagaaaagtcagcagcagctaaagcaggaaaattcccatttcccttcctgatgagcagagtggaagaaagagtggtgcaggtagggactggagccaggaggtgtaggcagtgggaggaagagaaagatgactgatccgtccagcgtgctcccaaggccctgtctcaacctcatctgagaacttaccacactctactacctattttacttgtcggtctctcccatcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 63 - DNA, 1719 bp, Artificial Sequence
  CN2439
gcggccgcacgcgtcttatcacttcctaattattttctaccttttactaacctatgctctggagatttataactattatatctgtgtggtgcaaacatcatataatggtgtgctactgcacattgcttcccaactctgcatgactctgatgtcacattgatagcttgaaattggccatggtgggtgtagctctacaatgaaaatcagcaaatgctacgaatcaagactttttttccctctaatgttgaacatttaagaacacattgtataatatcctaagaagatagtcaaaatgcacaatggcctccttctatcccaaataaccagccagataagcaagcatctgtaatgtccctcagaaaccaagtgcagccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 64 - DNA, 1764 bp, Artificial Sequence
  CN2451
gcggccgcacgcgttgctgaaccaattaaggccagtggtaccacctgagattttgaaaataaatcagtgggtttttatttcagctgtttggaaatgactaatagagtaatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaaatgaatacttatactcctttaaatactaattaagaggccctggtaaccatttatcagtaatattaatggcatgaaaattgaaaaagtatgactcaaaggtgaattcagggcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 65 - DNA, 1965 bp, Artificial Sequence
  CN2463
gcggccgcacgcgtttcttccactcctttccctgctgcaggaatcagtcccctggactctgatgtgtggctgggttggctgggttgccctgggcagacttattagcttggactctttcgcatgcaaaagatagcacctgtgtgactgtacatattgaaaatgcaattgcaggtgtatttgcaaattggattttgagggggaaatttaagcaaatccttttgaaagcaaatccctccaaggtgtgagtaataaagaaacaagaagaaaggtgtcaatcaaaaacctattaaaatctagtttccagagaagaagctgactgtctaatgacatttatgctttcatagctaggcatttaacagtcattttgtgtcagttgcttcctttatacatgtcacgtggcacatttttcagttttctgtatttctgagaccttaatcccaatcactcttaaatgcttgaaatgagcccatgttcctaaaaggagggggtttttgtccacctgaatttctctgcaggaatctacaatgatgcctcaagcatcagtatatgaagtcaaccactgtcattcatccaattcattcattccattcattcttttagttagccctaaaactccatggtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 66 - DNA, 1980 bp, Artificial Sequence
  CN2464
gcggccgcacgcgtcaactcgcttagagtcacctgctagaggtttctggttatttggagatttgctggctctgccatggacatgatggcatattcgtggatgatatggagagtgacaaaatggctgagtgtctaattattaatcaaatcacagattcatatctctctccccctcctccactagtttccttggagacacagttttttgtcaattttttgaattgcattaatattaactatgactttgaatcaggaagaatgcaaaatatctgaagagtagtatttatatcttccacgtgacatttgcatcacatggataaactttaaaaatgttcttgagaacacgggaaaataaggaaatagagttcaaaaataaagtatagaacttgttagactagtttctgtttaaaactcggtggattatacctctttgttttaaaccattagaacagcaatgattaaatatcagattccttaaagagtcaccagttccttttggaaagttttgattaaagcattgtatttattaaactgttctacccacacatctttccttcaagatgttgtttttcccacgtctactgttttattacatgacagttattgaaaggttagatgtggtttaatttctcaaggtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 67 - DNA, 1750 bp, Artificial Sequence
  CN2465
gcggccgcacgcgttggagaatacattcttgactcaatgaatactatcacttttttggttaaacagtccaaatttgacatccagaacatttctttaaagtcaccattttcctggaaacactctaattttgagatgtctagggctatatttagcttttgaacagtagtctttgaaaaaatatggttatgaaacagattgatttagaggagcattataattagctgccgaatacgttagaaggatagattattctacatgaatcacagagctcactttaagtgtcattccccattctaaactgccagcttaatttcttctcatttttcaccacgagagccttttttctattatgccaataataaagggttagttgtcatgaaataggcatacctttgtgaaataatgagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 68 - DNA, 1992 bp, Artificial Sequence
  CN2466
gcggccgcacgcgtactaaggaatagacaatgggttctttatgaatatgtgagcatgcctaaaagcaacaaaatcacacttttgttaaagcatttgtgtgtattttgcacaatttttaaaagtttaacctctgtagatcaatgtgcttccttaaaagactttaataaattaaagtgcagagagctttttttcaccaggcagaaaaagctgatgagacaggtttagtttaatctcaggcaatagaagtcattaaccattctctcagggattaaaatatagcttgcccttttcttctctcacttatcattagcatcactttaatctcctagcgtttcacactgctattttaatcattacttactgccttttgacaagtgcagaacatgctttttctcattgaggcaatatcctggttttagctgacaaatgtaggacataatgtaaaaatatcaaagtgacatctcttaaacacaccttcatcataaaaactctaaagttacaatattcctttgtctagtatttccatgaaaattatcatttgaaaggtttatatcttaaataaagaaaaaagtttcttccaaatacaacaaaaactagtttagagaaatagtatgctgaataaatgagatctaaaaatatgcacatacagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 69 - DNA, 2015 bp, Artificial Sequence
  CN2013
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgtagactgaggagacgcctcagaggaatttgtacggggaaaggtctgaggaaaaaggatcggatcccaatgggccgaactccttcaaaatgttcccgaggtccacgtgacgttccctcctcccacccagcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagagaaactcgggtttctaacttttaaagtgggattatatgtcttctccaatatatgggctccctgcttttataacatgctgctgttacatctaataaaaaccattttgaatgaaggcagcctttcatcgagctcgattcagccgggagcttagggaggggaggtcacttcataagggcttggggggggagttggagccacgagtcgtccagccggagccccgtgtggctgtgctccggcctcagaagcatccccggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 70 - DNA, 1993 bp, Artificial Sequence
  CN2025
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaagagctcgattcagccgggagcttagggaggggaggtcacttcataagggcttggggggggagttggagccacgagtcgtccagccggagccccgtgtggctgtgctccggcctcagaagcatccccggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 71 - DNA, 1786 bp, Artificial Sequence
  CN2229
gcggccgcacgcgtgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccacgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 72 - DNA, 1637 bp, Artificial Sequence
  CN2442
gcggccgcacgcgtttcccagccttaccttgttgcaggcctccagggtctgcagcggggtgaggcagggtctgggagatgcagtgaatgcaggaaggggtgtctgcctgacagccccatccctgcagattttgatggctccgtaaaaatgtctcctcataaactgtcaaagtgtcacaaattaattaactgttagctagagtgactcagtgtgggggggggtgggggagaatggctcagtgatgctgagcggggtggtggcggtgctgttctctgcgtgtgctgccttttgatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 73 - DNA, 1926 bp, Artificial Sequence
  CN2443
gcggccgcacgcgtacttggaggaaaggcagaggaaaatggtcactccagcagtgctgccaggacaccaggaccctgctccatacacctaaacctcagcttgcccttcccgtggaaataaggagatgaagcactcagaaaacacaatgcagtttcattttcagggtgggattttttccttctatgtatctcatgagtcactagattagcacctttccaattactgggaggcaaaagccctattatcatagaaaccctcctgcagagtgacggctctattctttctgacggcagcagccagtcccacagagagctgcagagctcagcaagctgggagcctgaagttccaggtaacttcatgtcctcgtttcaaaagtgaagtttaaagaactcacatcacagccttagcagacttctcacactccacagaacacgccaggacagcccaggaaggaaggctgaggaaggttcaggagcacatcccagaggctgcacacagggcccggggctgcctcgaacacactctgcaagggtctgcagcactgaacgttctgtctgagctgagggagaactgcatgacacagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 74 - DNA, 1979 bp, Artificial Sequence
  CN2444
gcggccgcacgcgttgccacttgaacaatgactgtcattcattataacttttgcaaacccaccaaaagctcctgatggcatgcatacccatgcatcacacgtgcatacgtgcacacatgccttttaaagaccccacatgggtgattgcaagggctgtgtttgacagttgtcatctcttaaaatgatgtaggttcctgaagttcctcatgcaggactgtcagcctgacagccttagcaagtcagtcctcgttctcaaatgggaaaatgacatgggagggccttttcctccctttttggtagatctgccattcatcagcatcattctcttctcagccacttgctcataaagccttccggaggcaattactcaggtgtacatgacaggcctacattcctgtcaggggtgtgggctgggttagagaggatgcgggtgacactctgttccagcagggaccatgcctgcccaccaggaagcacatgccgttctcagtagaattctcttttcagagtttaggaagccactagcgctataataactgcttgctgtggtttgtggtcaagagcaatcatagcactgattttatttttctaatttcattctgaaatctagacaggaggtgagactgcatcacgttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 75 - DNA, 2095 bp, Artificial Sequence
  CN2447
gcggccgcacgcgtaaacaagaacccgggggaatccaactgctcagcatgtgggaagggctctgcgcagcccgcccctgctcctggggccgcagggaaaagtgggtgaggccagcactctgtgcctgggatgccactgctgtcctgcgatgttgtgctgctcccccgcatggctgctgctacgggctcttgccacagctggacaccctcccctgagctccagtgccggacaactgttttctgagcatgctaagcaatgaccctctgctgacgggagaagttactttagaggggacagaacttaggaagacccaggtgtgctgtgaggcaagtggctccctccctgacccttactgctcagtgtgcaaggttaccaccttctcctcctccccacacccatcccccctctctcccaaggtgcccctcattgctgctgtcactcatcattagcagtcactgtcattgttaggacagcccctgtcaggctgcagagccaagggagggggcaggagaatgtcagatgactcacacaacctcaaaagagggggatggacacacaacacagaacctagctacaagaagcacctcgtgagactctggttcaaccctttctttttccagagaagaaagagcagcttagcagagtttcctgacaagtccctctgcttcccactagtcaggcttggagggaggctggcctaggagaaaggccaggccaggaagtcagcatgaggctgggcaggtgacaggtggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 76 - DNA, 2031 bp, Artificial Sequence
  CN2448
gcggccgcacgcgtaggttgagtggattgaggagaaaatgggaattttcatgtttttcaataaatttagctttaaatggaaggagagagatagtatctagatggaaatacaaaatcacaagataatctttctcacttttagacagaggagaaatcaccacataactggaaacactgaaaataggagaggggaaagatggctgacagagtcaggcttgacaggaggaggatggggtccaggacacagtgtcagaggaaagaggagtatagacaggaggaggtggggacacagttcccctactctgacaggaggcaaggtgggaataattggtactgatagatctaagtttatgggaggtgggaggaagttcatagagttcctacctgacaacctagattcatctgaaggaggagtgccagttaagagactgttacaagaagtatcgagaacctgagttgatgacattgggaacaaagctgcatacttgagacactgtaatcatttaacaaaaggtgaagttcacaccatacaagcactgcaagaatgtagagcaattagaattctcatagactgatggtggaaaagtaaaatggtacaaacactttggaaaatctttaggtggtttctcaaaaagttaaacaaagtcctaacatgtgactcagccacccactagtaagtatttacccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 77 - DNA, 2483 bp, Artificial Sequence
  CN2449
gcggccgcacgcgtattgatggagttggggctctgtgagtgtggggccctgtgattccttgcactaattccaggagatgggtctcctaacagcatgatcatgtggatgtcctgtgagccctggcggggctgtacagagagaacaaactgccgagcacagtgcagctgccttatcagatggggcacacaggcttgcacagtgagcatctcggcagcagccttggtagactgcagaccagagactcgtcccccactttccagagttgctaaattccaatgttttgagaaacacaagagaaagggttggtgggggtcctggaaaaatgatttagtgagtaggggttaagccgtatgttgtttcagttagaaaaatgaaaagttaaaggcgaaagatttgcacgatctgaagagaaaccagaatgacgagttaatggcacatgtttacatacgtaacaaacctgcatgctgtgcacatgtaccctaaaacttaaagtataataataataataagaaaaatgaaaagttacatttcttatatctgagcattgtctcattaatatgaagtagtcataagcaccttgtgttttggggatttaccttacaaaacactcacaatatctttcctcattatctggtcaccaaattaatttattcatcattcatatatccattcatccgatattggagaatctgctacagtcatagtgtagaggtgtacaatggtgactaagatgtagttccacgttcttacaattcttactagtgacgtggataggagataaggaaccgcagtgaaaaggatattgactgggcacaaaaagtttggggttttaatcctatttttgtccttcaactgctgggcaactttgggccaactacacaacctttcttggcttccctttcttcatttgtaaaattggactaaattatctttaatttcttactaacttctaaaatactttactgaagaaaagagacatacacatctgatgtaaatgcaagtatgtgtgtgtgtgtgtgtgtgtgtgtgtttaatgggggcatagctggctcctttattaggctttggtttttcttcatgtatctgatttccagaaatgagagtgaaaatacattttattttgctcccacgtgtttactgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 78 - DNA, 2100 bp, Artificial Sequence
  CN2450
gcggccgcacgcgtactggggtcacaaagaagaaccagggaacgctgttggggaacccatgcaatgaaatgtgaagggaaagagggcgggcagacgtccccaaacccgctcagcaaagacaagcttcgggcaaattcactcacgttgacgctgttcggatggagcaggccaattggaaaagccgggttcagacacgactctagagggaagagaagaaggcaagtgtgaggagggcgggcgtcggggagaagcgggcccgggtgtgctggcggacgtagggcctcaccgtcgcggtcctcagcgtctctgcggggtcacgggccaggctgcggggctctgagcgcctgcctcgccgtcccggggcttaacggctgctggagccactttataattagccccaaaccgaaggaggcgcgcgcgccccaatcgccggcgggctgcagctgacgcaggccctacgccagccccgcgccgacgccttcgcgggaggggtcggtcccccagcacccgcgcgccgctccccccgccgagctcgggtgggggccacagggggcgctcggtggctccccacacggccaggtgacaaacccaaggcagttaggaagtttggggagagtgagataggatcaagtttctccgcataaagtcactgaaattgcaccgtgggatgcgggctccagggagggaagcgaggtgggggcgtttggaaggatagatggactggcttagacttctggaatgtgctggagacccagcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 79 - DNA, 1760 bp, Artificial Sequence
  CN2467
gcggccgcacgcgtcccactctcacagcctagggagaagaggaggtgatggaggaggaggtgacaggccccctccacccccaccccacgcccacccccacgtggccccacagctgtctgtctgtgtcaaatgttctaagcagaggttgggggcttcactccccacacaaagagagttcagagctgtcagcacctttcccacggtacactaggtgctgaggaacaggtgataattacagccctgtcaccctcatcacctctgagctggatgagtttttttctaaaaacaaatgttgtgctgcctgaagatcaaagttggagcttaggttggggaaatacacacatgcaggctgctgctcttttccatgaagcacagggaattggcttggaaaacaccttggagatggctgtcttggcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 80 - DNA, 1962 bp, Artificial Sequence
  CN2421
gcggccgcacgcgtgactctacctgctgtaaaggcaaatgaatgatcctttgattaaaaggcactctacagagcggcacaggcacagacagaaacctagggaaaggcaaagaggagtgaaaactggaggcttcctttccatcctgtcgtcaagagcaagagaccatcgtcaggacctctctggagagagtcagagcctgggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaatgctcttctcactctgctctgggtctcccttccgtccctccctctctccctccctcccttccttcttctttcctctgcttctctttgtcatctcttccaatcctcacatctgataagcctgtataccctcataagattcctaattgtgggatatgcttttaagcgtccatccaggccaagatctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 81 - DNA, 2074 bp, Artificial Sequence
  CN2231
gcggccgcacgcgtgtggaggttgggtgtttccatcacctggctaacagagatcacaatctccttttgttcatcttagggataaggtttgtccatagctcgacttgtctggatttctctgtccatcacatgcctatgtacgctccccacacctctctcctagtggctccttggtttaaaatgccccagatctgcagctcacagaagaacatttggctactcccgtttttaatctactacttctgtaatggggctcgggagctgggctgggtcccccgaacctgtgggcagctggcagcctgaccctgggctgagtaccaggcatggggaggccagaaggaaggggcagattgagccagagaggtaagactgcagagagccaaataggaggggaggaggccagagcctgagggtccagatggggctggctgggcacaggagggcatctgatgcaatccgccaggagagatgccagccggccgggtgctgcagaggctctgttcttgaagagtctagtgcctgctctattaattctggctctgtgggaattggggaagatttccctatgaggtctcctgagccccagcaacctccccaaaggccttagctcataaaagcacctgtgtctgaacaagcagggggtggggacatagaacacctgggttggtggctgcctggggaagggcaagctgaagtatcttgtcagtataaacacagtacctcagccctgcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 82 - DNA, 2182 bp, Artificial Sequence
  CN2236
gcggccgcacgcgtgggaagaatagtgagttctggggtggagggttttgattttttaaagtggaaagtcaagaaatgcaccacggagaaggtggcattgagtgaaaacctcaaagtggtttgagagaaagtggtgcagatatgcggacagcgcttcaggcagagaaaataacctgggatgtctgagaaaagcaaagaagctggggctactgcagtaagagcaagagagttaagaataggtgtagccagagagttatcagaatataagattgcatagggcatttttaggccaatataaggactggcatttttaggtcatgatgagaactggcatttactctgcctaaaataggaaaccactggagcattttgagcagaggagttgaactgtctgatttaaaattttaaaaggttctggctaccttgtgagaagagactgaagtaagatatgcatggaagtaggtcatcagttattaggttattgcaataatctaggcaagagagatggatcagggtgataacagtaaacttcatgagagcatttcagttgcaaatatattccagatttgctgaccagatttgataaggaatatagaaaaagaggaaagaacaagagataacagagtgtaggcagaaaagagaaaaggttcaaagactgagccctgcagtatttcaacattaagagcttgaattgcagaataggaaccatctcaaggctttaaatacaagtgctgaacactctcaaatttatatttgtatcccagacttctctaaaattcaagttctaaaatccagttggttactttatgtctccacttggaaacaagtcttgaagcagtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 83 - DNA, 2278 bp, Artificial Sequence
  CN2237
gcggccgcacgcgttgaggctctcccagattctgaagctgaccggctcacagacggcagtagttctttgtcgaatggggagcacagagctggatatgcagcagtaaatcatgacacaattattgaagcccagccagtgccccctggcacatcagcacaaaaggctaaaatcattgatcttactcaagcattaatgttgggacaagggaaaaaacttaacatctatacagattctaaatatgcattccttgtggttcatgctcataccgccatcaggaaagaaaggggattacttactagcaagcactcccctatagagcatggacctgaaattctgcagctattgaaggcaatacacctgccaaaggccatagctatcattcattgtagggggcatcaaagggacttaacccctatagcacaagggaatggaaaggctgacggaagccaaagcaaaagccctcagcgtgcaatcccaacagatcctagcactgcttcctttctacaattccccagtagaacccaaatacacaccacaggaagaacagctaataaaaaaagcaagggagacaaaaacaaggatcctggtggcatatggagccaaaaatatatctccctcaggcagcccagtggagagttataaaagccctgcatgactctttccatgtggggagggatgcggctctggccatggtaaacaagttctttactgggtttaactcagcttcaatggctaagcaggtctgtcaagcctgctcactgtgtgcatttaacaatccaggcatcaaaatgccccctctaataaaaccagtccaaaggaggggaacttatccaggggaagactggcagttagacgtcatccatatgccagcttgcagaggatacaagcttttgttaatactaatagatacttttaggttgggtcaaaggttaccgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 84 - DNA, 1926 bp, Artificial Sequence
  CN2440
gcggccgcacgcgtcatcccgctggccatctgcatcagcccgacccccacaggatagaagactggcacatccaagttgctccttccacaccctctccccactgaggcctcgtctcctccgccctgtggccaattgaaggtgaactcctaacgtgcattctgcctgggatcccagaggttgtggcagccagatgagtcacctcacaaatacagcacgtgttcctcgggggcggcggtgggaggaaacacgtgaagagctctctctgtgataatagaccatttagagcttcagatggcgaaaacaagctgatcaggtgcaggccctccaggaggctggcaggtgactcccagccagcacgccctccatcgcacgttggggaagctggaaagagcctggtggctgccgggcctccctctggcatcctctctccctccaaggctgtgtgctccccaccttgtctctccttttttcattccattgctctgttctgtttctgtctacccattcgtcttttcttcctcttctctttctgttgccattctctttctcttctcctctcaacatgtccttctctctacccgtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 85 - DNA, 1805 bp, Artificial Sequence
  CN2441
gcggccgcacgcgttgactgtgaagtgtctgtctgaactcataatcagcttttagctggtcccaaacagtgagtgctaccagctccattaatcccactgacagggatttatctttctcagctgaatgaatgatgctcccagccttgccccagaaatgtgggctgggctattcaacacttgtcaccagcaagctcttttcctgaggctgcatactccagccacccaggtgactgagtactttgtcatcttcttctcagagcagtggcagaaccacaaagcaaactcccacccatgacccacccacccaccttctgacctagtttggatgctccaagccataatgataaagtaatgttgtgaagagggaaagaggtttgcagtcagacagcctgaacccaggttctgccatgtacaggctgaagcttggcaagcctctgcacctctctgaggtccatttgcacgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 86 - DNA, 2012 bp, Artificial Sequence
  CN2445
gcggccgcacgcgttcccacatctgtttctctggctctgcctcttctgcctccttccatctcttatagggagccttgtgatgacattgggccaaccaaatattccaggatcatgcctgcatctctcaagatccttaacttaatcatatcagcaaagtccctgcaccatgtgaggtaacatattcacagtagtaggtgctcaataaatagatactaaagatagcagcattttttattagaccttctcacagcttctggggattagaatgagacatctttggcagccattgttctccccatcatgggtaacatgcagtgggcgcttccagtgccaggatttcggtgggtttaactggagtgtgcagataagtagttggcgtcaagaactcctgctgcttcccttgctggaaagatggggtggctttgaatgacatcccagaatctgattcttccgtctgcggaatgtgcataggatgtctgaatcacactattgtcgtgagaaatgaggtagaatattctctgatgtggcaagcttaggccagcatctagcccaattccttgtatataatggaaattcttaagatgttagggttcccctctccctctctctcctttttcctcgcctacaaaggaagacatcttttgtgaggcctccacagcattcttgatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 87 - DNA, 1776 bp, Artificial Sequence
  CN2446
gcggccgcacgcgttccattgcagcttctctctctctctctctctctctgagtcatctttacaagcccctgaaacaatggtatttaaggagggagctttctcaacacctgaaatccctacagaaaggttgagcttccttggtaaagcatttacctgctgggggagggtgcatggagtgacatggttttcacatttgagaaaggtaagtgggagggaatatcagacaactgcagagtgcagattatcatttaaataaaatccccatggggctcgctggcagcattaaaattgctcctgcctctgtggtgctgtcacagaacagcctctctcctttattctgctttaccatagaaagaggcagtgtggtgcagtggaaataccccagggctgaatcctaggattcaagtctgatccatcatctctgggctgtgtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 88 - DNA, 1726 bp, Artificial Sequence
  CN2457
gcggccgcacgcgttgggactcagcaacttgactaacatcacagacacagttctcagatttatatatcctaattccaaacccaatattcttactactagaatatgctggaccgttttactcctaacttggttaagaagccaaatataggaggaaattaacaaatcaaggtagttttagttactcattggtctgtgggttcacagcattttaatggagccaactttgctcttaagatgagtaagcagttggtatgactcaggaatctctaagaatgtttctaccttaaaaggcaggtttttgtttcttaatcctttaacaattaatattttaattaagggaacattggatagacattgtaaggtgcaaaatacctctagaagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 89 - DNA, 1641 bp, Artificial Sequence
  CN2458
gcggccgcacgcgtagttcttgctttctccaatttgtttatttttatgtgacctagctttatacattgcaatttaaaggaaatgaaaaacagttttgttattttcttgtgttatctgggagattatcttattcttttagggttcaagtgcatagagttggtgcaatcagccacagtttatgacacagtataatttatgcttcttgcattctgttttaaatttattttcaccttttattttccttccttttatccctttgtattatgtctaatatcacctcatcttcaccccaatccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 90 - DNA, 1648 bp, Artificial Sequence
  CN2459
gcggccgcacgcgttgctcatgcacaatcactacttctatctcttcatcaatggtaacacgattctgacttccaacagaatggacactttgcaagttttgaaatgtatgaaaaggaaaaatatgtctttttctgcttatggcttttttagtcaatgttatattatgcactttatttatgtaattttgtcttgctatagtttgtttgcctttattgacgaataataaatatactagtatgtgaacacaccactctttaattatttagtctacttttatttccaggtgttggatctttttcatcgagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 91 - DNA, 1729 bp, Artificial Sequence
  CN2232
gcggccgcacgcgtggatgcccaagccaacatggagggaagggggggggggggggtgcggactgaatcccaaaggccccaagacgggtggttactcactgccttctgtttgggactggctaaggccgccaagttcactcagcccactcagtagaagcaagatgagccacatggcctggcctcctggaccccgccccggggaatggcagttggccctctacagggcagttgggggcgcgaggtgggggctgtgggcagcgcagctgtgtgccgggaagcgtttgtgctgctcggtgaaagctttgaaactgtgaaaggtttgaaggacttgctggggtttgaaatctgagaccaaagaagggaaaagggaggacaggcagttgagggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 92 - DNA, 1866 bp, Artificial Sequence
  CN2233
gcggccgcacgcgttgtctcatcagtggtccttctgtatcattacgcctgggaacagccaaagtgacatctctgcagacccttccagttttaattctatttaagttttacacggcttgggaagtcccctctatgactgtggagaagccaggatagattgggccacgctgctgctgaagacttcaagggagcagttaaatctggggctgtcacactgaattggtggcttctgtttgagccatctgttcctgccacggagcatcccatcccatcctgcctgcctggcagggtcccactcccatcattatccttcaaagagctttggatgagaattctgaacatcctcaattgatgcacagggggggagggtattggtgagtgaatggttccagatgacaagtttcacccctggcatggttaataagtgagggagggcacatgaaatataatgacatgatctttcagagtttttcccagttgagctctgtgggcagttattcctctgtcacagagcgatctctatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 93 - DNA, 1765 bp, Artificial Sequence
  CN2452
gcggccgcacgcgtacctcaggtcccacatatactgtggtctatcacctggttactctaaaatgccccaactcaccttcttccttaaagcagctcccactcctaacatttgtatctcaggctgattattcaggtttagaaatttcaaggtcatctatgactcactgttttgatcatgtcagtcacaaagttctaaagcttctgctttggaatgtttcttgtactgaatgcttccttccaattctcattgccagcaccagcctccagatctttatcaagtcatagctgggttgttgtaacagcttcccagcagagatggcatgaaggaaggcaactaaacttttctgagcacctcatatgtatgtaatgttttcaatgcactatcatatttaactctcataacaataccacgaagcaggtatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 94 - DNA, 1682 bp, Artificial Sequence
  CN2453
gcggccgcacgcgtggggttacctttgggaaaataaactcttattaaagtgtcacatcttggtggaaaggcttttaatttaatgattgctatctacagggaaaaatcacttataaaaatcttactatttctcccaaaagacaccaggttgagaacattaggacatcattctttcatcaatatttcctttgtatttcttagtattttacggttttccttaatacttcttattccttcaaaagctatacctccatactatatatttgaactgaaatgtctgattttagaacaagtcacatgtcatttttattttgtctttaaaaacttctgacactgagagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 95 - DNA, 1707 bp, Artificial Sequence
  CN2454
gcggccgcacgcgtctcagtatcttatttcttagtcacactttaaaactgaagagattagtaattttttctatgtcaatatgtgaaatatgcaatacaaaattgtacaaacccatttaattccattctctgtcttttgtcaatcaaacatcctccctgataagagctcaattatttgattaattataaatcgctattttttgatatgatattcacagtagaataatttataatagacaaaattacaaacaataagatcttggtgttcagtgggatttatggtgttttcatgcaatggaataccgaactgacacaaaagtcatgttagagaagaatatttatagatgtggggaaaagttcatgagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 96 - DNA, 2002 bp, Artificial Sequence
  CN2455
gcggccgcacgcgtgtggtgatgctggacaaggcaaggagtgggcagaggcgaggactgtggtccagctgaattttcagaggccctctcatgtgcgcccactctttggaatcattctcatagataccctatgggaagcccaccccccagcctggccccgcgtcacactgagtcatatctaatcataggctcccagggtgatttccttacctgcttttttctgtgtttttaagctcttcattacttcctaattgtcctgattttagttctttctcgttcttgtcaaacacccatttagaaaatgagtcatcattaactgcacgccactcttcacttagaattagacccatttcaccctttctctctctctttttttcctcttcaaaaaaactgggggattaaaaaaattacacttttctgacatcctttttttaatctcttttgctagcgcaaatgctattggctgctcaactgccttaagggtttcttctgcagcccaaatgaacagctttgtgcttccagcctttgatagctcctgggggcgttgatcactgagtcaaacctccaggtctcagcctcccaagtggcctgctcaggcatgagtcaaatcaagactagaccatcaggggacaagaagctactgctcaccttgcaagtgagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 97 - DNA, 1662 bp, Artificial Sequence
  CN2460
gcggccgcacgcgtgtgtcagctccaggacgagccccgccagcctccaccctgtccaaccaggccctactcctcccctgcagatctgtggtatcttctgagccctaaaaacacgcctccagccagcttcctgtcctccacccggcctctcggggctcatcccatctattattcacagcacaaaatggatttttaatttgagaaatgaaatgactctcccaagtggccggggtggcagggagggggtggaggaaggccggagccgccgtggccgccacagccgccacggtgctgactcaggttcatcttggaaagctcggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 98 - DNA, 1803 bp, Artificial Sequence
  CN2461
gcggccgcacgcgtagagggatgcaaatgaggctgtgggggctggggacaccccttcctgtcagggtctccatggcacattgcctgggactgcaaggaagggcgagcctgggacaggcagagaagcctgaatggcctggagggccacttccttggctgccagggtcctgggcctgcagtcccctcccccagcactccatcatcatttcaagtgattggagctgaaggggatcgttagctaattaaagctgaggccactaattgtcccttttgaagagagagcagggctgtgcaggggaggagacagagggtctggggagggggatattggcaggcggggggctgggaacagggccatggccccttgtggggccttcttccagactgtgtgtttgaggggtcaggcattgtcagaagctccttaaagtgggtaaaggactagagaagcagatttggcgctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 99 - skipped

SEQ ID NO: 100 - DNA, 2102 bp, Artificial Sequence
  CN2628
gcggccgcacgcgtcacttgggtgccttgactaaataaagatggattccatttttaatggtcagttccagtgccttccaattcctttgagggcgtcgtttggcaggctcaagtacagtagttgaggagaaaccatgaccagggcaattacttagggttcttttaagcactggtttagtggagattttttttttttttttttttgccgagccattataattcgtgaataataaagaacaagagtgttcagattgcttagggcatggcacgttcagaaaagatcgggcatccttccaaagacagcggggcttcgggaaacgataaagcgctatgatttttaaaagtcatacgcagaattcagaagcagatactacagaggagtgcctgcaagtaggtctgctcgctctgatgggtctgaagcgggagagtgggggtggggcgggggagtaaatgttccctgctttgttgctattaacaccttggcccttgtcaccaggtcctttgtaaatgttggcagctgttattagatgcgttatcaagcaccggtgtacacagcttactttacaaatcacccacccgcagatgccttacaaacctccacagccatcaaaatgcaccccaggacttcctcgaaataccatttcctccccaagaagaaccagcagcttctggctccactgagttctgtacctaagggctacttgcctctcaaggccagagaaccacgaatccagtagttcaccgagcttctctgttcctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 101 - DNA, 2378 bp, Artificial Sequence
  CN2641
gcggccgcacgcgtggacttttggtatagcattggaaatgtaaatgagctaaatacctaataaaaatggaaaaaaaaataaaataaaagtagcattaaccaaaaaaaaacaaaaaaagaatatcacttactgttgcatttccttctttatgcagttctttcctacataaagtcttggataaaatgagaaaggcataatgtacctaagaactaagcaaagctaatgctagttagcaccacaggaatgctgacctctcattacagaacaattcaacaagaactcattcaccaacagtcagggttgagattctccccacctgcctgcagtatattatagttgaaatatggggccttggtaggttaagagactactgtaaagcttctagcactattaattgttcactctacccatgccacatctccccaggagatcagggaatattgtattgtggtgctccaaaagctcgttatgcaacgtacaaattatgttatgatgaaaggcaacaacaaaacctctgtaaggagtgttatcatttctctcttttcattaagtcacaatgcagaaactgagtaatctgaacaagtacctttaaagtaagagacttagaaagtggctggctacatgtaaagccgttcatgtgcttgcagttgtgtacaggagcaggctgctcacctgttacggagagccctttgttaagagcctttgtgagtactaaaagctttaggtttccttttcttttttaagctcttgctttaacagtgtatttctatgttcacctttcaggtaatgactgtctttggtctttttatcaaagagggaacaataacaccgcatggtattattttcacaagtattatagagtatatatttcacaagtttggtaccctttttatatcatatctttttattttagttatgtacctgattccaataaaaatcaagtaatgttgattgcaaaccccatctgtagtcatagaaatattacaatgatggacatgtagttagtttttcacttctcaaatactgtccatttcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 102 - DNA, 2300 bp, Artificial Sequence
  CN2642
gcggccgcacgcgtaaacaaagagccgccttccattcttctgagatgtctttagacatcttgcttacccctgttacacagccccacagtcattaccaagttcaatcatctaatgtgggatcccaggaggtctgcattaaccatgaagcaatgaaaggtagagtgatcctgtatatggaccaactgtcttccccaccagactgaaagctccctggggtcagagacaactcttactcatcacagaggtaccagtatccagactatgctgtctcttcccaacctctgcttgctgtcaagcctgaagctagtcaggcatctacctctgcctggctgcagttctttctcttgaagaaaggattttttttttttttggtttcataatgattagtttttttcattatcaaaacctccctgagtcacaggaggtaacctaatatcaaaacctccctgagtcataaacaaccattgctactgctacattcaccattatggaaaaaaaaggctggtctgaaacttagcctcagtgttctttctgccacctaaggaggaggttgttgcatttatttatttggggggaggggtctagtcatagcatacatgttgaggtcagaaaacactttataggaactgatttattccttctacggtctgagtcctgggaacgaaactcgagtaatctgacttgagcataagcacctttagctatgagccatcttgctagcccacaatgtgtagggggcagtgctgatgtcaaggtactgttgcccaattggttcttgatctgtcagtaaagaaagccataggctaattgctgggtggaaggtacagtcgtgacttccaggttccgggagggaaacgggatgcaaggaaggggggagggagttcgacatgctttggagagaggagaacaaagcaaccatgtgaggtagtaatacactgtgacaactacaggcaggtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 103 - DNA, 2255 bp, Artificial Sequence
  CN2643
gcggccgcacgcgtgaatgtattggattataacatgcactagtattaagtataaaagtatgttcaagaaaatttaaagttattttaaagactgatgtaaggaaagaaggaggaagttagtccattaaatcacactgagcaagagctaacttctacagagtagtcattaagaggtaattttacaatctaaattttttttttttaaaggatgctttcctttgttgtggtgtcaaggtgagaaactatgtagatttgattccgtaaatatctatggtaaggcaagtgacagggtgaactcagctgagctgatggaaatagttagaacatagaaacagtcaataactattcattttgtaaaaaaaaaaaataataataaaatagctccttcaagtcctgaagcgtaataaaatattagaaaacacaatggaaaaatgaggcactcacagaagaaatacagttggctctaaagcatagaaagtctcaattatttcaaaataatagaaaggtactttaagacagtgaacccggagtggtctctgggaacaggcagcttggtcctcaatctggcagtcagtgctaactacactgtgatcctggaaaagatatgctttacttcctcattcaaaaactcagaattatagtgctgcctgtgccctgatctacctgatgccccgccctacctgcctttgggggttgcaggtaaggtggtgtggaatcaggtgagaaatgtcacagcaagcacctctgaaagctgctgcaagctcctttaatacgctcacccgtgtcccattggtcccaagaaagcatctcttctaagcagcagttcctgattggctggcattgtctgtgtcagcaatccttggtctcttaggcagtcctcaattaggtcctcctgtagaaggtgactttgccgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 104 - DNA, 1905 bp, Artificial Sequence
  CN2629
gcggccgcacgcgtagaatatgtcatatgcccagagtctggtacttatgagggtcatagtattatggtcaatccgacaaccactcactctgagtcaggcagagtgtgcaaagtgcactcatctgtgtcacttattgtcatatgtgcacaggtgagtgattaaaagcacaagtgtgcaccgcagctccaacatgtcatactgcggcctcagacaaagaaagcaacctctctaagcctagggtgttcttgtctatcaagtcgagggttataaaggaaaacaccatggggaagaattaaatgcaaacctccccccacaccacgcttatcaaatggctgctggcatgatgccttggtttaattcttacacagtcacataccaacgggtaaggctctcagagagtgttatataattaagacaagattcttgtgtttaagcagggtggaaaaagaaatacttactggaacacagacgtacaaaacaaacaagtttctagggaagaacgaagctgcagcactgtgtgggaacacacagaagaattagactgtatgcaggagaggggatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 105 - DNA, 1956 bp, Artificial Sequence
  CN2630
gcggccgcacgcgttgacaaatgttatggcgagcccacgtttctttcaggctgtatcttagtgctatgaaatccatattgcttcacgatcagcacttatcaagattaaagaaaaacaaactccatttatttttcctttattgacagattcatggcctacatccctgtgaataaatgagctggcaagaaggggaatgggacagccagtaagcttggtgacataaattacccttgatagggaaggctgatgtgggcatgtcatcaaattaatagctgaatgctaaggaccattacatttagcactgtttgccatatccttctatataataatgcctgagaataatgtgtaattaactctgtggctgactgacaaggccaaatgcagggcaatcaatcatattgtataaacacacgtccctgagtacacatgggaggcaaagggtgacttaaaatgtctcagcactttctgcctagattatggctctgagcttttgtgaggccagcagtcatagagactcttatctatagatctaagctgcctacagcatcttggtatatagtcttccttgctatttatcttgaccttttacttgtgttctttgagggggtgacagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 106 - DNA, 2044 bp, Artificial Sequence
  CN2745
gcggccgcacgcgtatcttcaggtgtaccagcactgtgtaccagtctgtattctgtggggttggctgactccagcccacgctgtgtctgtaaatgaagttttattggaacacagtttagtgcttcctgttggtgatatagcacagagctgagtagctccaagagcaattatatagctctaaaagctgaggctgtttactttctgacctgctacagaataaggcttagacctcctgagtgagtgttctcaaatagcgatgctgagtggaattacctcaacagattcaacattgttcctgaagtacattcattatcatttactggtgttttaattggctttccttaaaaataaaacagaattaaagctgttacatgtagaattgtttatacagcagaacccgtatggtgttccttgaaggactgtgtttgagagaatcgcctttagagtgagaattacaaagacagtgggtcacgtagattaatggttgagatcctctgacctaagtaactgctacttttagctttattctattttcaactcggatctttgtttttgttttttactaagcaagtataagatggtaccaaaaaggatgttggctttccgaaggaaacagtttgccaccagttttttatattttcttctgtgccctgtgcgtgcctgccttttcactgctctagctagtaacgcccacgcttgggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 107 - DNA, 1958 bp, Artificial Sequence
  CN2746
gcggccgcacgcgttcatctgcagtcaatgaaggtctttattatcactaagtacgtcttatgagttttatatattgactttccttaagtaagccaagaacttataaaaagatcactcaaatagtatttgctataatcctcacacaattttaagagcaggaggcaggaggtctgtaatgtgaaaacaggaaagcagagagaggcaggaggtgaagcagatctgggtaattttagctgttggtagagaaatcatgaacacaccctggctttagtctgctgaagtggaaagctgtatacttattgttggagcacaaaagcagacagcatgaattgactatggtggtaaacaacttcagccctaacacaaatgaaactggccagtgactcattaggcatttagtgtttttactaagttttagttttatgaaaactaattccagtctagttattgaaatactccatgggttagttacttagagttcaatggaaaccattgcttttcttatttttttttttttttgtaaagcaattaaccattgctttaaaaagatttttgctggctaagaaaacagttgatgttctgggagaaaggaaaacctgactctcacttcatgagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 108 - DNA, 2054 bp, Artificial Sequence
  CN2631
gcggccgcacgcgttccgtaaagagcacaaaacccaaatatttgggtgcatgggagatagatttcaactaaatacttttaatcatgcaattccaatagcacttaaattttttcaaggcatgtataaagggagaatagactaattcaatttcaaagtgcaagaaaaacatcaaaaactaaattcagaaagtttgcatgggaagaaaaaaaaacacaaattaatctcacttgtgattattctaaatacatttgaacaagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttattcataatgggggaacataaagtgaacttctgcacaccagaagtcaggttgtttgcttgctaataccattcccactcaatatcccatggaagttggatgactcgacctgcatcccaaggagtagttaccatcactaatgaggagaagcatttggaaggaagacagaattctgtagaccaaacgaagacctggaggctagtggaaagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 109 - DNA, 2112 bp, Artificial Sequence
  CN2747
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgtaggcttcttctgcaatgtggagtttctagatccttcctatgatccaacccctcagacttcacatcctctccatcaccaggacagacgatcccattgagtaacatctggcttttgccttctgtgggcatatggctgtagctatcacagaagcatacagatctgctaggttcttgtgggcagcacacccatggtaccttttaaagggcctgctgtattcgctctttaacactcagcgtggcatcaacccagtggaaatgaactgggagctccctgcaggttctgtgctcccttggcaggcacagccttcccaccccacaccttcccctggagcattgagctctgcgggaacacaggcttgctgaaggaaccagcagggccatcgggagcactgggcctgggagggagatcaacagcacaaagtagagccagcacaaagtggagctgagagaagccttggagcatgaacaacatatctgcaccccaagaaaagcccttaaccgaagagcagagccatggcacttaaggagctcgattcagccgggagcttagggaggggaggtcacttcataagggcttggggggggagttggagccacgagtcgtccagccggagccccgtgtggctgtgctccggcctcagaagcatccccggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 110 - DNA, 1980 bp, Artificial Sequence
  CN2632
gcggccgcacgcgtagagagggagcaatttctttcagcaacggaaataggctttgttgtattttttaagtccccataaaaatgtaaagttctaacatctcaaattaccttatgttccacaggaatattaggtgttagcggatactctgcaggaaatgaaaaatgattacttgaaaccatttaattggctttcactcaacaaataccaaagtgcaatttgtggcctgatttgaatgctaatgcctgaattacaggcatggaatcaaagtgctagatgctttcagtgcagaatgccagtgcttaattaagttttccagtaactgtgtacagcaataggattcacagcgagtgcgcggatatttaaggttatttatttctgctatgtgaagtatttttcccttggaatctttgttgtacaaattgacatttaccaggtggtgaaaaagtttacaatcttgacaaattctttcattcaaattaatgttttccttatcaatcattccaaacttaaatgcctgtattattaggcacacagttggcatttaaaagtatggattaaggaactatgaatgcatctgatttgttactctgtataagcctataccaaatcaaattggttgtggttttccagaattttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 111 - DNA, 2134 bp, Artificial Sequence
  CN2644
gcggccgcacgcgtagtttcgtgtagttttaaaaaccctatttattcttttcttgtgaaatgtttcaaatgaattcaaagaaaatatgatgttttatttctttacaaaaggtgataaatattatttttatgaataatattcacattgggcccctatgtggacgctgaatagccctagtgtggagtaaggtacaaccttactggaggaaggatatcactatggggctgggctttgaggtctcagaagcccaagtcaattctctcactgctgcctgtggttcaagataaagaactctcagctccttctctagcatcaagtctgcctctacactgccttgtttcctgccataataataacagactaaccctctgaattgtaagccagccccaattaaatgttttctttataagagttgttgtggtaatggtgtactttcacagaaataaaatcctaactaagacattttccaaattgtatacccttgattctttttagttcttgtattgctctaggtagaacttcaaatactatattgaaaagacattggtagagtgggcagccttctcctattcctgatttgagttgcctctggtggagcaaggggtttatgcctggatagaatgacttattatagaatgacttagaatgaatggggttcttggtgctggtatggcctctggttgtgaaggctctggggaagtattgctcattcatgaggtgggggcttctctaaaaatcttcatgctagtatagcctctcggggatcagggtctgctggaagtgtgcataagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 112 - DNA, 1876 bp, Artificial Sequence
  CN2748
gcggccgcacgcgtactcctctggtatactgcagaaaatccttccaaaaatcttcacaacagtgaggtttttaaacaggttggactcaaagcatggagaacggaaggagaaacacagtgggggcagaaattccagaggcaagctgagctggaaaacacatggccataatcagggggtggggaaacaaagcattcccacaggacagtgggaggcacagagcaacccaatgtcacttatacaccttatagaaatcctggcagagccacctacagactagcttattaatatcccaatgttgttttcaggagagactaaaattctaaaacgctttttaaatgatgtgttttgtatacacaagttttcacccacaccacttacagttttccaataaaagcctatctaaataaacagtttaaattcgttacacggggctgcatatttcagttctgaaatcaaagatgggaatcaatttgaattatgactgaaatcaccatttagaaaatggtctcatactggatgggttgttctcagagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 113 - DNA, 2054 bp, Artificial Sequence
  CN2633
gcggccgcacgcgttgatgtccaggttggctgttcaccaaggacacccagagagactgtatggtcttagagagtctgagtggtagcacatatttgaaatagaaagcaccaccctacccccagatggtctctgcctcctccacctactgaggtgacatctgcacttcctatgtaatctttactcacacctcttacaaagacagcatgtcgtcatcctgaccccagcagccagccgagctgggctttccagaagtcttcaagctggaggagtgggaggcaccatggcctgctcaaggggaacagagtgaattgatgccagacggtggcaggtggctattttcacttagcagttagtggctaatgaaagaggtgtgtgggtatctttctccccagaggccattcatttctctccagctgaaagagcaatggactgatctgaggcaggaccaaaagttcaaaggaattatgacagattggcccagcacccctgagtttatctgaataatgacgcataagcaccatccagtgttgacaaagctctgcaggctgtgaaggcttaaggggagccttggagctaagcacagagggcaatgatgttgcttaaatgtgcacaggtctggattaaagtggagtagttcttaggctgctcatgtcttcttaatcttcagctaggattgagactagcctccatgacacaggggatccatgacagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 114 - DNA, 2042 bp, Artificial Sequence
  CN2634
  misc_feature (22)..(22): n is a, c, g, or t
gcggccgcacgcgttcctgcgnccgcacgcgtgctcagttgccagcacacaaatgatgtcacatgagcatctataactccagttctaggagatctgacaccctcttctgaccttcattggcaccaggctctcagtgtttacacatacatgcaggcaaaaaacactcatacacacaaaataataaagaaatttaaaattaagaaagagaaaatcatagttttagtagcttttctcttccatttgtttgatttcttgccttatgacattgtttattgtgttcattggtgactgtctttacactgaggattctatgaatgggcagtttggaacacttctaacgctttgtactctgtcactggcatgctcacagtgtataccacctaccacagaggaacatccattgtttaaaaagcagttcaggttattttgaaacactaatacaaaataattttcagctgtaacgatgggaatataacaatttccttgactacaaagagaatataacaagtgcctatgattcaaatgcaaaagacaccgaatatccatcagtgcttttgaaaatggcaggacttactgtattgacacttaacaagcttttttttttttttgtctgaagcttgtttttaaatgttggaattttagttgaaaaatctatgtcactcttttactgcagttttctgctttcataacattttcaagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 115 - DNA, 1891 bp, Artificial Sequence
  CN2635
gcggccgcacgcgtgcttatatgtgtgggcaatatgtatatattgttactttatatgtatgtgtataaagagagactgttaacatgttttcagaaactcttaaatacatataaaggcatacacaaatatctgcagaggtaattatattaaagtgcagagtttagctactatgttttgcatttgtaaaatgccatagaaacatcagctcattaattttgttcacctcgtaggactattattcagaatctttattcagccagggagggcattttgtatttagctattacttggctctctcccttttgtcataccatggcaggtagtctatacttaaacctgaaatttttagtgtcaaacttgtggcaaacaaagtccccttgttccttagctaatgagcacaaggttggggtttacatactatcactaggctttaaggtgaggaccgttgtccagcagaggggcctgtcatttttgagaggtatttgtgtattttataaaccagccaactgtctttcttttcttttccaccatcactcatttccttccagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 116 - DNA, 1864 bp, Artificial Sequence
  CN2609
gcggccgcacgcgttctcatcctatgcctttcttctaggtacttcagaaagaatctttcaaatcaataactgtgttctatccacagaggtttaatggaggagcaggaggctggaacgagatgaaggaaatgaattgttccagagatgaaaaagctcccaggcatatgtcataagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaatgagtttctcatttttttaatttaaattacttttgagctacttctagactgcagcagagttgataaaccacccagttcatatactcaaacctcccaactcctccacagctcagccaaaatttcccaaggacttgttttacccaaatcccagctccggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 117 - DNA, 1892 bp, Artificial Sequence
  CN2610
gcggccgcacgcgttgtttcacaaactacaggttcactggagcacagccatgccaacatggcatctaaggctggtatgacctcgagacccctccagcactccttcgttcagccatagaacaaggagacacagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattactggttaggatttctctgtttgctgagtgcattaacttttagcgcttcccatcactcattcttcttcacaccctcttgtcagttacaaatgtgcaggtggagagccacgaaaaaaccctgcagcaacggcaacagaaatcacagcaacaacaacaaatgaacagaaaggatagaaagcagatactcaaagatggagaaaaagatactcccgagaggctgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 118 - DNA, 1979 bp, Artificial Sequence
  CN2749
gcggccgcacgcgtaacctcagtggtttcagccaccatggaagagagagagacaggagtcagaatgtaaaggcatggggtttggtggaccagagagagagattctcagtgcagtgggaattgggctgctgggcagaatatacatcccatagactactcccccaccccctacggtgtaagaaaaaagacatctctatgacttggcttgcacaaactttcttgtgtgctacctttgcaacagcagatctcaggcaggtgggtggatgggtgtggaggaggagaaagcagaaagacaaaaggaagtgagtggggggaactcttgggaacctgttggcagagcgctctgagactctgaagctaagggaccatgttatgttaatctattctctaattgctcagcaacaaacacctcccacccgctgcaaggaagggcaggacaagactattccaagatctgcttagaaacaatgtcagtcacagcagagggcgcaaaagaaatggacctctttttagcataattgaaagacactacttacaactgaggggtctgagttgctgacagcaaccgccttagctaggaggaaaagctgaggggtaggctgtgctcaatgggagggtttgcaggctgactgttcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 119 - DNA, 1967 bp, Artificial Sequence
  CN2626
gcggccgcacgcgtagcctgaattccacactacagctcttatattagaaaagaaactggatgaaaaaagatgggtctcagagaagatacttggagcaatagccacacaagagttgctcaagtatttccatcagcctaaatgactacacacctatttgccaccagctggcctcagctctgctctgaatgcccaccatcaactctctgagaaagtgcccctgactacactcagtgaagctgtgagttggtggctgccttttccccctgcaagataacaaggtaggccttacctcagacttaactcaaggtaaagtttaattgctctaacctgctctttgatgtgtgctgaggaggtgggcgccgtgtgggtacaagttgctatttatacagacctgacaagcgcatcccatcagcacccgctcctttgttctcttccaggcttttctgattctccaaacttaacatcctgcactcccccaagttgtttaatcgttccaaagtttgtaagacaagcaagggaatagtggccttcacatctgcacttgcagcccaaatatggtgcactgtgagagatccttaatttggagaggaaagcctgctgctgaccacctgcctcttagaactgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 120 - DNA, 2037 bp, Artificial Sequence
  CN2611
gcggccgcacgcgtttcctggacactgaattctgagagacagaggtaggagaggggaaccatgggatgtgacgatttcaaagatttaccctgggcactgtataagaaaacgttttccaaagtgtctgacactgggtctgactgacacaaggcaccagcaaatgtcacctctcttccctgtgtgtcacacaacttcatttctcttgaaactaaaacttctcttctagttgcccttgacttccaatgtcaccgttctctagcccataccccattactaatcctccccgcccggttccgcctacgtcccttttgttcccactaaagatggctttgctttttcacctgtttaagtcttcctgcctcccacactgatgaattcagttagactgaaaaaaaaaaaaagaggcctcttcctccataaaaccccaattaatcaatcattcacgactgagcaatttggggtgatggtatttagtgggaagaactgctccagcactgtcaggcctgggatgtgacactaacagcagtgagacctaagaggagtaatttggcttttttcatatctcagtttgctcactcacagtaccttaactcttaagaccggtaagagtgaaaattacccccagaggaatcctagggcaaagacagccaggtgatcatcagtataaactaaggatgcctgttcataccagcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 121 - DNA, 1848 bp, Artificial Sequence
  CN2750
gcggccgcacgcgtaggaactggacacacaaagcctttctcgttttttcttcctccctgtgtttcggtccaggggtcctaagtgtgtagctcccggggagatgtgggactgttctcacccagaaagaggaactagaaccacaagagcctaaccccccacggtgggaagacactctgtggcatcaccgggaggggaggccaggtgtgggcaactgcagctatttctaatccaccagaggaaagagagctttgactgcttgaaaagcaggctgattgtcaggggcggaacccagctcagagctgtcagggaagctcctggcagggggtggagacagggggcttggactgacaggcctgtgattttgaaagaaatgtctttaagagagtgacttaaagtaggagagaaatattattattaggtaatgcctgtcaggttatatttttttttgatgtgttcttggtatttcataatcattttgcctttagatggtaagcccttcctggtgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 122 - DNA, 1983 bp, Artificial Sequence
  CN2614
gcggccgcacgcgttaaaccttggactgagcccacaggatgctctggaaggaccttgggaaaccgacacacaaagcctgtctcggttttgttttgttttgttttgtcctccctgtgtcgcatccacaagtcccaagcgtgtagctcctgggaagctatgtggctgttttcaccaaggatgacttctcagagactgacacccataagagaggcccctccacccgagggtgggaggactctctgtggcatcaccgggaggggaggccaggtgtgggcactgcagctatttctaatccaccagaggaaagagagctttgactgcttgaaaagcaggctgattgtcaggggcggaacccagctcagagctgtcagggaagctcctggcagggggtggagacagggggcttggactgacaggcctgtgattttgaaagaaatgtctttaagagagtgacttaaagtaggagagaaatattattattaggtaatgcctgtcaggttatatatttttttttgatgtgttcttggtatttcataatcattttgcctttagatggtaagcccttcctggtcctgggccatgaactatgcccctcgggtctcaccccagccttggacatgcaatgggcaccaggaggtagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 123 - DNA, 53 bp, Artificial Sequence
  minBglobin promoter
gggctgggcataaaagtcagggcagagccatctattgcttacatttgcttctg

SEQ ID NO: 124 - DNA, 68 bp, Artificial Sequence
  minCMV promoter
gaggtaggcgtgtacggtgggaggcctatataagcagagctcgtttagtgaaccgtcagatcgcctgg

SEQ ID NO: 125 - DNA, 68 bp, Artificial Sequence
  mutated minCMV promoter
gaggtaggcgtgtacggtgggaggcctatataagcagagctggtttagtgaaccgtcagatcgcctgg

SEQ ID NO: 126 - DNA, 120 bp, Artificial Sequence
  minRho promoter
gattcagccgggagcttagggaggggaggtcacttcataagggcctggggggggagttggagccacgagtcgtccagccggagccccgtgtggctgagctccggcctcagaagcatcccc

SEQ ID NO: 127 - DNA, 120 bp, Artificial Sequence
  minRho* promoter
gattcagccgggagcttagggaggggaggtcacttcataagggcttggggggggagttggagccacgagtcgtccagccggagccccgtgtggctgtgctccggcctcagaagcatcccc

SEQ ID NO: 128 - DNA, 867 bp, Artificial Sequence
  Hsp68 minimal promoter
caggaacatccaaactgagcagccggggtcccccccaccccccaccccgccccacgcggcaactttgagcctgtgctgggacagagcctctagttcctaaattagtccatgaggtcagaggcagcactgccattgtaacgcgattggagaggatcacgtcaccggacacgcccccaggcatctccctgggtctcctaaacttggcggggagaagttttagcccttaagttttagcctttaacccccatattcagaactgtgcgagttggcgaaaccccacaaatcacaacaaactgtacacaacaccgagctagaggtgatctttcttgtccattccacacaggccttagtaatgcgtcgccatagcaacagtgtcactagtagcaccagcacttccccacaccctccccctcaggaatccgtactctccagtgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcaggtcaccagacgctgacagctactcagaaccaaatctggttccatccagagacaagcgaagacaagagaagcagagcgagcggcgcgttcccgatcctcggccaggaccagccttccccagagcatccctgccgcggagcgcaaccttcccaggagcatccctgccgcggagcgcaactttccccggagcatccacgccgcggagcgcagccttccagaagcagagcgcggcgcc

SEQ ID NO: 129 - DNA, 720 bp, Artificial Sequence
  SYFP2
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaa

SEQ ID NO: 130 - DNA, 720 bp, Artificial Sequence
  EGFP
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 131 - DNA, 1299 bp, Artificial Sequence
  FlpO
atggctcctaagaagaagaggaaggtgatgagccagttcgacatcctgtgcaagaccccccccaaggtgctggtgcggcagttcgtggagagattcgagaggcccagcggcgagaagatcgccagctgtgccgccgagctgacctacctgtgctggatgatcacccacaacggcaccgccatcaagagggccaccttcatgagctacaacaccatcatcagcaacagcctgagcttcgacatcgtgaacaagagcctgcagttcaagtacaagacccagaaggccaccatcctggaggccagcctgaagaagctgatccccgcctgggagttcaccatcatcccttacaacggccagaagcaccagagcgacatcaccgacatcgtgtccagcctgcagctgcagttcgagagcagcgaggaggccgacaagggcaacagccacagcaagaagatgctgaaggccctgctgtccgagggcgagagcatctgggagatcaccgagaagatcctgaacagcttcgagtacaccagcaggttcaccaagaccaagaccctgtaccagttcctgttcctggccacattcatcaactgcggcaggttcagcgacatcaagaacgtggaccccaagagcttcaagctggtgcagaacaagtacctgggcgtgatcattcagtgcctggtgaccgagaccaagacaagcgtgtccaggcacatctactttttcagcgccagaggcaggatcgaccccctggtgtacctggacgagttcctgaggaacagcgagcccgtgctgaagagagtgaacaggaccggcaacagcagcagcaacaagcaggagtaccagctgctgaaggacaacctggtgcgcagctacaacaaggccctgaagaagaacgccccctaccccatcttcgctatcaagaacggccctaagagccacatcggcaggcacctgatgaccagctttctgagcatgaagggcctgaccgagctgacaaacgtggtgggcaactggagcgacaagagggcctccgccgtggccaggaccacctacacccaccagatcaccgccatccccgaccactacttcgccctggtgtccaggtactacgcctacgaccccatcagcaaggagatgatcgccctgaaggacgagaccaaccccatcgaggagtggcagcacatcgagcagctgaagggcagcgccgagggcagcatcagataccccgcctggaacggcatcatcagccaggaggtgctggactacctgagcagctacatcaacaggcggatctga

SEQ ID NO: 132 - DNA, 1056 bp, Artificial Sequence
  iCre
atggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaa

SEQ ID NO: 133 - DNA, 50 bp, Artificial Sequence
  SP10 insulator
gaagctacccctaacacactattctacacacagaaaatgctcttcactag

SEQ ID NO: 134 - DNA, 150 bp, Artificial Sequence
  3x SP10 insulator
gaagctacccctaacacactattctacacacagaaaatgctcttcactaggaagctacccctaacacactattctacacacagaaaatgctcttcactaggaagctacccctaacacactattctacacacagaaaatgctcttcactag

SEQ ID NO: 135 - DNA, 246 bp, Artificial Sequence
  WPRE3
ataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgg

SEQ ID NO: 136 - DNA, 609 bp, Artificial Sequence
  WPRE
gcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccg

SEQ ID NO: 137 - DNA, 204 bp, Artificial Sequence
  BGHpA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatg

SEQ ID NO: 138 - DNA, 479 bp, Artificial Sequence
  HGHpA
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 139 - DNA, 78 bp, Artificial Sequence
  P2A
ggcagcggcgccaccaacttcagcctgctgaagcaggccggcgacgtggaggagaaccccggccccggagctagcgga

SEQ ID NO: 140 - protein, 21 aa, Artificial Sequence
  T2A
  MISC_FEATURE (1)..(3): (GlySerGly) residues can be added to 5′ end to improve cleavage efficiency
GSGEGRGSLLTCGDVEENPGP

SEQ ID NO: 141 - protein, 24 aa, Artificial Sequence
  E2A
  MISC_FEATURE (1)..(3): (GlySerGly) residues can be added to 5′ end to improve cleavage efficiency
GSGQCTNYALLKLAGDVESNPGPP

SEQ ID NO: 142 - protein, 25 aa, Artificial Sequence
  F2A
  MISC_FEATURE (1)..(3): (GlySerGly) residues can be added to 5′ end to improve cleavage efficiency
GSGVKQTLNFDLLKLAGDVESNPGP

SEQ ID NO: 143 - DNA, 130 bp, Artificial Sequence
  Exemplary Plasmid Backbone 1, Left ITR
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 144 - DNA, 141 bp, Artificial Sequence
  Exemplary Plasmid Backbone 1, Right ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 145 - DNA, 146 bp, Artificial Sequence
  Exemplary Plasmid Backbone 2, Left ITR
catgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 146 - DNA, 149 bp, Artificial Sequence
  Exemplary Plasmid Backbone 2, Right ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctg

SEQ ID NO: 147 - protein, 743 aa, Artificial Sequence
  PHP.eB capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 148 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 149 - DNA, 747 bp, Artificial Sequence
  tet-Transactivator version 2
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 150 - DNA, 1930 bp, Homo sapiens
ggagagagagaggacagagagcaagtcactcccggctgcctttttcacctctgacagagcccagacaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagccatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaacaattcaagtga

SEQ ID NO: 151 - DNA, 3703 bp, Artificial Sequence
  rAAV-hAADC-vector gene insert
tctgcgcgctcgctcgctcactgaggccgcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctggttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattccccggggatccaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagttatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaggaattaattcgatatcaagctatccaacacactggtagggataacagggtaatctcgaggcaagcttgggcccggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagat

SEQ ID NO: 152 - DNA, 1816 bp, Homo sapiens
cggacctccacactgagccatgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtcatggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaggtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctct

SEQ ID NO: 153 - DNA, 2921 bp, Homo sapiens
ccggctcggagtgtgatctaagcaggtcgcgtaccttcctcaggtgactccggccacagcccattgtccgcggccaccggcggagtttagccgcagacctcgaagcgccccggggtccttcccgaacggcagcggctgcggcgggtccatggagaagggccctgtgcgggcaccggcggagaagccgcggggcgccaggtgcagcaatgggttccccgagcgggatccgccgcggcccgggcccagcaggccggcggagaagcccccgcggcccgaggccaagagcgcgcagcccgcggacggctggaagggcgagcggccccgcagcgaggaggataacgagctgaacctccctaacctggcagccgcctactcgtccatcctgagctcgctgggcgagaacccccagcggcaagggctgctcaagacgccctggagggcggcctcggccatgcagttcttcaccaagggctaccaggagaccatctcagatgtcctaaacgatgctatatttgatgaagatcatgatgagatggtgattgtgaaggacatagacatgttttccatgtgtgagcatcacttggttccatttgttggaaaggtccatattggttatcttcctaacaagcaagtccttggcctcagcaaacttgcgaggattgtagaaatctatagtagaagactacaagttcaggagcgccttacaaaacaaattgctgtagcaatcacggaagccttgcggcctgctggagtcggggtagtggttgaagcaacacacatgtgtatggtaatgcgaggtgtacagaaaatgaacagcaaaactgtgaccagcacaatgttgggtgtgttccgggaggatccaaagactcgggaagagttcctgactctcattaggagctgagcttcattcagtgtgtgtgcgttggttgccgatcgtactgccagtagcattgtctgtctgtccggtcttgtttgtacattccattttcaattgttacagatgtgaactttattccttgtcactaattatatttaaaattatttctaggaagtcaaataaatataataaagggttgagcctctactttcttcttgccacctttttgtggcaatattaaagtgaactgctaatagtgtaagtatgtgcacaaaaccactgccagataaccagaggggcctgggaagggagaagaattagtgtatttttttcaaatagtacagtaatttgcctcataagcataggagcattgggaatgagagggaactgtgcccagtatactgttttttttcttcctccaataaaagtggtgtagtgccgaaagtgctaaaatatttagtgcggtattgctctgtgaattcaagttcaacagacttcactttggtcatgtttattaaaccaccagtgacatttaaaaatatatttttagcagtcgtaatgttagtcaccaagggaaggtggtggaatgtctatgtttttgattttactgtgagttaaaaaggcacatttctaccttctattgtttttaaattcaagaatagggaattagttcctggtgttgtttacgagtgtattctcgtgtcaacatacagggatttagacatttaactctctgtgccttgataagaatatcatttagagtgtagatacttttgcctttttaaaaaagccattattttatgagacttagtactcacactgcaaataactagtcagctcagttttaactttataggtttattgagtttcctttgtgtgatccatgtagatgcctcaaaatgtttcttcttcttcttttttttaatcttataagatatttttctaagtatttccagaaacatttgagagtgcccatcattttcaggtctgcagaaccatagcttccacgcacctgaacgagcacagaatgaactgacggtggaagacattatgagctgtgtccaacgttttaaccaaagcgtatcgtaccaacgatctgtgaaaatgcactggaagcttctggtcccggtttcctttgtggtctatgtgggtcttgtcctcattgtaactccgtatagatggtataggtattttaatcctggaagctgttgccttattaatgattatcttaaaatttcctccattggggcagcgtgggccaaattaaaacaaacaaaaccgcaactcctccacagaaacacaaacacagttattccatgaagtttagtatttggttgacatagtgctcttcaaattcatcccattaccctaaaagtaataactttgatgcttgctttaactttagtcccatctctgccactttgatgctatttgggttatgatggggcaagatggcagaggtattgggtttttttgtttttttccattcctctctacttctgtttcctagctttttctttctggagtttaagtacagtgatggttggcttgagtacctttttaaatctagcccagtataaacattagcctgcttaatatttagacatttataggtagaattctgagcactcaactcatgtttggcattttaaagtaaaaacaagtgtgacttcgaggaccaaagaaattgtcagctatacatttatctttatgaactcatttatattcctttttaatgactcgttgttctaacatttcctagaagtgttcttataaaggtctaatgtatccacaggctgttgtcttattagtaaatgcaaagtaatgactttgtctgttttactctagtctttagtacttcaaaattaccttttcatatccatgatcttgagtccatttgggggatttttaagaatttgatgtatttcaatacactgttcaaaattaaattgtttaattttatgtatgagtatgtatgttcctgaagttggtcctatttaaattattaaactattgtaactttaaaaaaaaaaaaaaaaaaaaa

SEQ ID NO: 154 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 155 - protein, 449 aa, Homo sapiens
MELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 156 - protein, 487 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 157 - RNA, 614 nt, Homo sapiens
uugcugugugaggcagaaccugcgggggcaggggcgggcugguucccuggccagccauuggcagaguccgcaggcuagggcugucaaucaugcuggccggcguggccccgccuccgccggcgcggccccgccuccgccggcgcacgucugggacgcaaggcgccgugggggcugccgggacggguccaagauggacggccgcucagguucugcuuuuaccugcggcccagagccccauucauugccccggugcugagcggcgccgcgagucggcccgaggccuccggggacugccgugccgggcgggagaccgccauggcgacccuggaaaagcugaugaaggccuucgagucccucaaguccuuccagcagcagcagcagcagcagcagcagcagcagcagcagcagcagcagcagcagcagcagcagcaacagccgccaccgccgccgccgccgccgccgccuccucagcuuccucagccgccgccgcaggcacagccgcugcugccucagccgcagccgcccccgccgccgcccccgccgccacccggcccggcuguggcugaggagccgcugcaccgaccgugaguuugggcccgcugcagcucccuguc

SEQ ID NO: 158 - RNA, 21 nt, Homo sapiens
cuucgagucccucaaguccuu

SEQ ID NO: 159 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 160 - DNA, 1353 bp, Artificial Sequence
  GCaMP6m
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaagggagctacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 161 - DNA, 1353 bp, Artificial Sequence
  GCaMP6s
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttccacatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggctttctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 162 - DNA, 1353 bp, Artificial Sequence
  GCaMP6f
atgggttctcatcatcatcatcatcatggtatggctagcatgactggtggacagcaaatgggtcgggatctgtacgacgatgacgataaggatctcgccaccatggtcgactcatcacgtcgtaagtggaataagacaggtcacgcagtcagagctataggtcggctgagctcactcgagaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacctgccggaccaactgactgaagagcagatcgcagaatttaaagaggaattctccctatttgacaaggacggggatgggacaataacaaccaaggagctggggacggtgatgcggtctctggggcagaaccccacagaagcagagctgcaggacatgatcaatgaagtagatgccgacggtgacggcacaatcgacttccctgagttcctgacaatgatggcaagaaaaatgaaatacagggacacggaagaagaaattagagaagcgttcggtgtgtttgataaggatggcaatggctacatcagtgcagcagagcttcgccacgtgatgacaaaccttggagagaagttaacagatgaagaggttgatgaaatgatcagggaagcagacatcgatggggatggtcaggtaaactacgaagagtttgtacaaatgatgacagcgaagtga

SEQ ID NO: 163 - RNA, 19 nt, Artificial Sequence
  First RNA sequence for targeting huntingtin gene
aaggacuugagggacucga

SEQ ID NO: 164 - RNA, 20 nt, Artificial Sequence
  First RNA sequence for targeting huntingtin gene
aaggacuugagggacucgaa

SEQ ID NO: 165 - RNA, 21 nt, Artificial Sequence
  First RNA sequence for targeting huntingtin gene
aaggacuugagggacucgaag

SEQ ID NO: 166 - RNA, 22 nt, Artificial Sequence
  First RNA sequence for targeting huntingtin gene
aaggacuugagggacucgaagg

SEQ ID NO: 167 - RNA, 23 nt, Artificial Sequence
  First RNA sequence for targeting huntingtin gene
aaggacuugagggacucgaaggc

SEQ ID NO: 168 - RNA, 19 nt, Artificial Sequence
  Second RNA sequence for targeting huntingtin gene
ucgagucccucaaguccuu

SEQ ID NO: 169 - RNA, 20 nt, Artificial Sequence
  Second RNA sequence for targeting huntingtin gene
uucgagucccucaaguccuu

SEQ ID NO: 170 - RNA, 21 nt, Artificial Sequence
  Second RNA sequence for targeting huntingtin gene
cuucgagucccucaaguccuu

SEQ ID NO: 171 - RNA, 22 nt, Artificial Sequence
  Second RNA sequence for targeting huntingtin gene
ccuucgagucccucaaguccuu

SEQ ID NO: 172 - RNA, 23 nt, Artificial Sequence
  Second RNA sequence for targeting huntingtin gene
gccuucgagucccucaaguccuu

SEQ ID NO: 173 - RNA, 19 nt, Artificial Sequence
  Second RNA sequence for targeting huntingtin gene
cuucgagucucaaguccuu

SEQ ID NO: 174 - RNA, 19 nt, Artificial Sequence
  Second RNA sequence for targeting huntingtin gene
acgagucccucaaguccuc

SEQ ID NO: 175 - RNA, 72 nt, Artificial Sequence
  pre-miR451a
cuugggaauggcaaggaaggacuugagggacucgaagacgagucccucaaguccucucuugcuauacccaga

SEQ ID NO: 176 - RNA, 69 nt, Artificial Sequence
  Pre-miR155
ugcugaaggacuugagggacucgaagguuuuggccacugacugaccuucgagucucaaguccuucagga

SEQ ID NO: 177 - RNA, 21 nt, Artificial Sequence
  Double stranded RNA for targeting human hungtingtin gene of exon 1
augaaggccuucgagucccuc

SEQ ID NO: 178 - RNA, 21 nt, Artificial Sequence
  Double stranded RNA for targeting human hungtingtin gene of exon 1
ggcgacccuggaaaagcugau

SEQ ID NO: 179 - RNA, 21 nt, Artificial Sequence
  Double stranded RNA for targeting human hungtingtin gene of exon 1
uggcgacccuggaaaagcuga

SEQ ID NO: 180 - RNA, 21 nt, Artificial Sequence
  Double stranded RNA for targeting human hungtingtin gene of exon 1
auggcgacccuggaaaagcug

SEQ ID NO: 181 - RNA, 18 nt, Artificial Sequence
  Double stranded RNA for targeting human hungtingtin gene of exon 1
cgaccaugcgagccagca

SEQ ID NO: 182 - RNA, 18 nt, Artificial Sequence
  Double stranded RNA for targeting human hungtingtin gene of exon 1
agucgcugaugaccggga

SEQ ID NO: 183 - RNA, 18 nt, Artificial Sequence
  Double stranded RNA for targeting human hungtingtin gene of exon 1
acgucguaaacaagagga

SEQ ID NO: 184 - RNA, 21 nt, Artificial Sequence
  Double stranded RNA for targeting human hungtingtin gene of exon 1
gucgaccaugcgagccagcac

SEQ ID NO: 185 - RNA, 21 nt, Artificial Sequence
  Double stranded RNA for targeting human hungtingtin gene of exon 1
auagucgcugaugaccgggau

SEQ ID NO: 186 - RNA, 21 nt, Artificial Sequence
  Double stranded RNA for targeting human hungtingtin gene of exon 1
uuacgucguaaacaagaggaa

SEQ ID NO: 187 - RNA, 75 nt, Artificial Sequence
  Double stranded RNA for targeting human hungtingtin gene of exon 1
aaaacucgagugagcgcugaaggccuucgagucccucaugagggacucgaaggccuucaucgccuacuaguaaaa

SEQ ID NO: 188 - RNA, 75 nt, Artificial Sequence
  Double stranded RNA for targeting human hungtingtin gene of exon 1
aaaacucgagugagcgcugaaggccuucgagucuuuuaugagggacucgaaggccuucaucgccuacuaguaaaa

SEQ ID NO: 189 - RNA, 75 nt, Artificial Sequence
  Double stranded RNA for targeting human hungtingtin gene of exon 1
aaaacucgagugagcgcaugaaggccuucgagucccucgagggacucgaaggccuucauccgccuacuaguaaaa

SEQ ID NO: 190 - RNA, 75 nt, Artificial Sequence
  Double stranded RNA for targeting human hungtingtin gene of exon 1
aaaacucgagugagcgcaugaaggccuucgagucuuuugagggacucgaaggccuucauccgccuacuaguaaaa

SEQ ID NO: 191 - RNA, 85 nt, Artificial Sequence
  Double stranded RNA for targeting human hungtingtin gene of exon 1
cucgagugagcgcucccggucaucagcgacuauuccguaaagccacagauggggauagucgcugaugaccgggaucgccuacuag

SEQ ID NO: 192 - RNA, 86 nt, Artificial Sequence
  Double stranded RNA for targeting human hungtingtin gene of exon 1
cucgagugagcgaugcuggcucgcauggucgauacuguaaagccacagaugggugucgaccaugcgagccagcaccgccuacuaga

SEQ ID NO: 193 - RNA, 86 nt, Artificial Sequence
  Double stranded RNA for targeting human hungtingtin gene of exon 1
cucgagugagcgcuccucuuguuuacgacgugaucuguaaagccacagaugggauuacgucguaaacaagaggaacgccuacuagu

SEQ ID NO: 194 - RNA, 163 nt, Artificial Sequence
  Double stranded RNA for targeting human hungtingtin gene of exon 1
gcguuuagugaaccgucagaugguaccguuuaaacucgagugagcgaugcuggcucgcauggucgauacuguaaagccacagaugggugucgaccaugcgagccagcaccgccuacuagagcggccgccacagcggggagauccagacaugauaagauacauu

SEQ ID NO: 195 - RNA, 163 nt, Artificial Sequence
  Double stranded RNA for targeting human hungtingtin gene of exon 1
gcguuuagugaaccgucagaugguaccguuuaaacucgagugagcgcucccggucaucagcgacuauuccguaaagccacagauggggauagucgcugaugaccgggaucgccuacuagagcggccgccacagcggggagauccagacaugauaagauacauu

SEQ ID NO: 196 - protein, 4 aa, Artificial Sequence
  Change from AAV9 to PHP.eB
SAQA

SEQ ID NO: 197 - protein, 11 aa, Artificial Sequence
  Change from AAV9 to PHP.eB
SDGTLAVPFKA

SEQ ID NO: 198 - protein, 7 aa, Artificial Sequence
  AAV-BR1
NRGTEWD

SEQ ID NO: 199 - protein, 7 aa, Artificial Sequence
  AAV-PHP.S
QAVRTSL

SEQ ID NO: 200 - protein, 7 aa, Artificial Sequence
  AAV-PHP.B
TLAVPFK

SEQ ID NO: 201 - protein, 7 aa, Artificial Sequence
  AAV-PPS
DSPAHPS

SEQ ID NO: 202 - DNA, 200 bp, Artificial Sequence
  Core-eHGT_621h
atacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaa

SEQ ID NO: 203 - DNA, 600 bp, Artificial Sequence
  3xCore-eHGT_621h
atacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaaatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaaatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaa

SEQ ID NO: 204 - DNA, 176 bp, Artificial Sequence
  core2_eHGT_351h
gcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagag

SEQ ID NO: 205 - DNA, 528 bp, Artificial Sequence
  3xcore2_eHGT_351h
gcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagaggcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagaggcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagag

SEQ ID NO: 206 - DNA, 160 bp, Artificial Sequence
  core2_eHGT_367h
tccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactg

SEQ ID NO: 207 - DNA, 480 bp, Artificial Sequence
  3xcore2_eHGT_367h
tccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactg

SEQ ID NO: 208 - DNA, 428 bp, Artificial Sequence
  core_eHGT_441h
gcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccac

SEQ ID NO: 209 - DNA, 1284 bp, Artificial Sequence
  3xcore_eHGT_441h
gcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccacgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccacgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccac

SEQ ID NO: 210 - DNA, 235 bp, Artificial Sequence
  core2_eHGT_444h
ggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaa

SEQ ID NO: 211 - DNA, 705 bp, Artificial Sequence
  3xcore2_eHGT_444h
ggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaaggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaaggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaa

SEQ ID NO: 212 - DNA, 222 bp, Artificial Sequence
  core2_eHGT_445h
gtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggc

SEQ ID NO: 213 - DNA, 666 bp, Artificial Sequence
  3xcore2_eHGT_445h
gtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggcgtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggcgtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggc

SEQ ID NO: 214 - DNA, 246 bp, Artificial Sequence
  core2_eHGT_450h
ctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaag

SEQ ID NO: 215 - DNA, 738 bp, Artificial Sequence
  3xcore2_eHGT_450h
ctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaagctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaagctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaag

SEQ ID NO: 216 - DNA, 245 bp, Artificial Sequence
  core3_eHGT_450h
cagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatca

SEQ ID NO: 217 - DNA, 735 bp, Artificial Sequence
  3xcore3_eHGT_450h
cagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatcacagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatcacagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatca

SEQ ID NO: 218 - DNA, 228 bp, Artificial Sequence
  core2_eHGT_452h
gtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctg

SEQ ID NO: 219 - DNA, 684 bp, Artificial Sequence
  3xcore2_eHGT_452h
gtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctggtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctggtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctg

SEQ ID NO: 220 - DNA, 250 bp, Artificial Sequence
  Core-eHGT_447h
actacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaactt

SEQ ID NO: 221 - DNA, 750 bp, Artificial Sequence
  3xCore-eHGT_447h
actacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaacttactacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaacttactacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaactt

SEQ ID NO: 222 - DNA, 249 bp, Artificial Sequence
  core2_eHGT_743m
agcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttctt

SEQ ID NO: 223 - DNA, 747 bp, Artificial Sequence
  3xcore2_eHGT_743m
agcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttctt

SEQ ID NO: 224 - DNA, 194 bp, Artificial Sequence
  core2_eHGT_779m
aagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaat

SEQ ID NO: 225 - DNA, 582 bp, Artificial Sequence
  3xcore2_eHGT_779m
aagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaataagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaataagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaat

SEQ ID NO: 226 - DNA, 200 bp, Artificial Sequence
  Core2_eHGT_780m
cagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttatt

SEQ ID NO: 227 - DNA, 600 bp, Artificial Sequence
  3xCore2_eHGT_780m
cagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattcagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattcagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttatt

SEQ ID NO: 228 - DNA, 2769 bp, Artificial Sequence
  CN2485
gcggccgcacgcgtagggaggaactaaagtccattcatatgtccatattctcaatttgggaaaaaatgtggctttgtcttcatcctcttttcctgagtgtataaggccagaagccaaaggcaagagcaggacattccaggagcctttatgattgctctaagtcacatctgctctcgagctcccattccactgtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctgccattctcttcggaggtgagcacctcctgctcgggaggaactgacagcccctttcagaaatgtgaaggcagtgaagtcccaaaccagaaacagtgtgaccacctaggctcacccaaggaccgtcctgtgcttctgggcaaccaatggatgtagggggttaggaggcagtggggattcgtcagagctcgggctgggcataaaagtcagggcagagccatctattgcttacatttgcttctgggatccagatctttcgaagctagccaccatgaatgcctcagagttccgcaggaggggaaaagagatggttgactacatggcaaactatatggaaggcatagaagggaggcaagtttatccagatgtagaaccggggtacttgcgacctttgattccagcagcagctcctcaggtaagtactagcagctacaatccagctaccattctgcttttattttatggttgggataaggctggattattctgagtccaagctaggcccttttgctaatcatgttcatacctcttatcttcctcccacaggaaccagatacatttgaagacataataaatgatgttgaaaagataatcatgcccggggttacacattggcatagcccatacttctttgcttactttcctactgcttcctcttaccctgcaatgctagcagatatgctgtgtggagctatagggtgtattgggttctcttgggctgctagccctgcttgtactgagctggagaccgtgatgatggattggcttggaaaaatgctggagctgcccaaagcattcctgaatgagaaagcaggagagggtggaggtgtaattcaaggatcagctagtgaagctacccttgtagcattgctggcagcacggactaaagtcatccatcgccttcaagcagcttcccctgagctcactcaggctgcgataatggaaaagctggttgcctactcttctgatcaagcacactcctcagttgaacgagcagggctcattggaggcgttaagctgaaggctatcccttctgatgggaattttgctatgagagcctcagctctgcaggaagcactggaacgtgataaagctgctggcttgatccctttcttcatggtggcaacgttgggaaccaccacatgctgttcctttgataatcttctggaggtgggccccatctgcaataaagaggacatctggcttcatgttgacgcagcctatgctggatctgcttttatttgtcctgaatttaggcatttgctcaatggtgtggagttcgctgatagtttcaatttcaatccgcataaatggctgcttgtcaattttgattgttccgccatgtgggttaagaaacgcactgatcttacaggagcttttcgcctggaccctacctatttgaagcattcccaccaagactctggactgattacggactatagacactggcagattcctctgggtagacgattccgctccctcaagatgtggtttgttttccgaatgtatggagtcaagggtctgcaagcatacatccggaaacatgttcagctttcacatgaatttgaatcacttgtccgacaagacccccgcttcgagatctgtgtagaggtcatattgggccttgtgtgtttccgactgaaagggagtaataaagtcaatgaggccctcttgcagagaatcaattcagcgaaaaagatacacttggtaccctgccatctgagagataaattcgtcctgagattcgcaatttgcagccggaccgtagaatctgctcatgtgcagcgggcctgggaacacatcaaagagttggcggcagatgttctccgagctgagagggagtagtgaggcgcgccgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 229 - DNA, 2871 bp, Artificial Sequence
  CN2486
gcggccgcacgcgtagggaggaactaaagtccattcatatgtccatattctcaatttgggaaaaaatgtggctttgtcttcatcctcttttcctgagtgtataaggccagaagccaaaggcaagagcaggacattccaggagcctttatgattgctctaagtcacatctgctctcgagctcccattccactgtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctgccattctcttcggaggtgagcacctcctgctcgggaggaactgacagcccctttcagaaatgtgaaggcagtgaagtcccaaaccagaaacagtgtgaccacctaggctcacccaaggaccgtcctgtgcttctgggcaaccaatggatgtagggggttaggaggcagtggggattcgtcagagctcgggctgggcataaaagtcagggcagagccatctattgcttacatttgcttctgggatccagatctttcgaagctagccaccatgaatgcctcagagttccgcaggaggggaaaagagatggttgactacatggcaaactatatggaaggcatagaagggaggcaagtttatccagatgtagaaccggggtacttgcgacctttgattccagcagcagctcctcaggtaagtactagcagctacaatccagctaccattctgcttttattttatggttgggataaggctggattattctgagtccaagctaggcccttttgctaatcatgttcatacctcttatcttcctcccacaggaaccagatacatttgaagacataataaatgatgttgaaaagataatcatgcccggggttacacattggcatagcccatacttctttgcttactttcctactgcttcctcttaccctgcaatgctagcagatatgctgtgtggagctatagggtgtattgggttctcttgggctgctagccctgcttgtactgagctggagaccgtgatgatggattggcttggaaaaatgctggagctgcccaaagcattcctgaatgagaaagcaggagagggtggaggtgtaattcaaggatcagctagtgaagctacccttgtagcattgctggcagcacggactaaagtcatccatcgccttcaagcagcttcccctgagctcactcaggctgcgataatggaaaagctggttgcctactcttctgatcaagcacactcctcagttgaacgagcagggctcattggaggcgttaagctgaaggctatcccttctgatgggaattttgctatgagagcctcagctctgcaggaagcactggaacgtgataaagctgctggcttgatccctttcttcatggtggcaacgttgggaaccaccacatgctgttcctttgataatcttctggaggtgggccccatctgcaataaagaggacatctggcttcatgttgacgcagcctatgctggatctgcttttatttgtcctgaatttaggcatttgctcaatggtgtggagttcgctgatagtttcaatttcaatccgcataaatggctgcttgtcaattttgattgttccgccatgtgggttaagaaacgcactgatcttacaggagcttttcgcctggaccctacctatttgaagcattcccaccaagactctggactgattacggactatagacactggcagattcctctgggtagacgattccgctccctcaagatgtggtttgttttccgaatgtatggagtcaagggtctgcaagcatacatccggaaacatgttcagctttcacatgaatttgaatcacttgtccgacaagacccccgcttcgagatctgtgtagaggtcatattgggccttgtgtgtttccgactgaaagggagtaataaagtcaatgaggccctcttgcagagaatcaattcagcgaaaaagatacacttggtaccctgccatctgagagataaattcgtcctgagattcgcaatttgcagccggaccgtagaatctgctcatgtgcagcgggcctgggaacacatcaaagagttggcggcagatgttctccgagctgagagggagggttcaggttacccgtatgatgtcccggattacgctggcagctacccatacgatgtacccgactatgccggcagttatccctacgacgtccctgactacgcatagtgaggcgcgccgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 230 - DNA, 2818 bp, Artificial Sequence
  CN2739
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaagagctcgattcagccgggagcttagggaggggaggtcacttcataagggcttggggggggagttggagccacgagtcgtccagccggagccccgtgtggctgtgctccggcctcagaagcatccccggatccagatctttcgaagctagccaccatgaatgcctcagagttccgcaggaggggaaaagagatggttgactacatggcaaactatatggaaggcatagaagggaggcaagtttatccagatgtagaaccggggtacttgcgacctttgattccagcagcagctcctcaggtaagtactagcagctacaatccagctaccattctgcttttattttatggttgggataaggctggattattctgagtccaagctaggcccttttgctaatcatgttcatacctcttatcttcctcccacaggaaccagatacatttgaagacataataaatgatgttgaaaagataatcatgcccggggttacacattggcatagcccatacttctttgcttactttcctactgcttcctcttaccctgcaatgctagcagatatgctgtgtggagctatagggtgtattgggttctcttgggctgctagccctgcttgtactgagctggagaccgtgatgatggattggcttggaaaaatgctggagctgcccaaagcattcctgaatgagaaagcaggagagggtggaggtgtaattcaaggatcagctagtgaagctacccttgtagcattgctggcagcacggactaaagtcatccatcgccttcaagcagcttcccctgagctcactcaggctgcgataatggaaaagctggttgcctactcttctgatcaagcacactcctcagttgaacgagcagggctcattggaggcgttaagctgaaggctatcccttctgatgggaattttgctatgagagcctcagctctgcaggaagcactggaacgtgataaagctgctggcttgatccctttcttcatggtggcaacgttgggaaccaccacatgctgttcctttgataatcttctggaggtgggccccatctgcaataaagaggacatctggcttcatgttgacgcagcctatgctggatctgcttttatttgtcctgaatttaggcatttgctcaatggtgtggagttcgctgatagtttcaatttcaatccgcataaatggctgcttgtcaattttgattgttccgccatgtgggttaagaaacgcactgatcttacaggagcttttcgcctggaccctacctatttgaagcattcccaccaagactctggactgattacggactatagacactggcagattcctctgggtagacgattccgctccctcaagatgtggtttgttttccgaatgtatggagtcaagggtctgcaagcatacatccggaaacatgttcagctttcacatgaatttgaatcacttgtccgacaagacccccgcttcgagatctgtgtagaggtcatattgggccttgtgtgtttccgactgaaagggagtaataaagtcaatgaggccctcttgcagagaatcaattcagcgaaaaagatacacttggtaccctgccatctgagagataaattcgtcctgagattcgcaatttgcagccggaccgtagaatctgctcatgtgcagcgggcctgggaacacatcaaagagttggcggcagatgttctccgagctgagagggagtagtgaggcgcgccgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 231 - DNA, 2920 bp, Artificial Sequence
  CN2740
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaagagctcgattcagccgggagcttagggaggggaggtcacttcataagggcttggggggggagttggagccacgagtcgtccagccggagccccgtgtggctgtgctccggcctcagaagcatccccggatccagatctttcgaagctagccaccatgaatgcctcagagttccgcaggaggggaaaagagatggttgactacatggcaaactatatggaaggcatagaagggaggcaagtttatccagatgtagaaccggggtacttgcgacctttgattccagcagcagctcctcaggtaagtactagcagctacaatccagctaccattctgcttttattttatggttgggataaggctggattattctgagtccaagctaggcccttttgctaatcatgttcatacctcttatcttcctcccacaggaaccagatacatttgaagacataataaatgatgttgaaaagataatcatgcccggggttacacattggcatagcccatacttctttgcttactttcctactgcttcctcttaccctgcaatgctagcagatatgctgtgtggagctatagggtgtattgggttctcttgggctgctagccctgcttgtactgagctggagaccgtgatgatggattggcttggaaaaatgctggagctgcccaaagcattcctgaatgagaaagcaggagagggtggaggtgtaattcaaggatcagctagtgaagctacccttgtagcattgctggcagcacggactaaagtcatccatcgccttcaagcagcttcccctgagctcactcaggctgcgataatggaaaagctggttgcctactcttctgatcaagcacactcctcagttgaacgagcagggctcattggaggcgttaagctgaaggctatcccttctgatgggaattttgctatgagagcctcagctctgcaggaagcactggaacgtgataaagctgctggcttgatccctttcttcatggtggcaacgttgggaaccaccacatgctgttcctttgataatcttctggaggtgggccccatctgcaataaagaggacatctggcttcatgttgacgcagcctatgctggatctgcttttatttgtcctgaatttaggcatttgctcaatggtgtggagttcgctgatagtttcaatttcaatccgcataaatggctgcttgtcaattttgattgttccgccatgtgggttaagaaacgcactgatcttacaggagcttttcgcctggaccctacctatttgaagcattcccaccaagactctggactgattacggactatagacactggcagattcctctgggtagacgattccgctccctcaagatgtggtttgttttccgaatgtatggagtcaagggtctgcaagcatacatccggaaacatgttcagctttcacatgaatttgaatcacttgtccgacaagacccccgcttcgagatctgtgtagaggtcatattgggccttgtgtgtttccgactgaaagggagtaataaagtcaatgaggccctcttgcagagaatcaattcagcgaaaaagatacacttggtaccctgccatctgagagataaattcgtcctgagattcgcaatttgcagccggaccgtagaatctgctcatgtgcagcgggcctgggaacacatcaaagagttggcggcagatgttctccgagctgagagggagggttcaggttacccgtatgatgtcccggattacgctggcagctacccatacgatgtacccgactatgccggcagttatccctacgacgtccctgactacgcatagtgaggcgcgccgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 232 - DNA, 2751 bp, Artificial Sequence
  CN2765
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaagagctcgggctgggcataaaagtcagggcagagccatctattgcttacatttgcttctgggatccagatctttcgaagctagccaccatgaatgcctcagagttccgcaggaggggaaaagagatggttgactacatggcaaactatatggaaggcatagaagggaggcaagtttatccagatgtagaaccggggtacttgcgacctttgattccagcagcagctcctcaggtaagtactagcagctacaatccagctaccattctgcttttattttatggttgggataaggctggattattctgagtccaagctaggcccttttgctaatcatgttcatacctcttatcttcctcccacaggaaccagatacatttgaagacataataaatgatgttgaaaagataatcatgcccggggttacacattggcatagcccatacttctttgcttactttcctactgcttcctcttaccctgcaatgctagcagatatgctgtgtggagctatagggtgtattgggttctcttgggctgctagccctgcttgtactgagctggagaccgtgatgatggattggcttggaaaaatgctggagctgcccaaagcattcctgaatgagaaagcaggagagggtggaggtgtaattcaaggatcagctagtgaagctacccttgtagcattgctggcagcacggactaaagtcatccatcgccttcaagcagcttcccctgagctcactcaggctgcgataatggaaaagctggttgcctactcttctgatcaagcacactcctcagttgaacgagcagggctcattggaggcgttaagctgaaggctatcccttctgatgggaattttgctatgagagcctcagctctgcaggaagcactggaacgtgataaagctgctggcttgatccctttcttcatggtggcaacgttgggaaccaccacatgctgttcctttgataatcttctggaggtgggccccatctgcaataaagaggacatctggcttcatgttgacgcagcctatgctggatctgcttttatttgtcctgaatttaggcatttgctcaatggtgtggagttcgctgatagtttcaatttcaatccgcataaatggctgcttgtcaattttgattgttccgccatgtgggttaagaaacgcactgatcttacaggagcttttcgcctggaccctacctatttgaagcattcccaccaagactctggactgattacggactatagacactggcagattcctctgggtagacgattccgctccctcaagatgtggtttgttttccgaatgtatggagtcaagggtctgcaagcatacatccggaaacatgttcagctttcacatgaatttgaatcacttgtccgacaagacccccgcttcgagatctgtgtagaggtcatattgggccttgtgtgtttccgactgaaagggagtaataaagtcaatgaggccctcttgcagagaatcaattcagcgaaaaagatacacttggtaccctgccatctgagagataaattcgtcctgagattcgcaatttgcagccggaccgtagaatctgctcatgtgcagcgggcctgggaacacatcaaagagttggcggcagatgttctccgagctgagagggagtagtgaggcgcgccgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 233 - DNA, 2853 bp, Artificial Sequence
  CN2766
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaagagctcgggctgggcataaaagtcagggcagagccatctattgcttacatttgcttctgggatccagatctttcgaagctagccaccatgaatgcctcagagttccgcaggaggggaaaagagatggttgactacatggcaaactatatggaaggcatagaagggaggcaagtttatccagatgtagaaccggggtacttgcgacctttgattccagcagcagctcctcaggtaagtactagcagctacaatccagctaccattctgcttttattttatggttgggataaggctggattattctgagtccaagctaggcccttttgctaatcatgttcatacctcttatcttcctcccacaggaaccagatacatttgaagacataataaatgatgttgaaaagataatcatgcccggggttacacattggcatagcccatacttctttgcttactttcctactgcttcctcttaccctgcaatgctagcagatatgctgtgtggagctatagggtgtattgggttctcttgggctgctagccctgcttgtactgagctggagaccgtgatgatggattggcttggaaaaatgctggagctgcccaaagcattcctgaatgagaaagcaggagagggtggaggtgtaattcaaggatcagctagtgaagctacccttgtagcattgctggcagcacggactaaagtcatccatcgccttcaagcagcttcccctgagctcactcaggctgcgataatggaaaagctggttgcctactcttctgatcaagcacactcctcagttgaacgagcagggctcattggaggcgttaagctgaaggctatcccttctgatgggaattttgctatgagagcctcagctctgcaggaagcactggaacgtgataaagctgctggcttgatccctttcttcatggtggcaacgttgggaaccaccacatgctgttcctttgataatcttctggaggtgggccccatctgcaataaagaggacatctggcttcatgttgacgcagcctatgctggatctgcttttatttgtcctgaatttaggcatttgctcaatggtgtggagttcgctgatagtttcaatttcaatccgcataaatggctgcttgtcaattttgattgttccgccatgtgggttaagaaacgcactgatcttacaggagcttttcgcctggaccctacctatttgaagcattcccaccaagactctggactgattacggactatagacactggcagattcctctgggtagacgattccgctccctcaagatgtggtttgttttccgaatgtatggagtcaagggtctgcaagcatacatccggaaacatgttcagctttcacatgaatttgaatcacttgtccgacaagacccccgcttcgagatctgtgtagaggtcatattgggccttgtgtgtttccgactgaaagggagtaataaagtcaatgaggccctcttgcagagaatcaattcagcgaaaaagatacacttggtaccctgccatctgagagataaattcgtcctgagattcgcaatttgcagccggaccgtagaatctgctcatgtgcagcgggcctgggaacacatcaaagagttggcggcagatgttctccgagctgagagggagggttcaggttacccgtatgatgtcccggattacgctggcagctacccatacgatgtacccgactatgccggcagttatccctacgacgtccctgactacgcatagtgaggcgcgccgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 234 - DNA, 1748 bp, Artificial Sequence
  CN2514
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactggagctcgattcagccgggagcttagggaggggaggtcacttcataagggcttggggggggagttggagccacgagtcgtccagccggagccccgtgtggctgtgctccggcctcagaagcatccccggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 235 - DNA, 2068 bp, Artificial Sequence
  CN2555
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactggagctcgattcagccgggagcttagggaggggaggtcacttcataagggcttggggggggagttggagccacgagtcgtccagccggagccccgtgtggctgtgctccggcctcagaagcatccccggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 236 - DNA, 2636 bp, Artificial Sequence
  CN2907
gcggccgcacgcgtgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccacgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccacgcccacccctcctccagctctcaggagtcccattcccttcctcctctgtcgcccacacagcccccaccacctggccgcccacctgaaaaacttttccccaagttcttgagaagtcgcgtaggatccaggagagaaaaatcaaagtaataaaacctcatttaattcctaacggagggcagagctgcgagctgcaattatcttaatgctgagctatttttactctctctccatctctgtcttctgcctttctctgttgtgaggttcgctgtctctctctctgcctccctttgtctctccatctctcaaccatgttttctgtctctctttgctttctcctctctccccccaatctctctctcacgtcctgtctctgtgcagtctcttcctcctccttcatgtctgtccctctgtcagccagtcttccccacgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 237 - DNA, 2018 bp, Artificial Sequence
  CN2909
gcggccgcacgcgtgtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggcgtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggcgtggaggactttgagcttaaccctattaaatttaatctaatatattcagcagcctgtcacaatcttgttaaatctttattccgtcacccaccttattagggggccctcccactttagtcatcagcagattagtaagcatgccatttagatgtcatctaagttaatgaacctgactgagccagagtcacatggcagagtcacagaaactccttctgagctggcgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 238 - DNA, 2057 bp, Artificial Sequence
  CN2921
gcggccgcacgcgtggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaaggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaaggccacccccttccaccttgtgttcccgtgtcgtgtcgggtcctactcctcccatttctttgcccctgaagtgctgagagggggagagggtggagcccagccctccccctgcccactctagctgtcaatctgctgtctggcagaaagacatgaagcctcgtgacaactgtgggcaactttcccctgccccgcctcccgcccctgccctgccctcttcccctctgctccctgtcaagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 239 - DNA, 2036 bp, Artificial Sequence
  CN2982
gcggccgcacgcgtgtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctggtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctggtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 240 - DNA, 1934 bp, Artificial Sequence
  CN3044
gcggccgcacgcgtaagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaataagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaataagtttgagacaaattaaggaaaagcgggttgggaggagtctgaggctgtgggtggctgcctcgagaggctgctcacaacctgacatgtttcagatcttttgacacggtcatgcaagcaacagaaacttatatggtttttagtctcccaggtagcagggaaatgatgatgtgggaggattaggggggggaaaatgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 241 - DNA, 2099 bp, Artificial Sequence
  CN3038
gcggccgcacgcgtagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 242 - DNA, 1958 bp, Artificial Sequence
  CN3344
gcggccgcacgcgtatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaaatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaaatacaatagattaattttatctaacattaatagctggatgtataagaaacacccactctctagaaattaagacatgtcaccatagtcttatgacaagagtgagtctcttatttttgaactttctattgaacagttctcaccatcttaaatcaccctttgttatgagtttttaagtgtcttaagtagagaaggttaagaaagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 243 - DNA, 1958 bp, Artificial Sequence
  CN3281
gcggccgcacgcgtcagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattcagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattcagataaagtctggagccgggcccaggtacctgaggctcagagatgagcccgcagcgaagctgacaactgactcattcctccgacactctcccaccctcccagctaaggaggtgtctgatgtgaacatgttctgcctctttgttgcctgctgttgggggcgagggctttgggtggattttgaagagcaattatgcttattgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 244 - DNA, 2108 bp, Artificial Sequence
  CN3346
gcggccgcacgcgtactacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaacttactacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaacttactacttaattttctgtgaaactaacacttcttttctaattgcccttgacttcacattctttagcccattcttcgttactaatccattcttgctagttcctcctacattcctattgttcgtgttaaagatgacttttctattcacctgtgtaagtcttgctgcctcctacactgattaatttagctagattgaaaaggaggactagtgcttcctccataaaactccaattaatcaatcattttagaacttgagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 245 - DNA, 2116 bp, Artificial Sequence
  CN3566
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgtgcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagaggcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagaggcccttctttctgtgaagggtgacacacctcctagaaagaaatctgactcccctcattgtgtttttaaaataactctcattattcctcctctgaaggttattttcaaactctttataactcgatttttttttaaatgcaaagacaaggaatcaaatctgccaactttctatcagaggagctcgattcagccgggagcttagggaggggaggtcacttcataagggcttggggggggagttggagccacgagtcgtccagccggagccccgtgtggctgtgctccggcctcagaagcatccccggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 246 - DNA, 2090 bp, Artificial Sequence
  CN2912
gcggccgcacgcgtctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaagctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaagctgcaggagcgcccttccgctggagggtgggaggactgtctaaggcagtcacagcaaggagaggcacggtgtgggcactgctgctatttctaacctgctggagtgaagggaaccttgactacttgaaaagcaggctgattgtcaggggcggaaccgaactcagagctgtcagggccgtcacatggaaagtgcctggcagggggcggcgacagggggcttgggctgacagggctgtgattttgaaaggagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 247 - DNA, 2087 bp, Artificial Sequence
  CN2913
gcggccgcacgcgtcagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatcacagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatcacagggggcggcgacagggggcttgggctgacagggctgtgattttgaaagaaacgtctccaagaaagtgatttaaagtaggagagaaatattattattagataatggctgtcaggttattttttgatgtgttttctgagtatttcataatcatcttgcgcttagatggtaagcccttcctggctaggggccgtgtcccatcccatgcctctgtggctttcaccttcgggtgtgcaacaggtatcagagctcgggctgggcataaaagtcagggcagagccatctattgcttacatttgcttctgggatccagatctttcgaagctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccaagctgagcaaagaccccaacgagaagcgcgatcacatggtcctgctggagttcgtgaccgccgccgggatcactctcggcatggacgagctgtacaagtaagtcgacggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcacgtgcggaccgagcggccgc

SEQ ID NO: 248 - DNA, 2374 bp, Artificial Sequence
  CN2966
gcggccgcacgcgttccgtaaagagcacaaaacccaaatatttgggtgcatgggagatagatttcaactaaatacttttaatcatgcaattccaatagcacttaaattttttcaaggcatgtataaagggagaatagactaattcaatttcaaagtgcaagaaaaacatcaaaaactaaattcagaaagtttgcatgggaagaaaaaaaaacacaaattaatctcacttgtgattattctaaatacatttgaacaagcagcagctagtagtacatacaaagaaatagaaaacatgaagaccaggggagtttaattatatggttgcaaaaatggttcaacattgcaaagttgttaatactcagtggatggggagggagctgtgcaattatctccacaggtacagaaaaggcacttagtaaaatgtagccaactctttggcttctaaaaaaggagaaaatggtttacaaaaacaagcctccctggatgcttccttaatttgttcttattcataatgggggaacataaagtgaacttctgcacaccagaagtcaggttgtttgcttgctaataccattcccactcaatatcccatggaagttggatgactcgacctgcatcccaaggagtagttaccatcactaatgaggagaagcatttggaaggaagacagaattctgtagaccaaacgaagacctggaggctagtggaaagagctcgggctgggcataaaagtcagggcagagccatctattgcttacatttgcttctgggatccagatctttcgaagctagcaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaatgaggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 249 - DNA, 2279 bp, Artificial Sequence
  CN2203
gcggccgcacgcgccggtaccgaagctacccctaacacactattctacacacagaaaatgctcttcactaggaagctacccctaacacactattctacacacagaaaatgctcttcactaggaagctacccctaacacactattctacacacagaaaatgctcttcactagacgcgttagcaggagtacatcctcctctgttagaaaaacaactctgcggttctgggtcacttttcccatctttgtatagaaaagtaccgattattattgttcccccgggtgccttctccagcttaaaggaaaattccagccccagagatcttccgcagtgagtgacagctccttaatgtgaggagcagcgtgggcgtggtgggtgggatgtcagagccatcaaagccctcatttctgatccctctgtgatgtcggggttccctggcacgcccactgtcatgcctcagcacattggcatctgagagaggaaaggtgcagctgacaccctgacccccttctccccgacacggggtcagacagggtcccgcggtggcctggttgtgcagcactgacatgttgcctgggaacgaagagctcgattcagccgggagcttagggaggggaggtcacttcataagggcttggggggggagttggagccacgagtcgtccagccggagccccgtgtggctgtgctccggcctcagaagcatccccggatccgccaccatggtgcccaagaagaagaggaaagtctccaacctgctgactgtgcaccaaaacctgcctgccctccctgtggatgccacctctgatgaagtcaggaagaacctgatggacatgttcagggacaggcaggccttctctgaacacacctggaagatgctcctgtctgtgtgcagatcctgggctgcctggtgcaagctgaacaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggactaagaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 250 - DNA, 1922 bp, Artificial Sequence
  CN2700
gcggccgcacgcgtagggaggaactaaagtccattcatatgtccatattctcaatttgggaaaaaatgtggctttgtcttcatcctcttttcctgagtgtataaggccagaagccaaaggcaagagcaggacattccaggagcctttatgattgctctaagtcacatctgctctcgagctcccattccactgtgaaaaatacctagtcttgaactttggagttccaaattccacatgatcgacttcctcaaggcttttcacttgacgaacaaattgcatgccaagaaatcgtcaaaattctatcaagcttaattatgcaatttctgtctataaatgacatacctcctgtaccctatgacacccacctatgcactaataagatgtgacagctgcatcaccaacaaagcctcccccgcctgccattctcttcggaggtgagcacctcctgctcgggaggaactgacagcccctttcagaaatgtgaaggcagtgaagtcccaaaccagaaacagtgtgaccacctaggctcacccaaggaccgtcctgtgcttctgggcaaccaatggatgtagggggttaggaggcagtggggattcgtcagagctcgggctgggcataaaagtcagggcagagccatctattgcttacatttgcttctgggatccagatctttcgaagctagcgccaccatggtgagcaagggcgaggagaccacaatgggcgtaatcaagcccgacatgaagatcaagctgaagatggagggcaacgtgaatggccacgccttcgtgatcgagggcgagggcgagggcaagccctacgacggcaccaacaccatcaacctggaggtgaaggagggagcccccctgcccttctcctacgacattctgaccaccgcgttcgcctacggcaacagggccttcaccaagtaccccgacgacatccccaactacttcaagcagtccttccccgagggctactcttgggagcgcaccatgaccttcgaggacaagggcatcgtgaaggtgaagtccgacatctccatggaggaggactccttcatctacgagatacacctcaagggcgagaacttcccccccaacggccccgtgatgcagaagaagaccaccggctgggacgcctccaccgagaggatgtacgtgcgcgacggcgtgctgaagggcgacgtcaagcacaagctgctgctggagggcggcggccaccaccgcgttgacttcaagaccatctacagggccaagaaggcggtgaagctgcccgactatcactttgtggaccaccgcatcgagatcctgaaccacgacaaggactacaacaaggtgaccgtttacgagagcgccgtggcccgcaactccaccgacggcatggacgagctgtacaagtaatgaggcgcgccgcggccgcgaattcgatatcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggctcgagagatcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgagatctcacgtgcggaccgagcggccgc

SEQ ID NO: 251 - DNA, 93 bp, Artificial Sequence
  3xHA tag
tacccgtatgatgtcccggattacgctggcagctacccatacgatgtacccgactatgccggcagttatccctacgacgtccctgactacgca

(19) United States
(12) Patent Application Publication
(10) Pub. No.: US 2023/0302158 A1
(43) Pub. Date: Sep. 28, 2023

(54) ARTIFICIAL EXPRESSION CONSTRUCTS FOR MODULATING GENE EXPRESSION IN STRIATAL NEURONS

(71) Applicant: Allen Institute, Seattle, WA (US)

(72) Inventors: Tanya Daigle, Lake Forest Park, WA (US); Edward Sebastian Lein, Mercer Island, WA (US); Boaz P. Levi, Seattle, WA (US); John K. Mich, Seattle, WA (US); Bosiljka Tasic, Seattle, WA (US); Jonathan Ting, Lake Forest Park, WA (US); Hongkui Zeng, Seattle, WA (US)

(73) Assignee: Allen Institute, Seattle, WA (US)

(21) Appl. No.: 18/040,961

(22) PCT Filed: Aug. 13, 2021
(86) PCT No.: PCT/US2021/045995

§ 371 Date: Feb. 7, 2023

Related U.S. Application Data
(60) Provisional application No. 63/066,008, filed on Aug. 14, 2020.

(51) Int. Cl. A61K 48/00; C12N 9/88; C12N 15/86; C12N 15/113

(52) U.S. Cl. CPC A61K 48/0075; C12N 9/88; C12Y 401/01028; C12N 15/86; C12N 15/113; A61K 48/0058; C12N 2750/14144; C12N 2310/14

(57) ABSTRACT

Artificial expression constructs for modulating gene expression in striatal neurons are described. The artificial expression constructs can be used to express heterologous genes in striatal neurons including in striatal medium spiny neuron-pan, striatal medium spiny neuron-indirect pathway, striatal medium spiny neuron-direct pathway, striatal interneuron-cholinergic, and Drd3+ medium spiny neurons in olfactory tubercle. The artificial expression constructs can be used for many purposes, including to research and treat movement disorders such as Parkinson's disease and Huntington's disease.

### CROSS-REFERENCE TO RELATED APPLICATIONS

This is the 371 National Phase of co-pending international application no. PCT/US2021/045995, filed Aug. 13, 2021, which claims priority to U.S. Provisional Patent Application No. 63/066,008 filed on Aug. 14, 2020, each of which is incorporated herein by reference in its entirety as if fully set forth herein.

### STATEMENT REGARDING FEDERALLY SPONSORED RESEARCH OR DEVELOPMENT

This invention was made with government support under MH114126 awarded by the National Institutes of Health. The government has certain rights in the invention.

### REFERENCE TO SEQUENCE LISTING

The Sequence Listing associated with this application is provided in text format in lieu of a paper copy and is hereby incorporated by reference into the specification. The name of the text file containing the Sequence Listing is 2U76691.txt. The text file is 367 KB, was created on Feb. 1, 2023 and is being submitted electronically via Patent Center.

### FIELD OF THE DISCLOSURE

The current disclosure provides artificial expression constructs for modulating gene expression in striatal neurons. The artificial expression constructs can be used for many purposes, including in the research and treatment of movement disorders.

### BACKGROUND OF THE DISCLOSURE

To fully understand the biology of the brain, different cell types need to be distinguished and defined and, to further study them, artificial expression constructs that can label and perturb them need to be identified. In mouse, recombinase driver lines have been used to great effect to label cell populations that share marker gene expression. However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans.

A movement disorder is a neurological disturbance that involves one or more muscles or muscle groups. Movement disorders affect a significant portion of the population, causing disability as well as distress. Movement disorders include Parkinson's disease, Huntington's chorea, progressive supranuclear palsy, Wilson's disease, Tourette's syndrome, epilepsy, tardive dyskinesia, and various chronic tremors, tics and dystonias.

Parkinson's disease is a movement disorder of increasing occurrence in aging populations. It affects one percent of the population over the age of 60 in the United States, such that the cumulative lifetime risk of an individual developing the disease is 1 in 40. Symptoms include pronounced tremor of the extremities, bradykinesia, rigidity and postural change. A perceived pathophysiological cause of Parkinson's disease is progressive destruction of dopamine producing cells in the basal ganglia which include the pars compacta of the substantia nigra. Parkinson's disease is a progressive disorder which can begin with mild limb stiffness and infrequent tremors and progress over a period of ten or more years to frequent tremors and memory impairment, to uncontrollable tremors and dementia.

Huntington's disease (HD) is a dominantly inherited neurodegenerative genetic disorder that affects muscle coordination and leads to cognitive decline and psychiatric problems. HD is due to mutations in the gene encoding for huntingtin, and it is the most common genetic cause of abnormal involuntary writhing movements called chorea. The most prominent early effects in HD are in a part of the basal ganglia called the neostriatum, which is composed of the caudate nucleus and putamen. Symptoms of the disease can vary between individuals, but usually progress predictably. The earliest symptoms are often subtle problems with mood or cognition, followed by a general lack of coordination and an unsteady gait. In advanced stages of the disease, uncoordinated, jerky body movements become more apparent, along with a decline in mental abilities, as well as behavioral and psychiatric problems. Physical abilities are gradually impeded until coordinated movement becomes very difficult, and mental abilities generally decline into dementia. Although the genetic basis of the pathology is well known there is not yet a cure for HD.

### SUMMARY OF THE DISCLOSURE

The current disclosure provides artificial expression constructs that drive gene expression in striatal neurons. The artificial expression constructs can be used for many purposes, including in the research and treatment of movement disorders.

Particular embodiments of the artificial expression constructs utilize the following enhancers to drive gene expression within targeted central nervous system cell populations as follows:
striatal medium spiny neuron-pan: eHGT_608h, eHGT_609h, eHGT_633h, eHGT_634h, eHGT_635h, eHGT_636h, eHGT_351h, eHGT_367h, eHGT_450h, eHGT_447h, eHGT_744m, eHGT_782m, eHGT_785m, and eHGT_441h;
striatal medium spiny neuron-indirect pathway: eHGT_612h, eHGT_613h, eHGT_614h, eHGT_617h, eHGT_618h, eHGT_619h, eHGT_620h, eHGT_442h, eHGT_444h, eHGT_445h, eHGT_452h, and eHGT_784m;
striatal medium spiny neuron-direct pathway: eHGT_610h, eHGT_611h, eHGT_615h, eHGT_616h, eHGT_627h, eHGT_628h, eHGT_629h, eHGT_446h, eHGT_779m, eHGT_780m, eHGT_781m, and eHGT_783m;
striatal interneuron-cholinergic: eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_746m, eHGT_747m, eHGT_748m, eHGT_749m, eHGT_750m, and eHGT_751m;
Drd3+ medium spiny neurons in olfactory tubercle: eHGT_621h.

In particular embodiments, the artificial enhancer elements include a concatenated core of an enhancer. Examples include a core or concatenated core of eHGT_367h, eHGT_441h, eHGT_445h, eHGT_444h, eHGT_452h, eHGT_779m, eHGT_743m, eHGT_621h, eHGT_780m, eHGT_447h, eHGT_351h, and/or eHGT_450h. These artificial enhancer elements can provide higher levels and more rapid onset of transgene expression compared to a single full length original (native) enhancer.

In particular embodiments, the enhancer core includes the sequence as set forth in any one of SEQ ID NOs: 202, 204, 206, 208, 210, 212, 214, 216, 218, 220, 222, 224, and 226. In particular embodiments, these cores are concatenated and have 2, 3, 4, 5, 6, 7, 8, 9, or 10 copies of the core sequence. SEQ ID NOs: 203, 205, 207, 209, 211, 213, 215, 217, 219, 221, 223, 225, and 227 provide three-copy concatemers of the described enhancer cores.

Particular embodiments of the artificial expression constructs utilize core2_eHGT_367h, 3×core2_eHGT_367h, 3×core_eHGT_441h, 3×Core-eHGT_447h, and/or 3×core2_eHGT_351h to drive gene expression within striatal medium spiny neuron-pan.

Particular embodiments of the artificial expression constructs utilize 3×core2_eHGT_445h, 3×Core2_eHGT_444h, 3×core2_eHGT_452h, 3×core2_eHGT_450h, and/or 3×core3_eHGT_450h to drive gene expression within striatal medium spiny neuron-indirect pathway.

Particular embodiments of the artificial expression constructs utilize 3×core2_eHGT_779m and/or 3×Core2_eHGT_780m to drive gene expression within striatal medium spiny neuron-direct pathway.

Particular embodiments of the artificial expression constructs utilize 3×core2_eHGT_743m to drive gene expression within striatal interneuron-cholinergic.

Particular embodiments of the artificial expression constructs utilize 3×core-eHGT_621h to drive gene expression within Drd3+ medium spiny neurons in olfactory tubercle.

Particular embodiments provide artificial expression constructs including the features of vectors described herein including vectors: CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, and CN2700.

### BRIEF DESCRIPTION OF THE FIGURES

Some of the drawings submitted herein are better understood in color. Applicant considers the color versions of the drawings as part of the original submission and reserves the right to present color images of the drawings in later proceedings.

FIG. 1: Putative enhancers were selected from the Icahn School of Medicine Brain Open Chromatin Atlas (BOCA) associated with Fullard et al, 2018 Genome Research (doi:10.1101/gr.232488.117) or the snATAC-seq dataset available through the open access web portal called CATIas (Cis-element Atlas): catlas.org/mousebrain/#!/. These regions were cloned upstream of a minimal promoter in an AAV genomic backbone, which was used to generate recombinant adeno-associated viral vectors (rAAVs) or plasmid AAVs (pAAVs). These viral tools were delivered retro-orbitally to label specific striatal populations. In cells with a matching cell type, enhancers recruit their cognate transcription factors to drive cell type-specific expression. In other cells, viral genomes are present, but transcripts are not expressed.

FIGS. 2A,2B. (2A) Fluorescent reporter expression in mouse brain tissue following retro-orbital injection of CN2514 serotype PHPeB. Scale bar: 1 mm. (2B) Higher magnification view of SYFP2 expression in the boxed dorsal striatum region from FIG. 2A. Scale bar: 500 microns. Abbreviations: Ctx-cortex, Hipp-hippocampus, Str-striatum, SNr-substantia nigra pars reticulata, GPe-globus pallidus external. CP-caudoputamen, CB-cerebellum.

FIGS. 3A-3G. A C57BI/6J (wild-type) mouse was injected with 1.0E+12 viral genomes of CN2514 serotype PHP.eB virus via the retro-orbital sinus. Representative two-photon tomography (TissueCyte) images of the native SYFP2 fluorescence in coronal brain sections are shown four weeks post-injection. FIGS. 3A, 3C, 3E show the full coronal section at different planes in the brain (moving anterior to posterior) and (3B, 3D, 3F) show higher magnification view of the boxed areas in the corresponding FIGS. 3A, 3C, and 3E, respectively. Abbreviations: PALd=dorsal pallidum (3G) TissueCyte imaging datasets were processed using an established informatics pipeline (Oh et al., Nature 2014, 508: 207-214) and registered to the CCFv3.0 (Wang et al., Cell 2020, 181(4):936-953.e20). Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 4A, 4B. (4A) Fluorescent reporter expression in mouse brain tissue following retro-orbital injection of CN2555 serotype PHPeB. Scale bar: 1 mm. (4B) Higher magnification view of SYFP2 expression in the boxed dorsal striatum region from FIG. 4A. Scale bar: 500 microns.

FIGS. 5A-5G. A C57BI/6J (wild-type) mouse was injected with 1.0E+12 viral genomes of CN2555 serotype PHP.eB virus via the retro-orbital sinus. Representative two-photon tomography (TissueCyte) images of the native SYFP2 fluorescence in coronal brain sections are shown four weeks post-injection. FIGS. 5A, 5C, 5E show the full coronal section at different planes in the brain (moving anterior to posterior) and (5B, 5D, 5F) show higher magnification view of the boxed areas in the corresponding FIGS. 5A, 5C, 5E, respectively. (5G) TissueCyte imaging datasets were processed using an established informatics pipeline (Oh et al., Nature 2014, 508: 207-214) and registered to the CCFv3.0 (Wang et al., Cell 2020, 181(4):936-953.e20).

Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 6A, 6B. (6A) Fluorescent reporter expression in mouse brain tissue following retro-orbital injection of CN2025 serotype PHPeB. Scale bar: 1 mm. (6B) Higher magnification view of SYFP2 expression in the boxed dorsal striatum region from FIG. 6A. Scale bar: 100 microns.

FIGS. 7A-7G. A C57BI/6J (wild-type) mouse was injected with 1.0E+12 viral genomes of CN2025 serotype PHP.eB virus via the retro-orbital sinus. Representative two-photon tomography (TissueCyte) images of the native SYFP2 fluorescence in coronal brain sections are shown four weeks post-injection. FIGS. 7A, 7C, 7E show the full coronal section at different planes in the brain (moving anterior to posterior) and (7B, 7D, 7F) show higher magnification view of the boxed areas in the corresponding FIGS. 7A, 7C, and 7E, respectively. (7G) TissueCyte imaging datasets were processed using an established informatics pipeline (Oh et al., Nature 2014, 508: 207-214) and registered to the CCFv3.0 (Wang et al., Cell 2020, 181(4):936-953.e20).

Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 8A-8C. (8A) Fluorescent reporter expression in mouse brain tissue following retro-orbital injection of CN2233 serotype PHPeB. Scale bar: 1 mm. (8B) Higher magnification view of SYFP2 expression in the dorsal striatum region from FIG. 8A. Scale bar: 100 microns. (8C) Fluorescent reporter expression in rat brain tissue following injection of CN2233 serotype PHPeB into the lateral ventricle at P2 and tissue harvest at P19. Scale bar: 1 mm. Abbreviation: OT-olfactory tubercle.

FIGS. 9A-9G. A C57BI/6J (wild-type) mouse was injected with 1.0E+12 viral genomes of CN2233 serotype PHP.eB virus via the retro-orbital sinus. Representative two-photon tomography (TissueCyte) images of the native SYFP2 fluorescence in coronal brain sections are shown four weeks post-injection. FIGS. 9A, 9C, 9E show the full coronal section at different planes in the brain (moving anterior to posterior) and (9B, 9D, 9F) show higher magnification view of the boxed areas in the corresponding FIGS. 9A, 9C, 9E, respectively. (9G) TissueCyte imaging datasets were processed using an established informatics pipeline (Oh et al., Nature 2014, 508: 207-214) and registered to the CCFv3.0 (Wang et al., Cell 2020, 181(4):936-953.e20).

Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 10A, 10B. (10A) Fluorescent reporter expression in mouse brain tissue following retro-orbital injection of CN2236 serotype PHPeB. Scale bar: 1 mm. (10B) Higher magnification view of SYFP2 expression in the boxed dorsal striatum region from FIG. 10A. Scale bar: 100 microns.

FIGS. 11A-11G. A C57Bl/6J (wild-type) mouse was injected with 1.0E+12 viral genomes of CN2236 serotype PHP.eB virus via the retro-orbital sinus. Representative two-photon tomography (TissueCyte) images of the native SYFP2 fluorescence in coronal brain sections are shown four weeks post-injection. FIGS. 11A, 11C, 11E show the full coronal section at different planes in the brain (moving anterior to posterior) and (11B, 11D, 11F) show higher magnification view of the boxed areas in the corresponding FIGS. 11A, 11C, 11E, respectively. (11G) TissueCyte imaging datasets were processed using an established informatics pipeline (Oh et al., Nature 2014, 508: 207-214) and registered to the CCFv3.0 (Wang et al., Cell 2020, 181(4):936-953.e20). Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 12A, 12B. (12A) Fluorescent reporter expression in mouse brain tissue following retro-orbital injection of CN2237 serotype PHPeB. Scale bar: 1 mm. (12B) Higher magnification view of SYFP2 expression in the boxed dorsal striatum region from FIG. 12A. Scale bar: 500 microns.

FIGS. 13A-13G. A C57Bl/6J (wild-type) mouse was injected with 1.0E+12 viral genomes of CN2237 serotype PHP.eB virus via the retro-orbital sinus. Representative two-photon tomography (TissueCyte) images of the native SYFP2 fluorescence in coronal brain sections are shown four weeks post-injection. FIGS. 13A, 13C, 13E show the full coronal section at different planes in the brain (moving anterior to posterior) and (13B, 13D, 13F) show higher magnification view of the boxed areas in the corresponding FIGS. 13A, 13C, 13E, respectively. (13G) TissueCyte imaging datasets were processed using an established informatics pipeline (Oh et al., Nature 2014, 508: 207-214) and registered to the CCFv3.0 (Wang et al., Cell 2020, 181(4):936-953.e20). Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIG. 14A-14C: In vivo stereotaxic injection of CN2700 (packaged as serotype PHP.eB) into macaque caudate and putamen region. (14A) anti-DARPP-32 signal reveals Basal ganglia brain structures. (14B) Fluorescent reporter mTFP1 expression was detected in caudate and putamen. Scale bar: 1 mm. (14C) Higher magnification view of mTFP1 expression in the boxed putamen region from FIG. 14B. Scale bar: 500 microns. Abbreviations: GPi-globus pallidus internal segment.

FIGS. 15A-15C. (15A) Fluorescent reporter expression in mouse brain tissue following retro-orbital injection of CN2609 serotype PHPeB. Scale bar: 1 mm. (15B) Higher magnification view of SYFP2 expression in the dorsal striatum region (box) from FIG. 19A showing neuronal profiles. Scale bar: 250 microns. (15C) Higher magnification view of SYFP2 signal in the white boxed region in FIG. 15A and containing GPe and SNr structures. Prominent axon signal is detected in SNr but not GPe. Scale bar: 1 mm.

FIGS. 16A-16G. A C57Bl/6J (wild-type) mouse was injected with 1.0E+12 viral genomes of CN2609 serotype PHP.eB virus via the retro-orbital sinus. Representative two-photon tomography (TissueCyte) images of the native SYFP2 fluorescence in coronal brain sections are shown four weeks post-injection. FIGS. 16A, 16C, 16E show the full coronal section at different planes in the brain (moving anterior to posterior) and (16B, 16D, 16F) show higher magnification view of the boxed areas in the corresponding FIGS. 16A, 16C, 16E, respectively. (16G) TissueCyte imaging datasets were processed using an established informatics pipeline (Oh et al., Nature 2014, 508: 207-214) and registered to the CCFv3.0 (Wang et al., Cell 2020, 181(4):936-953.e20). Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 17A-17H. (17A) Fluorescent reporter expression in mouse brain tissue following retro-orbital injection of CN2631 serotype PHPeB. Scale bar: 1 mm. (17B) Higher magnification view of SYFP2 expression in the boxed dorsal striatum region from FIG. 17A. Scale bar: 100 microns. (17C) anti-GFP signal in mouse caudoputamen following retro-orbital injection of CN2631 serotype PHPeB. (17D) Anti-ChAT signal in the same region from FIG. 17A. (17E) Overlay of signal from FIGS. 17C and 17D showing colocalization of anti-GFP and anti-ChAT signal. Scale bar: 100 microns. (17F-17H) Enhancer: eHGT_743m; Animal: 557822; Vector: CN2631; Region: dorsal striatum. Multiplexed fluorescent in situ hybridization (mFISH) analysis of CN2631 labeling specificity in mouse dorsal striatum region showing 90% (9/10) of labeled neurons are ChAT positive striatal cholinergic interneurons. (17F) SYFP2 probe signal, (17G) overlay of SYFP2 and ChAT probe signals, and (17H) ChAT probe signal. Circles represent SYFP2+/ChAT+ cells and diamonds represent SYFP2+/ChAT− cells.

FIGS. 18A-18G. A C57Bl/6J (wild-type) mouse was injected with 1.0E+12 viral genomes of CN2631 serotype PHP.eB virus via the retro-orbital sinus. Representative two-photon tomography (TissueCyte) images of the native SYFP2 fluorescence in coronal brain sections are shown four weeks post-injection. FIGS. 18A, 18C, 18E show the full coronal section at different planes in the brain (moving anterior to posterior) and (18B, 18D, 18F) show higher magnification view of the boxed areas in the corresponding FIGS. 18A, 18C, 18E, respectively. (18G) TissueCyte imaging datasets were processed using an established informatics pipeline (Oh et al., Nature 2014, 508: 207-214) and registered to the CCFv3.0 (Wang et al., Cell 2020, 181(4):936-953.e20). Segmented pixel counts or voxels for each brain region were used for analysis and density dot plots of these data in all cortical (left=left hemisphere and right=right hemisphere) and subcortical structures are shown. Dark black, large circles=brain regions with most SYFP2 signal and small, light circles=brain regions with little to no SYFP2 signal. See FIG. 21 for full list of abbreviated brain structures.

FIGS. 19A-19E. (19A) Fluorescent reporter expression in mouse brain tissue following retro-orbital injection of CN2451 serotype PHPeB. Scale bar: 1 mm. (19B) Higher magnification view of SYFP2 expression in the boxed region from FIG. 19A. Scale bar: 1 mm. (19C) Fluorescent reporter expression in rat brain tissue following injection of CN2451 serotype PHPeB into the lateral ventricle at P2 and tissue harvest at P19. Scale bar: 1 mm. (19C-19E) Multiplexed fluorescent in situ hybridization (mFISH) analysis of CN2451 labeling specificity in mouse olfactory tubercle region showing 68/71 or 96% of labeled neurons are Drd3 positive neurons. (19C) SYFP2 probe signal, (19D) overlay of SYFP2 and Drd3 probe signals, and (19E) Drd3 probe signal. Circles represent SYFP2+/Drd3+ cells and diamonds represent SYFP2+/Drd3-cells.

FIGS. 20A, 20B. (20A) Vector designs for enhancer-driven AADC expression in striatal medium spiny neurons. (20B) Anti-HA antibody staining of the mouse striatum after retro-orbital injection of AAV virus packaged with PHP.eB. Image is a montage of a sagittal section of a mouse brain.

FIG. 21. Table of abbreviations of brain structures.

FIGS. 22A-22C. (22A) Enhancer names, lengths, and sequences including eHGT_608h (SEQ ID NO: 1); eHGT_609h (SEQ ID NO: 2); eHGT_621h (SEQ ID NO: 3); Core-eHGT_621h (SEQ ID NO: 202); 3×Core-eHGT_621h (SEQ ID NO: 203); eHGT_633h (SEQ ID NO: 4); eHGT_634h (SEQ ID NO: 5); eHGT_635h (SEQ ID NO: 6); eHGT_636h (SEQ ID NO: 7); eHGT_351h (SEQ ID NO: 8); core2_eHGT_351h (SEQ ID NO: 204); 3×core2_eHGT_351h (SEQ ID NO: 205); eHGT_367h (SEQ ID NO: 9); core2_eHGT_367h (SEQ ID NO: 206); 3×core2_eHGT_367h (SEQ ID NO: 207); eHGT_441h (SEQ ID NO: 10); core_eHGT_441h (SEQ ID NO: 208); 3×core_eHGT_441h (SEQ ID NO: 209); eHGT_612h (SEQ ID NO: 11); eHGT_613h (SEQ ID NO: 12); eHGT_614h (SEQ ID NO: 13); eHGT_617h (SEQ ID NO: 14); eHGT_618h (SEQ ID NO: 15); eHGT_619h (SEQ ID NO: 16); eHGT_620h (SEQ ID NO: 17); eHGT_442h (SEQ ID NO: 18); eHGT_444h (SEQ ID NO: 19); core2_eHGT_444h (SEQ ID NO: 210); 3×core2_eHGT_444h (SEQ ID NO: 211); eHGT_445h (SEQ ID NO: 20); core2_eHGT_445h (SEQ ID NO: 212); 3×core2_eHGT_445h (SEQ ID NO: 213); eHGT_450h (SEQ ID NO: 21); core2_eHGT_450h (SEQ ID NO: 214); 3×core2_eHGT_450h (SEQ ID NO: 215); core3_eHGT_450h (SEQ ID NO: 216); 3×core3_eHGT_450h (SEQ ID NO: 217); eHGT_452h (SEQ ID NO: 22); core2_eHGT_452h (SEQ ID NO: 218); 3×core2_eHGT_452h (SEQ ID NO: 219); eHGT_610h (SEQ ID NO: 23); eHGT_611h (SEQ ID NO: 24); eHGT_615h (SEQ ID NO: 25); eHGT_616h (SEQ ID NO: 26); eHGT_627h (SEQ ID NO: 27); eHGT_628h (SEQ ID NO: 28); eHGT_629h (SEQ ID NO: 29); eHGT_446h (SEQ ID NO: 30); eHGT_447h (SEQ ID NO: 31); Core-eHGT_447h (SEQ ID NO: 220); 3×Core-eHGT_447h (SEQ ID NO: 221); eHGT_622h (SEQ ID NO: 32); eHGT_623h (SEQ ID NO: 33); eHGT_624h (SEQ ID NO: 34); eHGT_625h (SEQ ID NO: 35); eHGT_630h (SEQ ID NO: 36); eHGT_631h (SEQ ID NO: 37); eHGT_735m (SEQ ID NO: 39); eHGT_736m (SEQ ID NO: 40); eHGT_737m (SEQ ID NO: 41); eHGT_738m (SEQ ID NO: 42); eHGT_739m (SEQ ID NO: 43); eHGT_740m (SEQ ID NO: 44); eHGT_741m (SEQ ID NO: 45); eHGT_742m (SEQ ID NO: 46); eHGT_743m (SEQ ID NO: 47); core2_eHGT_743m (SEQ ID NO: 222); 3×core2_eHGT_743m (SEQ ID NO: 223 eHGT_744m (SEQ ID NO: 48); eHGT_746m (SEQ ID NO: 49); eHGT_747m (SEQ ID NO: 50); eHGT_748m (SEQ ID NO: 51); eHGT_749m (SEQ ID NO: 52); eHGT_750m (SEQ ID NO: 53); eHGT_751m (SEQ ID NO: 54); eHGT_779m (SEQ ID NO: 55); core2_eHGT_779m (SEQ ID NO: 224); 3×core2_eHGT_779m (SEQ ID NO: 225); eHGT_780m (SEQ ID NO: 56); Core2_eHGT_780m (SEQ ID NO: 226); 3×Core2_eHGT_780m (SEQ ID NO: 227); eHGT_781m (SEQ ID NO: 57); eHGT_782m (SEQ ID NO: 58); eHGT_783m (SEQ ID NO: 59); eHGT_784m (SEQ ID NO: 60); and eHGT_785m (SEQ ID NO: 61). (22B) Vector names, lengths (between ITRs), and sequences including CN2438 (SEQ ID NO: 62); CN2439 (SEQ ID NO: 63); CN2451 (SEQ ID NO: 64); CN2463 (SEQ ID NO: 65); CN2464 (SEQ ID NO: 66); CN2465 (SEQ ID NO: 67); CN2466 (SEQ ID NO: 68); CN2013 (SEQ ID NO: 69); CN2025 (SEQ ID NO: 70); CN2229 (SEQ ID NO: 71); CN2442 (SEQ ID NO: 72); CN2443 (SEQ ID NO: 73); CN2444 (SEQ ID NO: 74); CN2447 (SEQ ID NO: 75); CN2448 (SEQ ID NO: 76); CN2449 (SEQ ID NO: 77); CN2450 (SEQ ID NO: 78); CN2467 (SEQ ID NO: 79); CN2421 (SEQ ID NO: 80); CN2231 (SEQ ID NO: 81); CN2236 (SEQ ID NO: 82); CN2237 (SEQ ID NO: 83); CN2440 (SEQ ID NO: 84); CN2441 (SEQ ID NO: 85); CN2445 (SEQ ID NO: 86); CN2446 (SEQ ID NO: 87); CN2457 (SEQ ID NO: 88); CN2458 (SEQ ID NO: 89); CN2459 (SEQ ID NO: 90); CN2232 (SEQ ID NO: 91); CN2233 (SEQ ID NO: 92); CN2452 (SEQ ID NO: 93); CN2453 (SEQ ID NO: 94); CN2454 (SEQ ID NO: 95); CN2455 (SEQ ID NO: 96); CN2460 (SEQ ID NO: 97); CN2461 (SEQ ID NO: 98); CN2628 (SEQ ID NO: 100); CN2641 (SEQ ID NO: 101); CN2642 (SEQ ID NO: 102); CN2643 (SEQ ID NO: 103); CN2629 (SEQ ID NO: 104); CN2630 (SEQ ID NO: 105); CN2745 (SEQ ID NO: 106); CN2746 (SEQ ID NO: 107); CN2631 (SEQ ID NO: 108); CN2747 (SEQ ID NO: 109); CN2632 (SEQ ID NO: 110); CN2644 (SEQ ID NO: 111); CN2748 (SEQ ID NO: 112); CN2633 (SEQ ID NO: 113); CN2634 (SEQ ID NO: 114); CN2635 (SEQ ID NO: 115); CN2609 (SEQ ID NO: 116); CN2610 (SEQ ID NO: 117); CN2749 (SEQ ID NO: 118); CN2626 (SEQ ID NO: 119); CN2611 (SEQ ID NO: 120); CN2750 (SEQ ID NO: 121); CN2614 (SEQ ID NO: 122); CN2485 (SEQ ID NO: 228); CN2486 (SEQ ID NO: 229); CN2739 (SEQ ID NO: 230); CN2740 (SEQ ID NO: 231); CN2765 (SEQ ID NO: 232); CN2766 (SEQ ID NO: 233); CN2514 (SEQ ID NO: 234); CN2555 (SEQ ID NO: 235); CN2907 (SEQ ID NO: 236); CN2909 (SEQ ID NO: 237); CN2921 (SEQ ID NO: 238); CN2982 (SEQ ID NO: 239); CN3044 (SEQ ID NO: 240); CN3038 (SEQ ID NO: 241); CN3344 (SEQ ID NO: 242); CN3281 (SEQ ID NO: 243); CN3346 (SEQ ID NO: 244); CN3566 (SEQ ID NO: 245); CN2912 (SEQ ID NO: 246); CN2913 (SEQ ID NO: 247); CN2966 (SEQ ID NO: 248); CN2203 (SEQ ID NO: 249); and CN2700 (SEQ ID NO: 250). (22C) Exemplary sequences of subcomponents for use with artificial expression constructs disclosed herein including Beta-Globin Minimal Promoter (pBGmin/minBGlobin/minBGprom) (SEQ ID NO: 123); minCMV Promoter (SEQ ID NO: 124); Mutated minCMV Promoter (Sacl RE site removed) (SEQ ID NO: 125); minRho Promoter (SEQ ID NO: 126); minRho* Promoter (SEQ ID NO:127); Hsp68 minimal Promoter (proHsp68) (SEQ ID NO: 128); SYFP2 (SEQ ID NO: 129); EGFP (SEQ ID NO: 130); Optimized Flp recombinase (FIpO) (SEQ ID NO: 131); Improved Cre recombinase (iCre) (SEQ ID NO: 132); SP10 insulator (SP10ins) (SEQ ID NO: 133); 3×SP10ins (SEQ ID NO: 134); 3×HA tag nucleotide sequence (SEQ ID NO: 251); WPRE3 (SEQ ID NO: 135); WPRE (SEQ ID NO: 136); BGHpA (SEQ ID NO: 137); HGHpA (SEQ ID NO: 138); P2A (SEQ ID NO: 139); T2A (SEQ ID NO: 140); E2A (SEQ ID NO: 141); F2A (SEQ ID NO: 142); Exemplary Plasmid Backbone 1—Left ITR (SEQ ID NO: 143); Exemplary Plasmid Backbone 1—Right ITR (SEQ ID NO: 144); Exemplary Plasmid Backbone 2—Left ITR (SEQ ID NO: 145); Exemplary Plasmid Backbone 2—Right ITR (SEQ ID NO: 146); PHP.eB capsid (SEQ ID NO: 147); AAV9 VP1 capsid protein (SEQ ID NO: 148); tet-Transactivator version 2 (tTA2) (SEQ ID NO: 149); Aromatic L-amino acid decarboxylase (AADC) mRNA (SEQ ID NO: 150); rAAV-hAADC-vector gene insert (SEQ ID NO: 151); Tyrosine hydroxylase (TH) mRNA (SEQ ID NO: 152); GTP cyclohydrolase I (CH1) mRNA (SEQ ID NO: 153); Glucocerebrosidase (SEQ ID NO: 154); GBA1 gene product Isoform 2 (SEQ ID NO: 155); GBA1 gene product Isoform 3 (SEQ ID NO: 156); Human Huntingtin gene of exon 1 (SEQ ID NO: 157); Target sequence of human huntingtin gene of exon 1 (SEQ ID NO: 158); GTPase HRas (SEQ ID NO: 159); GCaMP6m (SEQ ID NO: 160); GCaMP6s (SEQ ID NO: 161); and GCaMP6f (SEQ ID NO: 162).

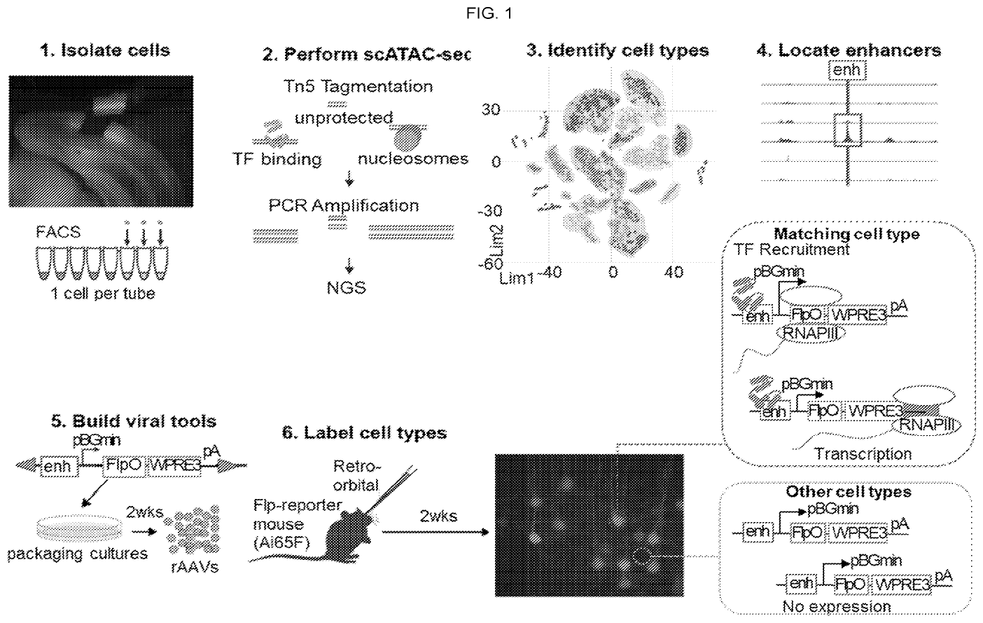

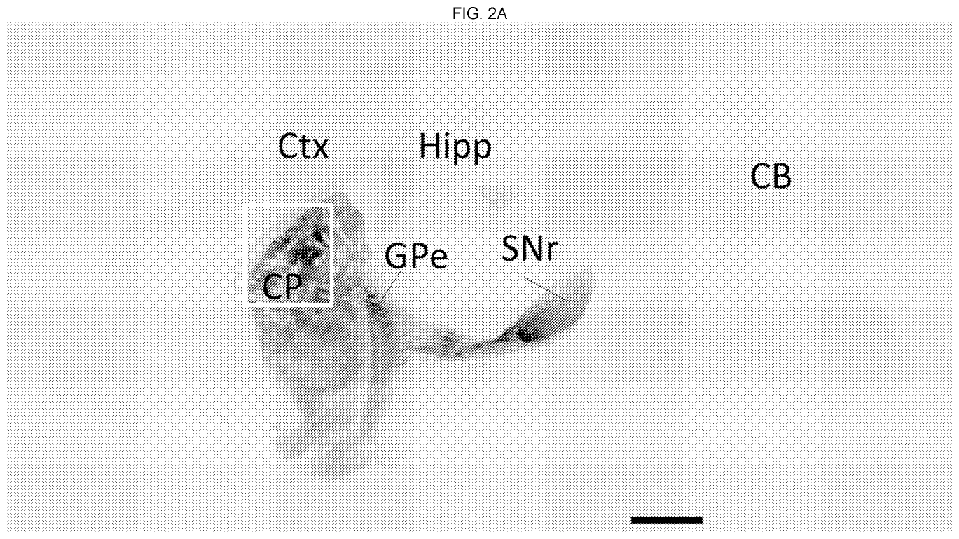

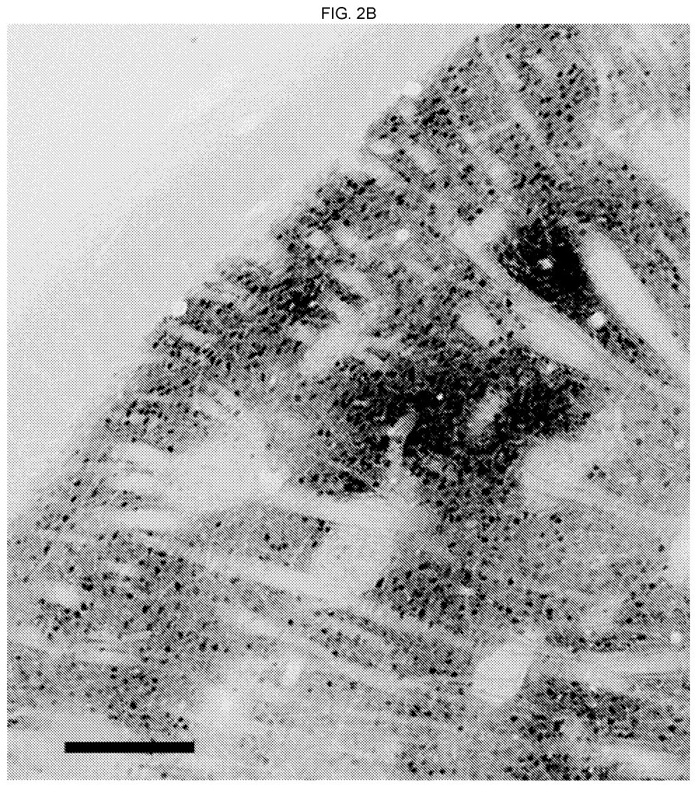

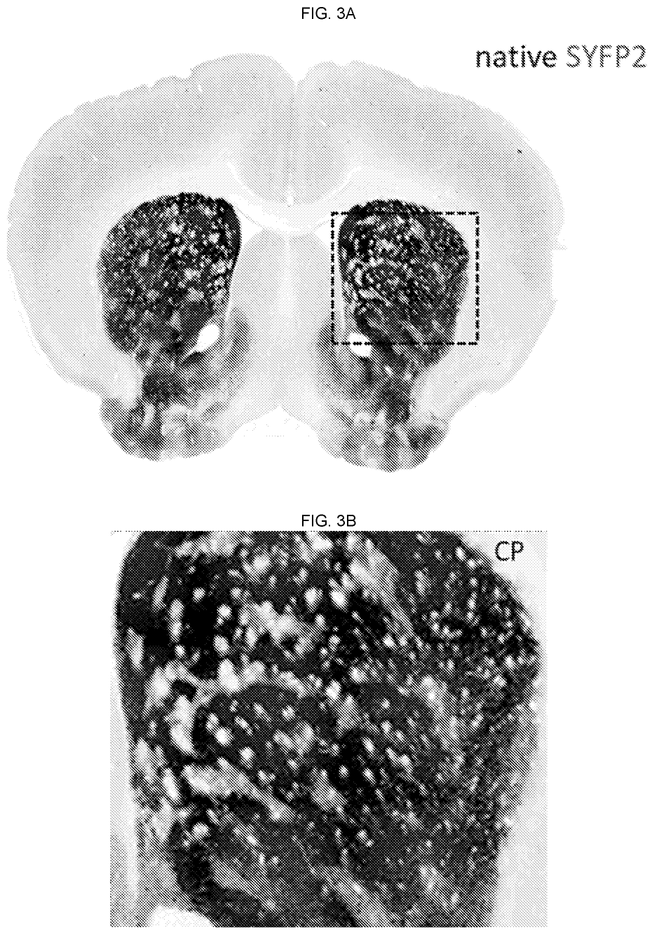

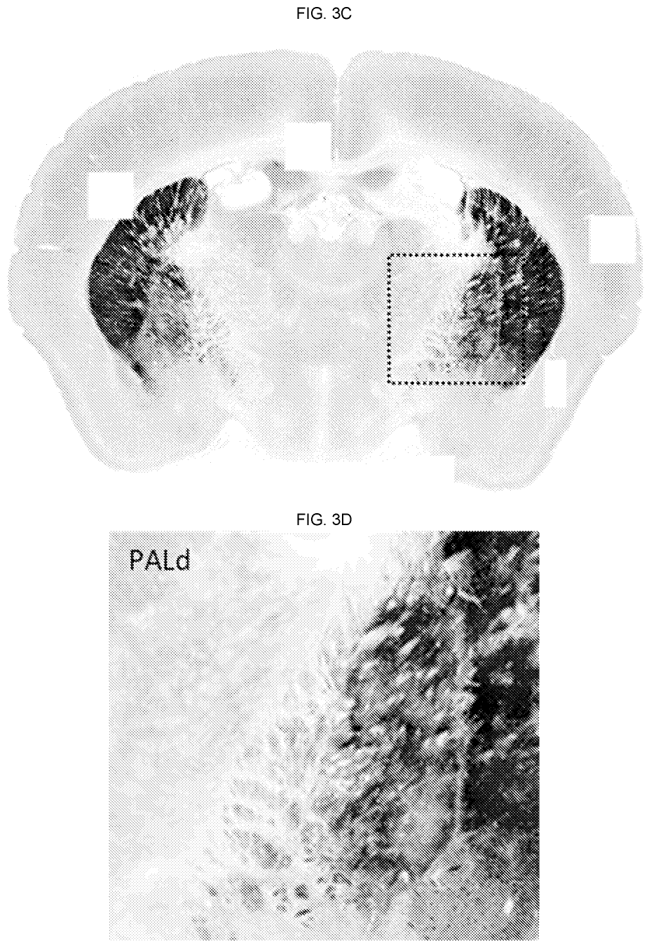

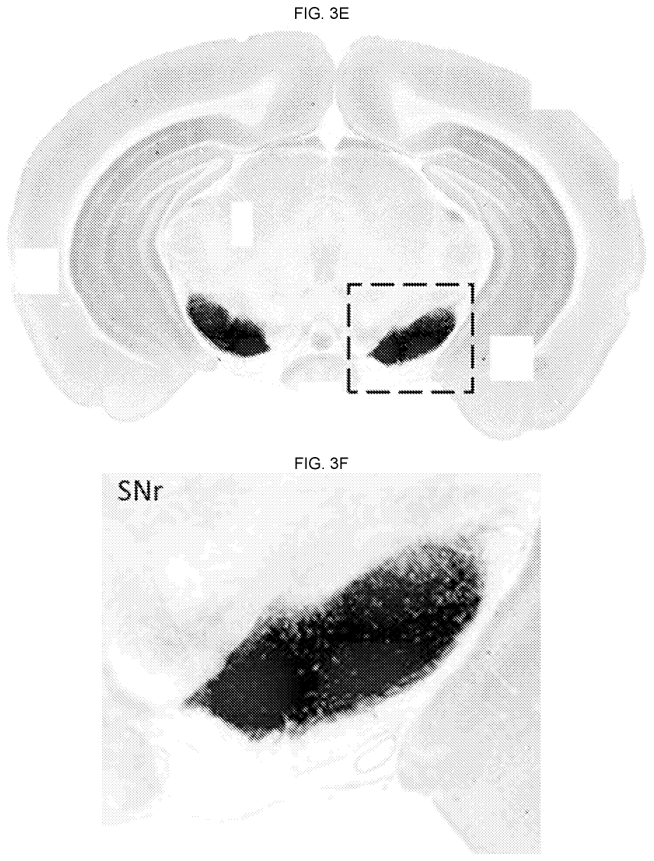

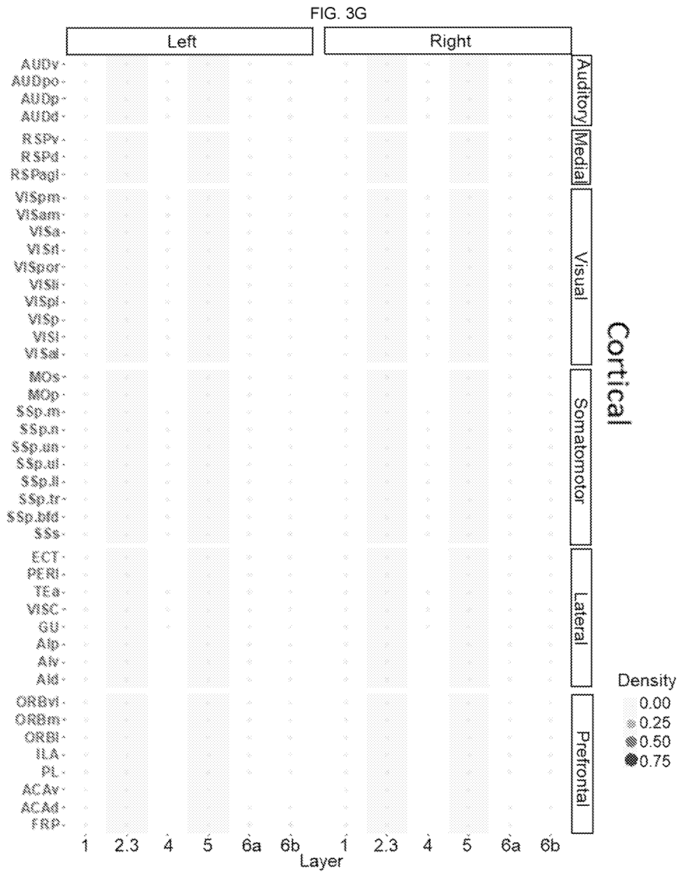

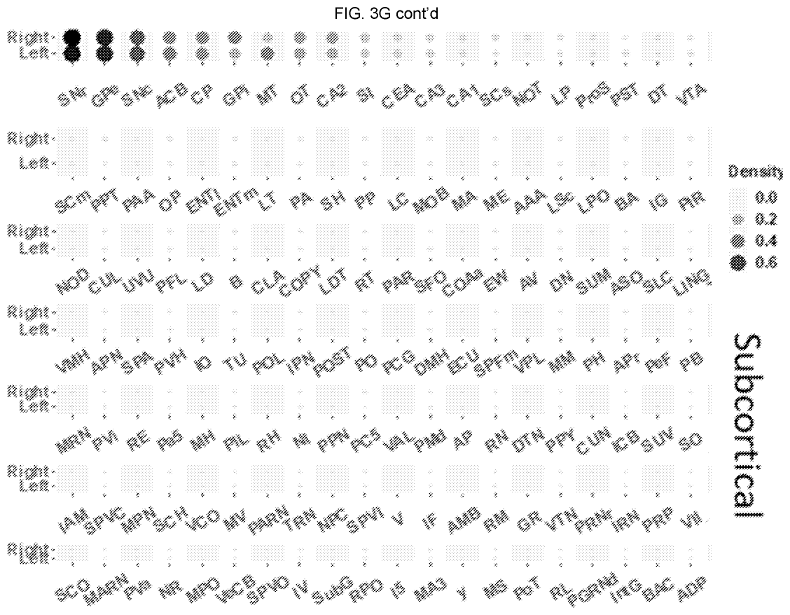

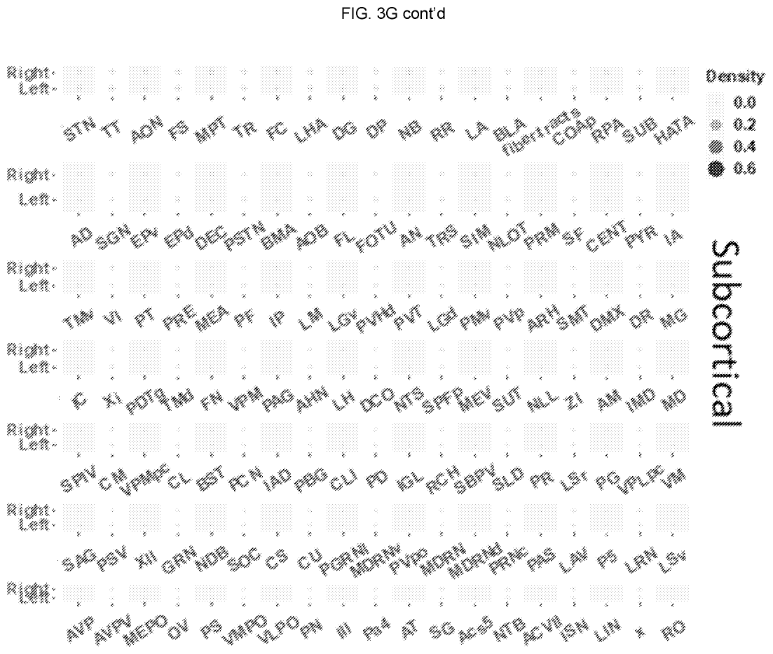

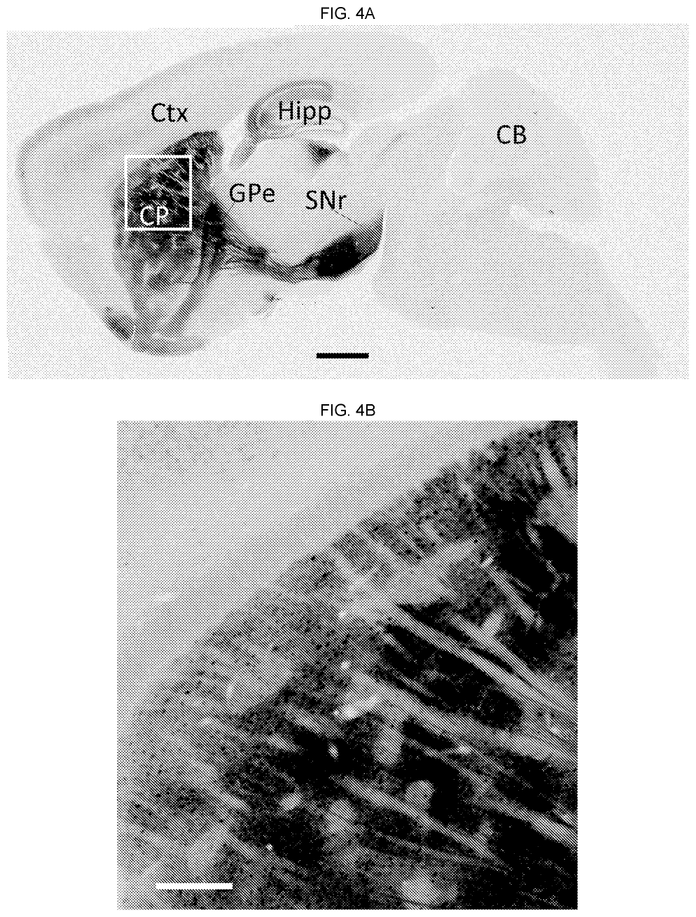

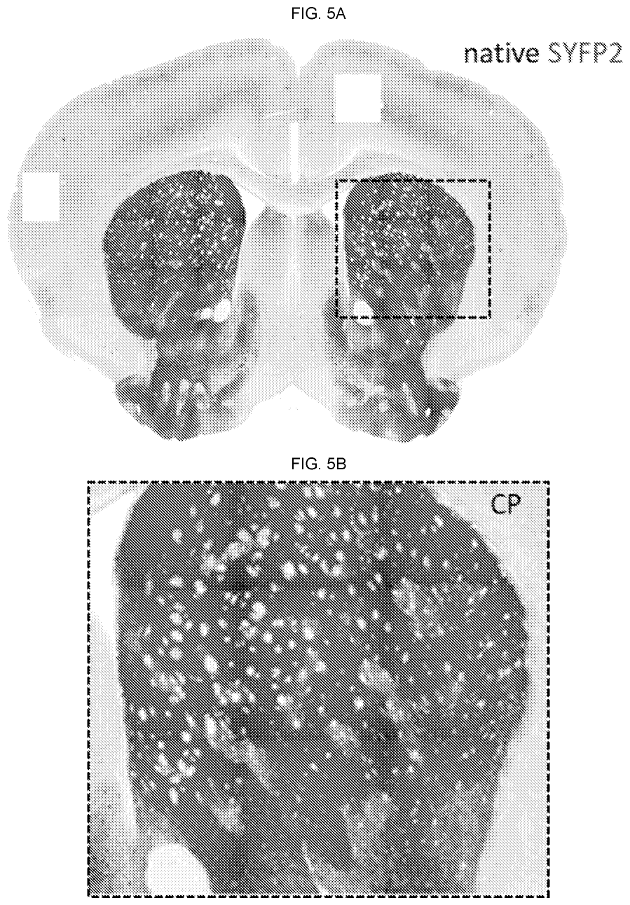

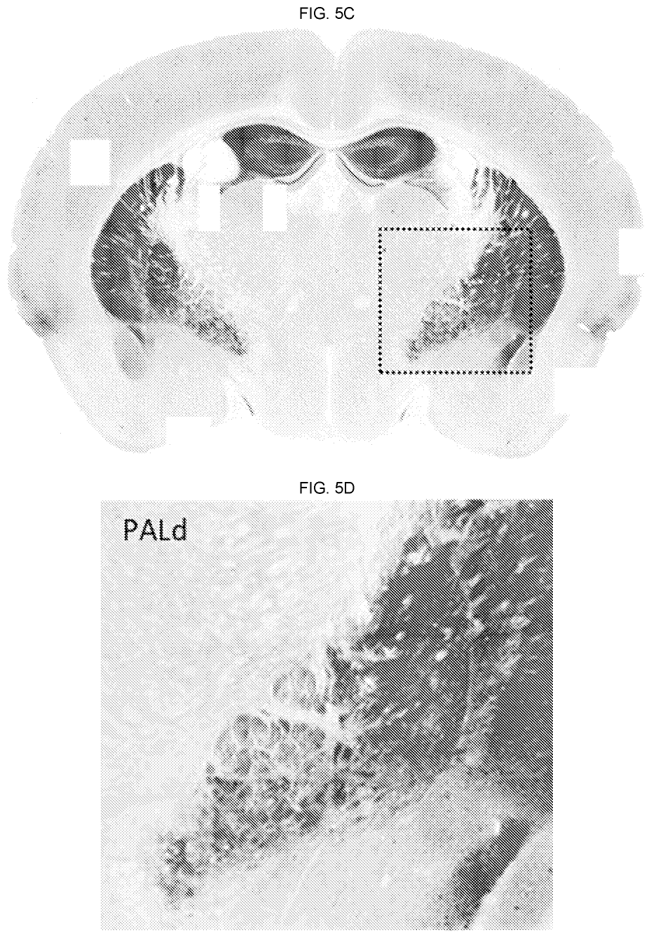

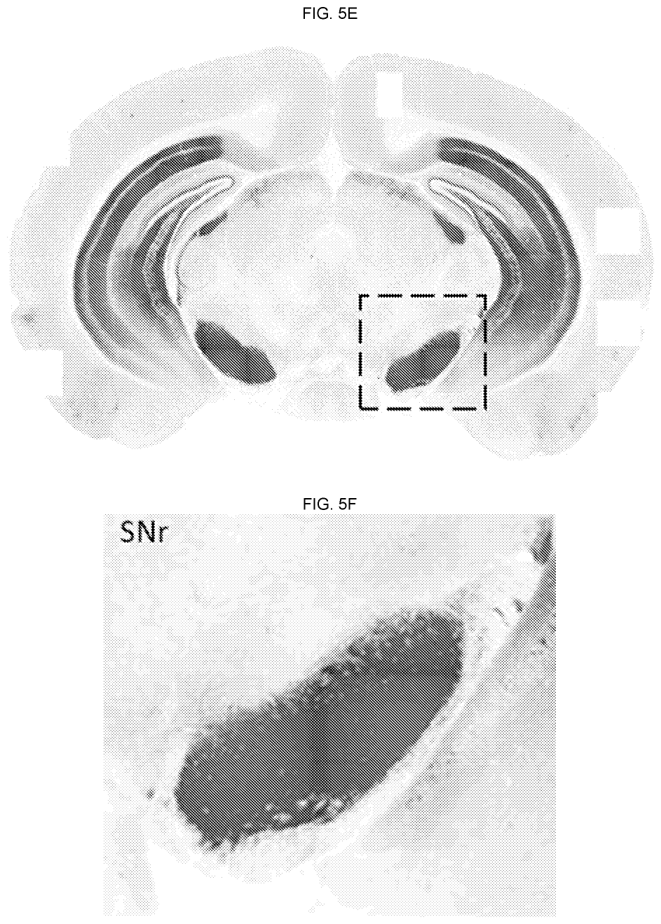

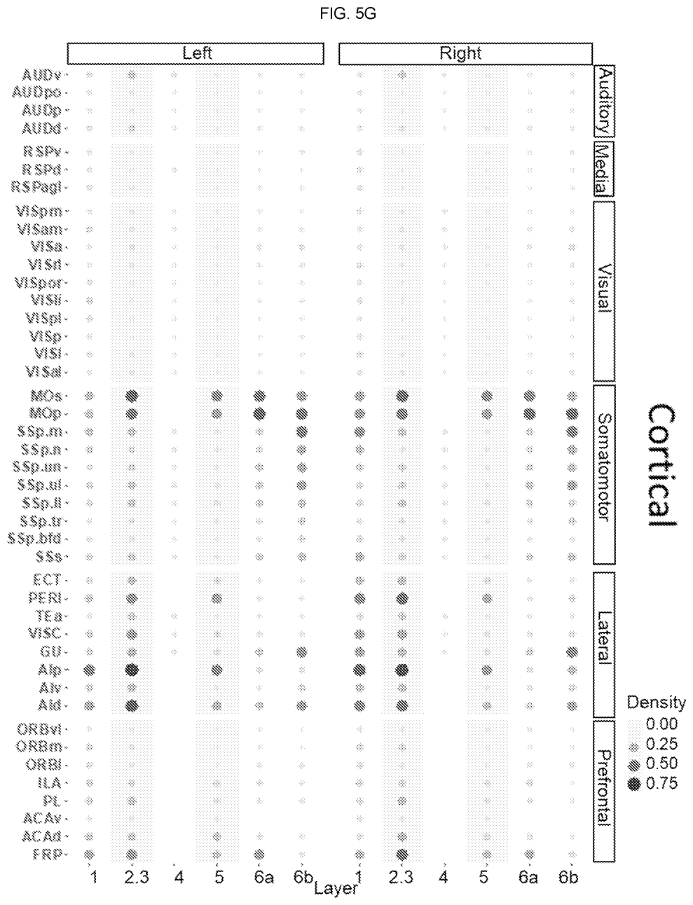

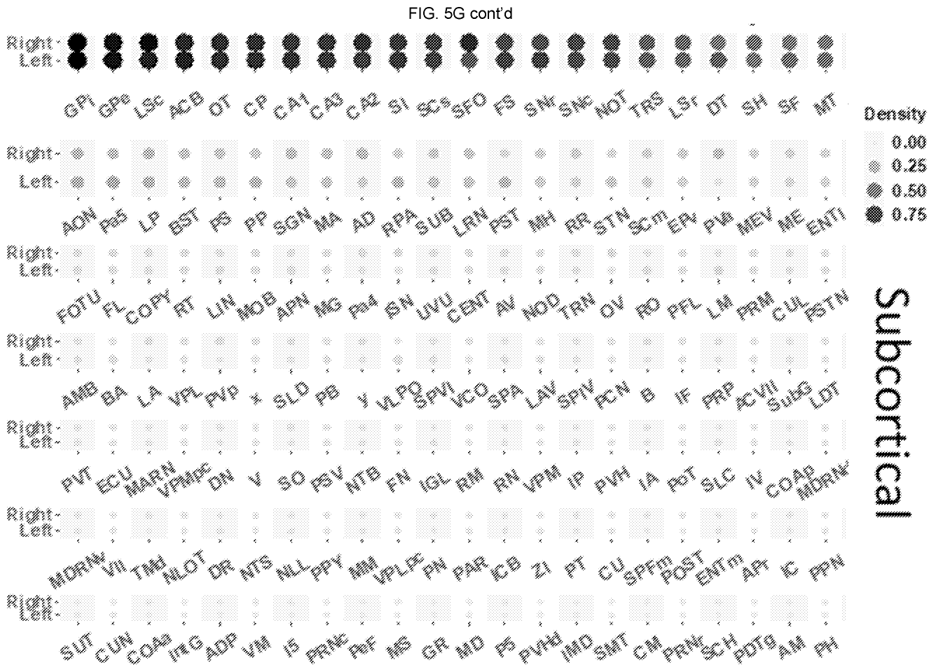

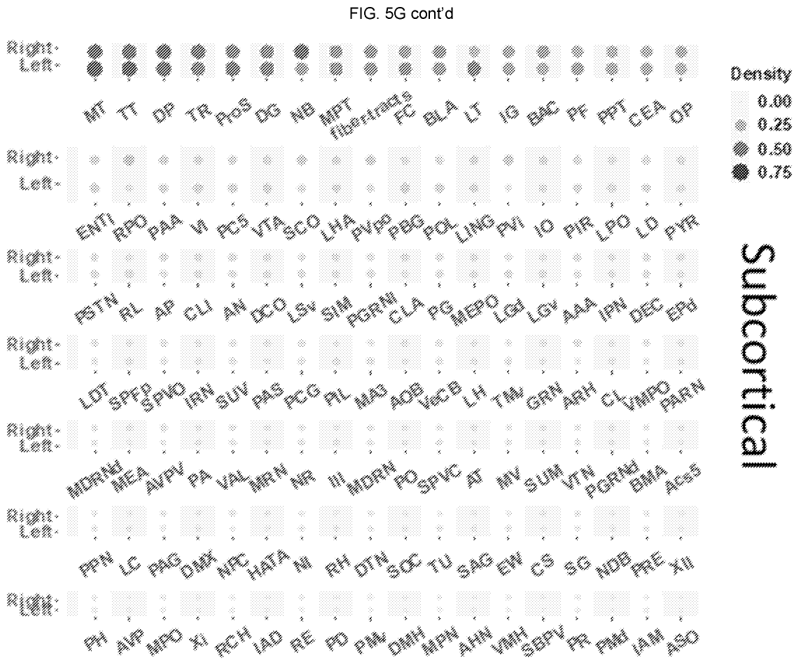

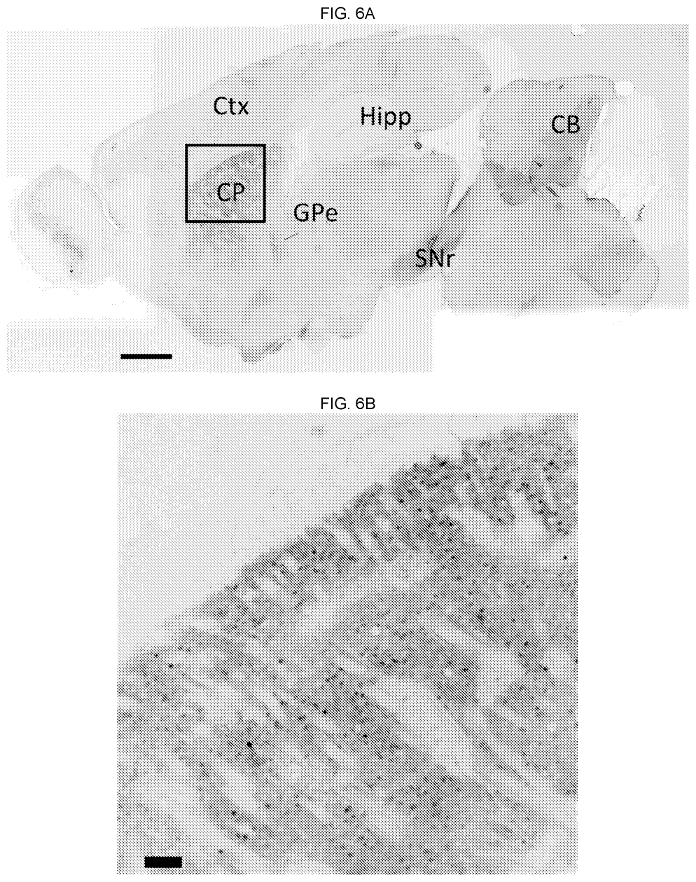

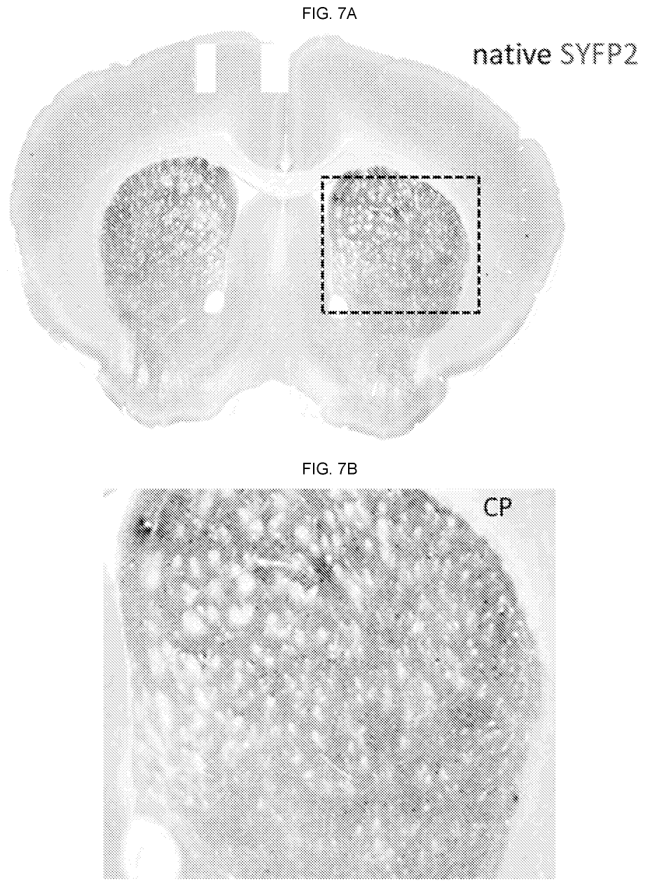

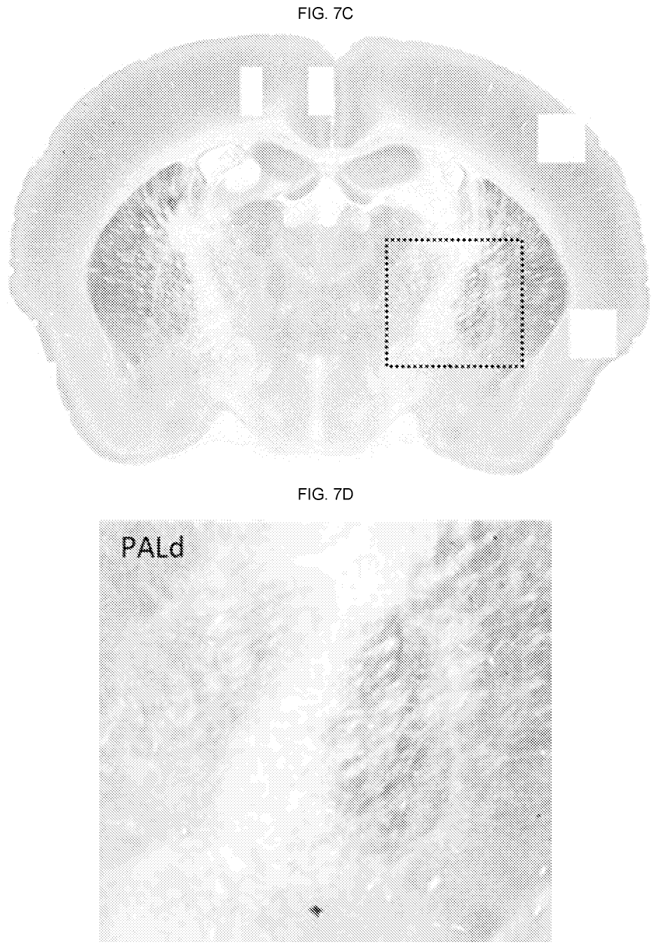

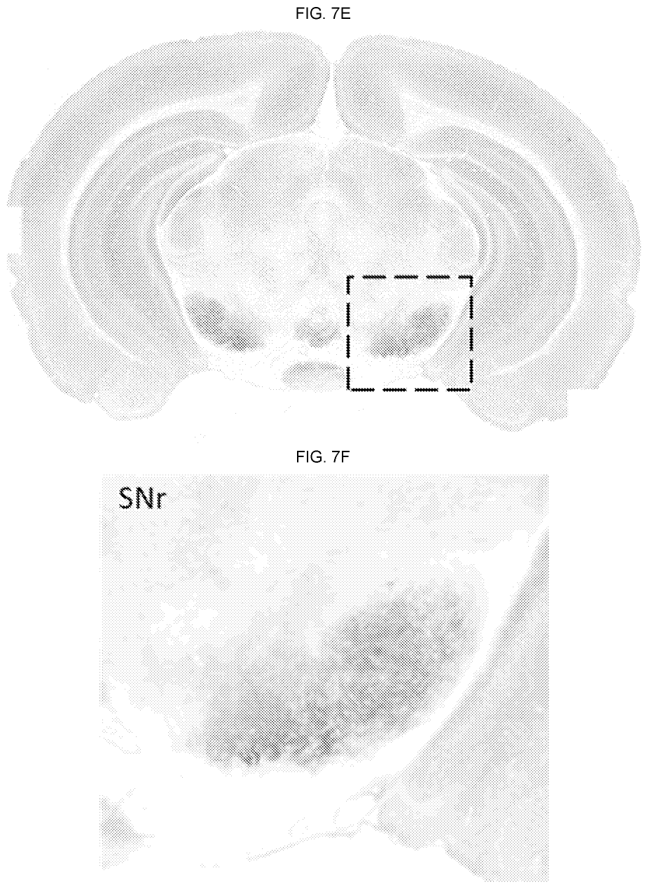

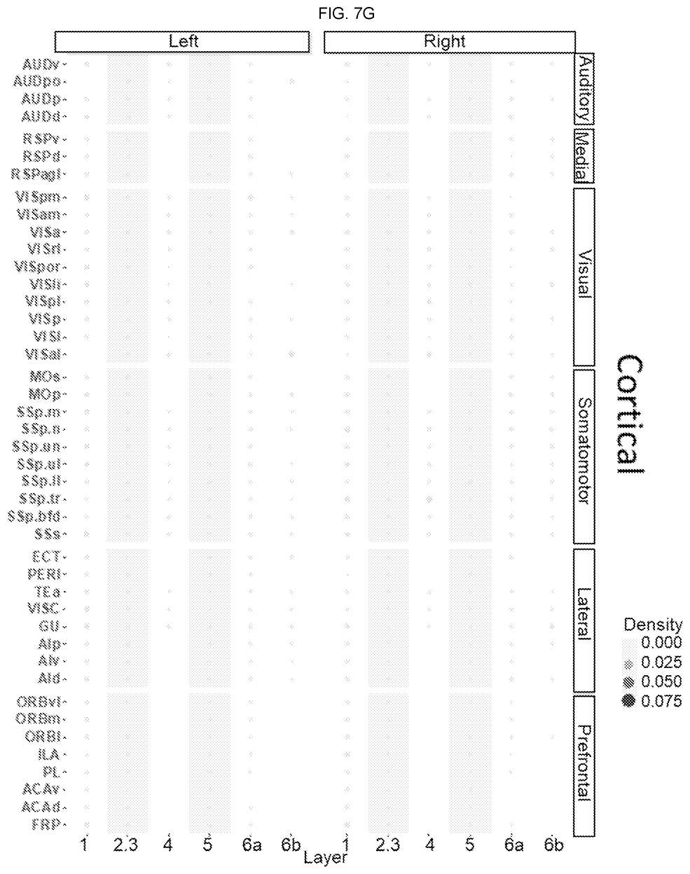

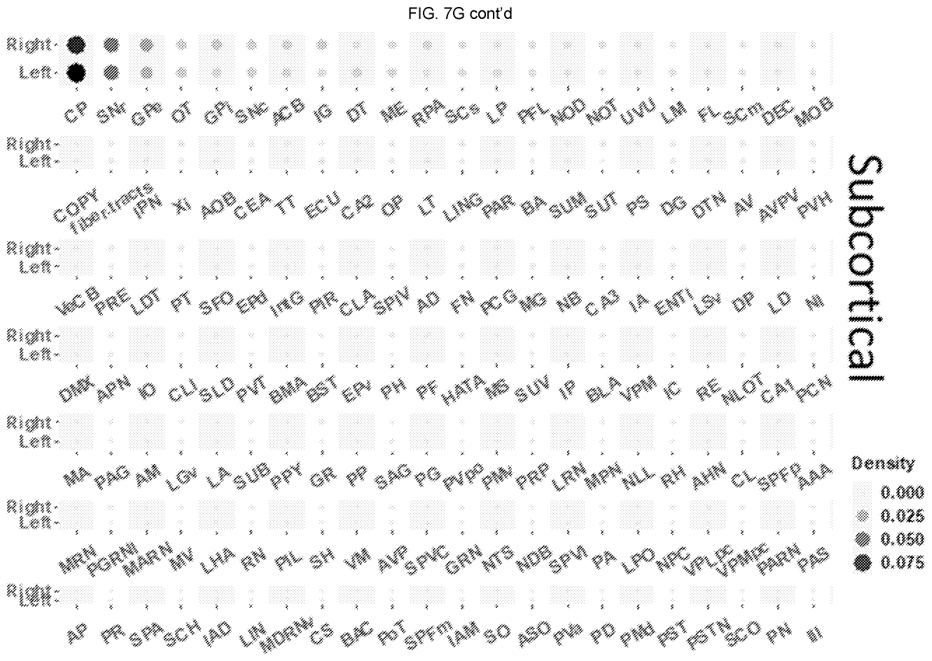

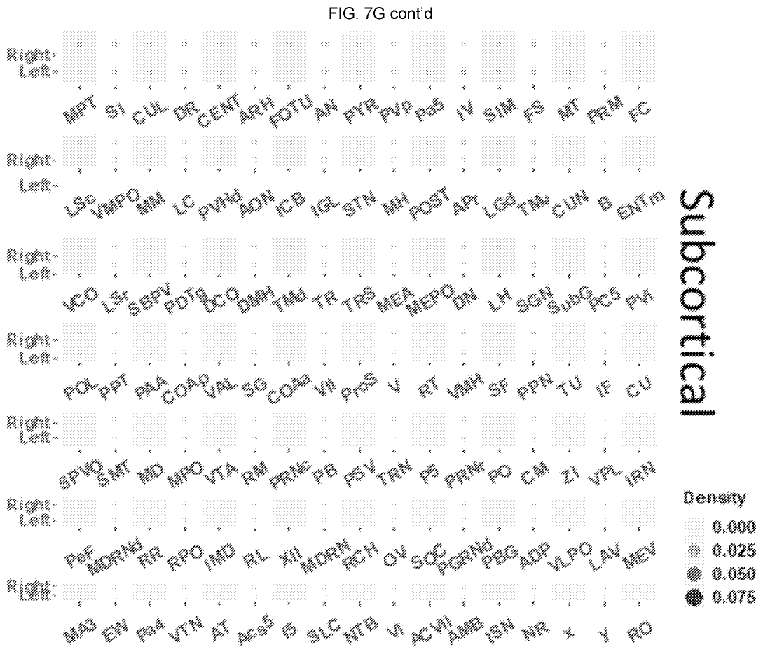

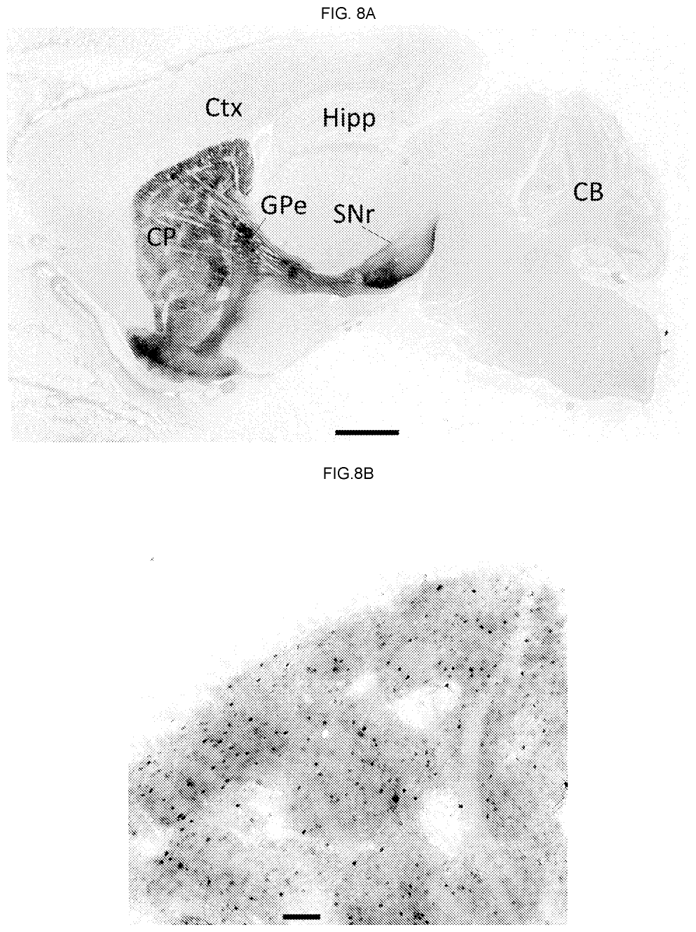

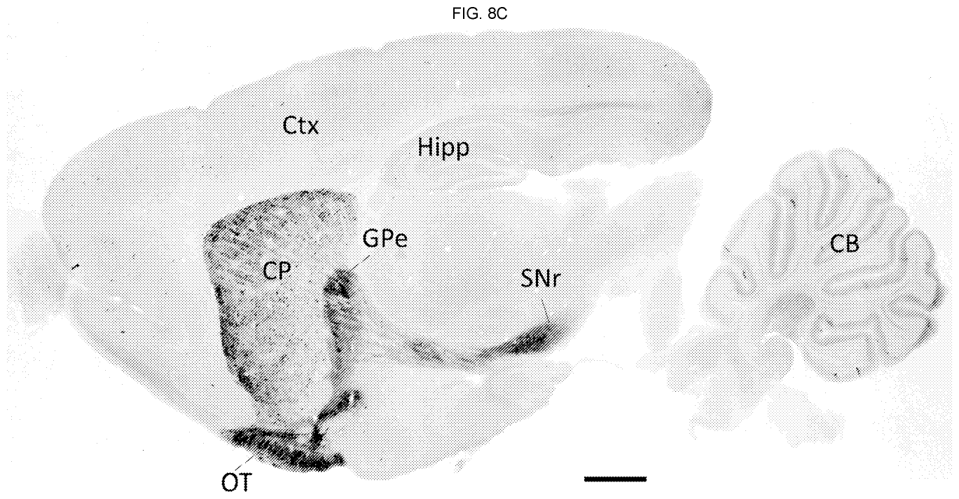

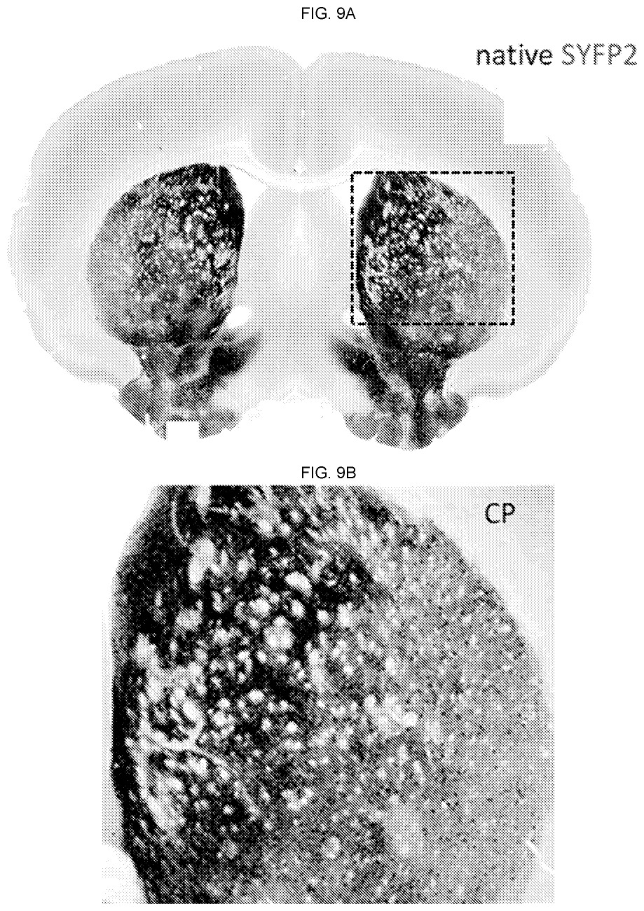

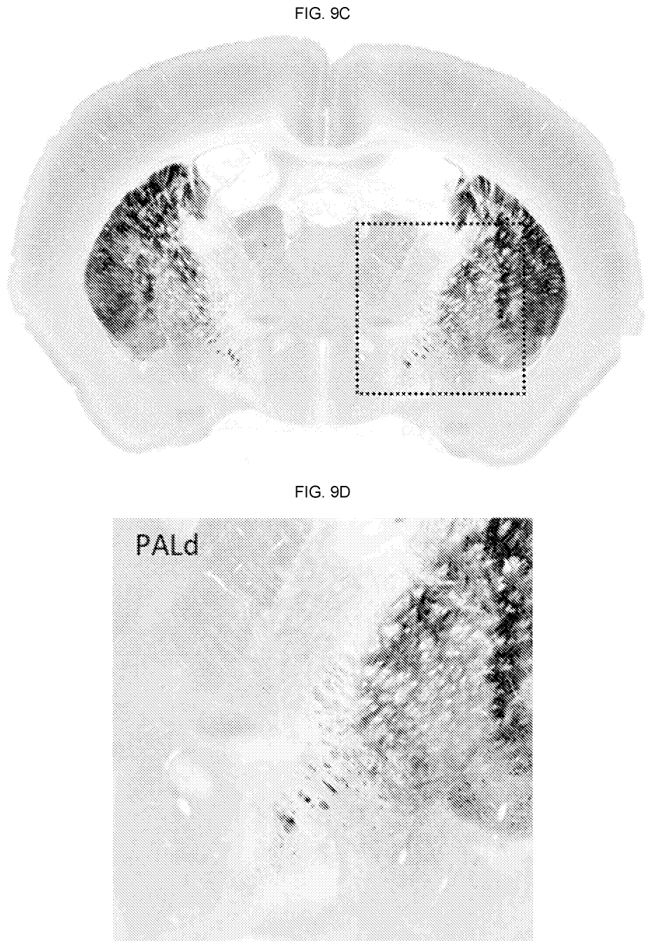

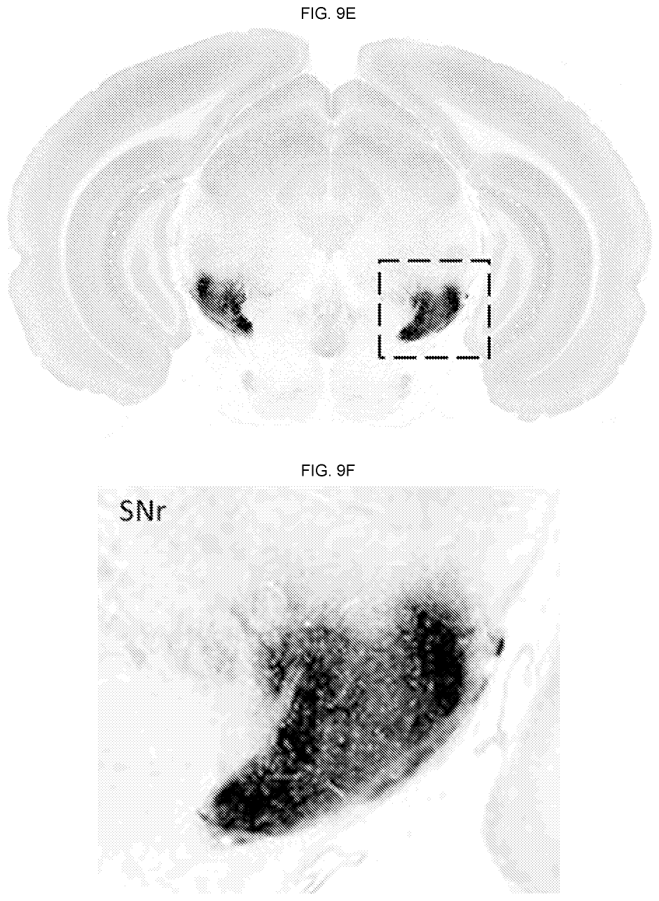

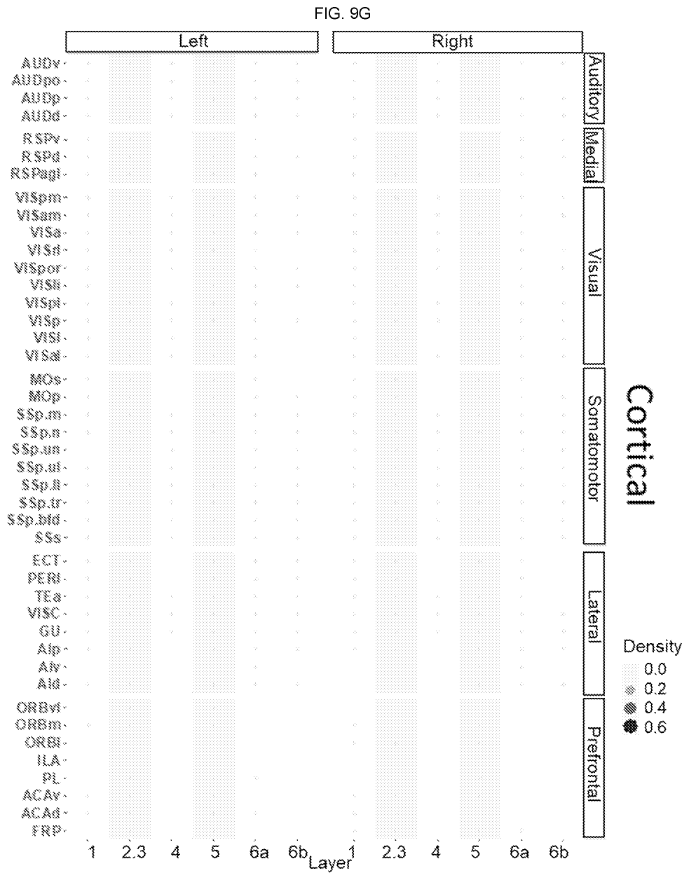

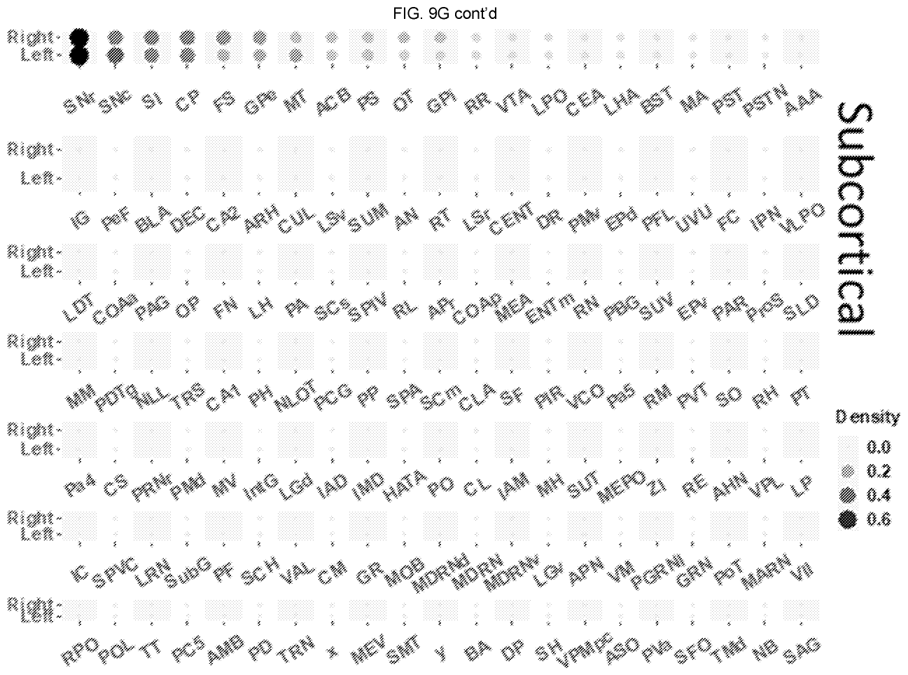

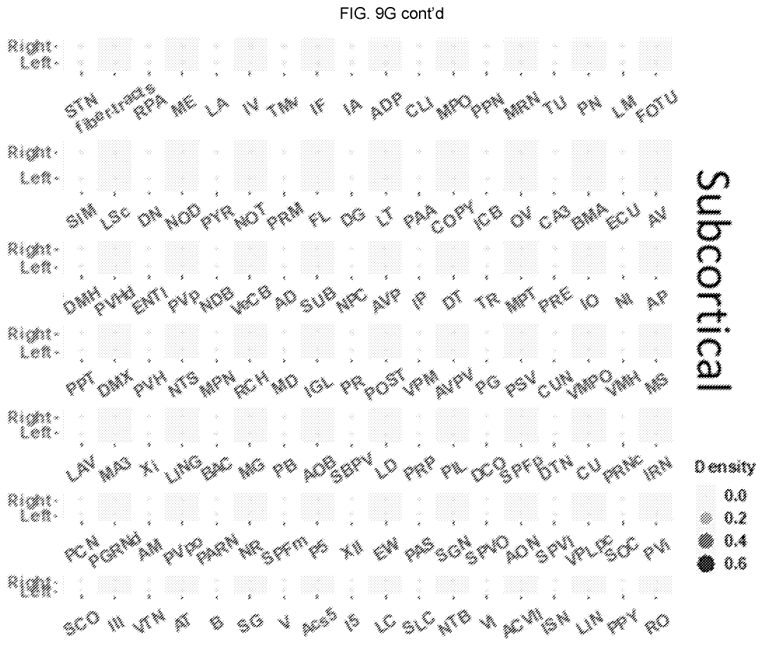

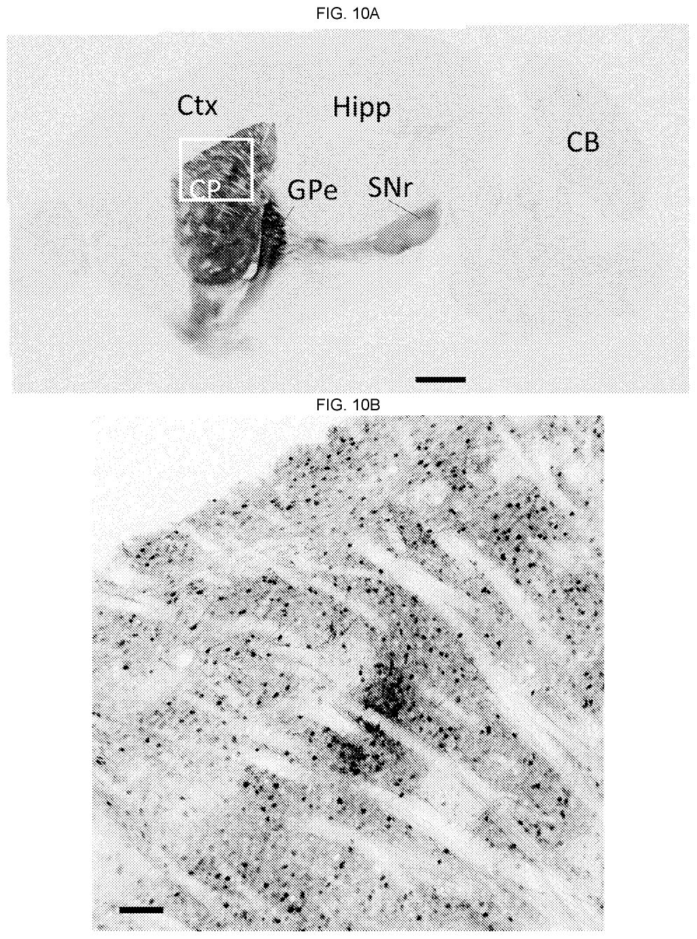

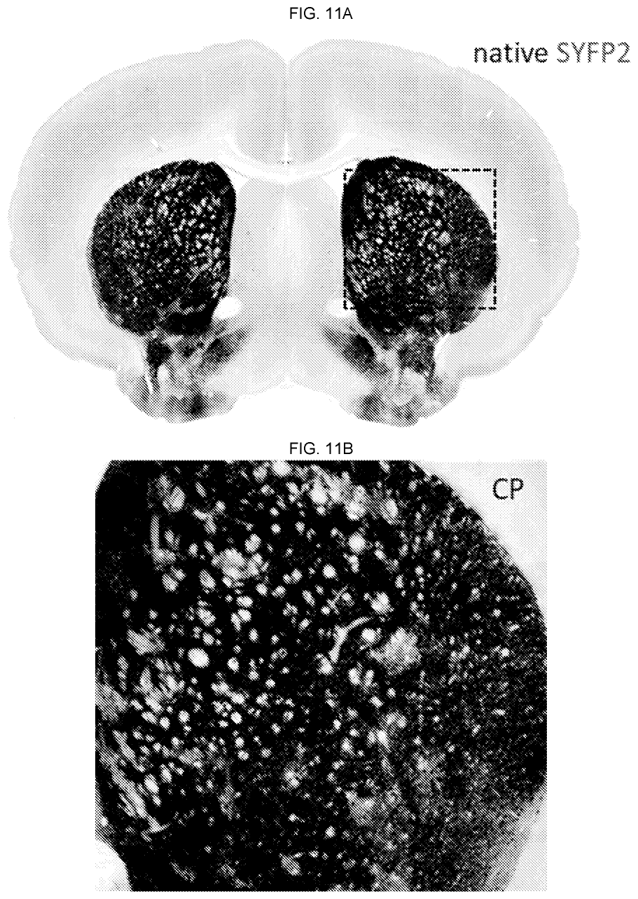

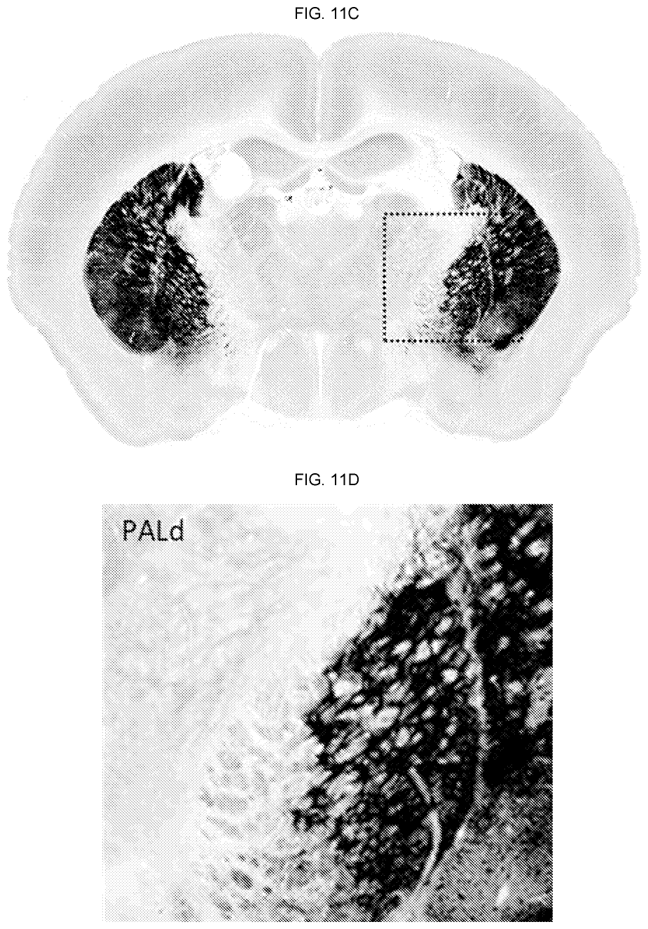

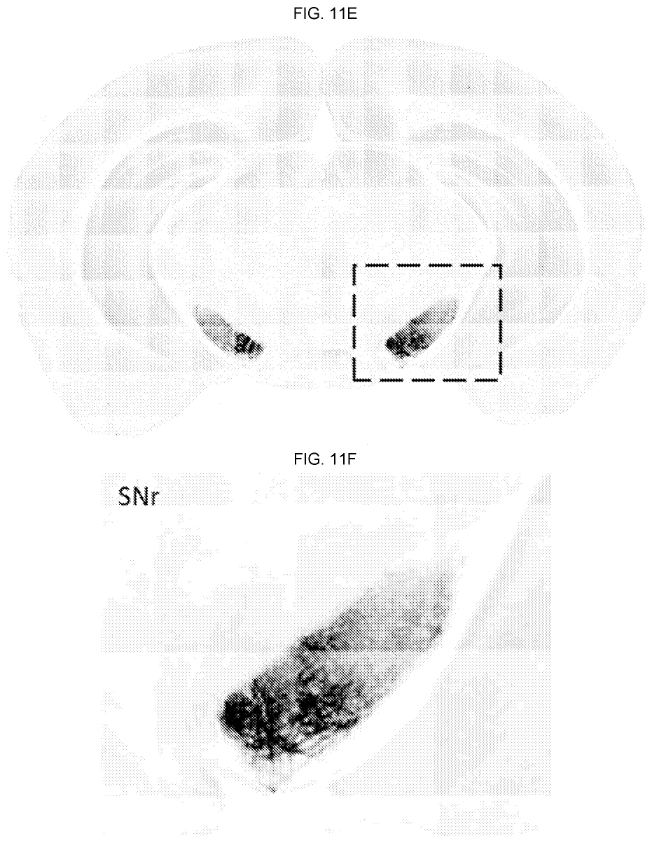

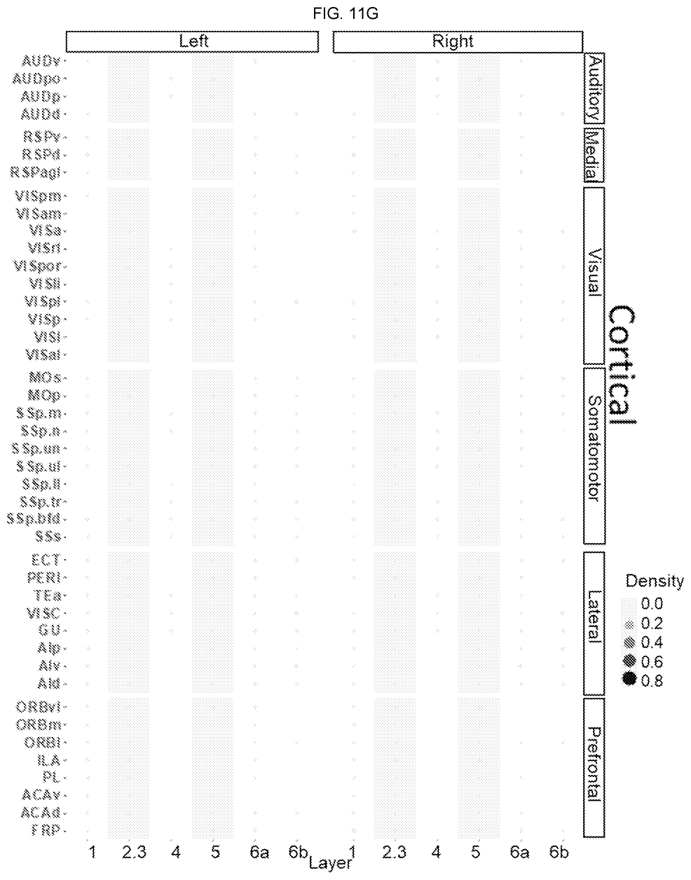

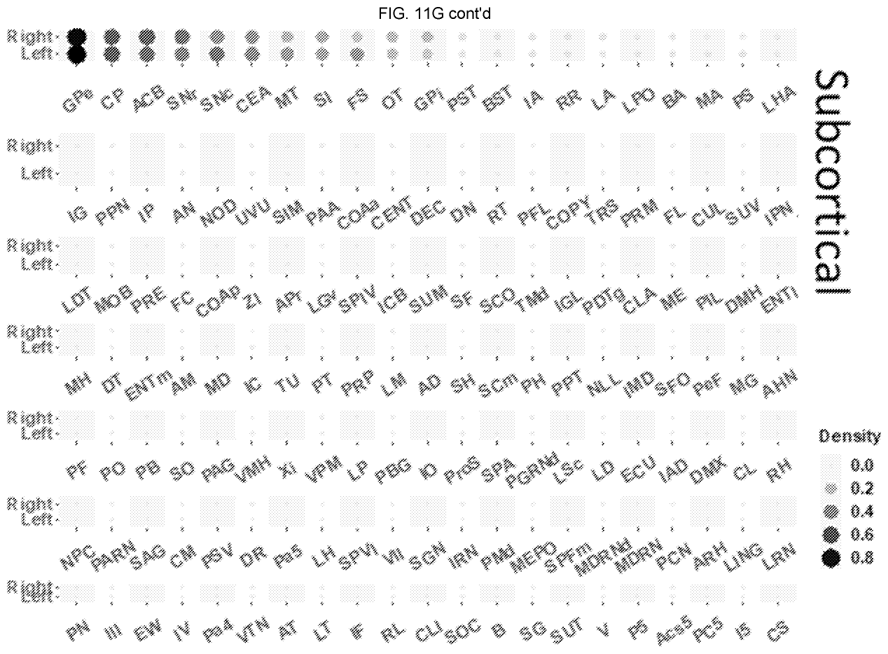

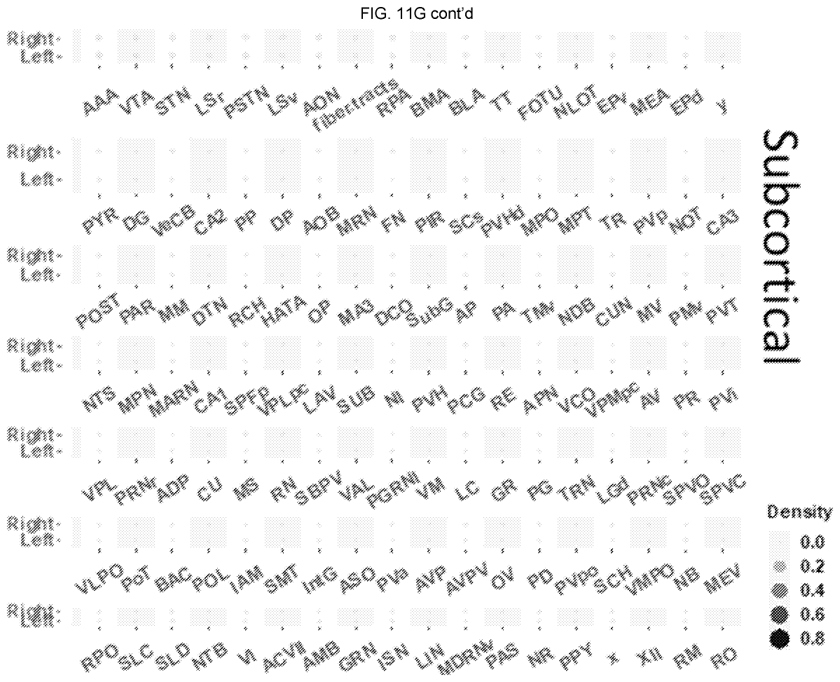

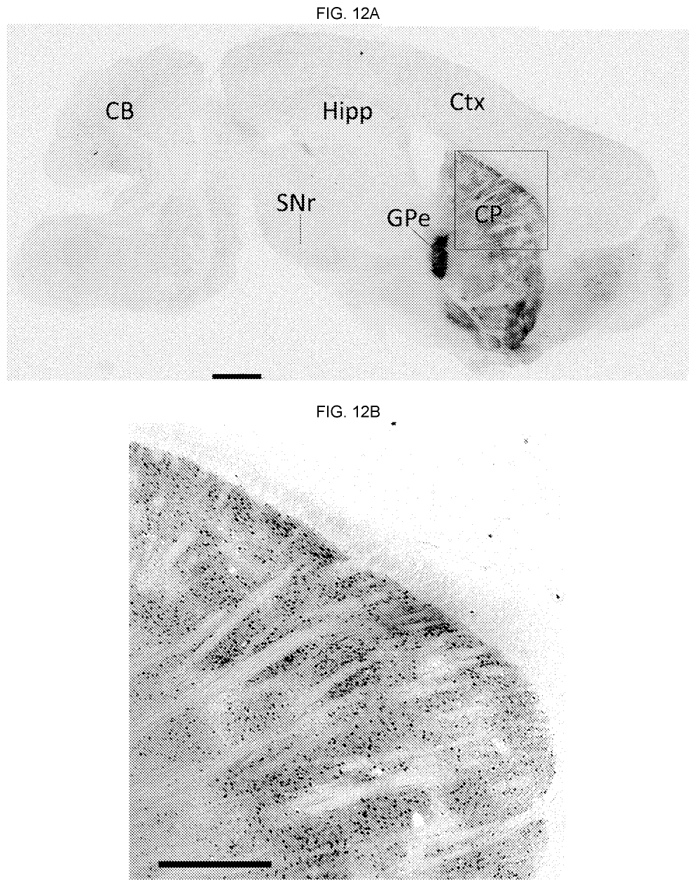

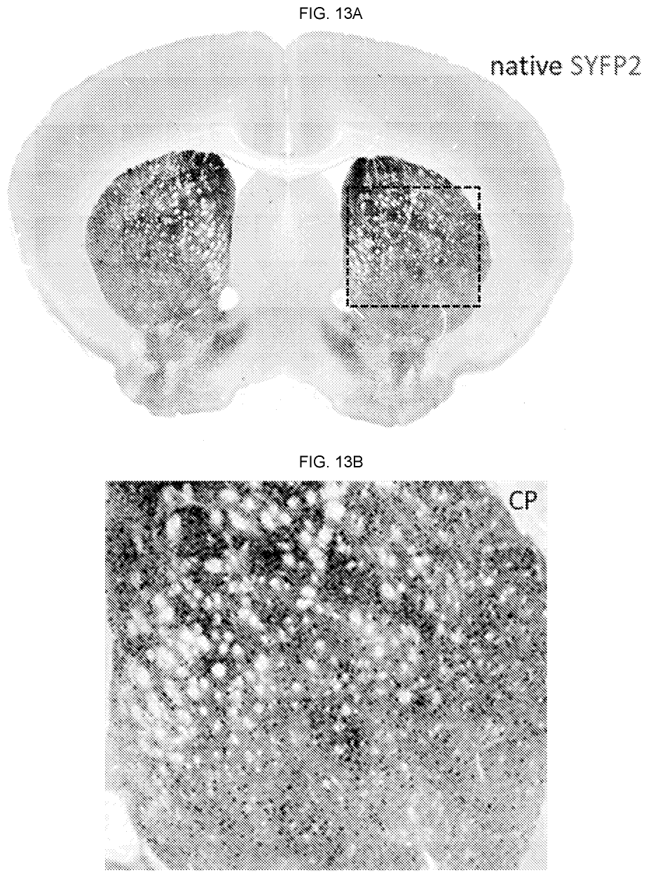

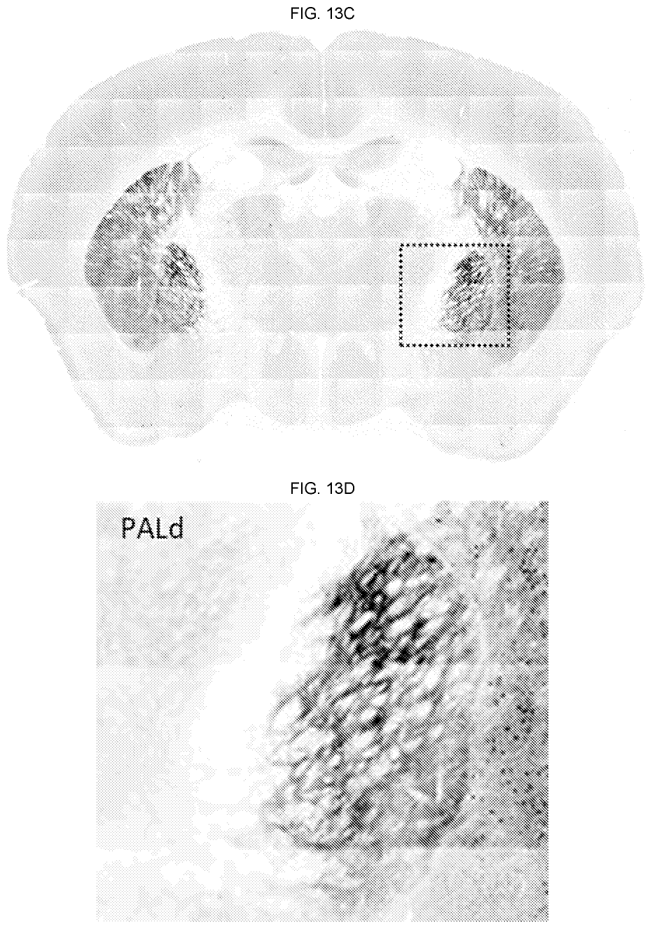

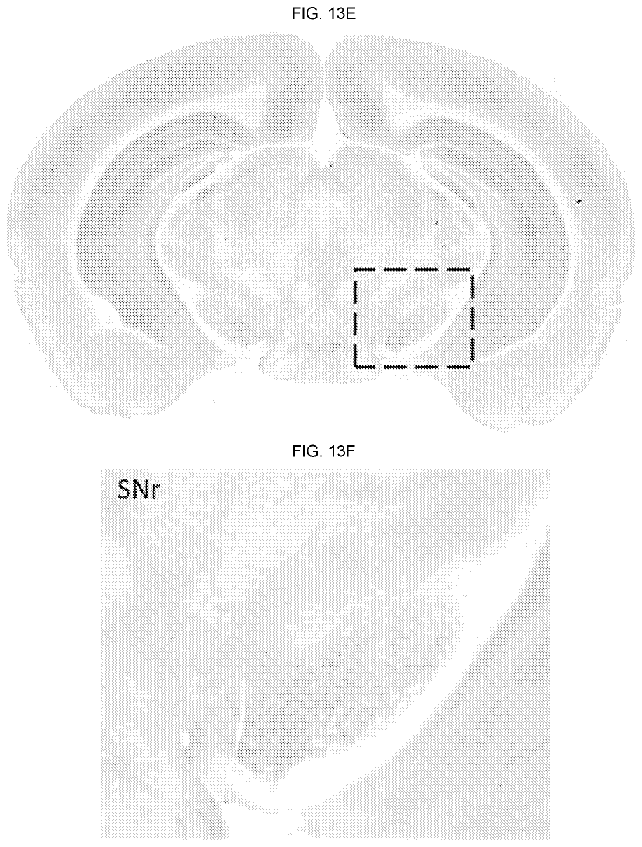

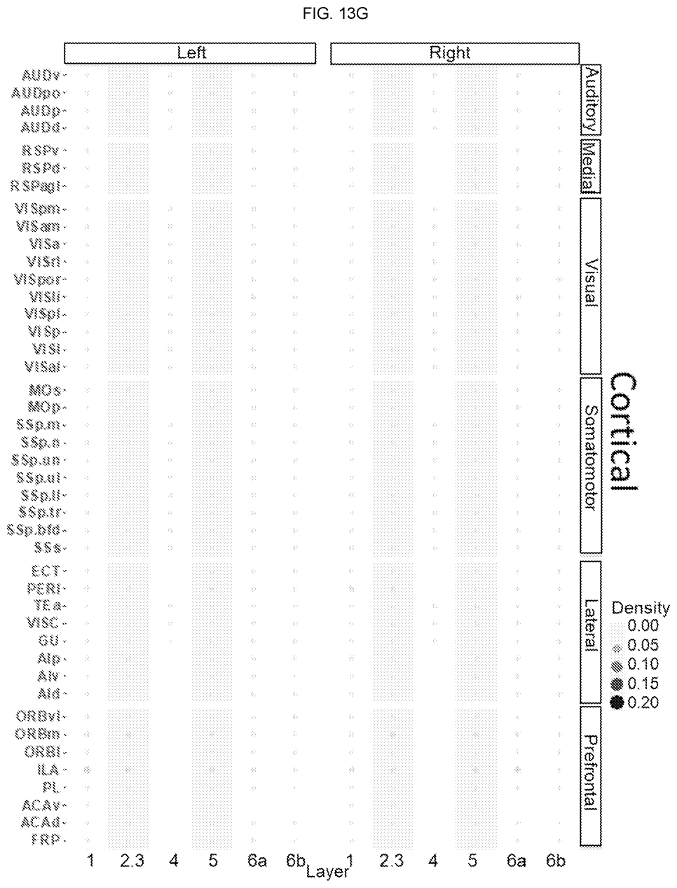

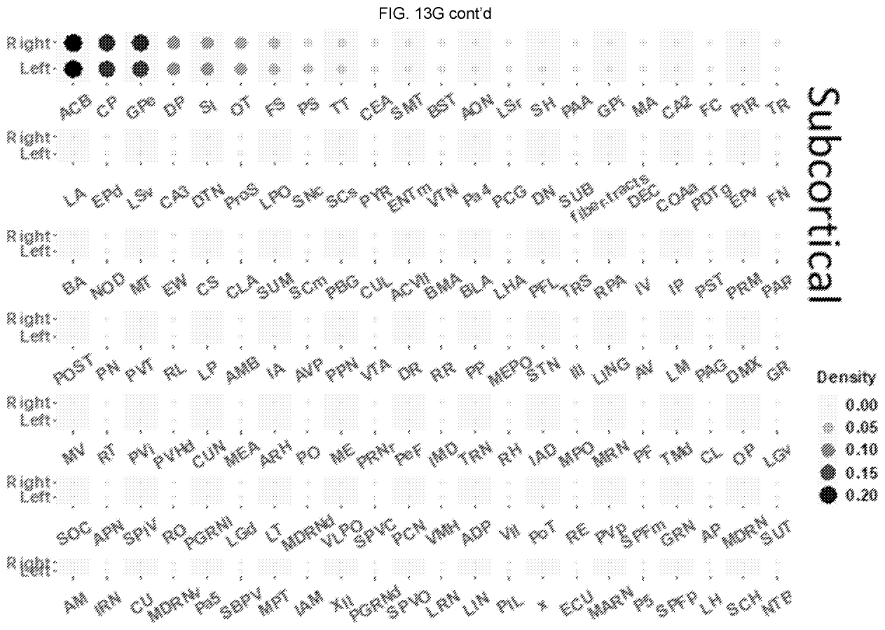

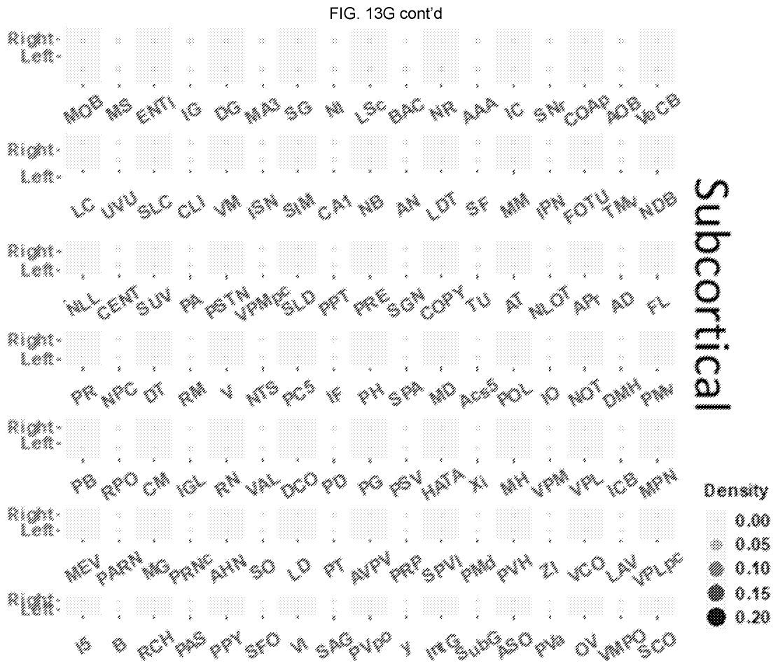

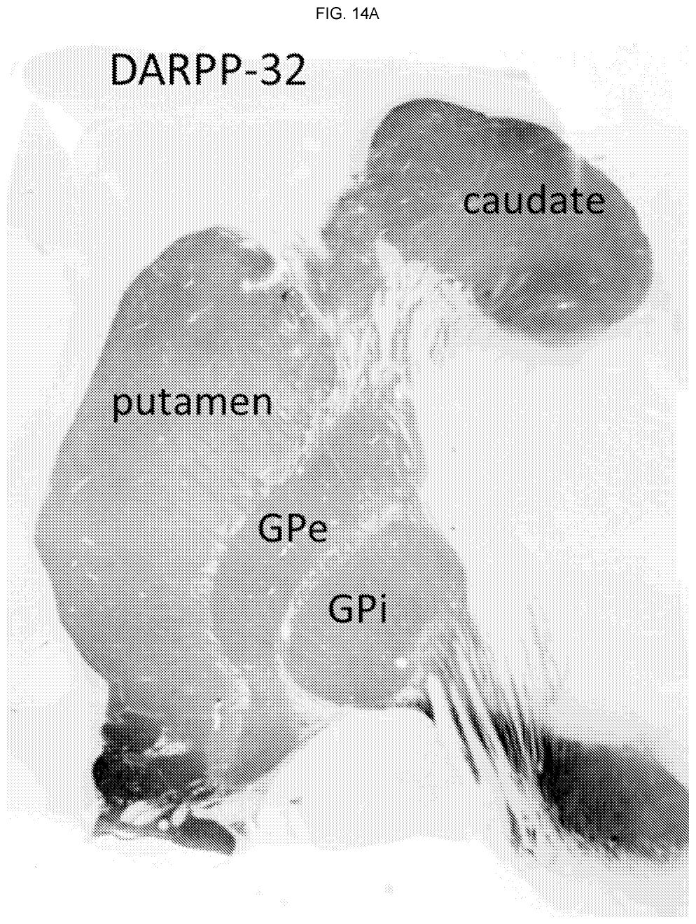

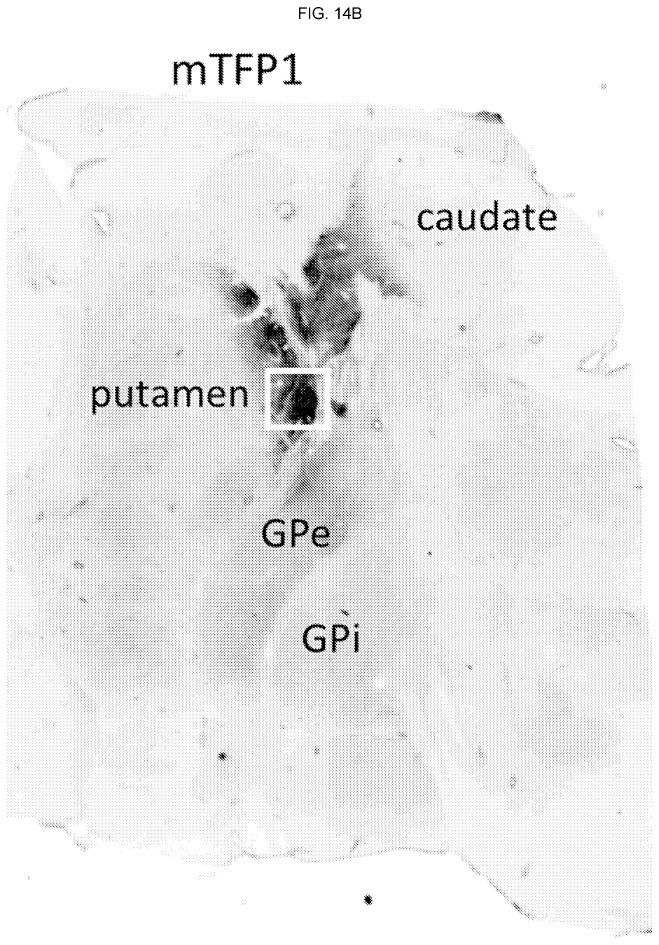

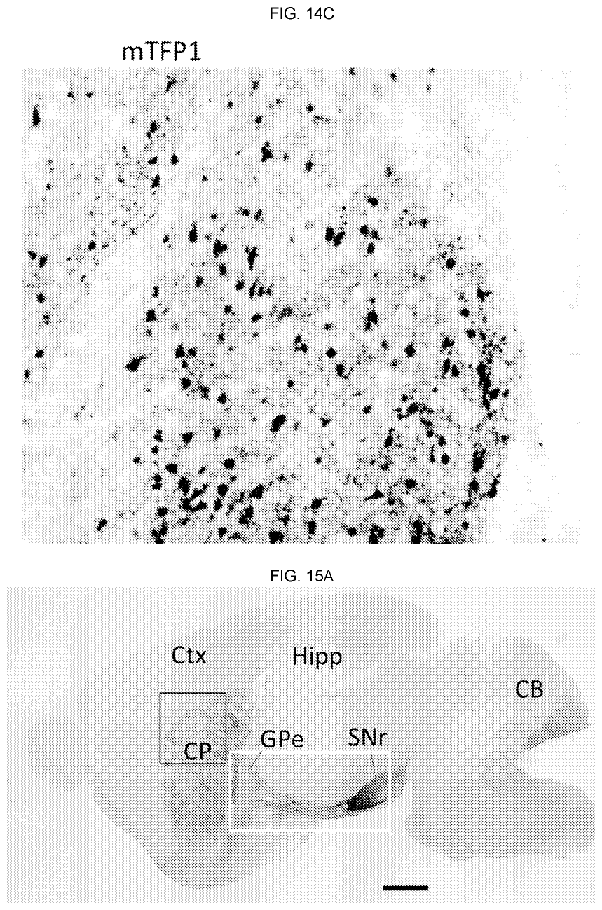

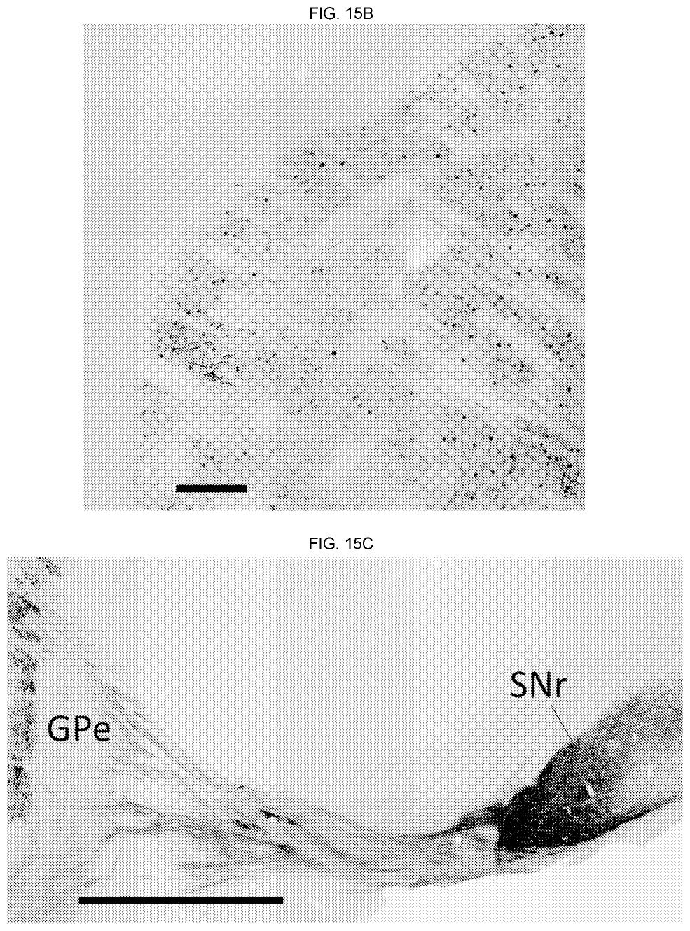

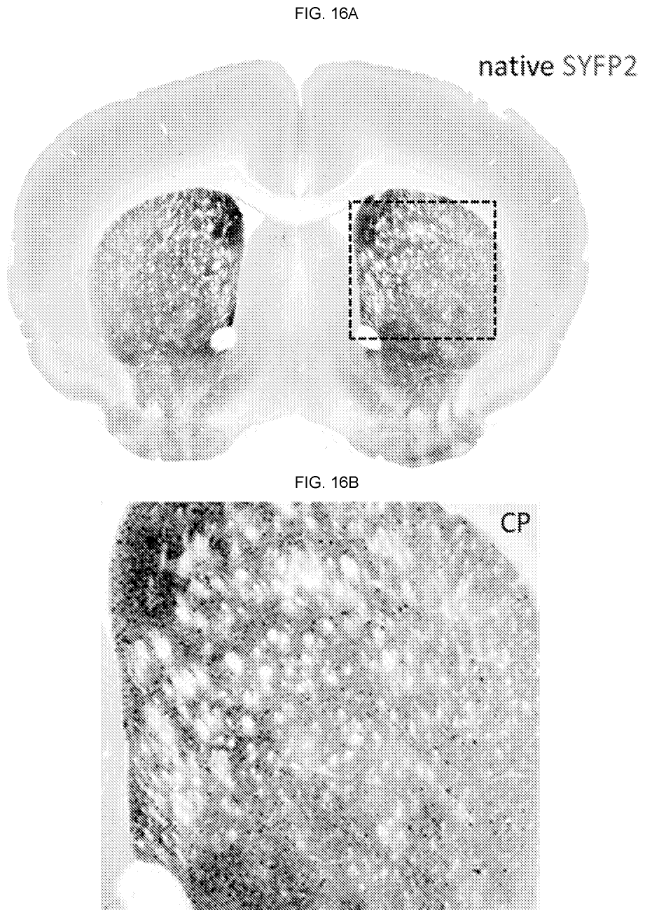

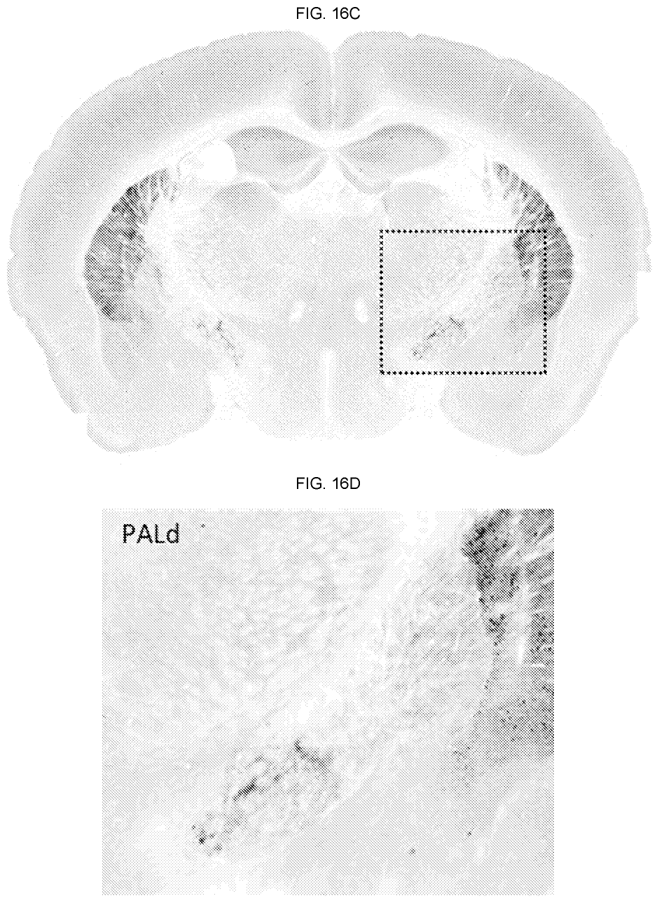

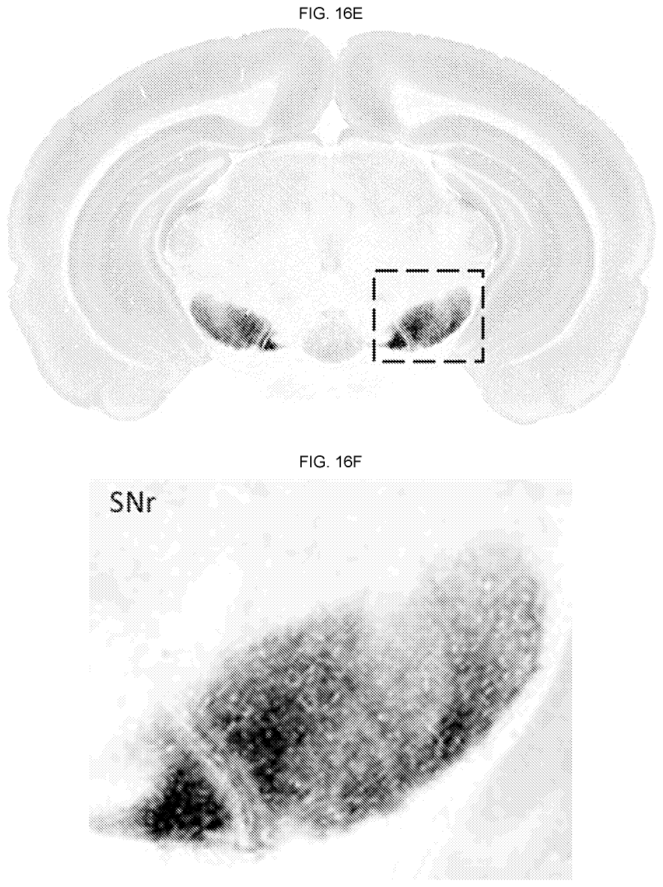

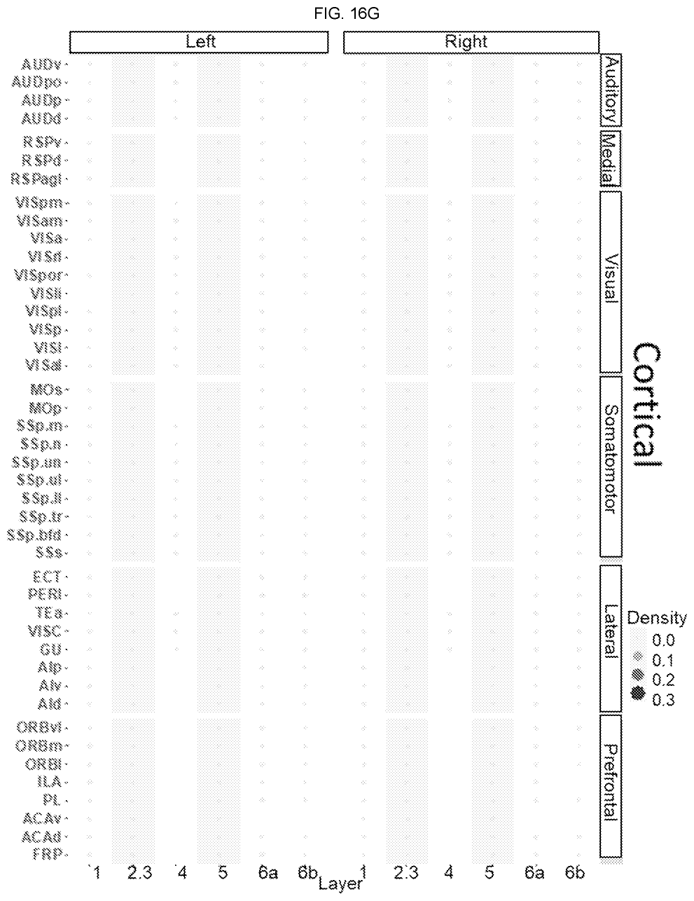

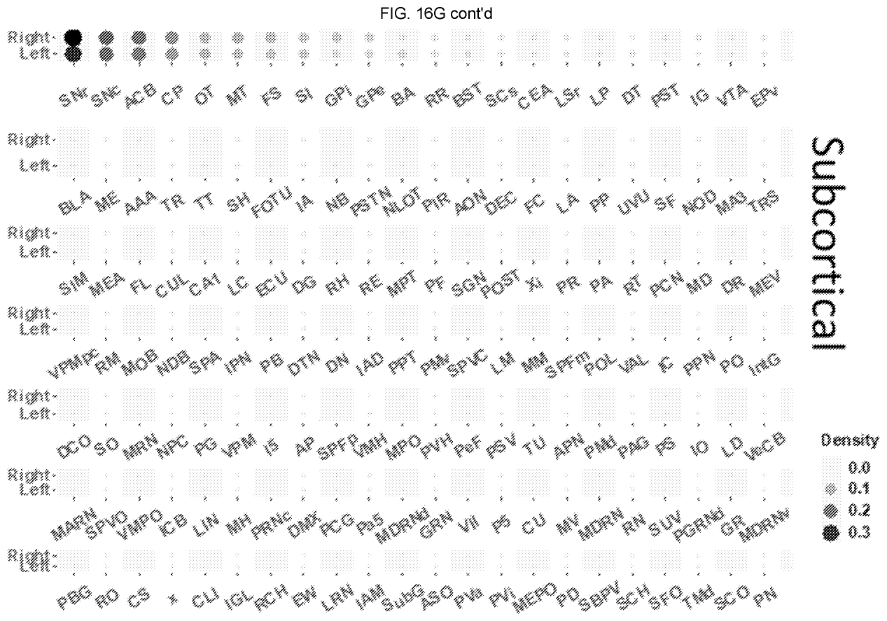

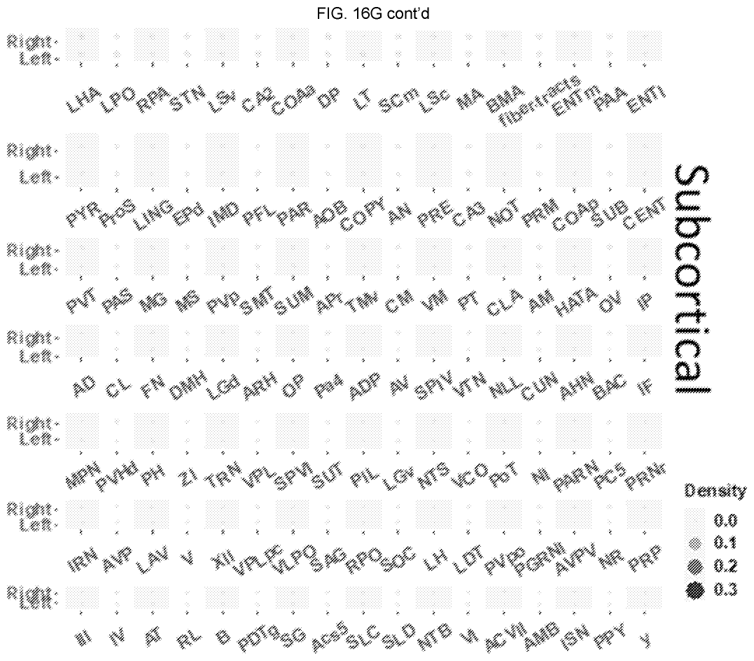

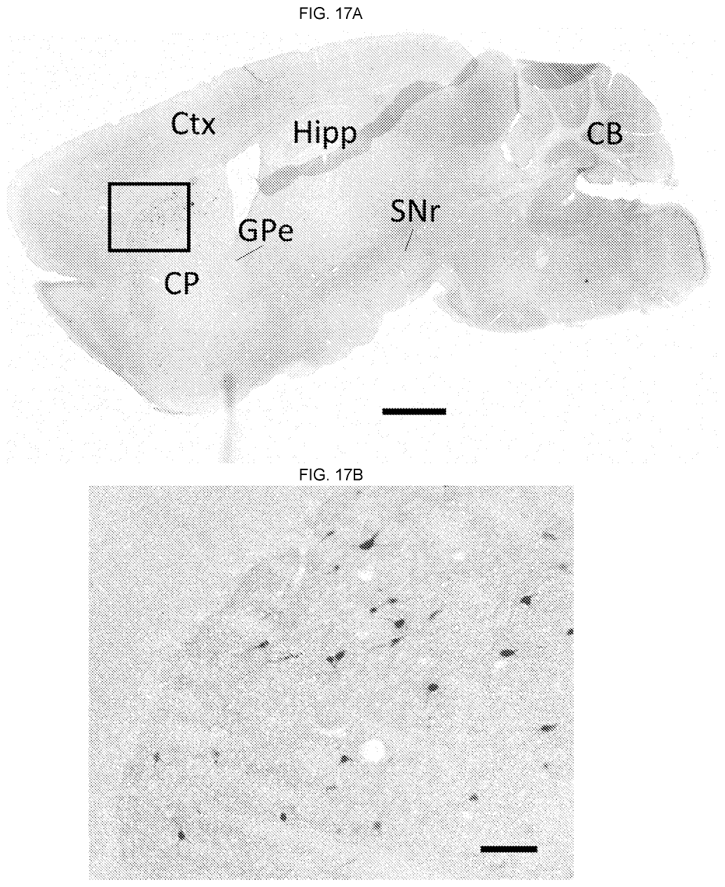

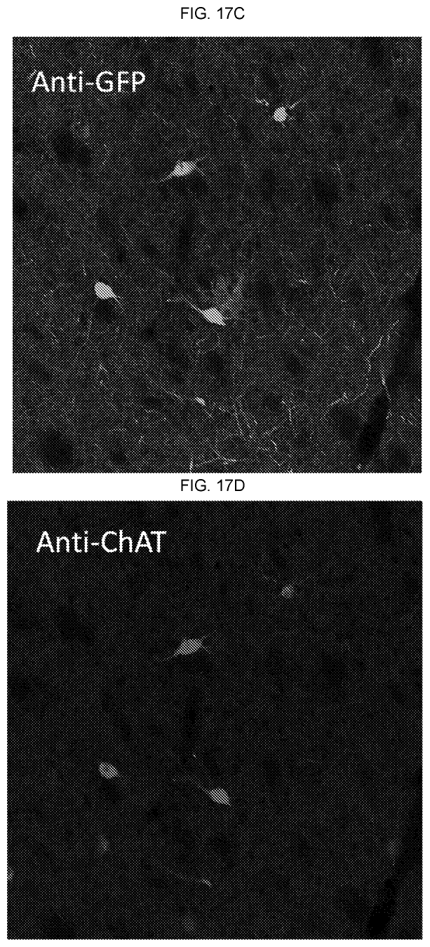

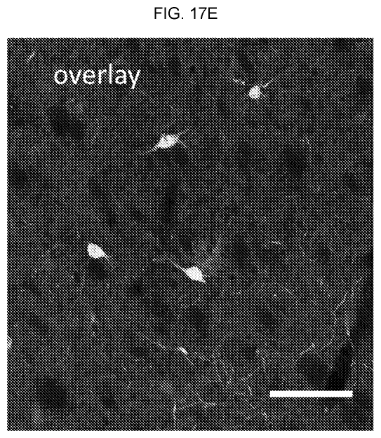

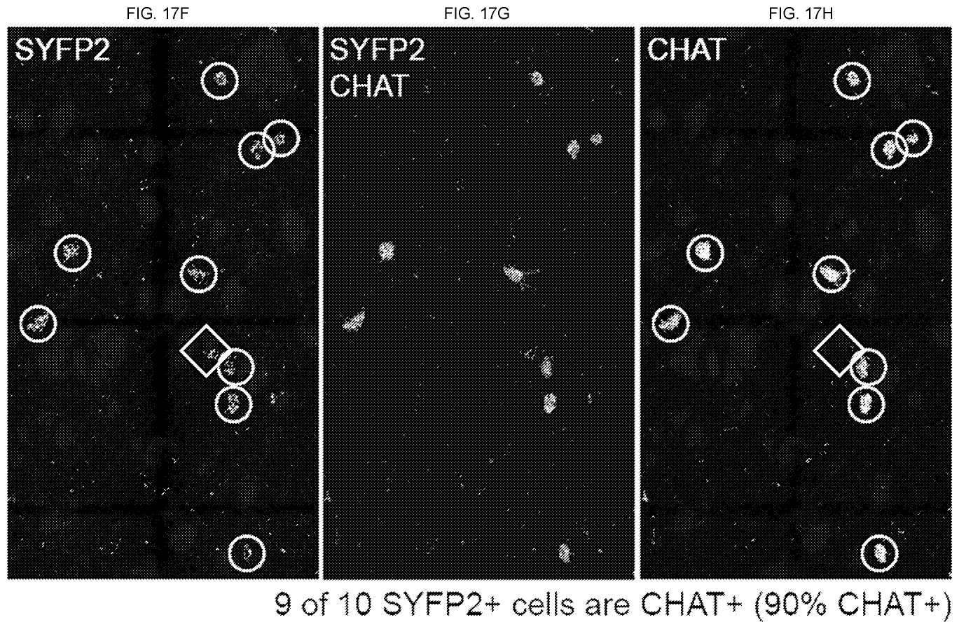

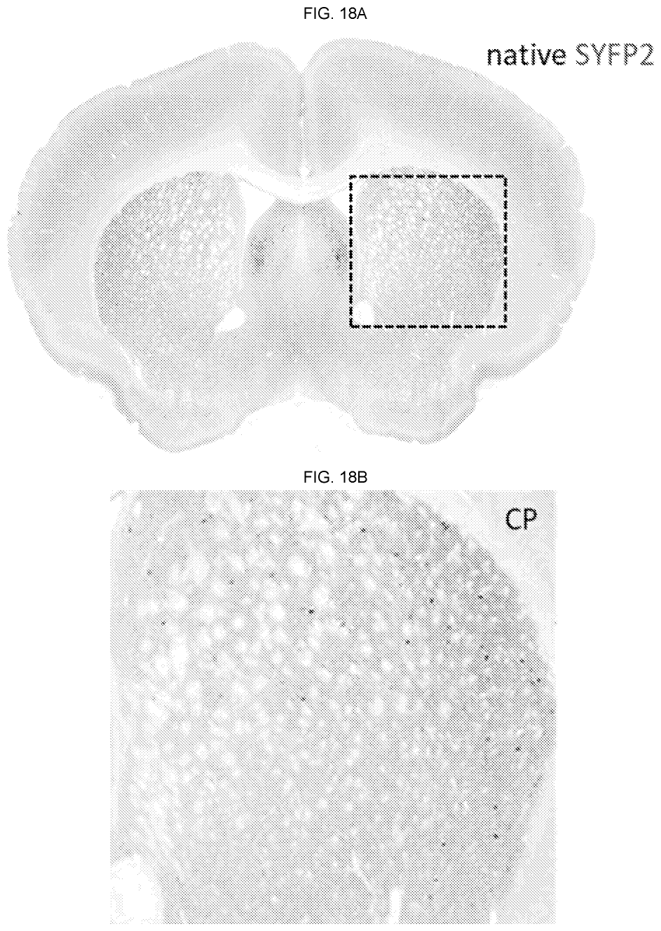

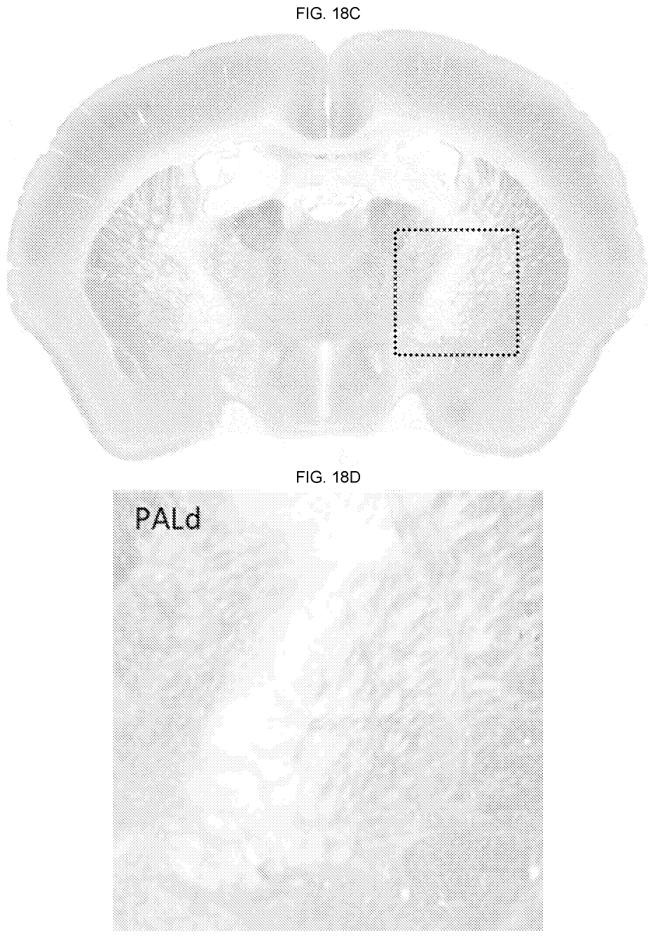

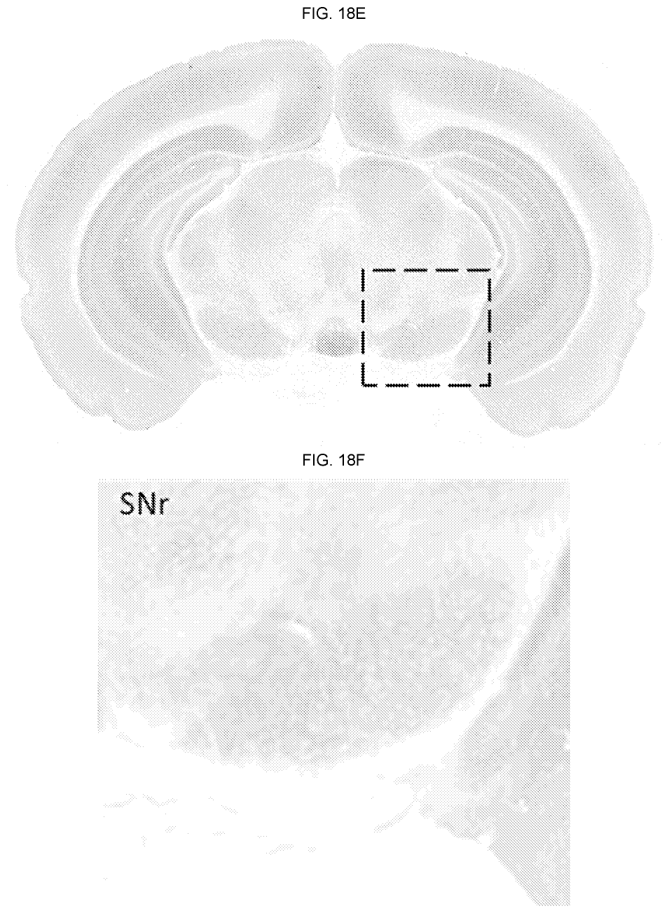

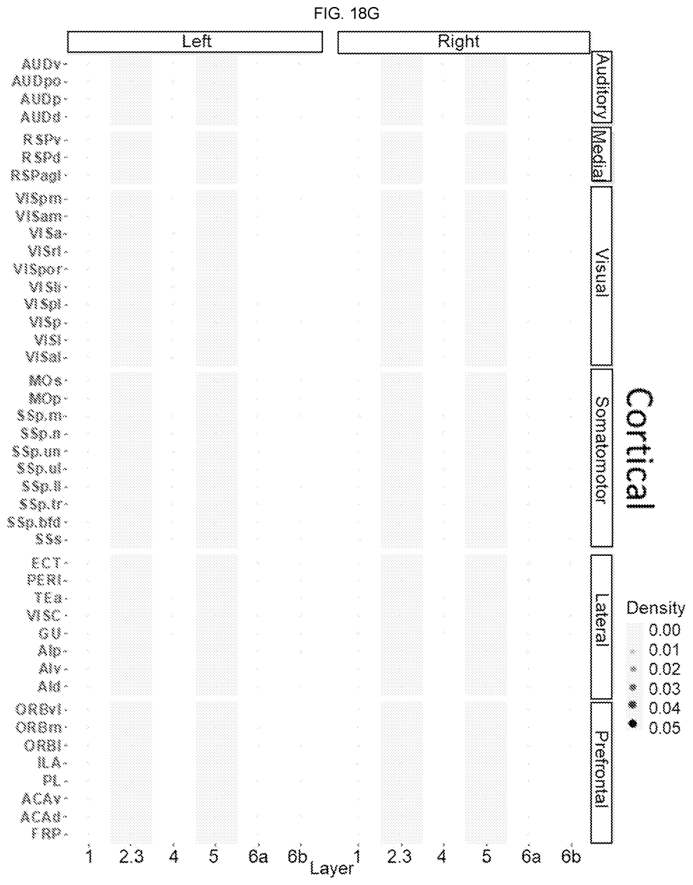

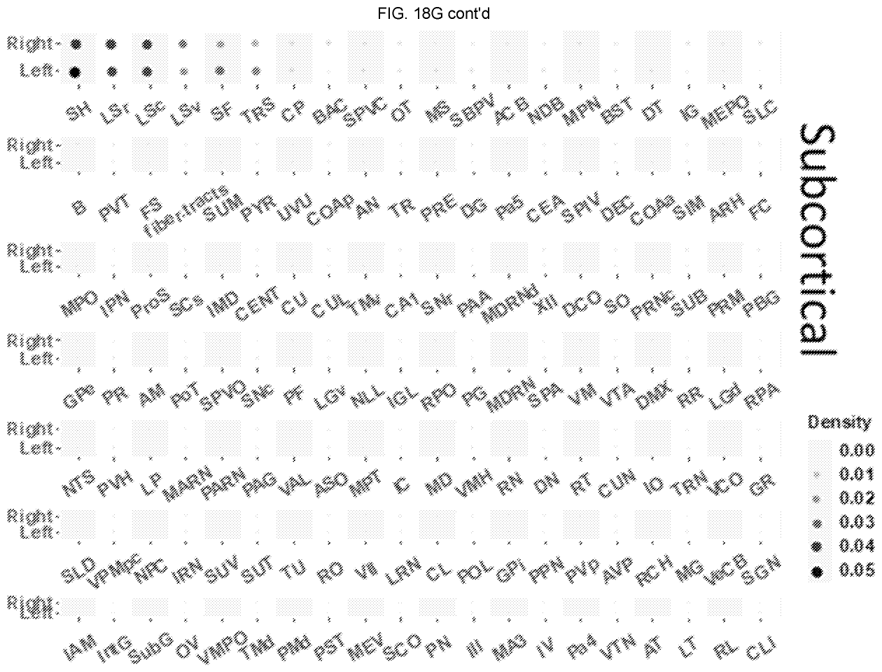

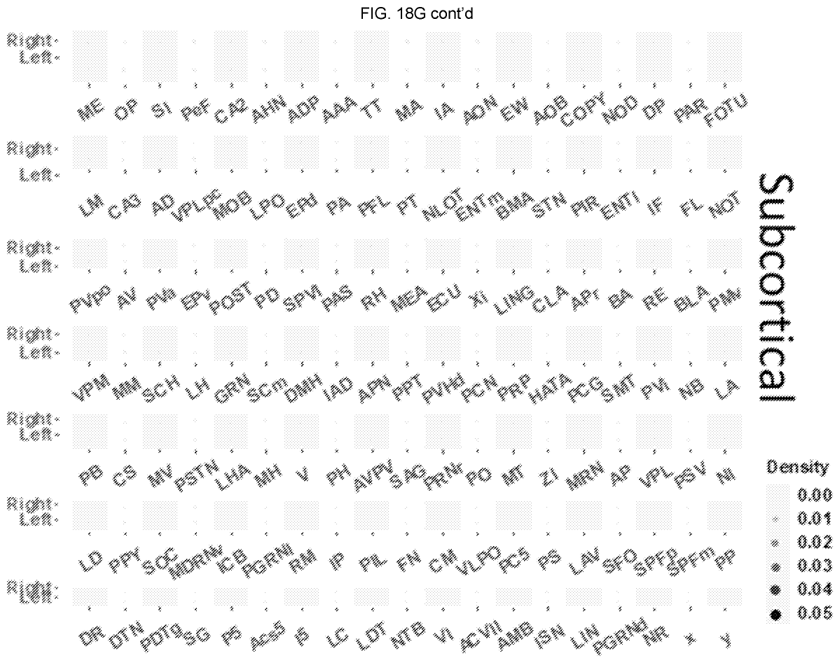

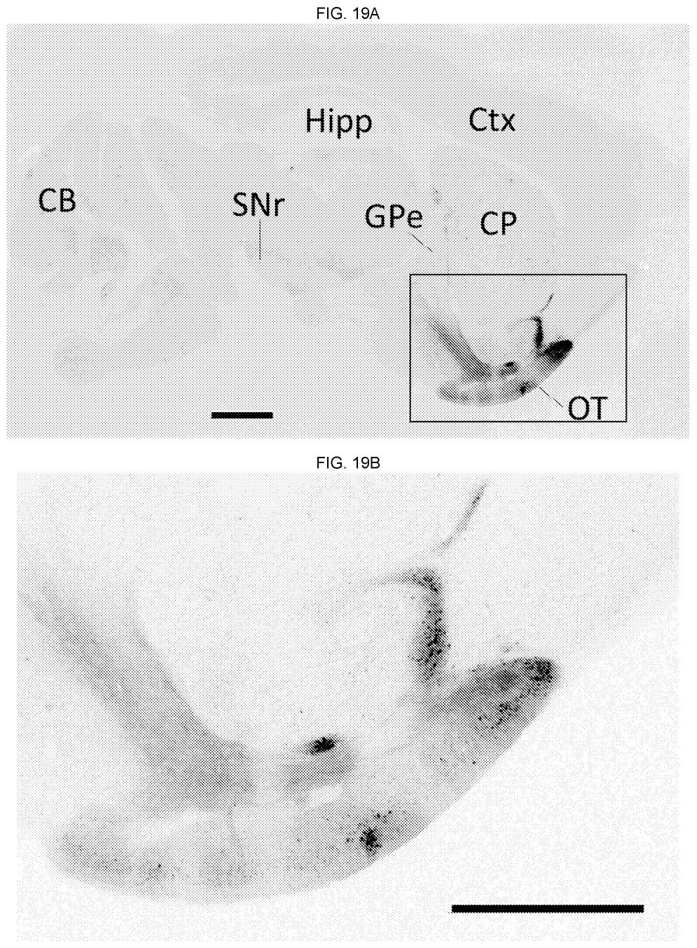

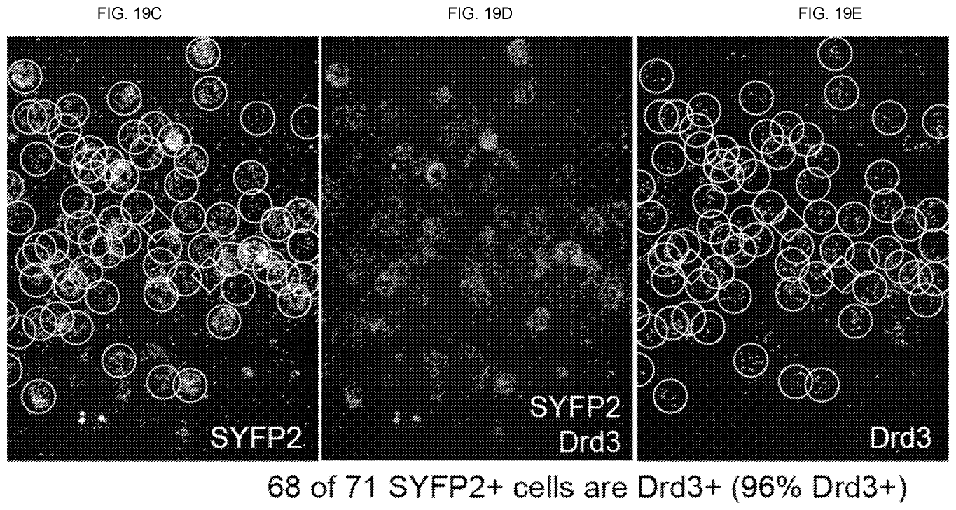

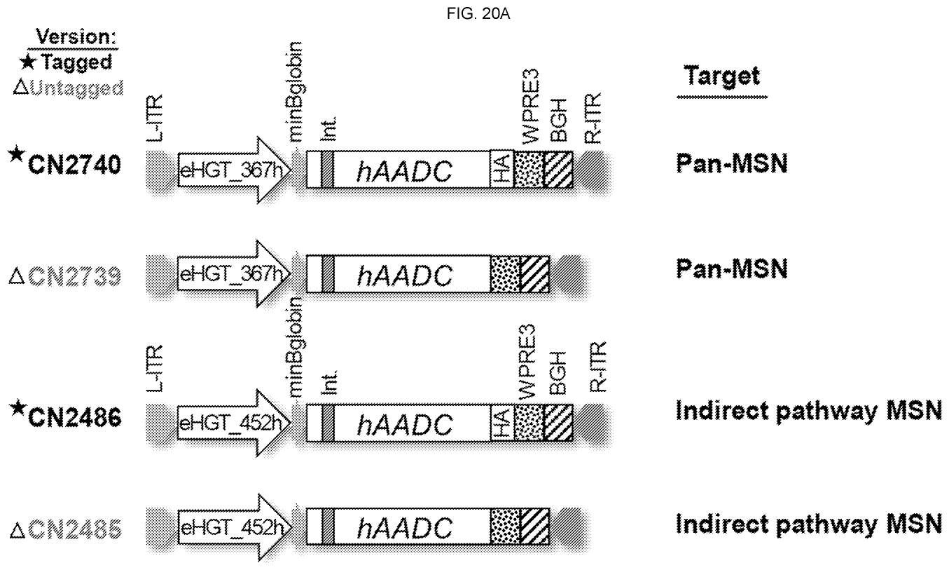

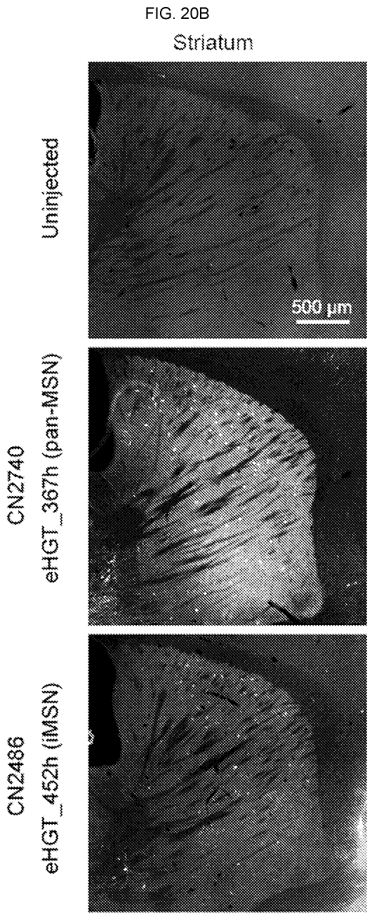

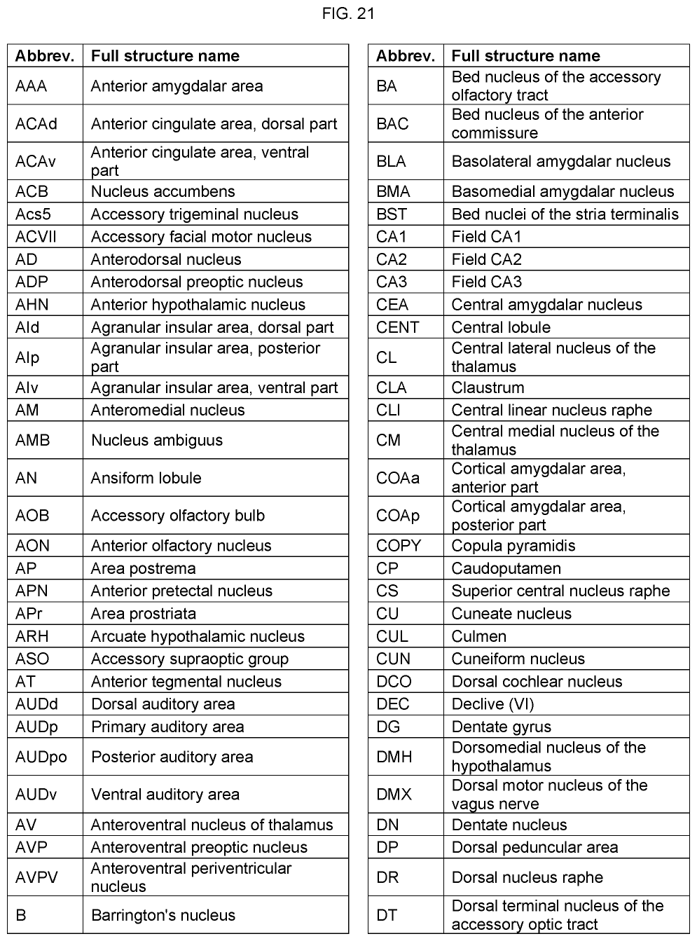

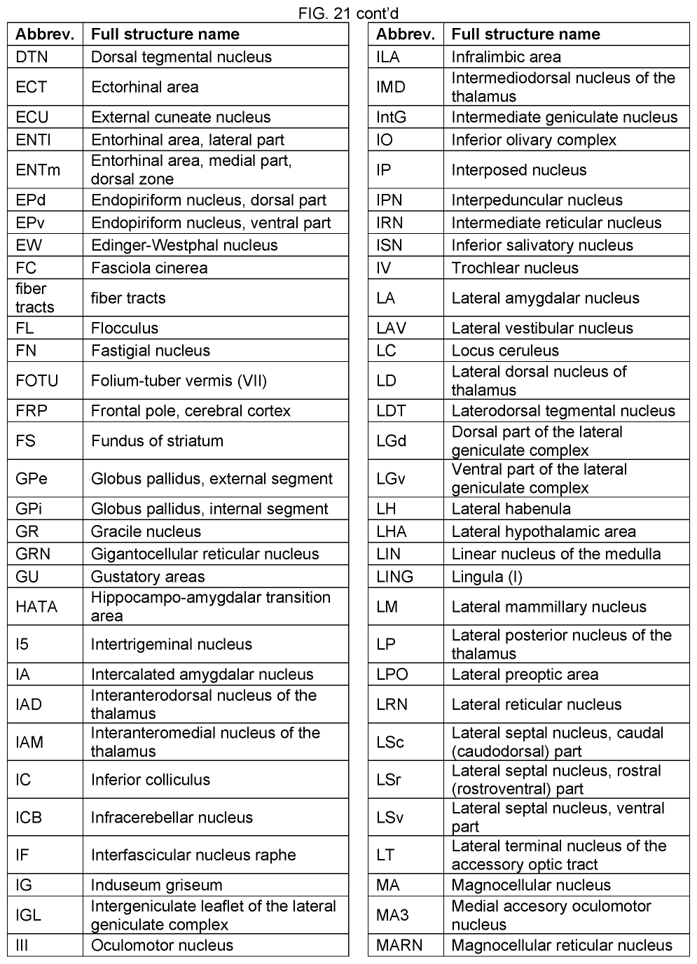

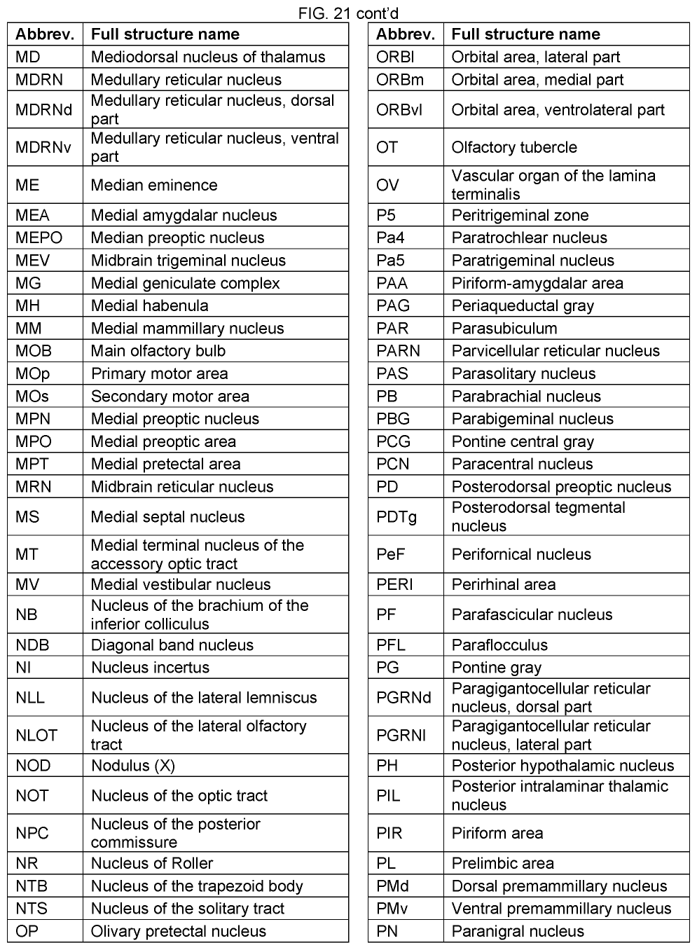

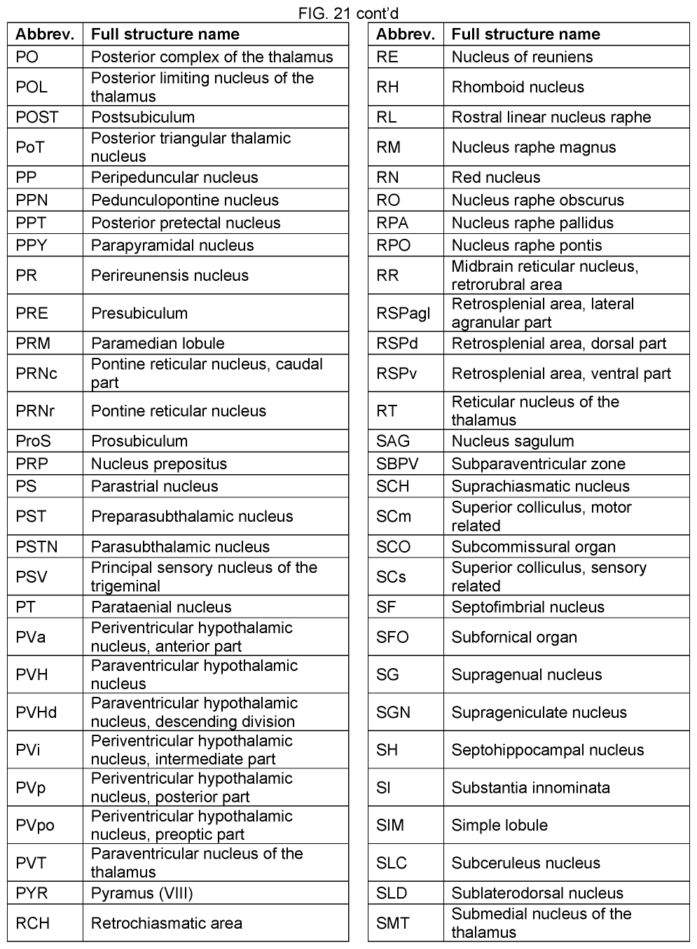

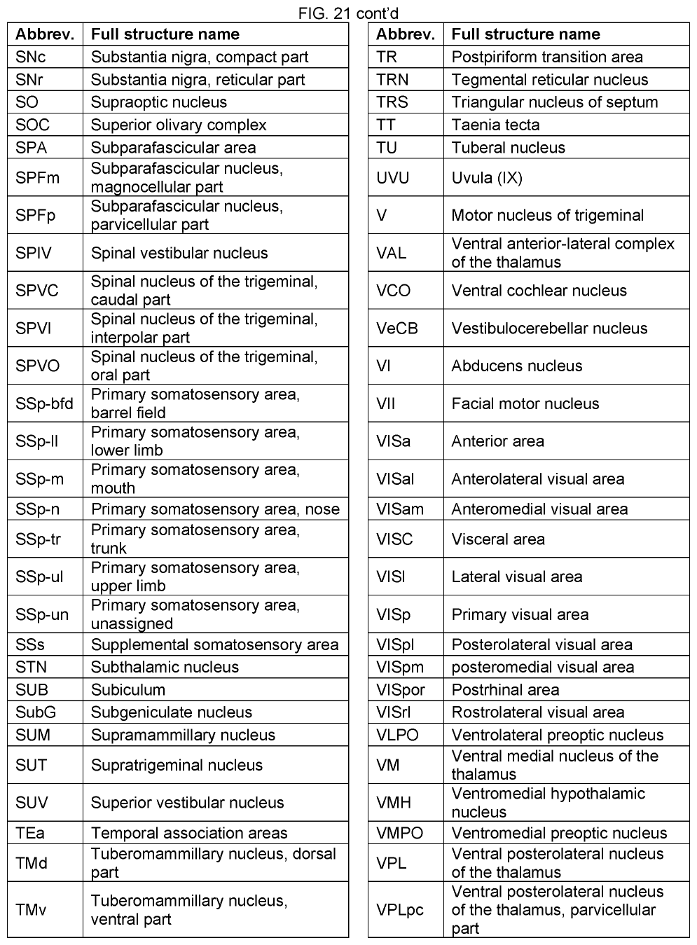

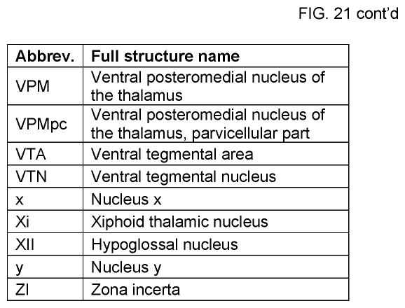

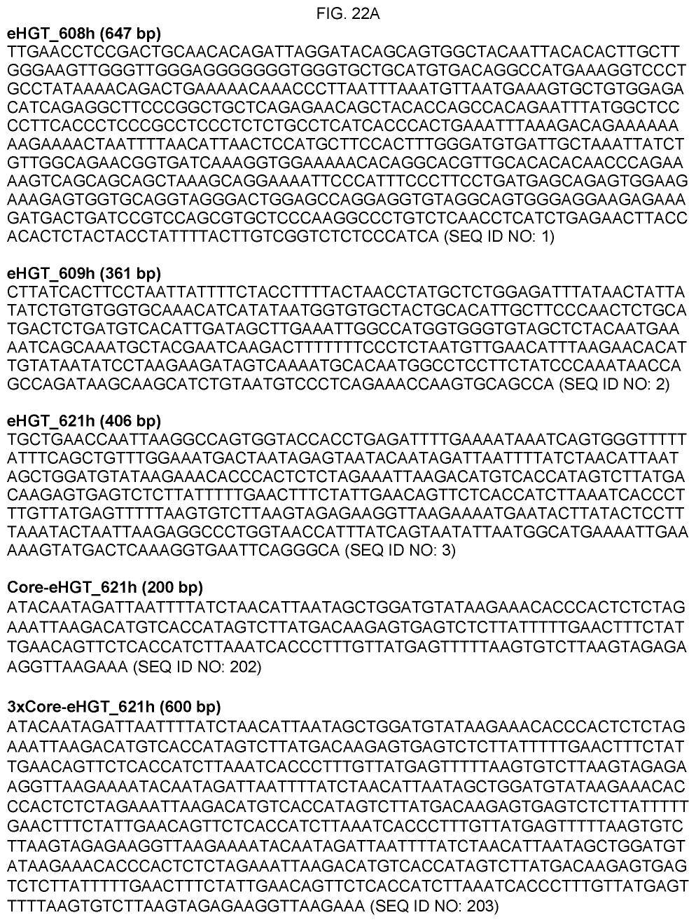

### DETAILED DESCRIPTION

To fully understand the biology of the brain, different cell types need to be distinguished and defined and, to further study them, artificial expression constructs that can label and perturb them need to be identified (Tasic, Curr. Opin. Neurobiol. 50, 242-249 (2018); Zeng & Sanes, Nat. Rev. Neurosci. 18, 530-546 (2017)). In mouse, recombinase driver lines have been used to great effect to label cell populations that share marker gene expression (Daigle et al., Cell 174, 465-480.e22 (2018); Taniguchi, et al., Neuron 71, 995-1013 (2011); Gong et al., J. Neurosci. 27, 9817-9823 (2007)). However, the creation, maintenance, and use of such lines that label cell types with high specificity can be costly, frequently requiring triple transgenic crosses, which yield a low frequency of experimental animals. Furthermore, those tools require germline transgenic animals and thus are not applicable to humans.

A movement disorder is a neurological disturbance that involves one or more muscles or muscle groups. Movement disorders affect a significant portion of the population, causing disability as well as distress. Movement disorders include Parkinson's disease, Huntington's chorea, progressive supranuclear palsy, multiple system atrophy (MSA), Aromatic L-amino acid decarboxylase (AADC) deficiency, Wilson's disease, Tourette's syndrome, epilepsy, tardive dyskinesia, and various chronic tremors, tics and dystonias.

Parkinson's disease is a movement disorder of increasing occurrence in aging populations. It affects one percent of the population over the age of 60 in the United States, such that the cumulative lifetime risk of an individual developing the disease is 1 in 40. Symptoms include pronounced tremor of the extremities, bradykinesia, rigidity and postural change. A perceived pathophysiological cause of Parkinson's disease is progressive destruction of dopamine producing cells in the basal ganglia which include the pars compacta of the substantia nigra. Parkinson's disease is a progressive disorder which can begin with mild limb stiffness and infrequent tremors and progress over a period of ten or more years to frequent tremors and memory impairment, to uncontrollable tremors and dementia. Although not a cure for Parkinson's disease, dopamine replacement, including treatment with the dopamine precursor levodopa (L-DOPA), is an effective therapeutic strategy.

In vivo, dopamine is synthesized from tyrosine by two enzymes, tyrosine hydroxylase (TH) and aromatic L-amino acid decarboxylase (AADC). In particular embodiments, TH can be a truncated form of the TH gene which avoids end-product feed-back inhibition by dopamine. Functional activity of TH depends on the availability of its cofactor tetrahydrobiopterin (BH4). GTP cyclohydrolase I (CH1) is the enzyme that catalyses the rate limiting step on the pathway of BH4-synthesis, to ensure that sufficient levels of the dopamine precursor, L-Dopa, are produced in vivo.

AADC is the enzyme responsible for the final step in the synthesis of dopamine. AADC deficiency is a rare genetic disorder believed to arise from mutation of the DDC gene (dopa decarboxylase). AADC deficiency results in severe developmental failures, global muscular hypotonia and dystonia, severe, long-lasting seizures known as oculo-gyric crises, frequent hospitalizations (including prolonged stays in intensive care), and the need for life-long care. Symptoms and severity vary depending on the type of underlying genetic mutation which abrogates AADC enzyme function. In particular embodiments, AADC includes human AADC (hAADC).

Mutations in GBA1, the gene encoding the lysosomal enzyme glucocerebrosidase (GCase), are linked to increased risk of Parkinson disease and Gaucher disease. Mutations in GBA1 may result in degradation of the enzyme and thus affect its function. Furthermore, the severity of PD symptoms correlate with the degree of enzyme activity reduction. GCase is a membrane-associated lysosomal hydrolase with 497 amino acids that cleaves, by hydrolysis, the beta-glucosidic linkage of glucocerebroside.

Tardive dyskinesia (TD) is a chronic disorder of the nervous system, characterized by involuntary, irregular rhythmic movements of the mouth, tongue, and facial muscles. The upper extremities also may be involved. These movements may be accompanied, to a variable extent, by other involuntary movements and movement disorders. These include rocking, writhing, or twisting movements of the trunk (tardive dystonia), forcible eye closure (tardive blepharospasm), an irresistible impulse to move continually (tardive akathisia), jerking movements of the neck (tardive spasmodic torticollis), and disrupted respiratory movements (respiratory dyskinesia).

Focal dystonias are a class of related movement disorders involving the intermittent sustained contraction of a group of muscles. The spasms of focal dystonia can last many seconds at a time, causing major disruption of the function of the affected area. Some of the focal dystonias are precipitated by repetitive movements; writer's cramp is the best-known example. Focal dystonia can involve the face (e.g., blepharospasm, mandibular dystonia), the neck (torticollis), the limbs (e.g., writer's cramp), or the trunk. Dystonia can occur spontaneously or can be precipitated by exposure to neuroleptic drugs and other dopamine receptor blockers (tardive dystonia).

A tic is an abrupt repetitive movement, gesture, or utterance that often mimics a normal type of behavior. Motor tics include movements such as eye blinking, head jerks or shoulder shrugs, but can vary to more complex purposive appearing behaviors such as facial expressions of emotion or meaningful gestures of the arms and head. Gilles de la Tourette syndrome (TS) is the most severe tic disorder. Patients with TS have multiple tics, including at least one vocal (phonic) tic. TS becomes apparent in early childhood with the presentation of simple motor tics, for example, eye blinking or head jerks. Initially, tics may come and go, but in time tics become persistent and severe, and begin to have adverse effects on the child and the child's family. Phonic tics manifest, on average, 1 to 2 years after the onset of motor tics.

Huntington's disease (HD) is a dominantly inherited neurodegenerative genetic disorder that affects muscle coordination and leads to cognitive decline and psychiatric problems. HD is due to mutations in the gene encoding for huntingtin, and it is the most common genetic cause of abnormal involuntary writhing movements called chorea. The most prominent early effects in HD are in a part of the basal ganglia called the neostriatum, which is composed of the caudate nucleus and putamen. Symptoms of the disease can vary between individuals, but usually progress predictably. The earliest symptoms are often subtle problems with mood or cognition, followed by a general lack of coordination and an unsteady gait. In advanced stages of the disease, uncoordinated, jerky body movements become more apparent, along with a decline in mental abilities, as well as behavioral and psychiatric problems. Physical abilities are gradually impeded until coordinated movement becomes very difficult, and mental abilities generally decline into dementia.

The problematic genetic mutation within the HTT gene involves a DNA segment of the huntingtin gene known as the CAG trinucleotide repeat. Normally, the CAG segment in the huntingtin gene of humans is repeated multiple times, i.e. 10-35 times. People with 36 to 39 CAG repeats may develop signs and symptoms of Huntington disease, while people with 40 or more repeats almost always develop the disorder. The increase in the size of the CAG repeat leads to the production of an elongated (mutated) huntingtin protein. This protein is processed in the cell into smaller fragments that are cytotoxic and that accumulate and aggregate in neurons. This results in the disruption of normal function and eventual death of neurons. Hypotheses on the molecular mechanisms underlying the neurotoxicity of polyglutamine-expanded HTT protein and its resultant aggregates have been wide ranging, but include, caspase activation, dysregulation of transcriptional pathways, increased production of reactive oxygen species, mitochondrial dysfunction, disrupted axonal transport and/or inhibition of protein degradation systems within the cell.

The current disclosure provides artificial expression constructs that drive gene expression in striatal neurons. The artificial expression constructs can be used for many purposes, including in the research and treatment of movement disorders.

Particular embodiments of the artificial expression constructs utilize the following enhancers to drive gene expression within targeted central nervous system cell populations as follows:
striatal medium spiny neuron-pan: eHGT_608h, eHGT_609h, eHGT_633h, eHGT_634h, eHGT_635h, eHGT_636h, eHGT_351h, eHGT_367h, eHGT_450h, eHGT_447h, eHGT_744m, eHGT_782m, eHGT_785m, and eHGT_441h;
striatal medium spiny neuron-indirect pathway: eHGT_612h, eHGT_613h, eHGT_614h, eHGT_617h, eHGT_618h, eHGT_619h, eHGT_620h, eHGT_442h, eHGT_444h, eHGT_445h, eHGT_452h, and eHGT_784m;
striatal medium spiny neuron-direct pathway: eHGT_610h, eHGT_611h, eHGT_615h, eHGT_616h, eHGT_627h, eHGT_628h, eHGT_629h, eHGT_446h, eHGT_779m, eHGT_780m, eHGT_781m, and eHGT_783m;
striatal interneuron-cholinergic: eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_746m, eHGT_747m, eHGT_748m, eHGT_749m, eHGT_750m, and eHGT_751m;
Drd3+ medium spiny neurons in olfactory tubercle: eHGT_621h.

In particular embodiments, the artificial enhancer elements include a concatenated core of an enhancer. Examples include a core or concatenated core of eHGT_367h, eHGT_441h, eHGT_445h, eHGT_444h, eHGT_452h, eHGT_779m, eHGT_743m, eHGT_621h, eHGT_780m, eHGT_447h, eHGT_351h, and/or eHGT_450h. These artificial enhancer elements can provide higher levels and more rapid onset of transgene expression compared to a single full length original (native) enhancer.

In particular embodiments, the enhancer core includes the sequence as set forth in any one of SEQ ID NOs: 202, 204, 206, 208, 210, 212, 214, 216, 218, 220, 222, 224, and 226. In particular embodiments, these cores are concatenated and have 2, 3, 4, 5, 6, 7, 8, 9, or 10 copies of the core sequence. SEQ ID NOs: 203, 205, 207, 209, 211, 213, 215, 217, 219, 221, 223, 225, and 227 provide three-copy concatemers of the described enhancer cores.

Particular embodiments of the artificial expression constructs utilize core2_eHGT_367h, 3×core2_eHGT_367h, 3×core_eHGT_441h, 3×Core-eHGT_447h, and/or 3×core2_eHGT_351h to drive gene expression within striatal medium spiny neuron-pan.

Particular embodiments of the artificial expression constructs utilize 3×core2_eHGT_445h, 3×core2_eHGT_444h, 3×core2_eHGT_452h, 3×core2_eHGT_450h, and/or 3×core3_eHGT_450h to drive gene expression within striatal medium spiny neuron-indirect pathway.

Particular embodiments of the artificial expression constructs utilize 3×core2_eHGT_779m and/or 3×Core2_eHGT_780m to drive gene expression within striatal medium spiny neuron-direct pathway.

Particular embodiments of the artificial expression constructs utilize 3×core2_eHGT_743m to drive gene expression within striatal interneuron-cholinergic.

Particular embodiments of the artificial expression constructs utilize 3×core-eHGT_621h to drive gene expression within Drd3+ medium spiny neurons in olfactory tubercle.

Particular embodiments provide artificial expression constructs including the features of vectors described herein including vectors: CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, and CN2700.

Aspects of the disclosure are now described with the following additional options and detail: (i) Artificial Expression Constructs & Vectors for Targeted Expression of Genes in Targeted Cell Types; (ii) Compositions for Administration (iii) Cell Lines Including Artificial Expression Constructs; (iv) Transgenic Animals; (v) Methods of Use; (vi) Kits and Commercial Packages; (vii) Exemplary Embodiments; and (viii) Closing Paragraphs. These headings are provided for organizational purposes only and do not limit the scope or interpretation of the disclosure.

(i) Artificial Expression Constructs & Vectors for Targeted Expression of Genes in Targeted Cell Types. Artificial expression constructs disclosed herein include (i) an enhancer sequence that leads to targeted expression of a coding sequence within a targeted central nervous system cell type, (ii) a coding sequence that is expressed, and (iii) a promoter. The artificial expression construct can also include other regulatory elements if necessary or beneficial. These headings do not limit the interpretation of the disclosure and are provided for organizational purposes only.

In particular embodiments, an “enhancer” or an “enhancer element” is a cis-acting sequence that increases the level of transcription associated with a promoter and can function in either orientation relative to the promoter and the coding sequence that is to be transcribed and can be located upstream or downstream relative to the promoter or the coding sequence to be transcribed. There are art-recognized methods and techniques for measuring function(s) of enhancer element sequences. Particular examples of enhancer sequences utilized within artificial expression constructs disclosed herein include eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, and eHGT_785m, and enhancer core core2_eHGT_367h, and concatenated cores, such as 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, and 3×core2_eHGT_743m.

In particular embodiments, a targeted central nervous system cell type enhancer is an enhancer that is uniquely or predominantly utilized by the targeted central nervous system cell type. A targeted central nervous system cell type enhancer enhances expression of a gene in the targeted central nervous system cell type.

When a heterologous coding sequence operatively linked to an enhancer disclosed herein leads to expression in a targeted cell type, it leads to expression of the administered heterologous coding sequence in the intended cell type.

When a heterologous coding sequence is selectively expressed in selected cells, it leads to expression of the administered heterologous coding sequence in the intended cell type and is not substantially expressed in other cell types, as explained in additional detail below. In particular embodiments, not substantially expressed in other cell types is less than 50% expression in a reference cell type as compared to a targeted cell type; less than 40% expression in a reference cell type as compared to a targeted cell type; less than 30% expression in a reference cell type as compared to a targeted cell type; less than 20% expression in a reference cell type as compared to a targeted cell type; or less than 10% expression in a reference cell type as compared to a targeted cell type. In particular embodiments, a reference cell type refers to non-targeted cells. The non-targeted cells can be within the same anatomical structure as the targeted cells and/or can project to a common anatomical area. In particular embodiments, a reference cell type is within an anatomical structure that is adjacent to an anatomical structure that includes the targeted cell type. In particular embodiments, a reference cell type is a non-targeted cell with a different gene expression profile than the targeted cells.

In particular embodiments, the product of the coding sequence may be expressed at low levels in non-targeted cell types, for example at less than 1% or 1%, 2%, 3%, 5%, 10%, 15% or 20% of the levels at which the product is expressed in targeted cell types. In particular embodiments, the targeted central nervous system cell type is the only cell type that expresses the right combination of transcription factors that bind an enhancer disclosed herein to drive gene expression. Thus, in particular embodiments, expression occurs exclusively within the selected cell type.

In particular embodiments, targeted cell types (e.g. neuronal, and/or non-neuronal) can be identified based on transcriptional profiles, such as those described in Tasic et al., Nature, 563, 72-78 (2018) and Hodge et al., Nature 573, 61-68 (2019). For reference, the following description of cell types and distinguishing features is also provided:

Targeted Striatal Cell Classes:

The striatum (Str) is important in translating cortical activity into voluntary motor actions. Regarding the striatum, corresponding structures in human/primate are called the putamen, caudate, and ventral striatum containing the nucleus accumbens. In rodent, the striatum includes the dorsal striatum plus the nucleus accumbens. Thus, in human/primate, the putamen and caudate collectively are the equivalent of the rodent dorsal striatum.

Striatal cell Classes and Subclasses:
Medium spiny neurons, pan: include 95% of striatal neurons and known to express GABA synthesis genes Gad1/GAD1 and Gad2/GAD2, as well as Ppp1r1b/PPP1R1B. Medium spiny neurons expressing Drd3 are herein referred to as Drd3+ medium spiny neurons.
Medium spiny neurons, direct pathway-projecting: include nearly 50% of striatal neurons and are enriched for expression of Drd1/DRD1, Pdyn/PDYN, and Slc35d3/SLC35D3. The major axon projection from direct pathway medium spiny neurons is to the substantia nigra pars reticulata (SNr) or to the inner division of the globus pallidus (GPi).
Medium spiny neurons, indirect pathway-projecting: include nearly 50% of striatal neurons and are enriched for expression of Drd2/DRD2, Adora2a/ADORA2A, Gpr6/GPR6, and Penk/PENK. The major axon projection from indirect pathway medium spiny neurons is to the external segment of the globus pallidus (GPe).
Striatal interneuron-cholinergic: A rare interneuron population including 1% of striatal neurons. These local interneurons have large somata and aspiny dendrites and are known to express Chat/CHAT and release the neurotransmitter acetylcholine.

Neocortical GABAergic neuron Subclasses:
All: Express GABA synthesis genes Gad1/GAD1 and Gad2/GAD2.
Lamp5, Sncg, Serpinf1, and Vip GABAergic neurons: Developmentally derived from neuronal progenitors from the caudal ganglionic eminence (CGE) or preoptic area (POA).
Sst and Pvalb GABAergic neurons: Developmentally derived from neuronal progenitors in the medial ganglionic eminence (MGE).
Lamp5 GABAergic neurons: Found in many neocortical layers, especially upper (L1-L2/3), and have mainly neurogliaform and single bouquet morphology.
Lamp5_Lhx6 GABAergic neurons: A subset of Lamp5 GABAergic neurons that co-express Lamp5 and Lhx6.
Sncg GABAergic neurons: Found in many neocortical layers, and have molecular overlaps with Lamp5 and Vip cells, but inconsistent expression of Lamp5 or Vip, with more consistent expression of Sncg.
Serpinf1 GABAergic neurons: Found in many neocortical layers, and have molecular overlaps with Sncg and Vip cells, but inconsistent expression of Sncg or Vip, with more consistent expression of Serpinf1.
Vip GABAergic neurons: Found in many neocortical layers, but especially frequent in upper layers (L1-L4), and highly express the neurotransmitter vasoactive intestinal peptide (Vip).
Sst GABAergic neurons: Found in many neocortical layers, but especially frequent in lower layers (L5-L6). They highly express the neurotransmitter somatostatin (Sst), and frequently block dendritic inputs to postsynaptic neurons. Included in this subclass are sleep-active Sst Chodl neurons (which also express Nos1 and Tacr1) that are highly distinct from other Sst neurons but express some shared marker genes including Sst. In human, SST gene expression is often detected in layer 1 LAMP5+ GABAergic neuron subtypes.
Pvalb GABAergic neurons: Found in many neocortical layers, but especially frequent in lower layers (L5-L6). They highly express the calcium-binding protein parvalbumin (Pvalb), express neuropeptide Tac1, and frequently dampen the output of postsynaptic neurons. Most fast-spiking GABAergic neurons express Pvalb strongly. Included in this subclass are chandelier cells, which have distinct, chandelier-like morphology and express the markers Cpne5 and Vipr2 in mouse, and NOG and UNC5B in human.
Meis2: A distinct subclass defined by a single type, only neocortical GABAergic neuron type that expresses Meis2 gene, and does not express some other genes that are expressed by all other neocortical GABAergic neuron types (for example, Thy1 and Scn2b). This type is found in L6b and subcortical white matter.

Neocortical Glutamatergic neuron Subclasses:
All: Express glutamate transmitters Slc17a6 and/or Slc17a7. They all express Snap25 and lack expression of Gad1/Gad2.
L2/3 IT glutamatergic neurons: Primarily reside in Layer 2/3 and have mainly intratelencephalic (cortico-cortical) projections.
L4 IT glutamatergic neurons: Primarily reside in Layer 4 and mainly have either local or intratelencephalic (cortico-cortical) projections.
L5 IT glutamatergic neurons: Primarily reside in Layer 5 and have mainly intratelencephalic (cortico-cortical) projections. Also called L5a.
L5 PT glutamatergic neurons: Primarily reside in Layer 5 and have mainly cortico-subcortical (pyramidal tract or corticofugal) projections. Also called L5b or L5 CF (corticofugal) or L5 ET (extratelencephalic). This subclass includes cells that are located in the primary motor cortex and neighboring areas and are corticospinal projection neurons, which are associated with motor neuron/movement disorders, such as ALS. This subclass includes thick-tufted pyramidal neurons, including distinctive cell types found only in specialized regions, e.g. Betz cells, Meynert cells, and von Economo cells.
L5 NP glutamatergic neurons: Primarily reside in Layer 5 and have mainly nearby projections.
L6 CT glutamatergic neurons: Primarily reside in Layer 6 and have mainly cortico-thalamic projections.
L6 IT glutamatergic neurons: Primarily reside in Layer 6 and have mainly intratelencephalic (cortico-cortical) projections.
L6 IT Car3 glutamatergic neurons: Most densely present in claustrum and endopyriform nucleus, and sparsely throughout L6 in many cortical areas including the primary visual cortex. These cells have mainly intratelencephalic (cortico-cortical) projections. Additional marker genes for claustrum enriched neurons include Gnb4 and Ntng2.
L6b glutamatergic neurons: Primarily reside in the neocortical subplate (L6b), with local (near the cell body) projections and some cortico-cortical projections from VISp to anterior cingulate, and cortico-subcortical projections to the thalamus.
CR neurons: A distinct subclass defined by a single type in L1, Cajal-Retzius cells express distinct molecular markers Lhx5 and Trp73.

The cerebellum is located at the posterior of the brain. The cerebellum processes inputs from the cerebral motor cortex, different brainstem nuclei, and sensory receptors. Two types of neurons play major roles in the cerebellar ciruit, including Purkinje cells and granule cells. The cerebellum also receives dopaminergic, serotonergic, noradrenergic, and cholinergic inputs.
Cerebellar Purkinje cells: large GABAergic neurons that are the only projection neurons and the sole output from the cerebellum. Their cell bodies form a single layer, so called ‘Purkinje cell layer’, and they express parvalbumin.
Deep cerebellar nucleus neurons: neurons located in the deep cerebellar nuclei structures. These include glutamatergic and GABAergic cells that express the gene Pvalb.

Non-neuronal Subclasses:
Astrocytes: Neuroectoderm-derived glial cells which express the marker Aqp4 and often GFAP, but do not express neuronal marker SNAP25. They can have a distinct star-shaped morphology and are involved in metabolic support of other cells in the brain. Multiple astrocyte morphologies are observed in mouse and human
Oligodendrocytes: Neuroectoderm-derived glial cells, which express the marker Sox10. This category includes oligodendrocyte precursor cells (OPCs). Oligodendrocytes are the subclass that is primarily responsible for myelination of neurons.
VLMCs: Vascular leptomeningeal cells (VLMCs) are part of the meninges that surround the outer layer of the cortex and express the marker genes Lum and Col1a1.
Pericytes: Blood vessel-associated cells that express the marker genes Kcnj8 and Abcc9. Pericytes wrap around endothelial cells and are important for regulation of capillary blood flow and are involved in blood-brain barrier permeability.
SMCs: Specialized smooth-muscle cells which are blood vessel-associated cells that express the marker gene Acta2. SMCs cover arterioles in the brain and are involved in blood-brain barrier permeability.
Endothelial cells: Cells that line blood vessels of the brain. Endothelial cells express the markers Tek and PDGF-B.
Microglia: hematopoietic-derived immune cells, which are brain-resident macrophages, and perivascular macrophages (PVMs) that may be transitionally associated with brain tissue or included as a biproduct of brain dissection methods. Microglia are known to express Cx3cr1, Tmem119, and PTPRC (CD45).

Olfactory Tubercle (OT): The olfactory tubercle is a multi-sensory processing center that is located on the ventral surface of the frontal lobe. The olfactory tubercle has outputs directed towards the thalamus, ventral pallidum, nucleus accumbens, and in some primates the orbitofrontal cortex.

Cortex (Ctx) The cortex is the outer layer of the brain (also referred to as the grey matter). The four lobes of the cortex include the frontal lobe, parietal lobe, temporal lobe, and occipital lobe.

Hippocampus (Hipp): The hippocampus is a C-shaped brain structure embedded deep into the temporal lobe. The hippocampus is the posterior part of limbic lobe while the frontal part is amygdala. The hippocampus is known to play a role in learning, memory and spatial navigation.

Caudoputamen (CP): The caudoputamen is also referred to as the dorsal striatum is one of the structures that form the basal ganglia.

Dorsal Pallidum (PALd): The dorsal pallidum is also referred to as the globus pallidus. The globus pallidus is a subcortical structure of the brain including an external (referred to as globus pallidus external segment (GPe)) and an internal (referred to as globus pallidus internal segment (GPi)) segment.

In particular embodiments, a coding sequence is a heterologous coding sequence that encodes an effector element. An effector element is a sequence that is expressed to achieve, and that in fact achieves, an intended effect. Examples of effector elements include reporter genes/proteins and functional genes/proteins.

Exemplary reporter genes/proteins include those expressed by Addgene ID #s 83894 (pAAV-hDIx-Flex-dTomato-Fishell_7), 83895 (pAAV-hDIx-Flex-GFP-Fishell_6), 83896 (pAAV-hDIx-GiDREADD-dTomato-Fishell-5), 83898 (pAAV-mDIx-ChR2-mCherry-Fishell-3), 83899 (pAAV-mDIx-GCaMP6f-Fishell-2), 83900 (pAAV-mDIx-GFP-Fishell-1), and 89897 (pcDNA3-FLAG-mTET2 (N500)). Exemplary reporter genes particularly can include those which encode an expressible fluorescent protein, or expressible biotin; blue fluorescent proteins (e.g. eBFP, eBFP2, Azurite, mKalama1, GFPuv, Sapphire, T-sapphire); cyan fluorescent proteins (e.g. eCFP, Cerulean, CyPet, AmCyanl, Midoriishi-Cyan, mTurquoise); green fluorescent proteins (e.g. GFP, GFP-2, tagGFP, turboGFP, EGFP, Emerald, Azami Green, Monomeric Azami Green (mAzamigreen), CopGFP, AceGFP, avGFP, ZsGreenl, Oregon Green™ (Thermo Fisher Scientific)); Luciferase; orange fluorescent proteins (mOrange, mKO, Kusabira-Orange, Monomeric Kusabira-Orange, mTangerine, tdTomato, dTomato); red fluorescent proteins (mKate, mKate2, mPlum, DsRed monomer, mCherry, mRuby, mRFP1, DsRed-Express, DsRed2, DsRed-Monomer, HcRed-Tandem, HcRedl, AsRed2, eqFP611, mRaspberry, mStrawberry, Jred, Texas Red™ (Thermo Fisher Scientific)); far red fluorescent proteins (e.g., mPlum and mNeptune); yellow fluorescent proteins (e.g., YFP, eYFP, Citrine, SYFP2, Venus, YPet, PhiYFP, ZsYellowl); and tandem conjugates.

GFP is composed of 238 amino acids (26.9 kDa), originally isolated from the jellyfish Aequorea victoria/Aequorea aequorea/Aequorea forskalea that fluoresces green when exposed to blue light. The GFP from A. victoria has a major excitation peak at a wavelength of 395 nm and a minor one at 475 nm. Its emission peak is at 509 nm which is in the lower green portion of the visible spectrum. The GFP from the sea pansy (Renilla reniformis) has a single major excitation peak at 498 nm. Due to the potential for widespread usage and the evolving needs of researchers, many different mutants of GFP have been engineered. The first major improvement was a single point mutation (S65T) reported in 1995 in Nature by Roger Tsien. This mutation dramatically improved the spectral characteristics of GFP, resulting in increased fluorescence, photostability and a shift of the major excitation peak to 488 nm with the peak emission kept at 509 nm. The addition of the 37° C. folding efficiency (F64L) point mutant to this scaffold yielded enhanced GFP (EGFP). EGFP has an extinction coefficient (denoted E), also known as its optical cross section of 9.13×10-21 m²/molecule, also quoted as 55,000 L/(mol·cm). Superfolder GFP, a series of mutations that allow GFP to rapidly fold and mature even when fused to poorly folding peptides, was reported in 2006.

The “yellow fluorescent protein” (YFP) is a genetic mutant of green fluorescent protein, derived from Aequorea victoria. Its excitation peak is 514 nm and its emission peak is 527 nm.

Exemplary functional molecules include functioning ion transporters, cellular trafficking proteins, enzymes, transcription factors, neurotransmitters, calcium reporters, channelrhodopsins, guide RNA, microRNA, nucleases, or designer receptors exclusively activated by designer drugs (DREADDs).

Ion transporters are transmembrane proteins that mediate transport of ions across cell membranes. These transporters are pervasive throughout most cell types and important for regulating cellular excitability and homeostasis. Ion transporters participate in numerous cellular processes such as action potentials, synaptic transmission, hormone secretion, and muscle contraction. Many important biological processes in living cells involve the translocation of cations, such as calcium (Ca2+), potassium (K+), and sodium (Na+) ions, through such ion channels. In particular embodiments, ion transporters include voltage gated sodium channels (e.g., SCN1A), potassium channels (e.g., KCNQ2), and calcium channels (e.g. CACNA1C)).

In particular embodiments, the amino acid sequence of human sodium channel protein type 1 subunit alpha includes UniProtKB: P35498; the amino acid sequence of human potassium voltage-gated channel subfamily KQT member 2 includes UniProtKB: 043526; and/or the amino acid sequence of human voltage-dependent L-type calcium channel subunit alpha-1C includes UniProtKB: Q13936.

Exemplary enzymes, transcription factors, receptors, membrane proteins, cellular trafficking proteins, signaling molecules, and neurotransmitters include enzymes such as aromatic L-amino acid decarboxylase (AADC including human AADC (hAADC)), tyrosine hydroxylase (TH), GTP cyclohydrolase I (CH1), glucocerebrosidase (GCase), lactase, lipase, helicase, alpha-glucosidase, amylase; transcription factors such as SP1, AP-1, Heat shock factor protein 1, C/EBP (CCAA-T/enhancer binding protein), and Oct-1; receptors such as transforming growth factor receptor beta 1, platelet-derived growth factor receptor, epidermal growth factor receptor, vascular endothelial growth factor receptor, and interleukin 8 receptor alpha; membrane proteins, cellular trafficking proteins such as clathrin, dynamin, caveolin, Rab-4A, and Rab-11A; signaling molecules such as nerve growth factor (NGF), platelet-derived growth factor (PDGF), transforming growth factor β (TGFβ), epidermal growth factor (EGF), GTPase and HRas; and neurotransmitters such as cocaine and amphetamine regulated transcript, substance P, oxytocin, and somatostatin.

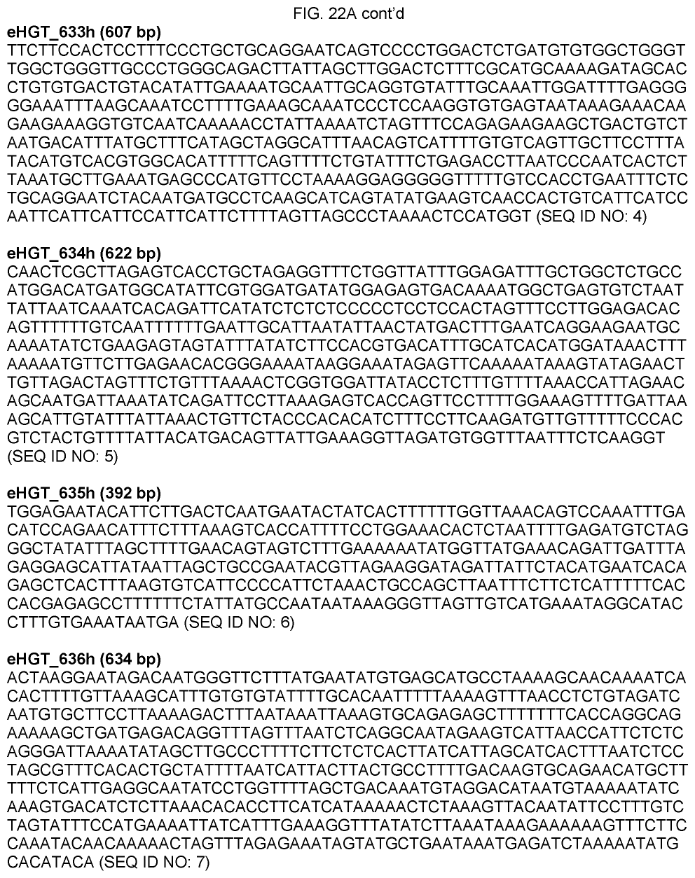

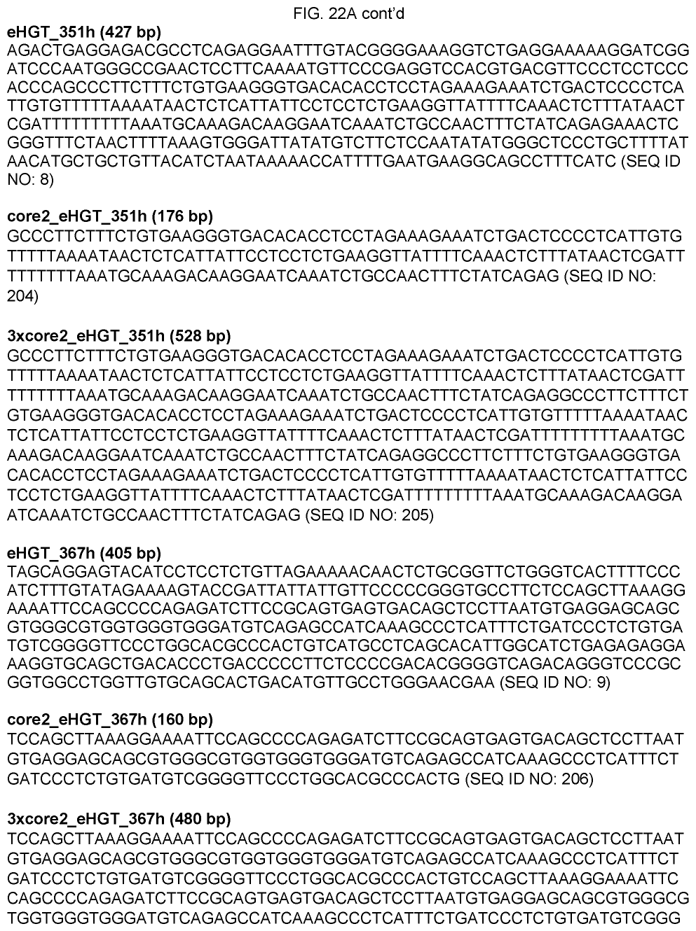

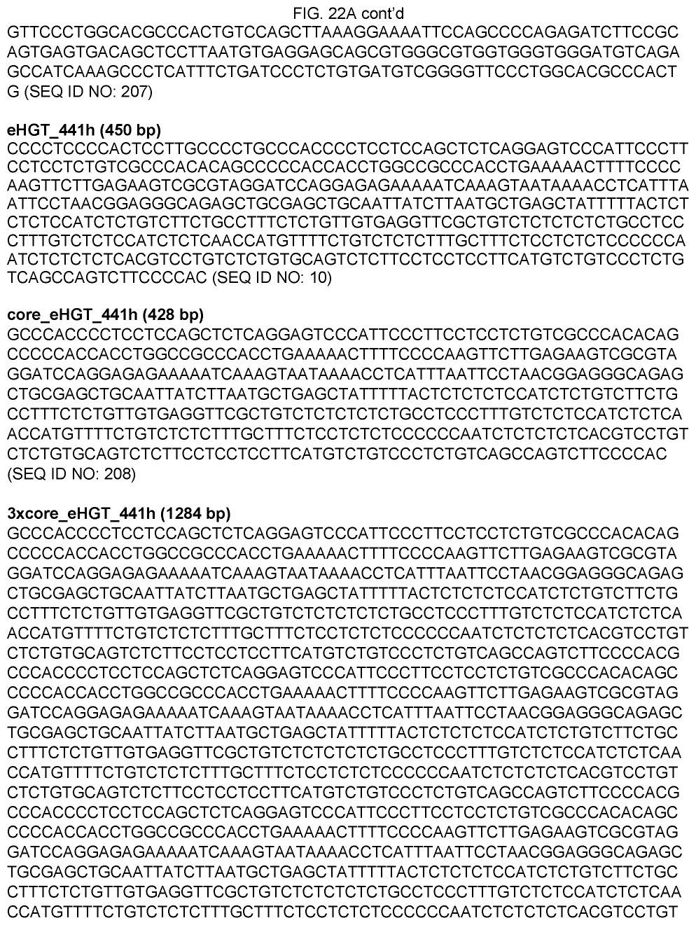

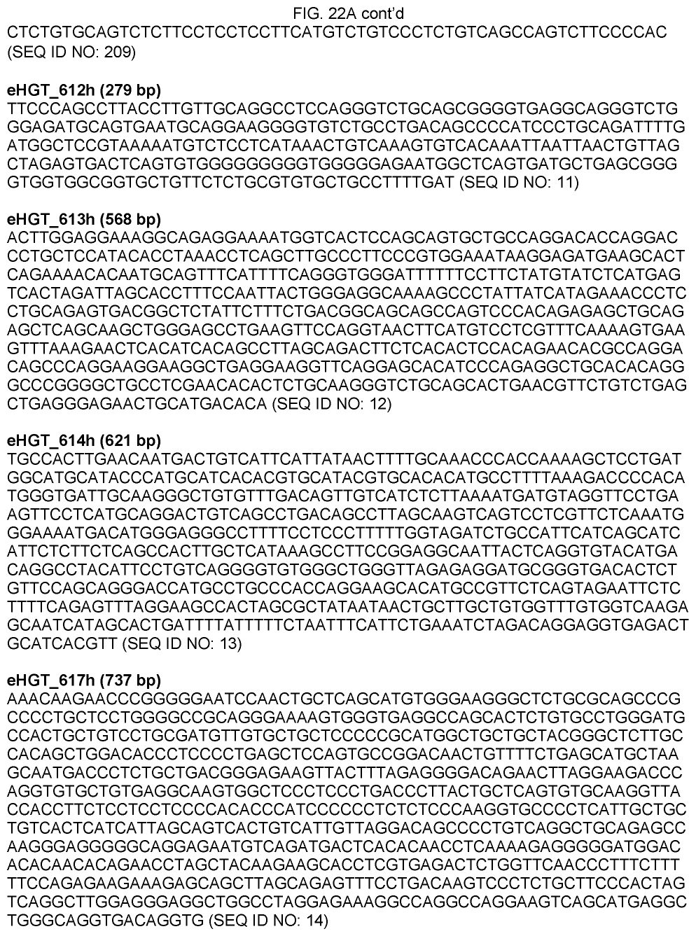

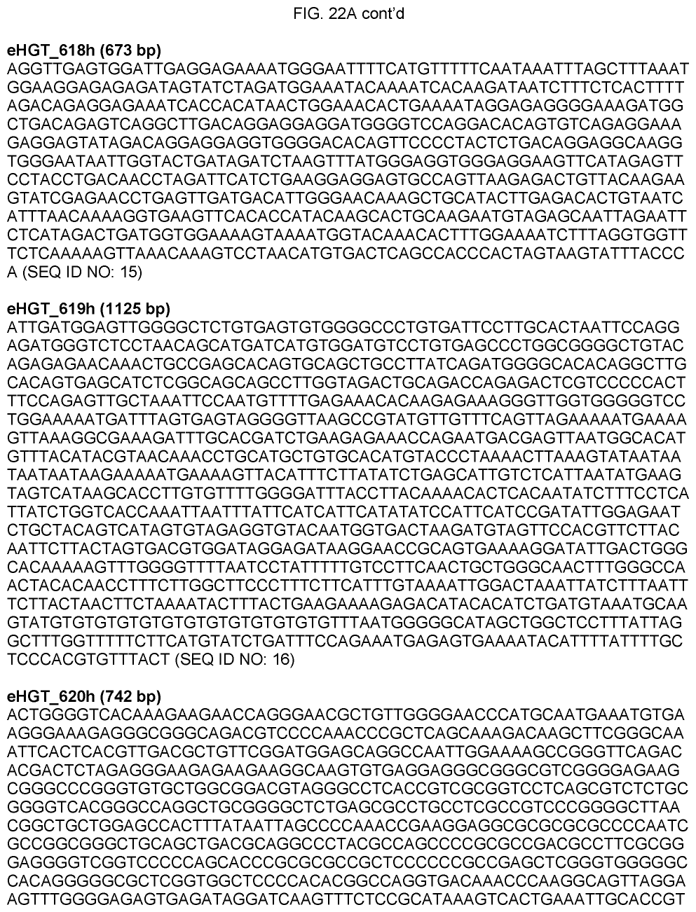

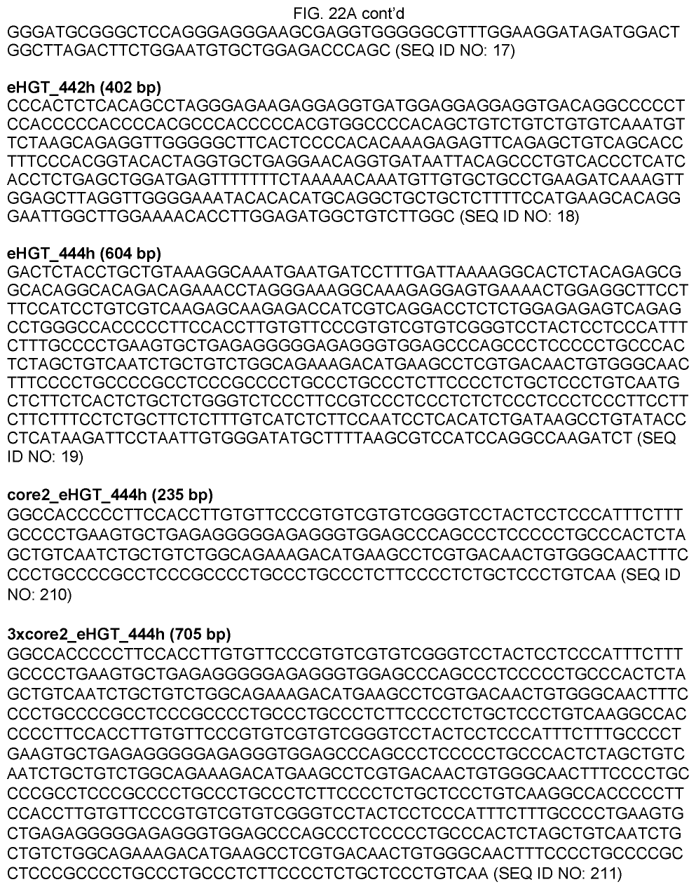

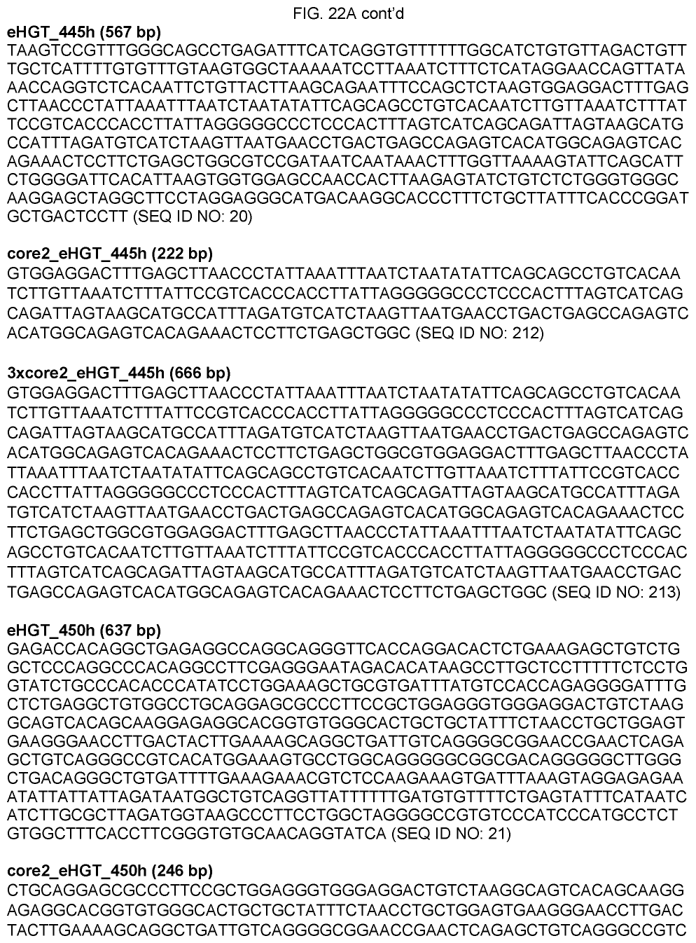

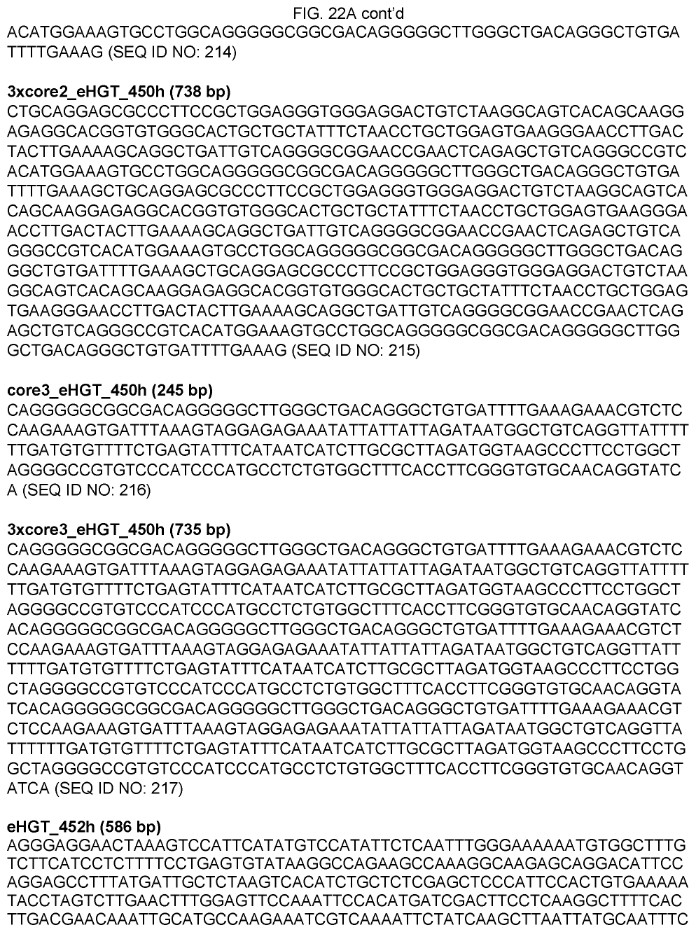

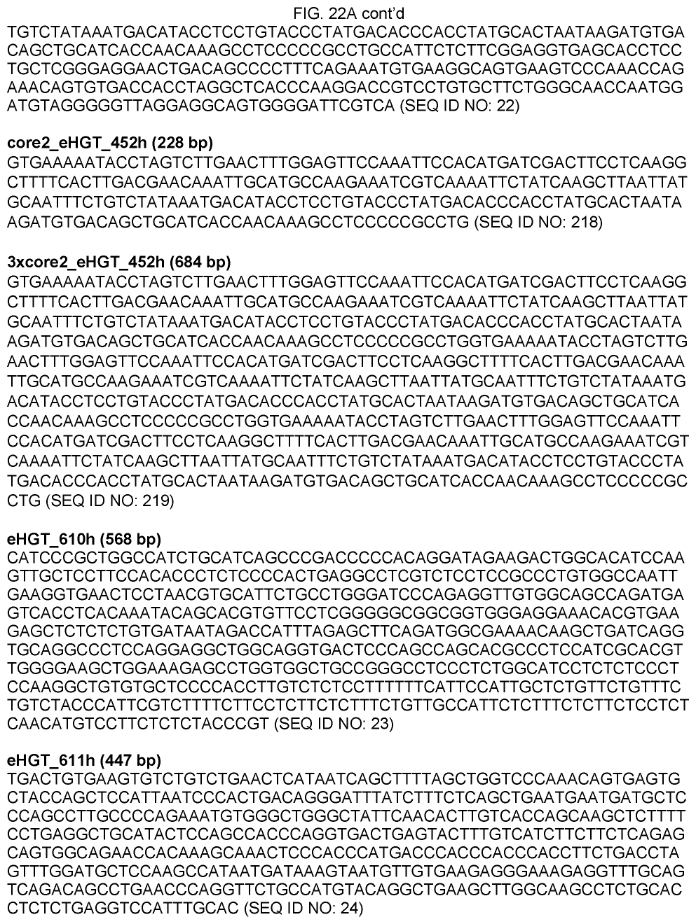

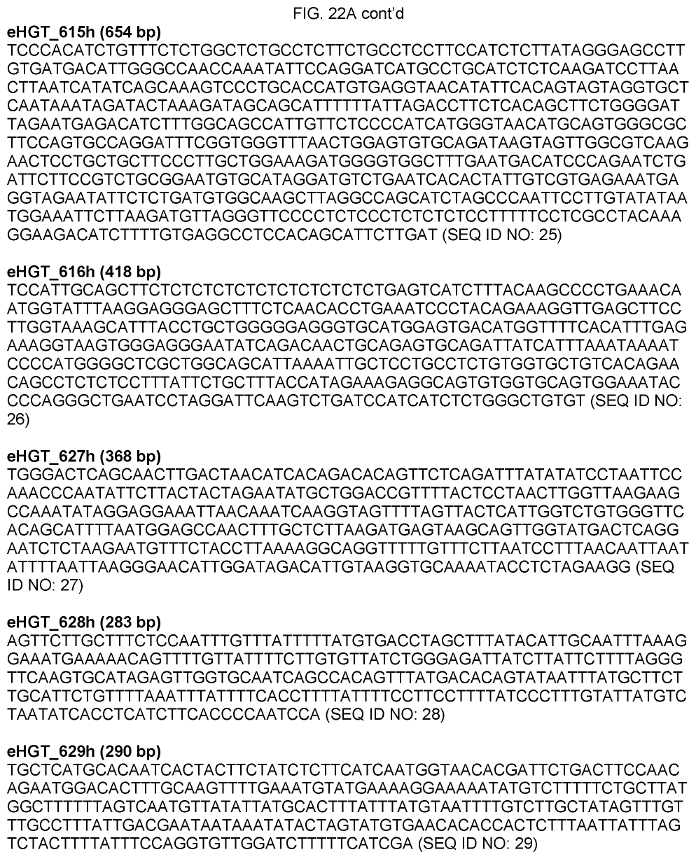

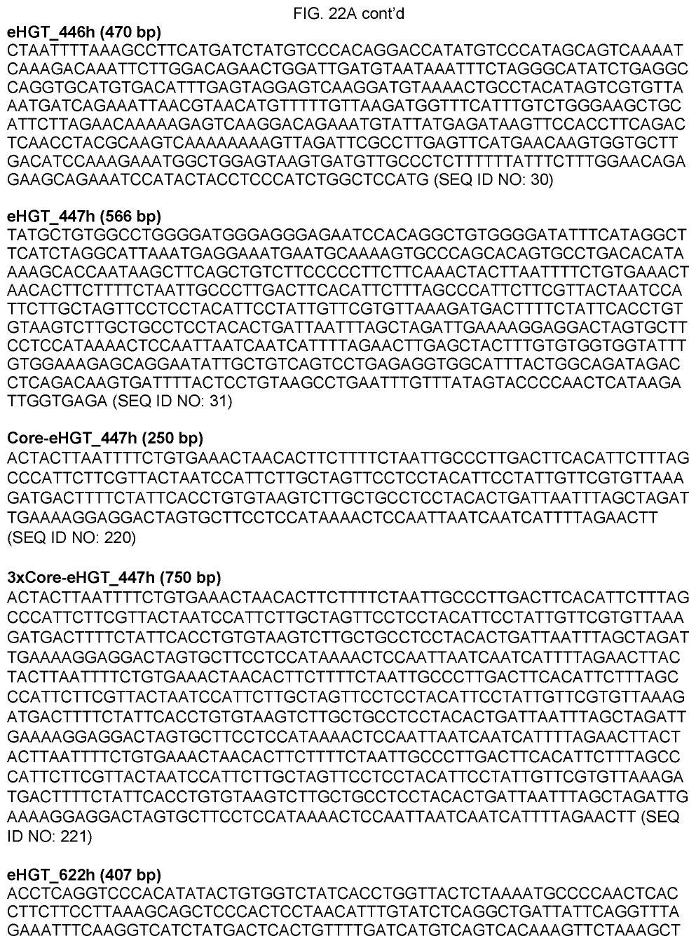

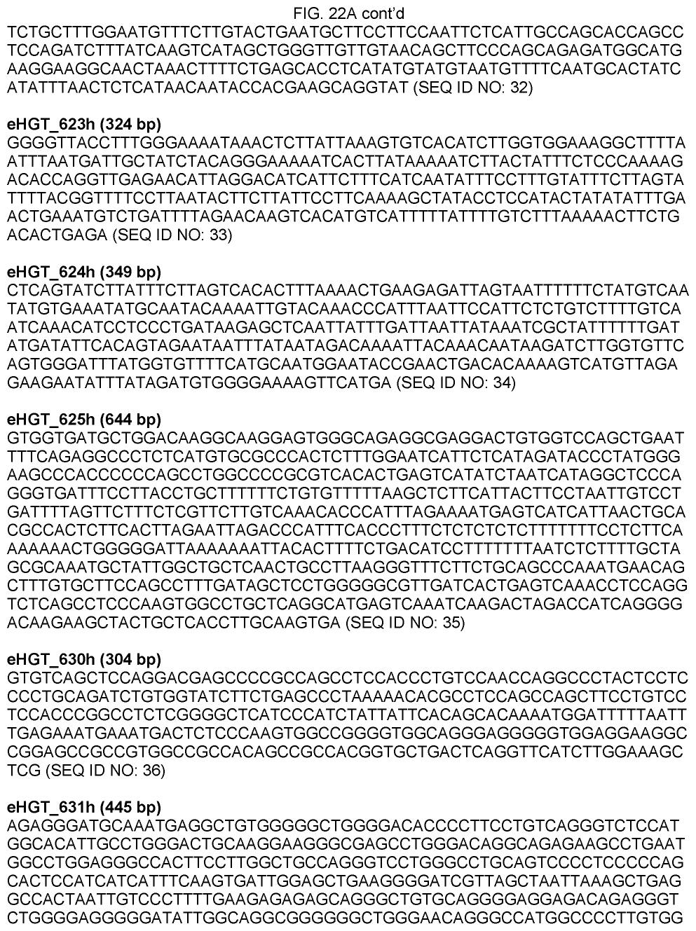

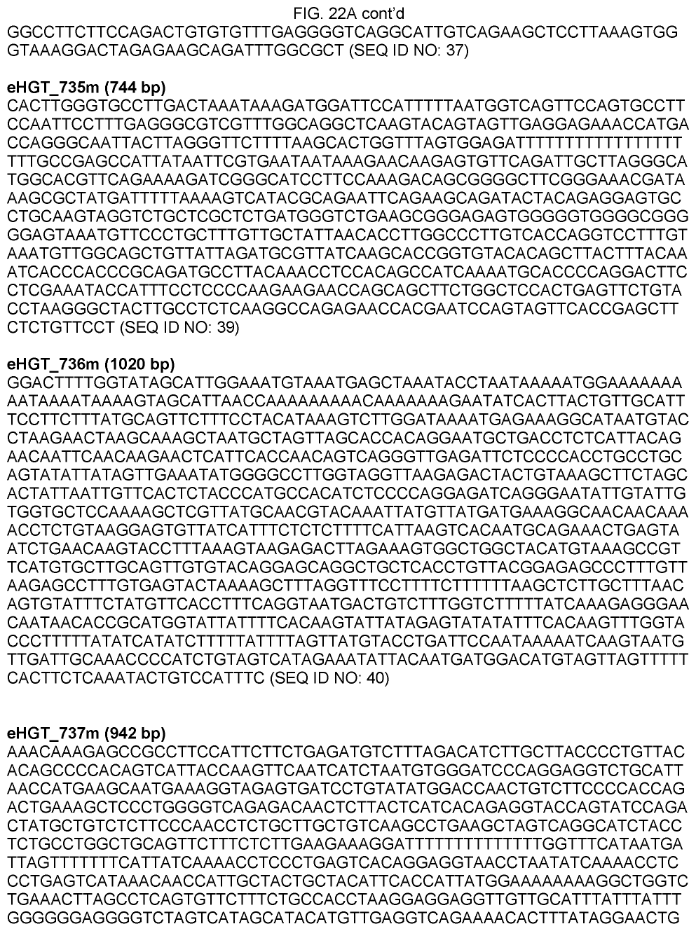

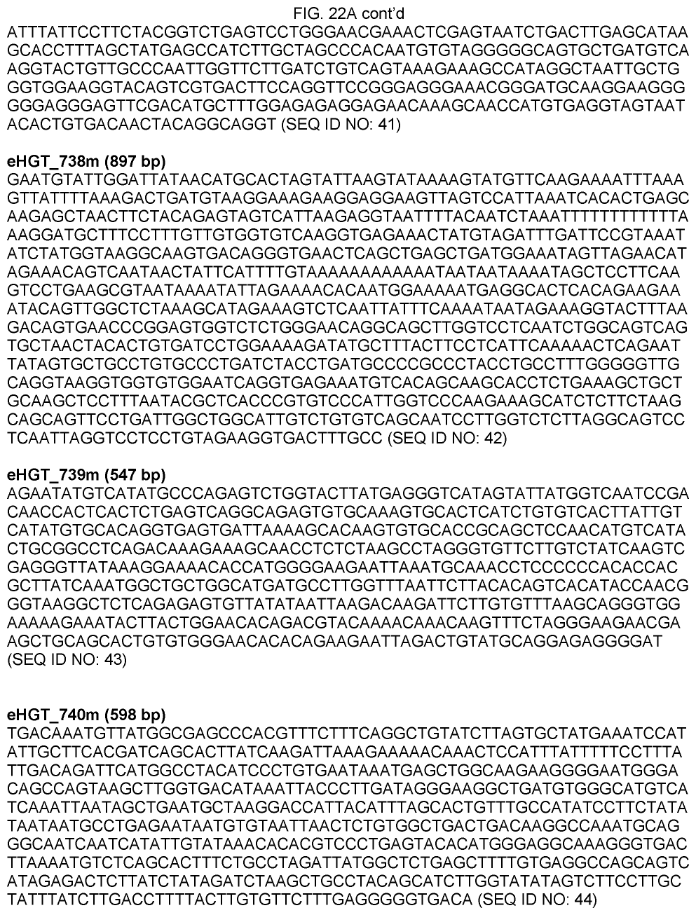

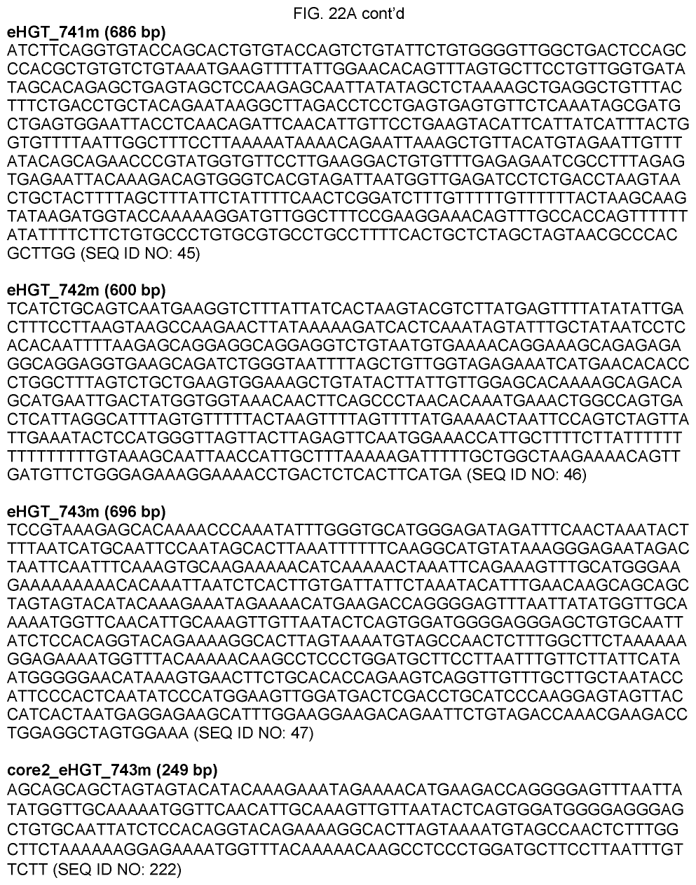

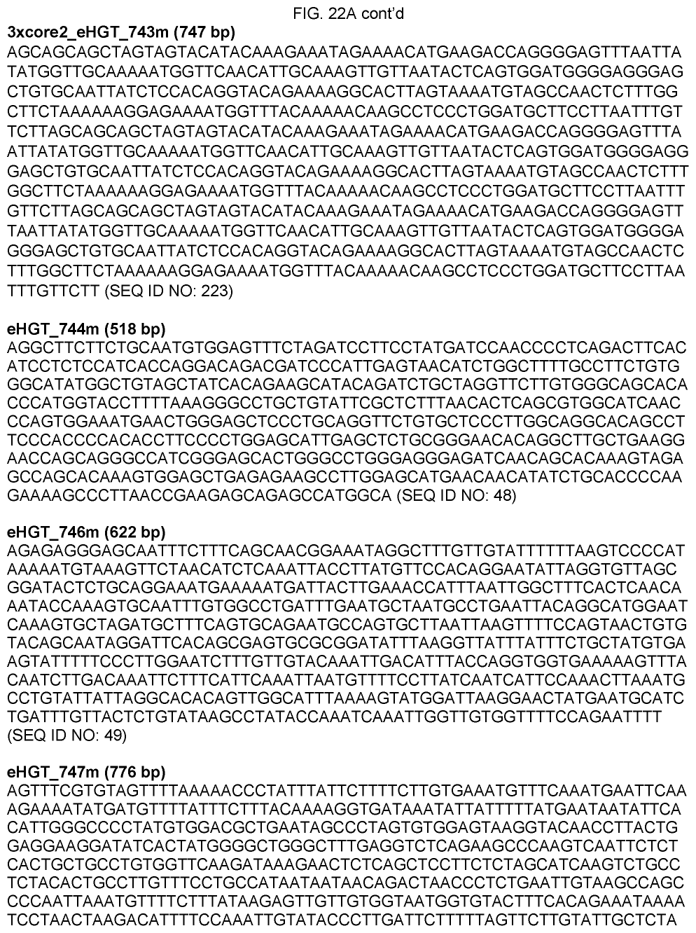

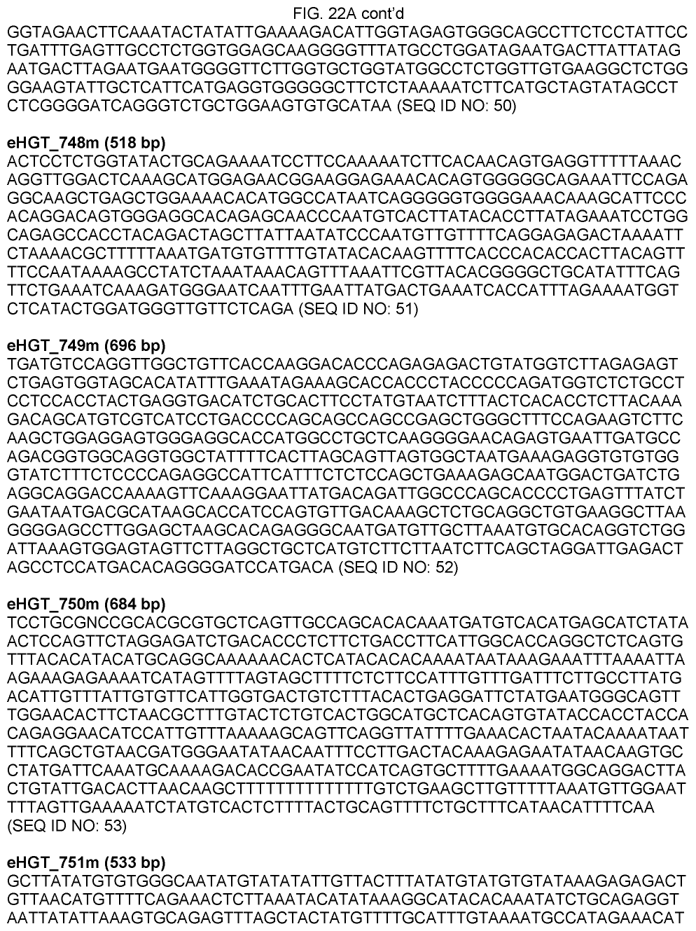

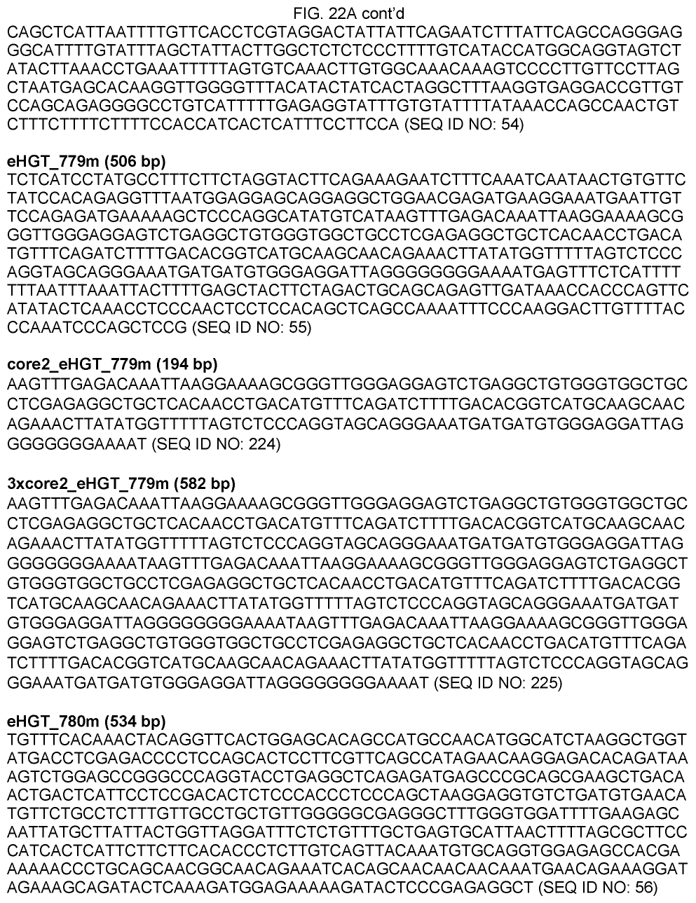

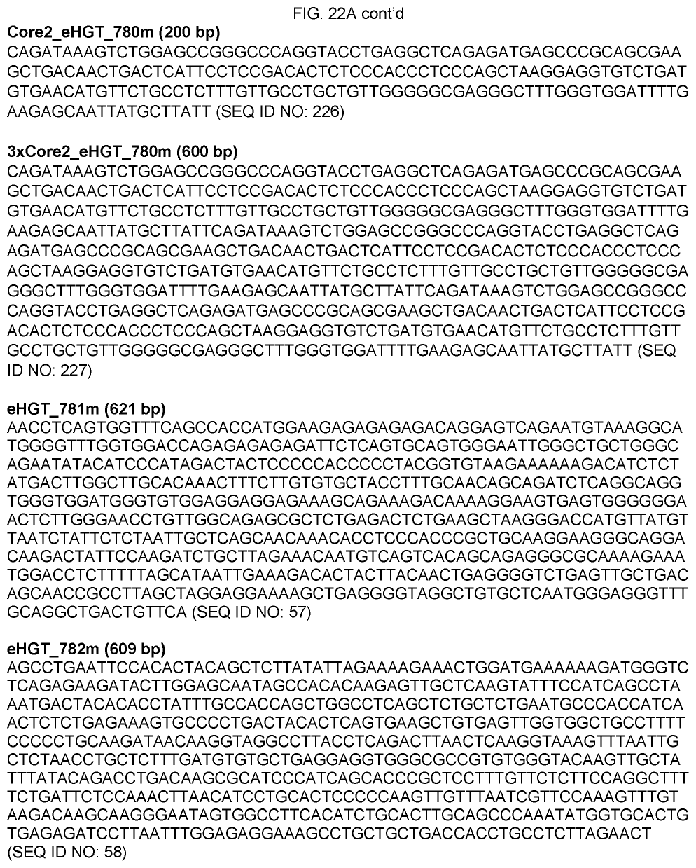

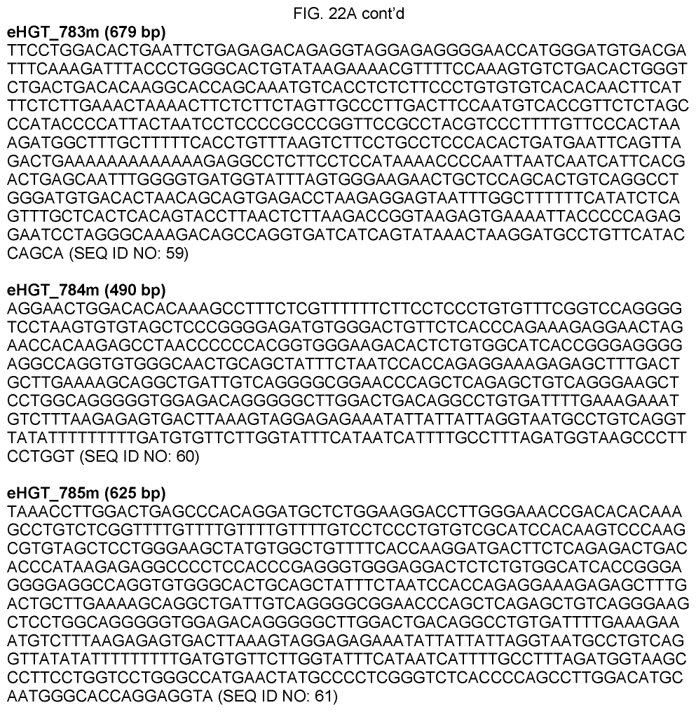

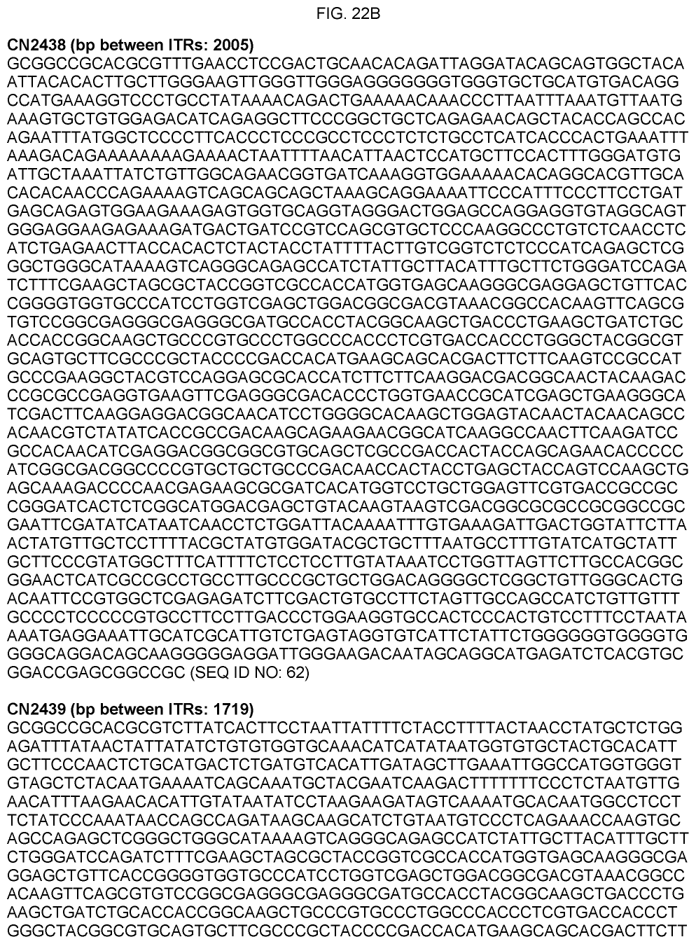

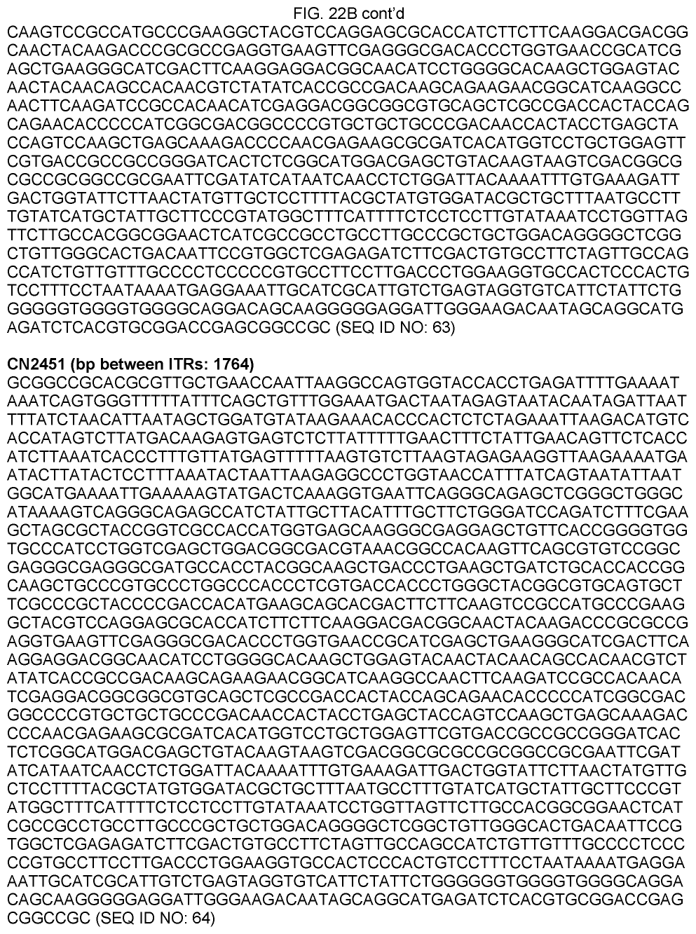

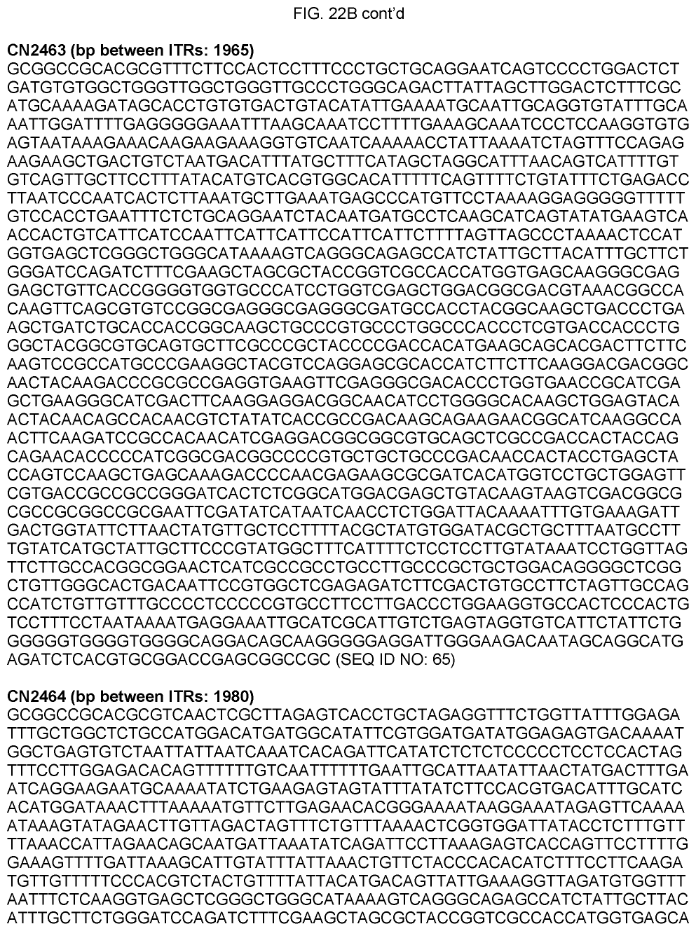

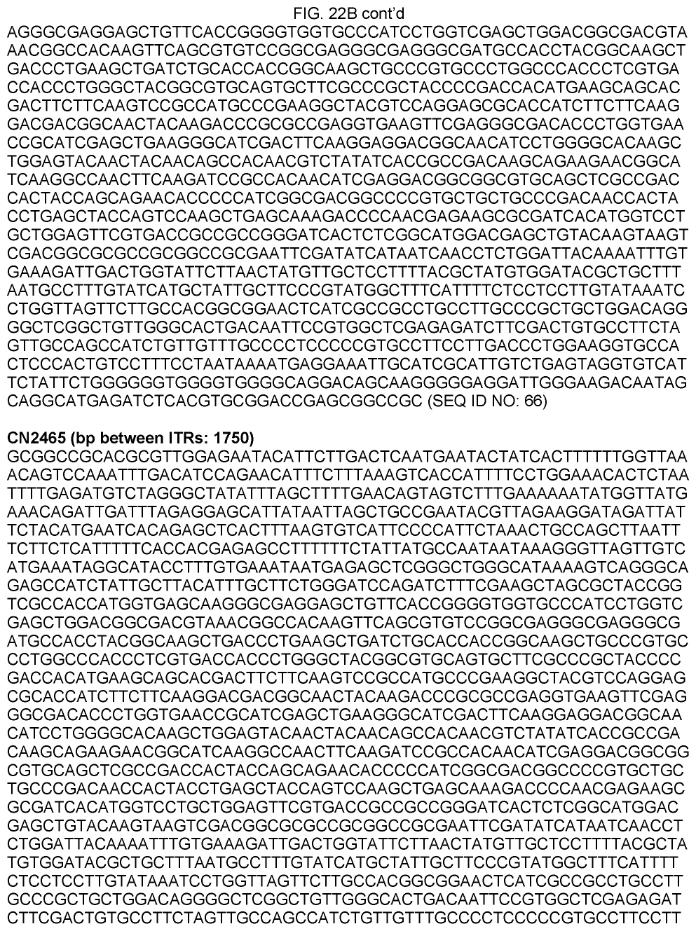

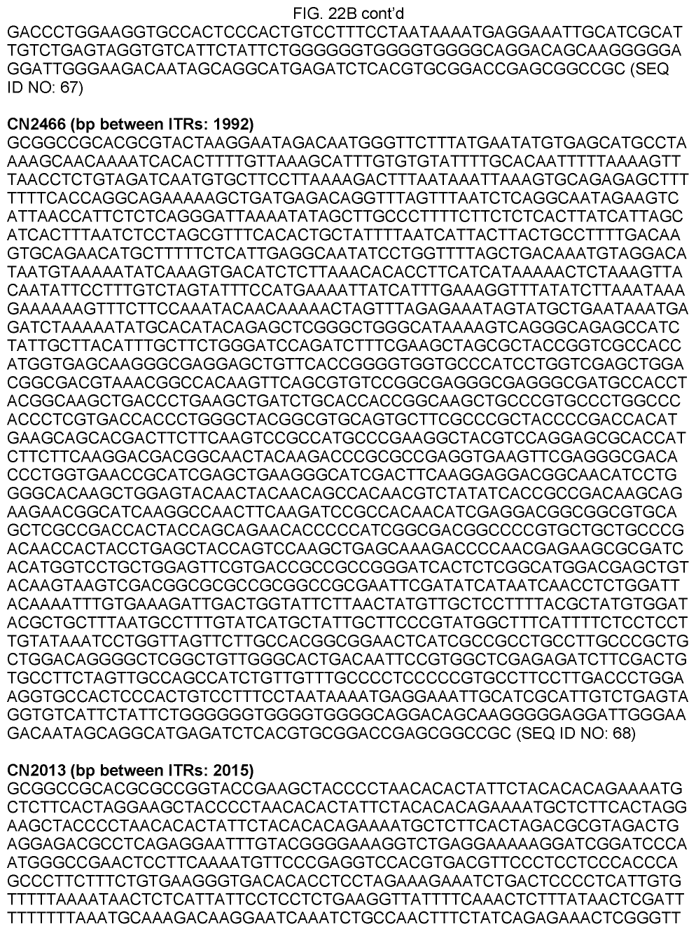

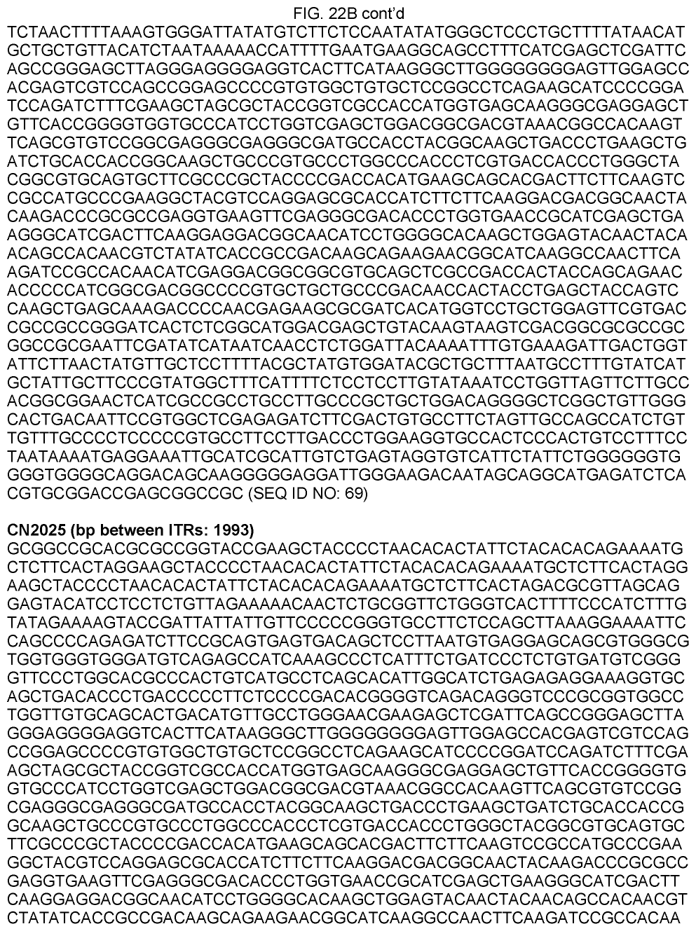

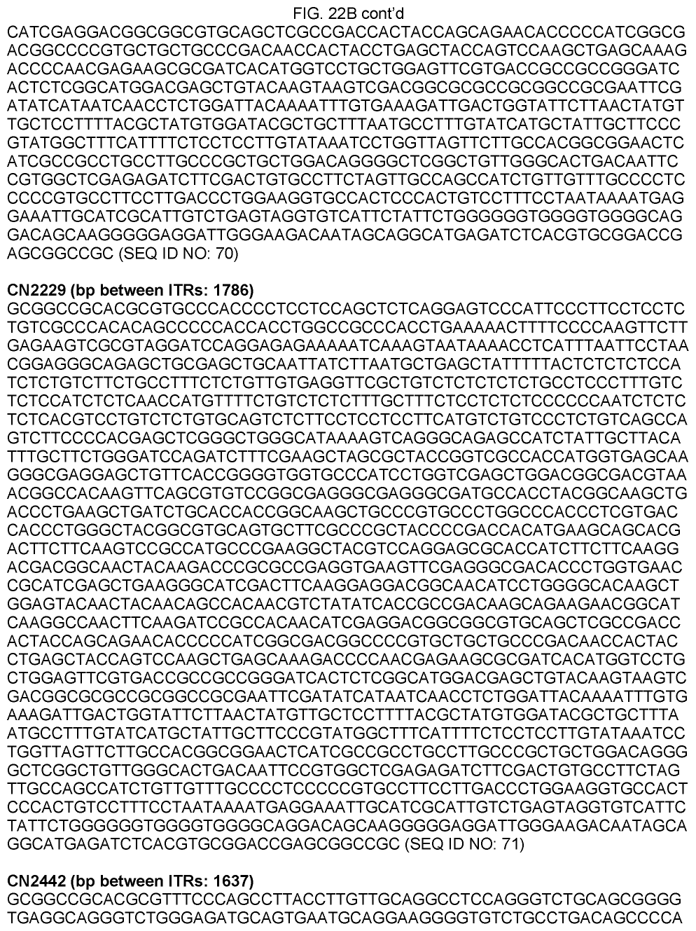

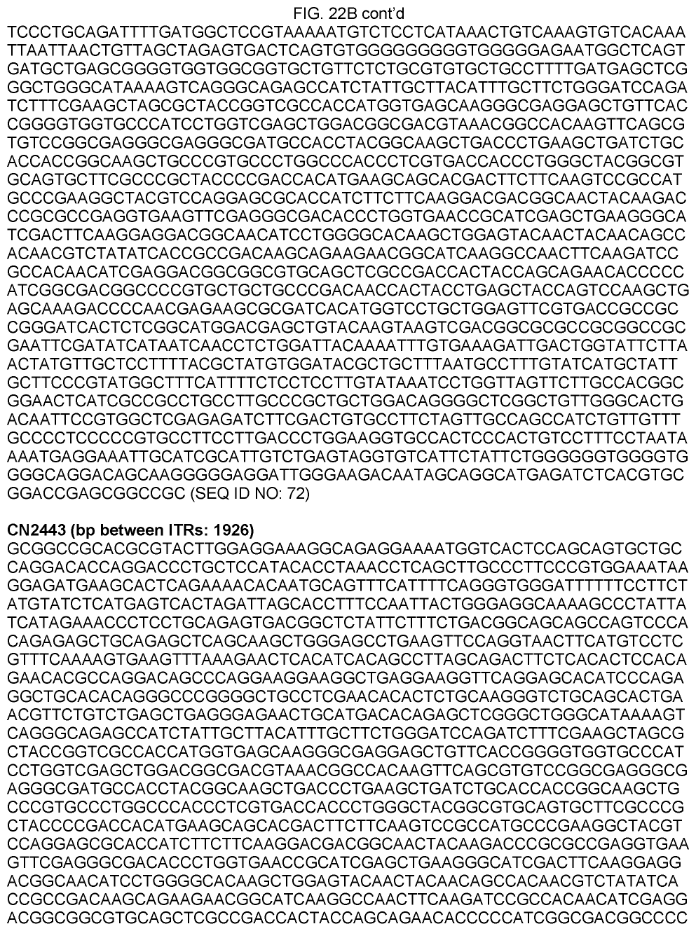

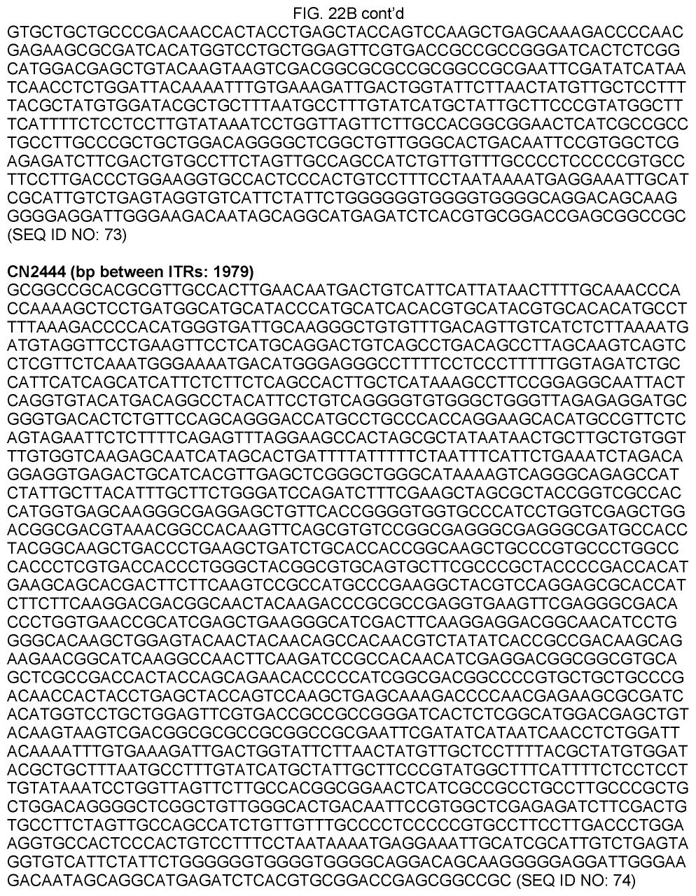

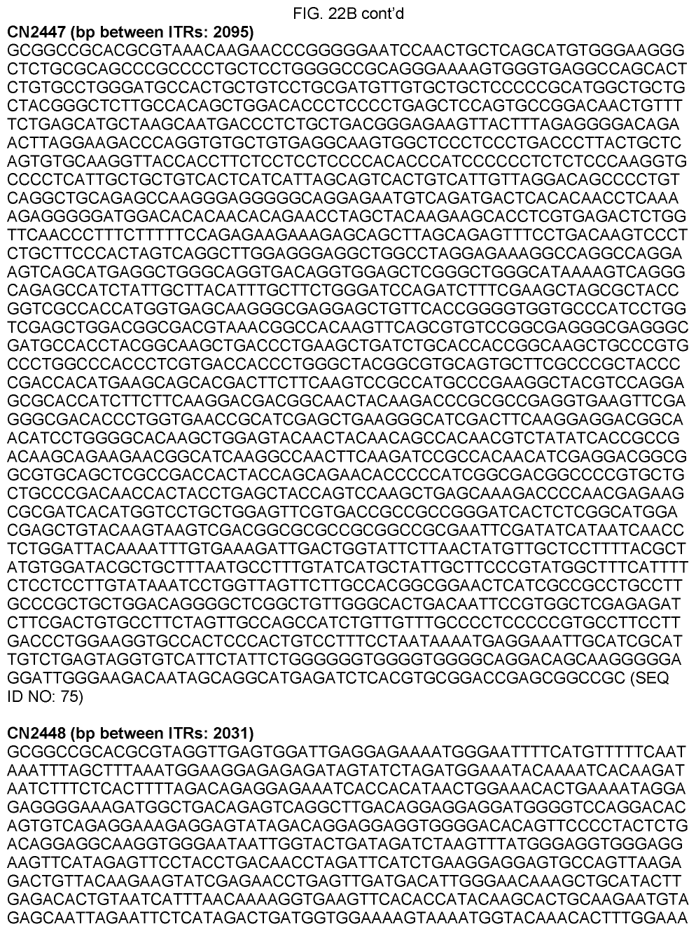

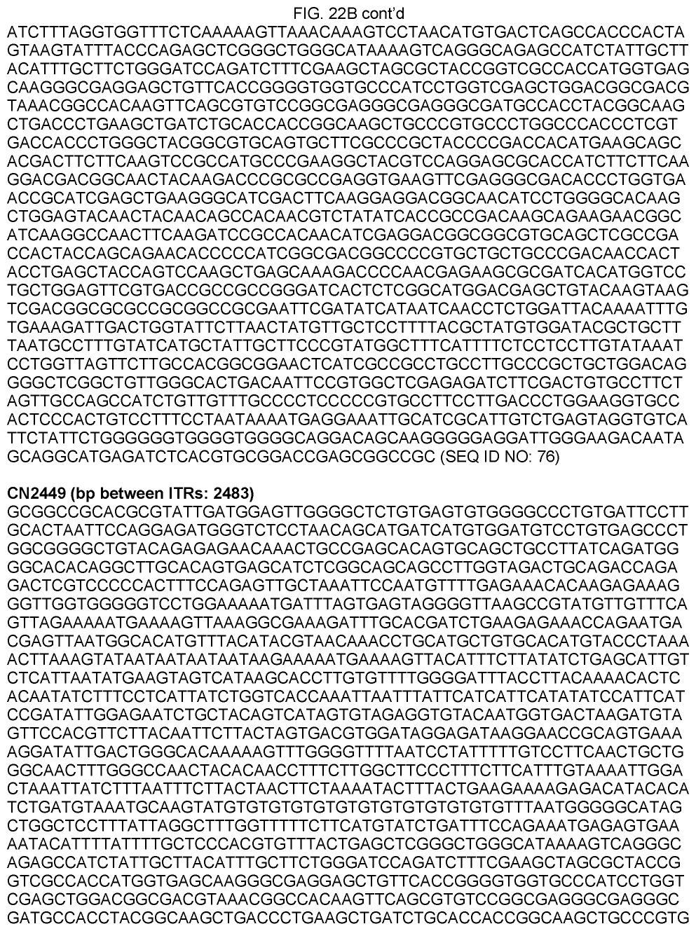

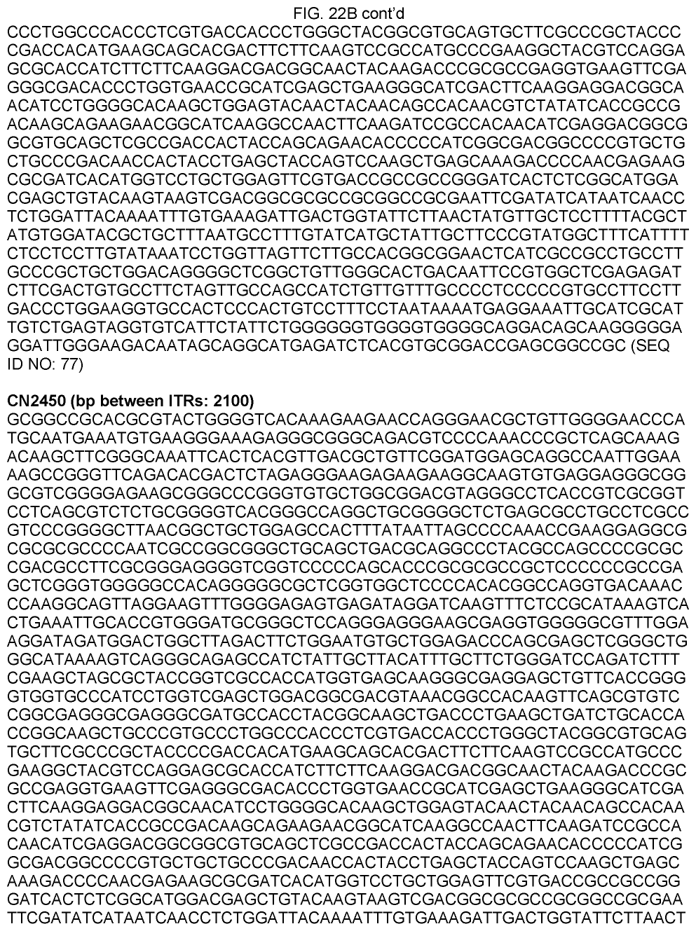

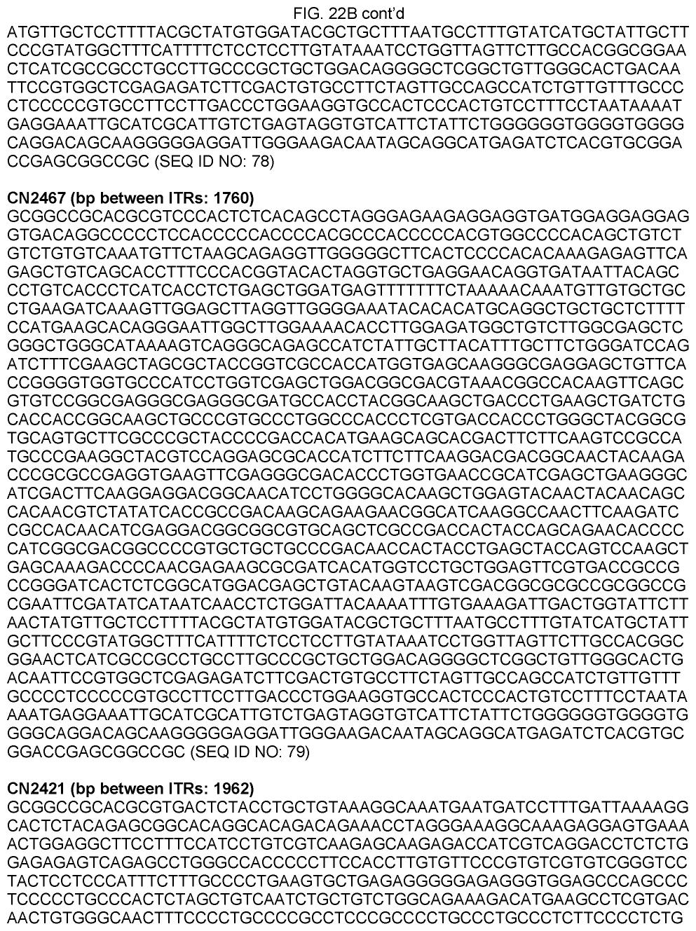

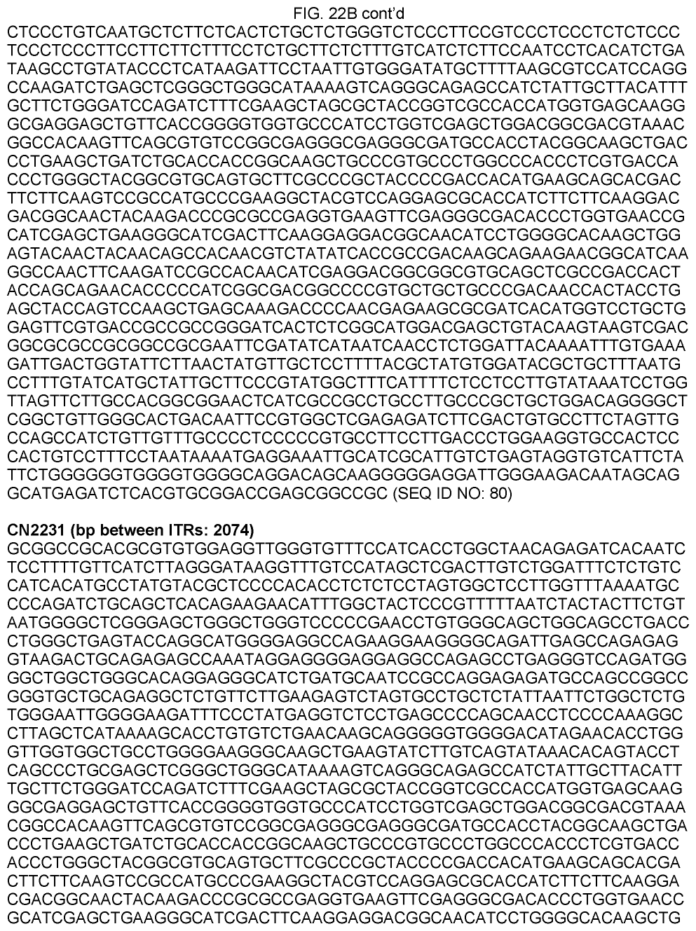

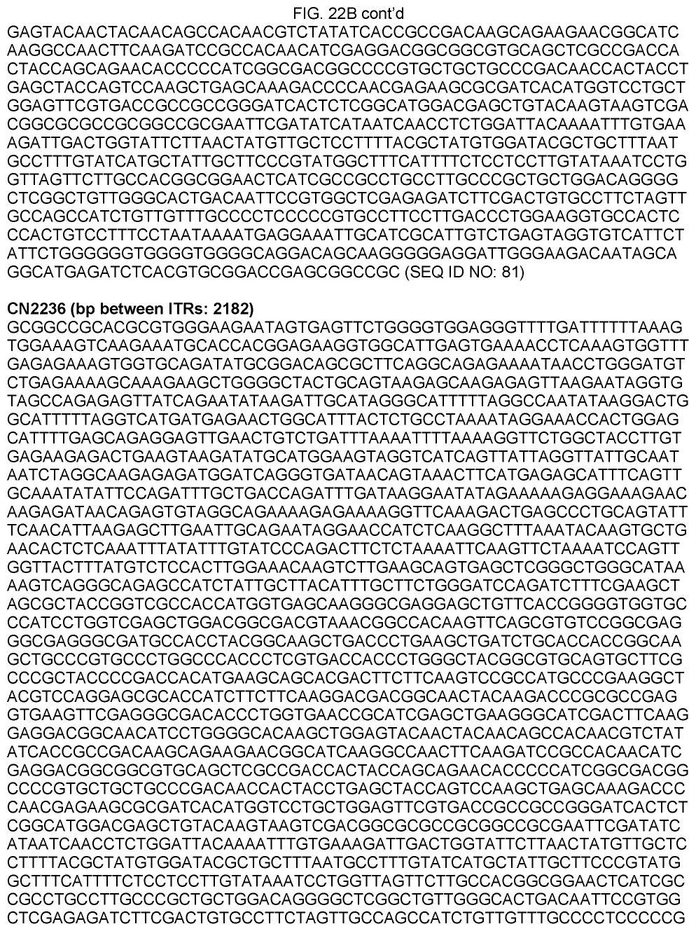

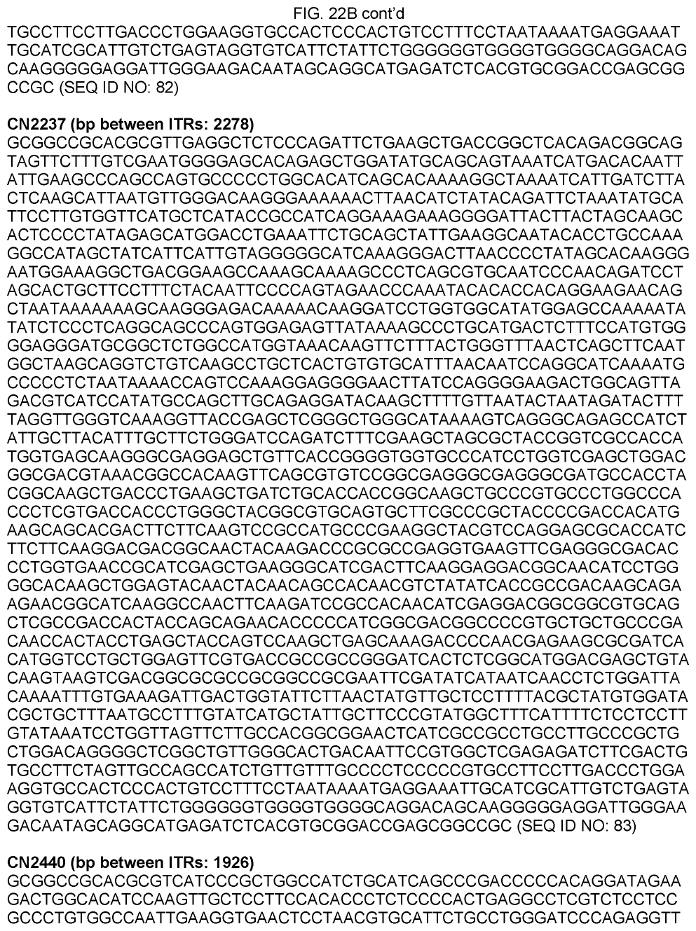

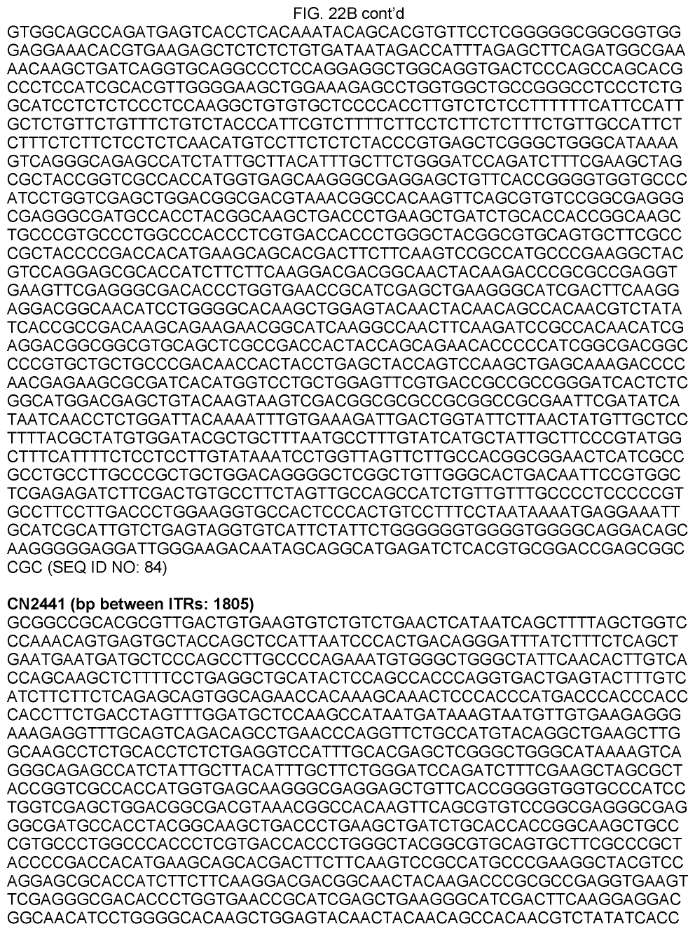

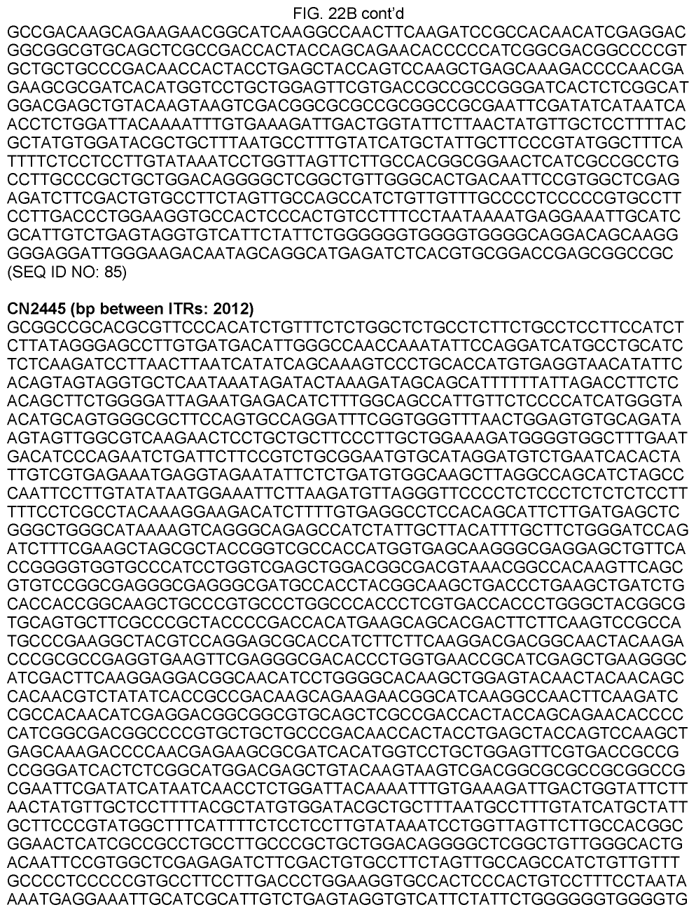

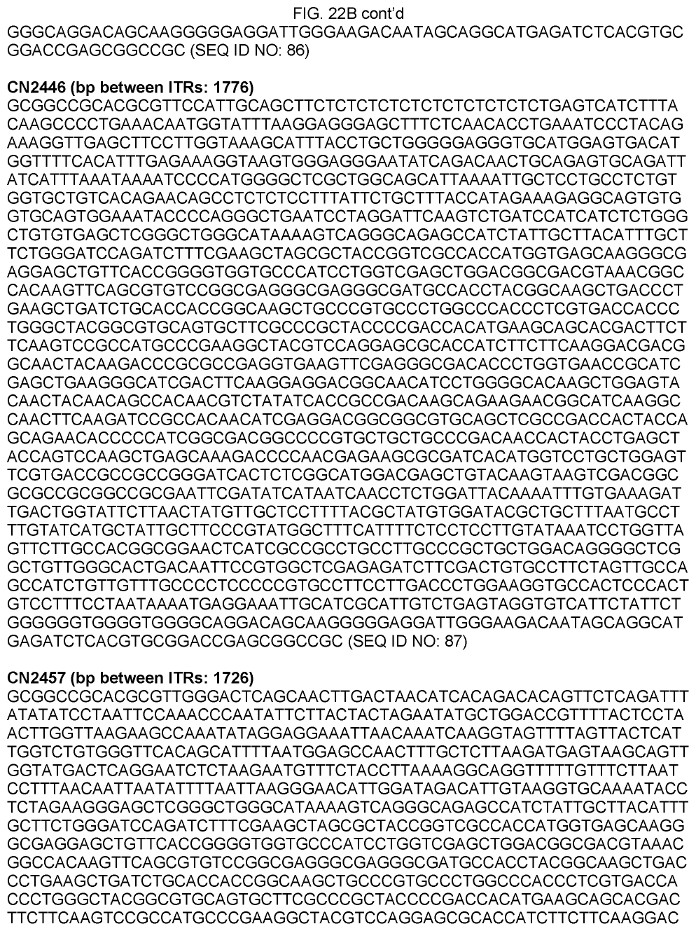

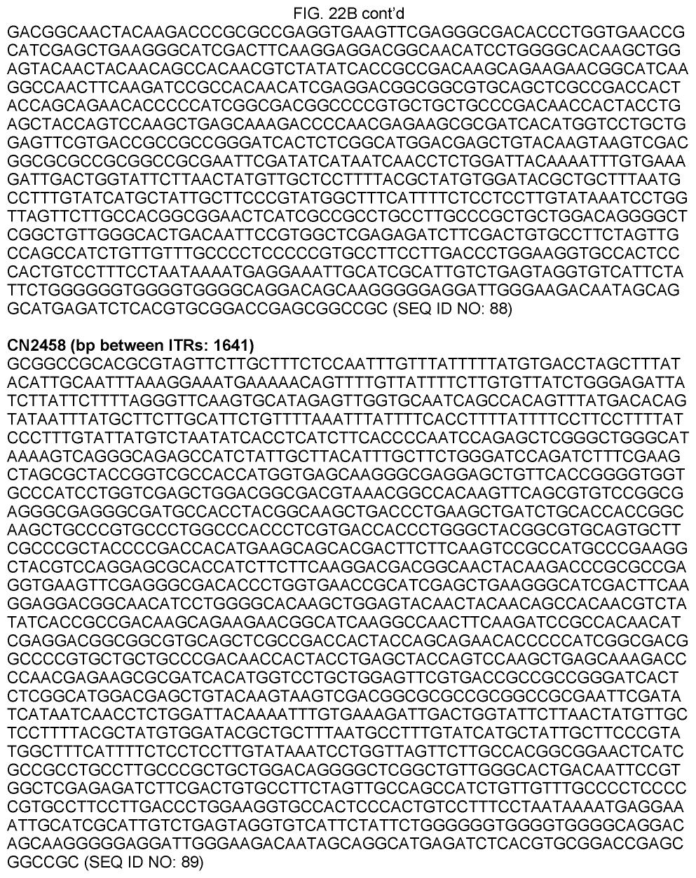

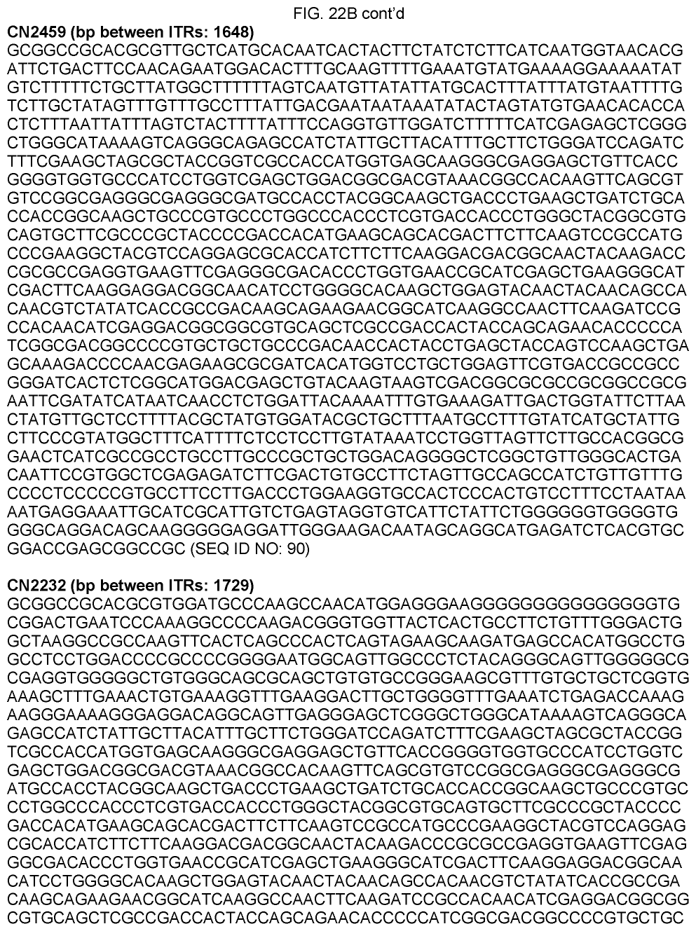

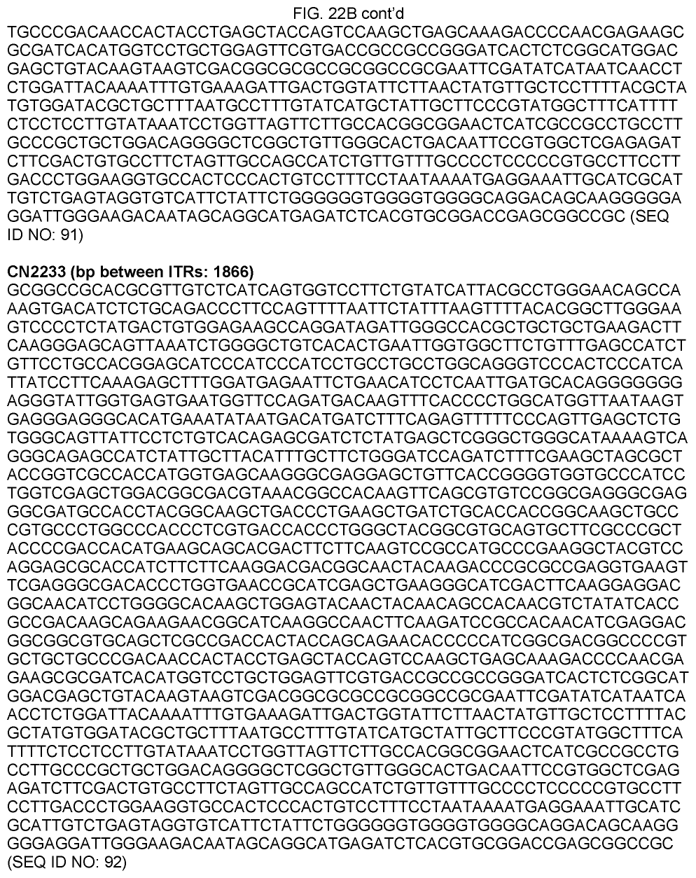

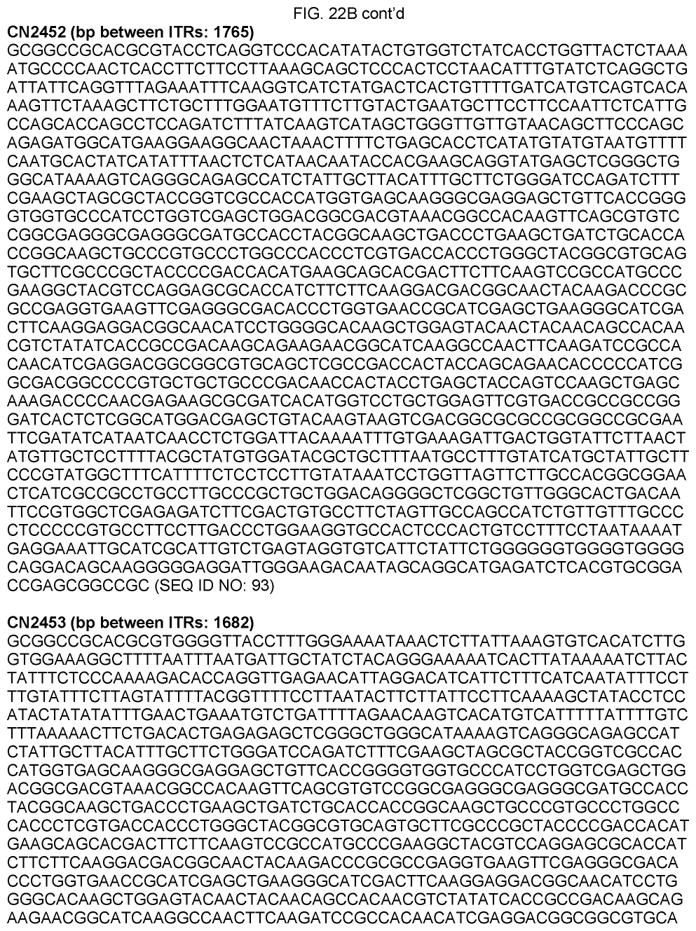

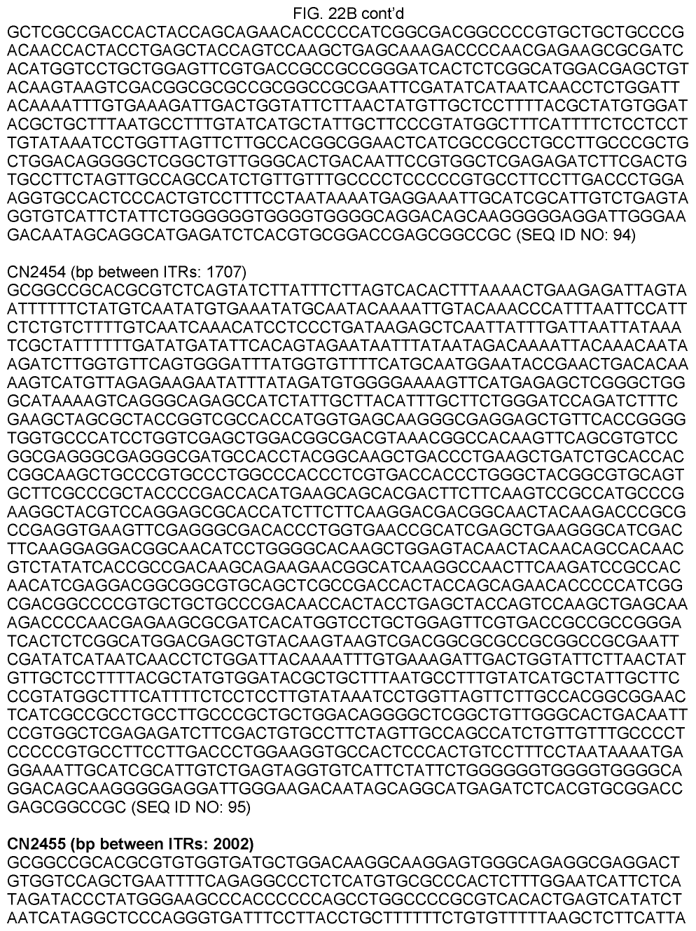

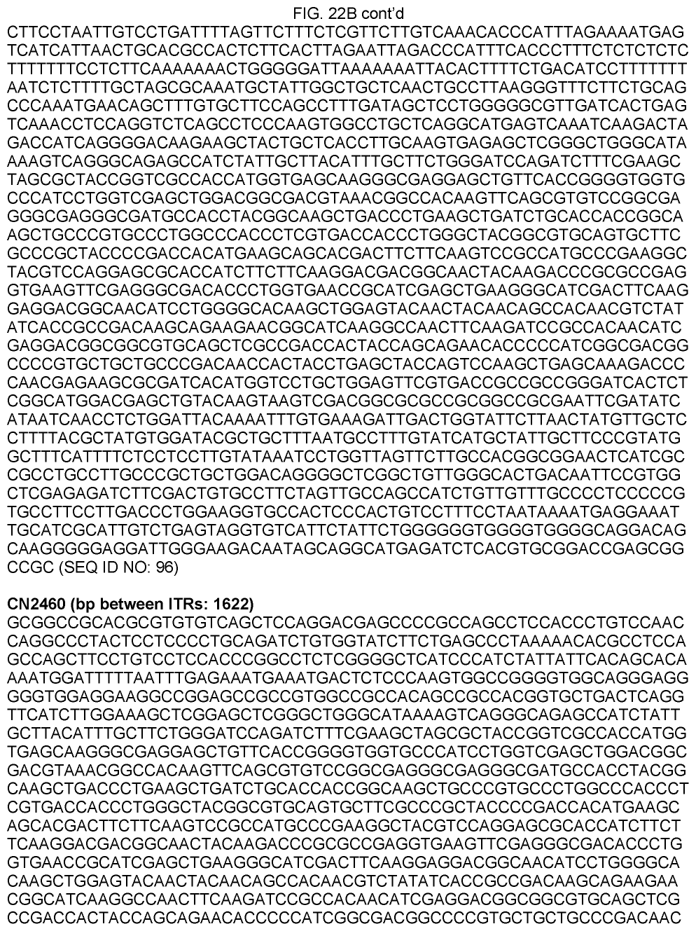

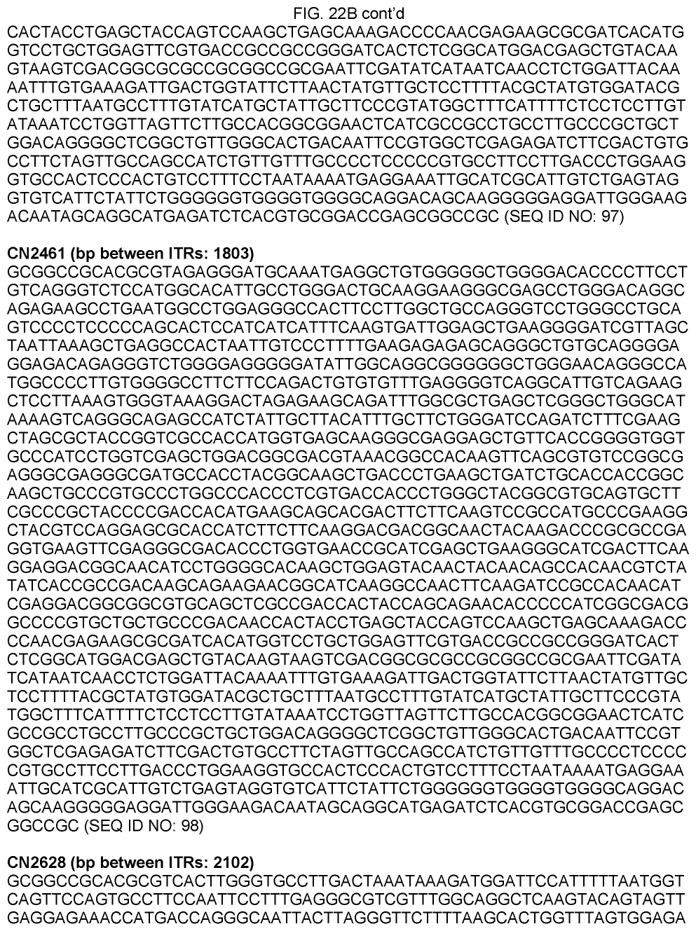

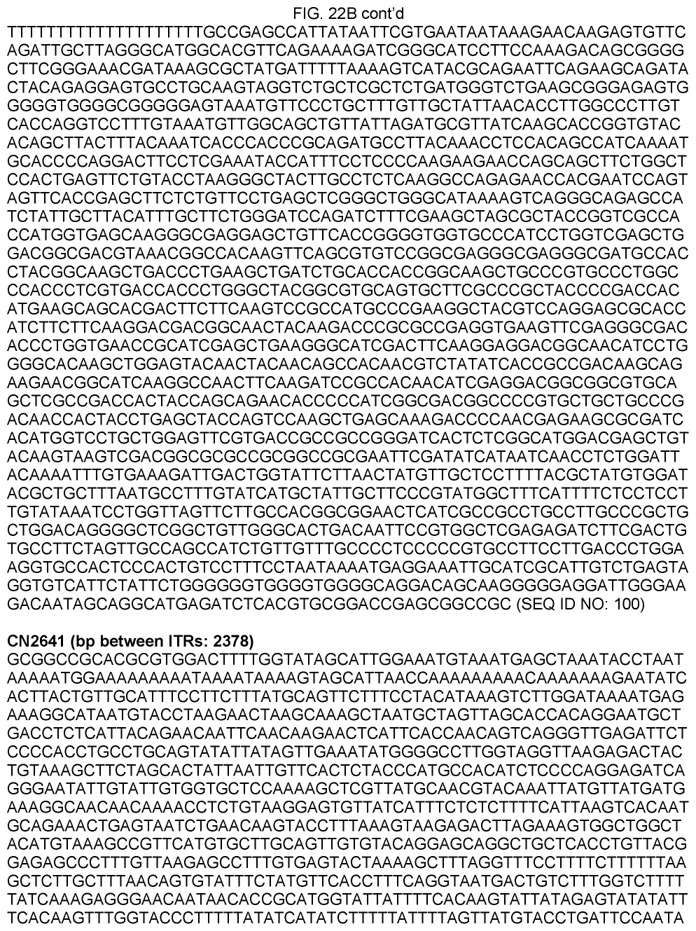

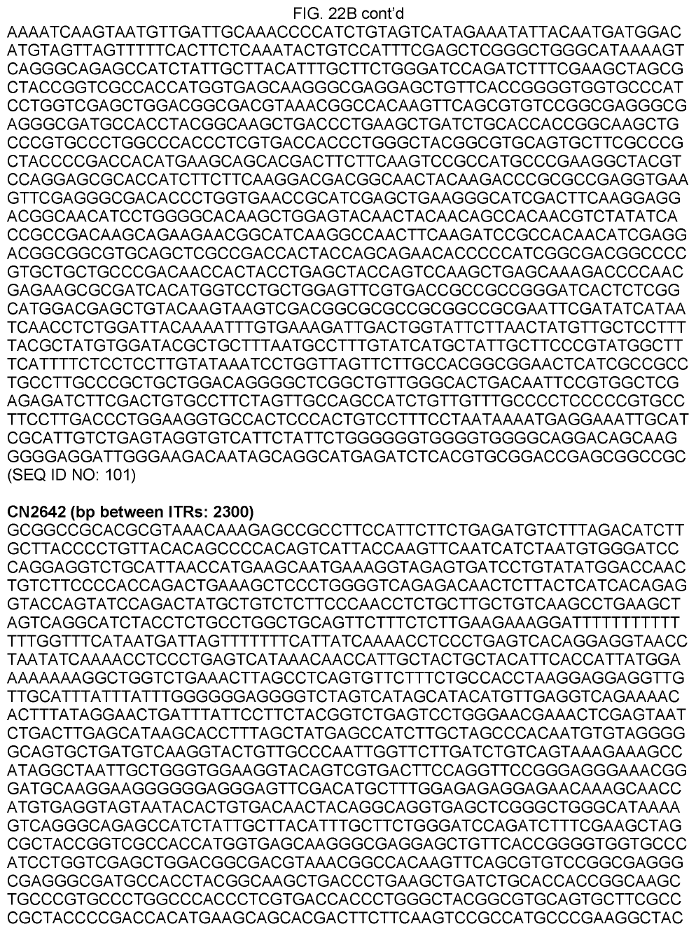

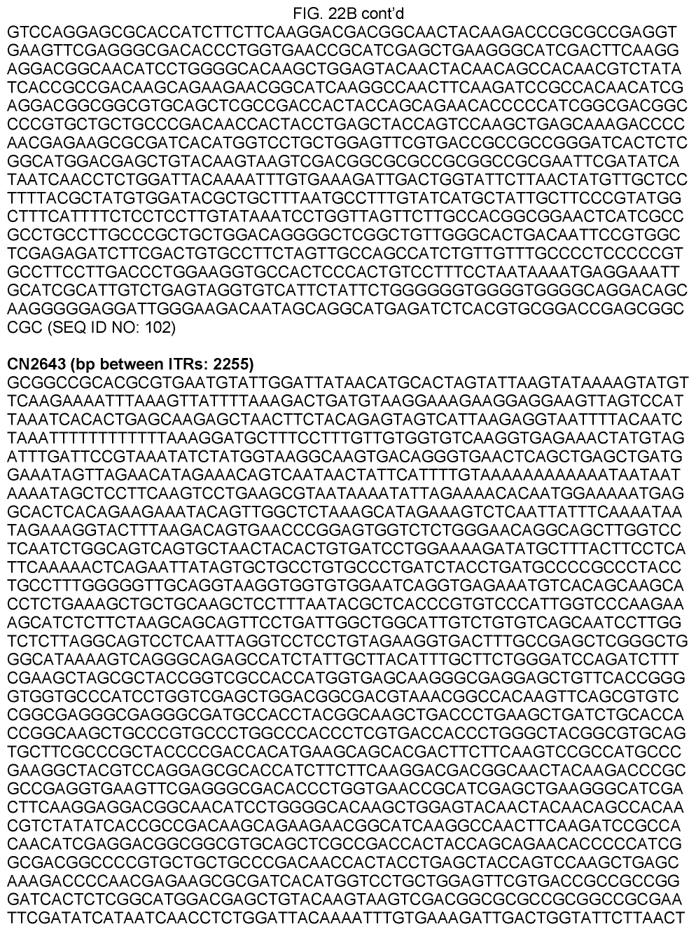

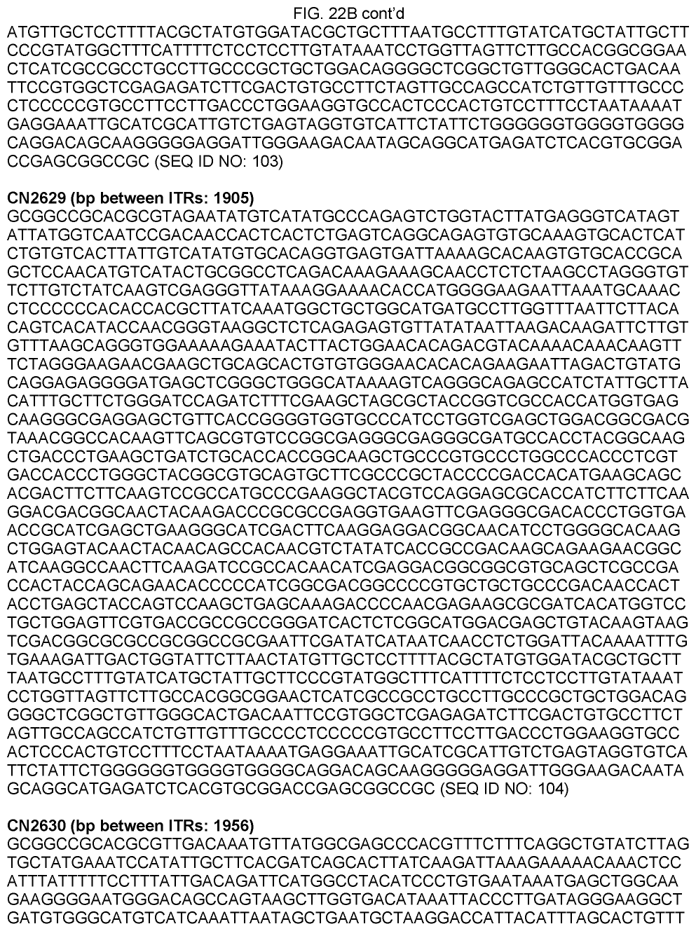

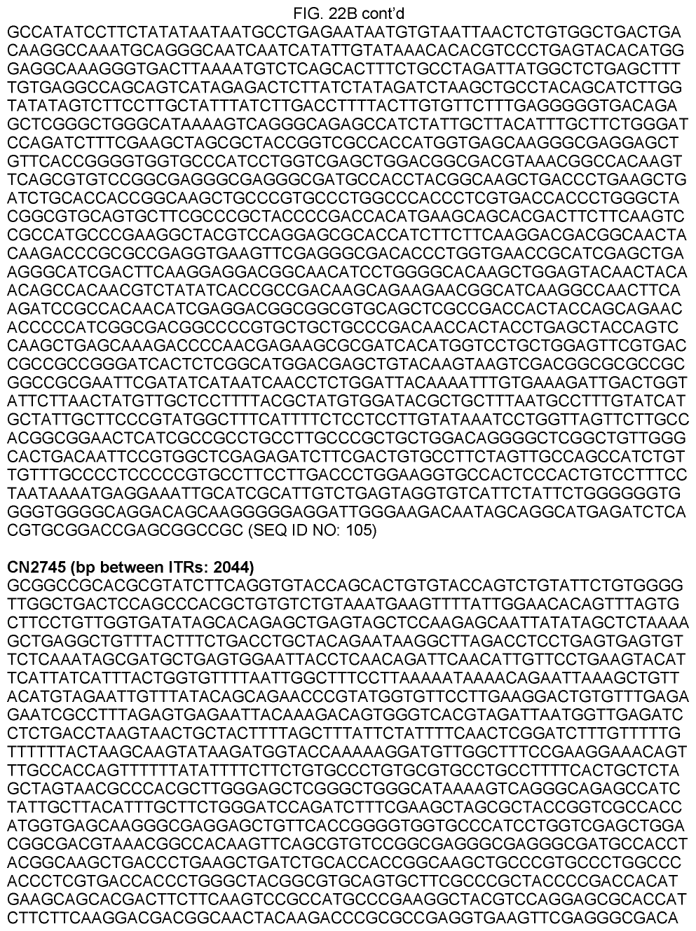

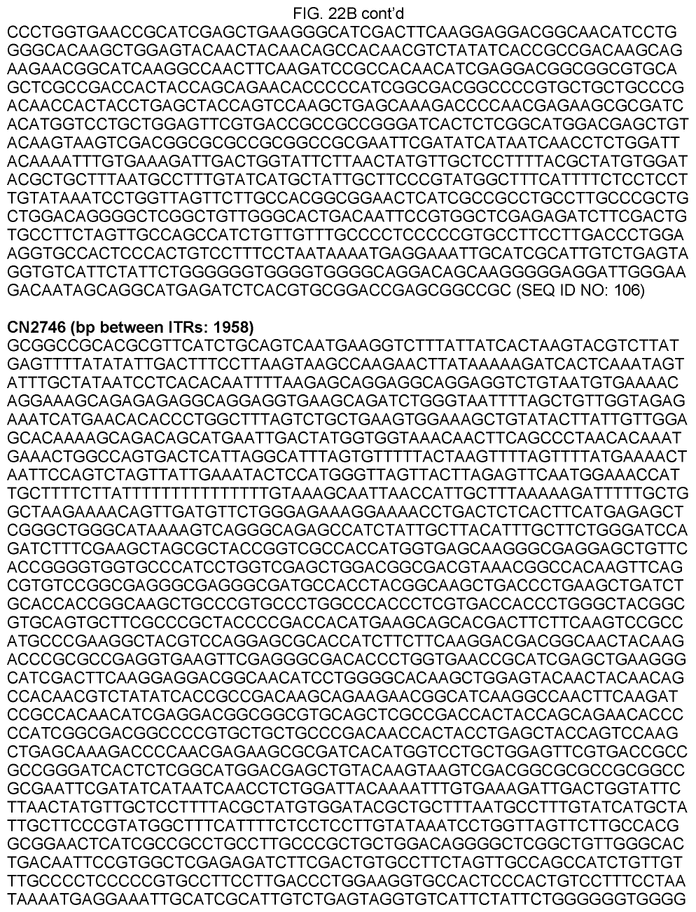

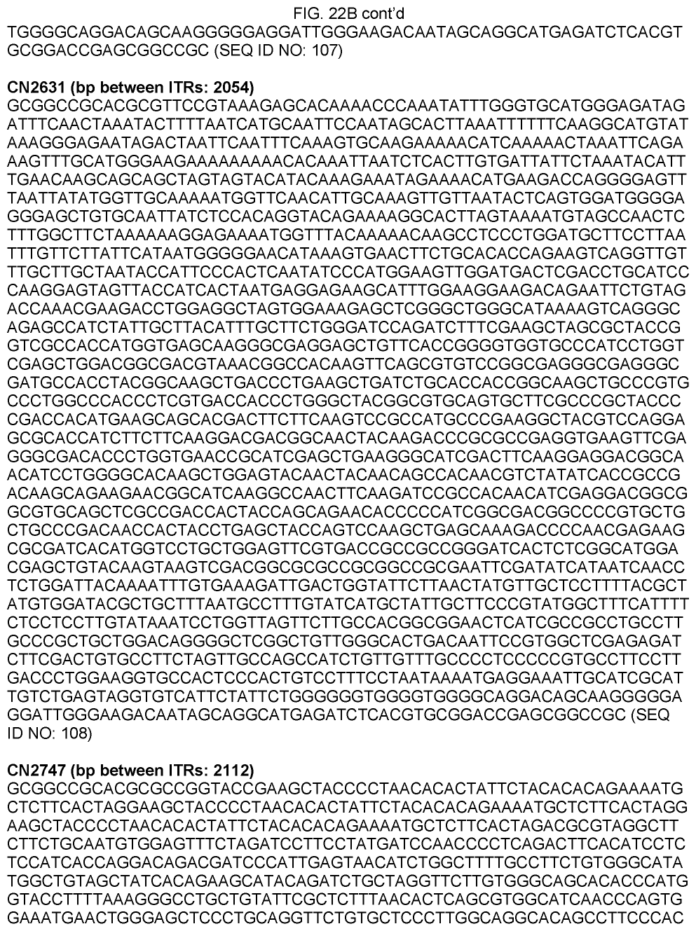

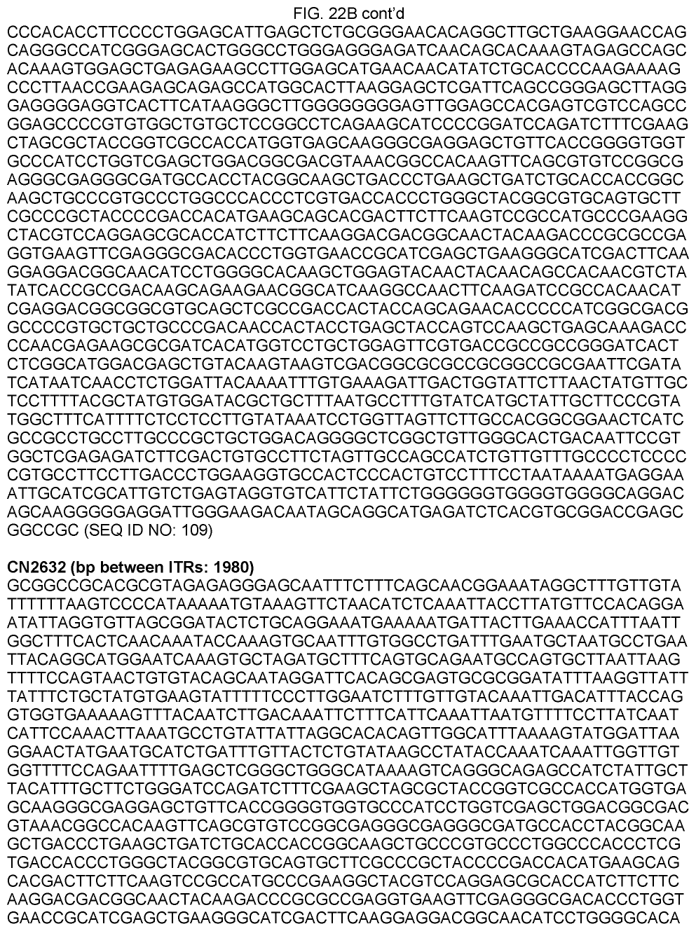

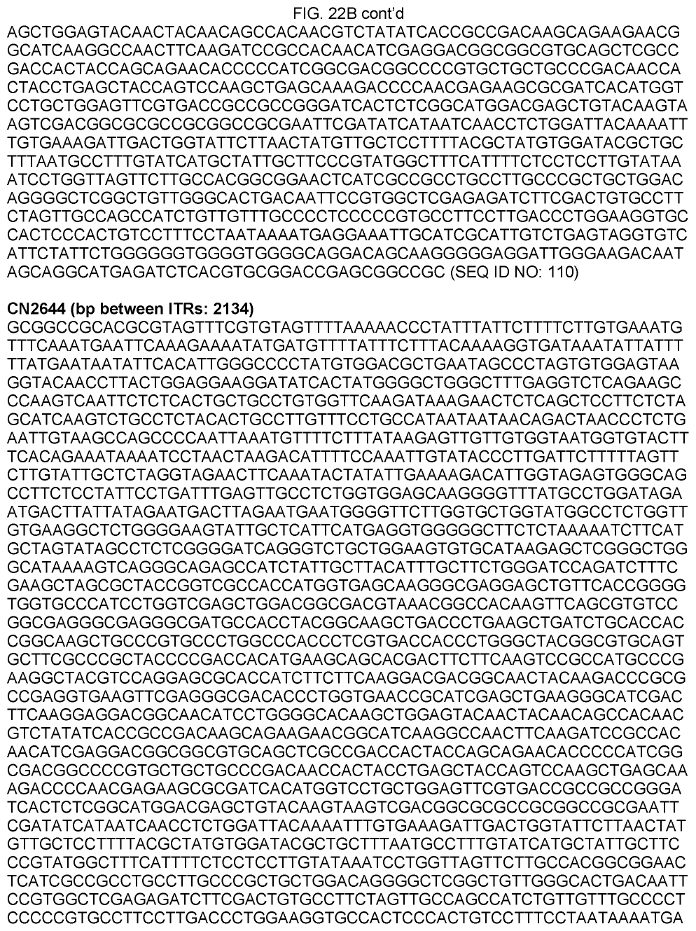

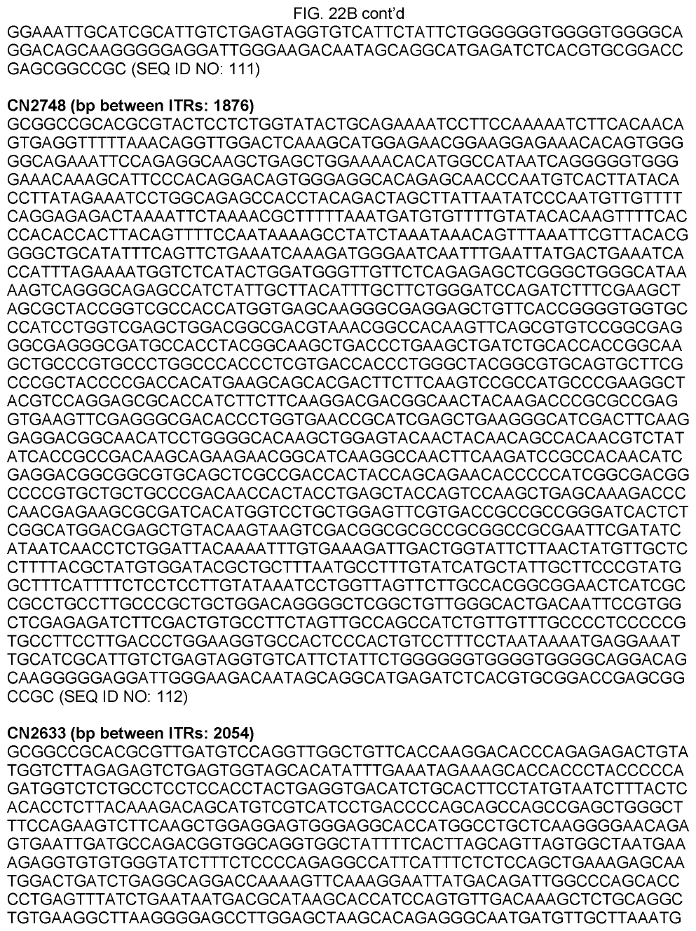

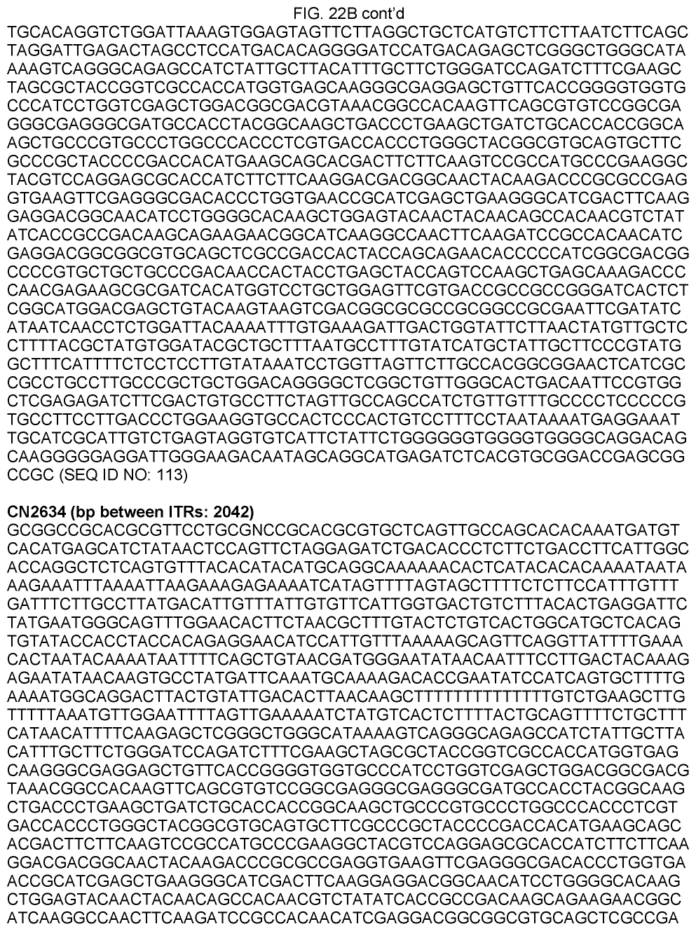

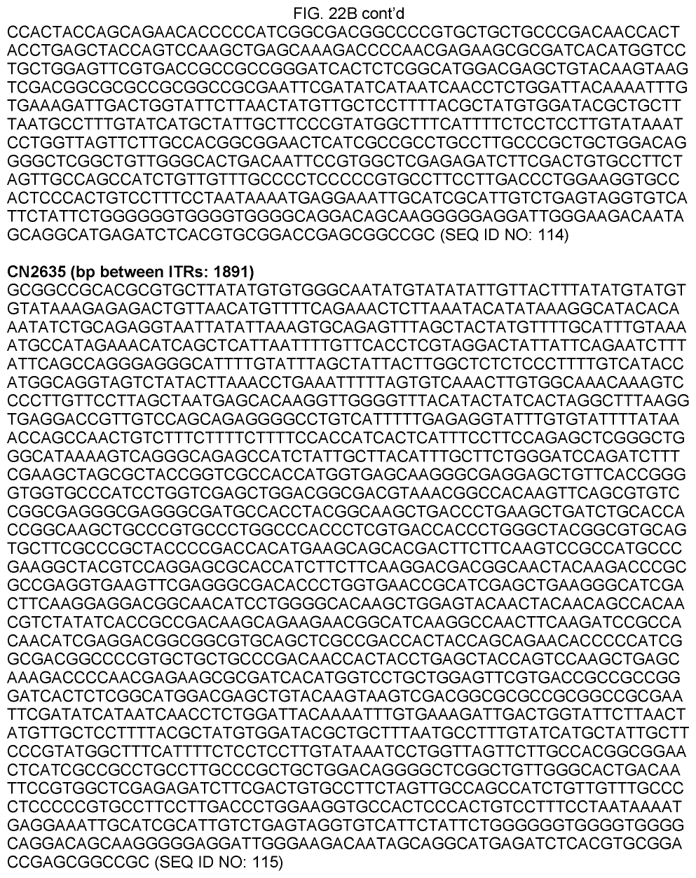

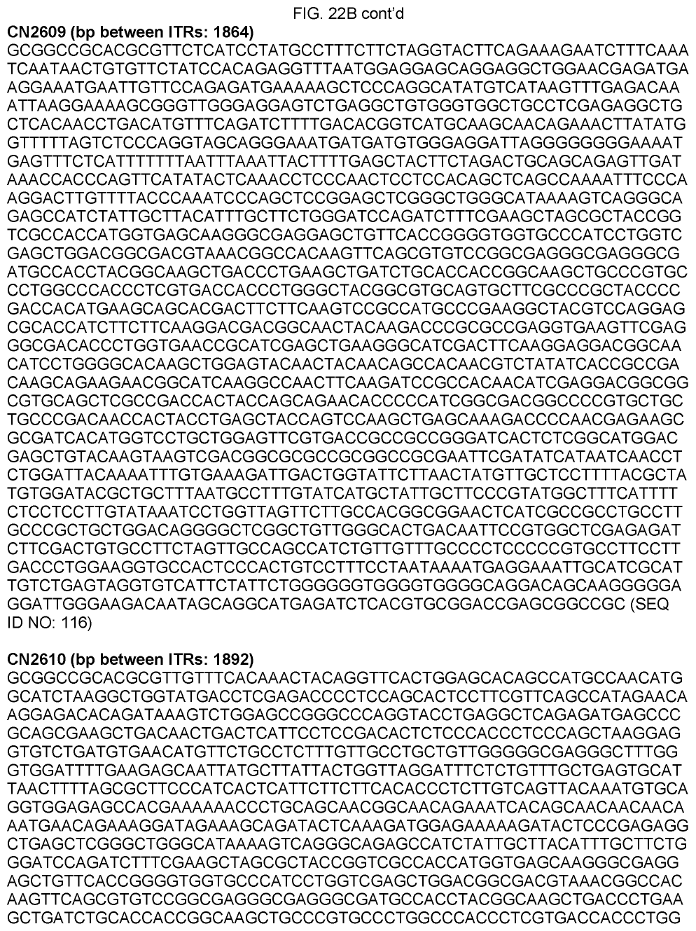

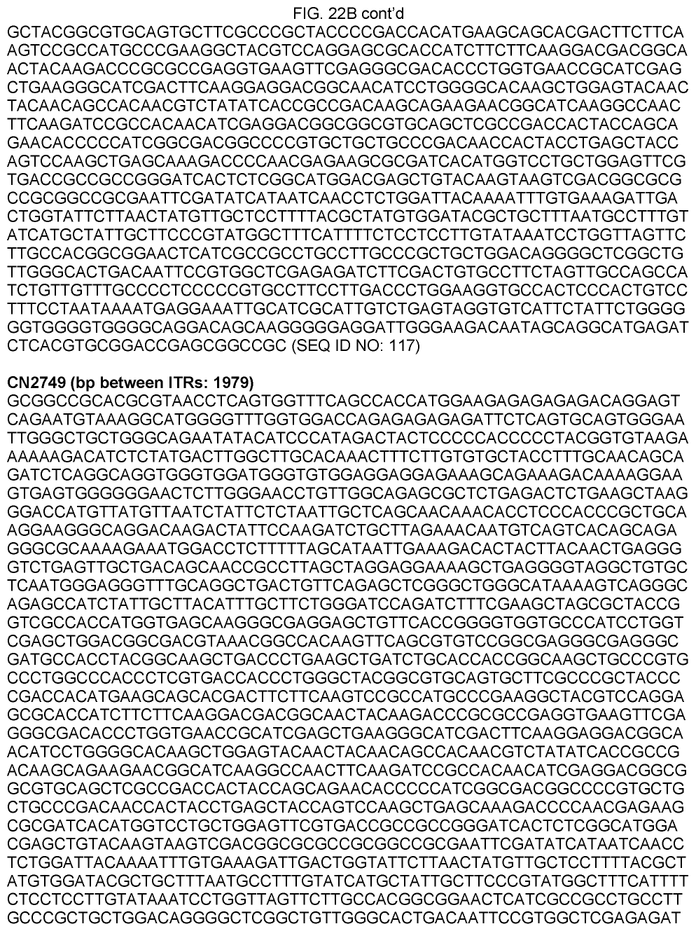

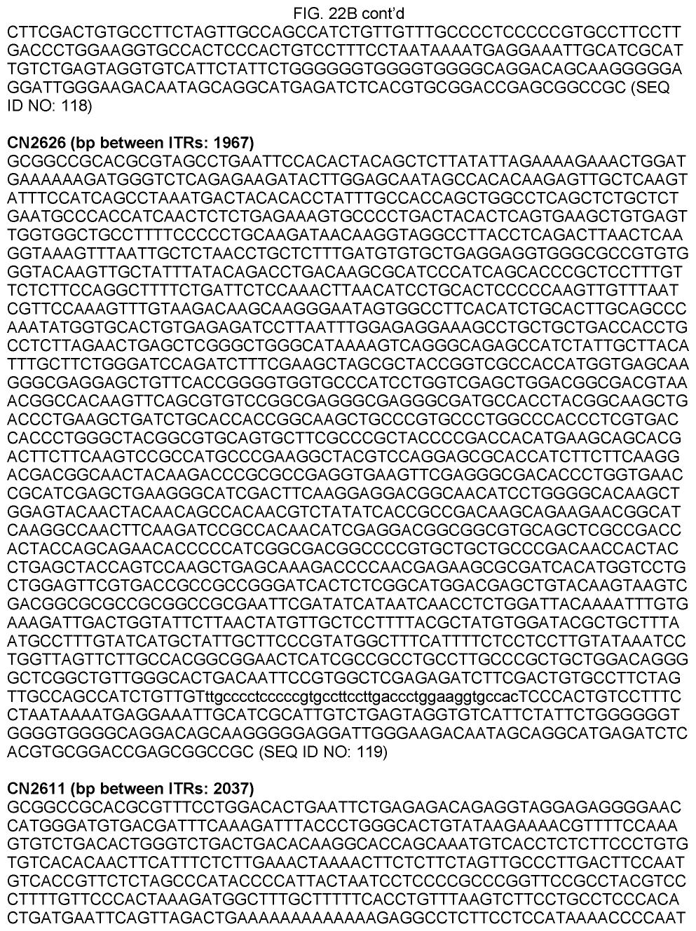

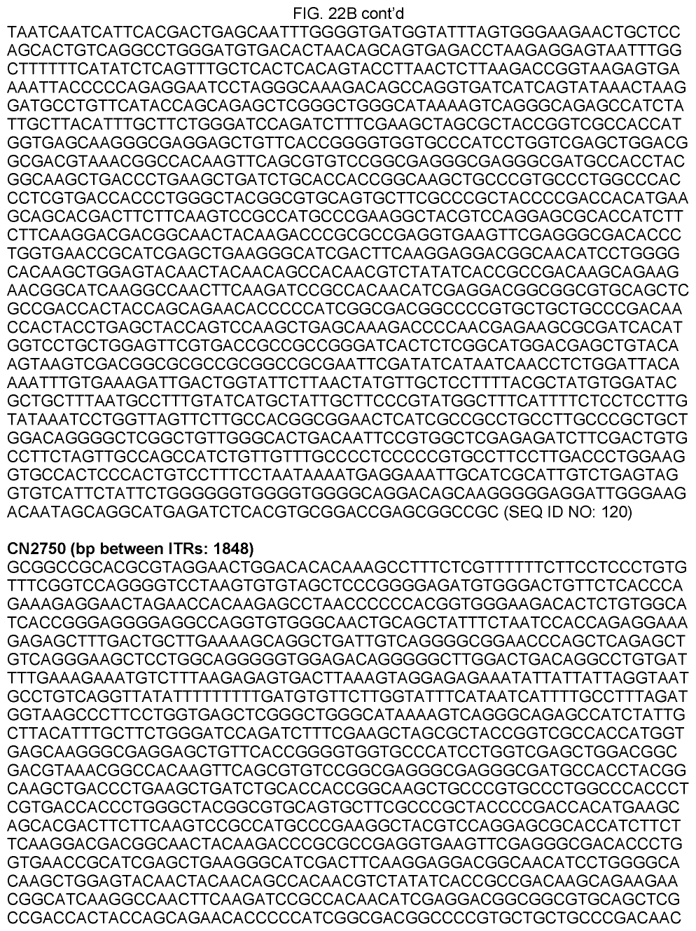

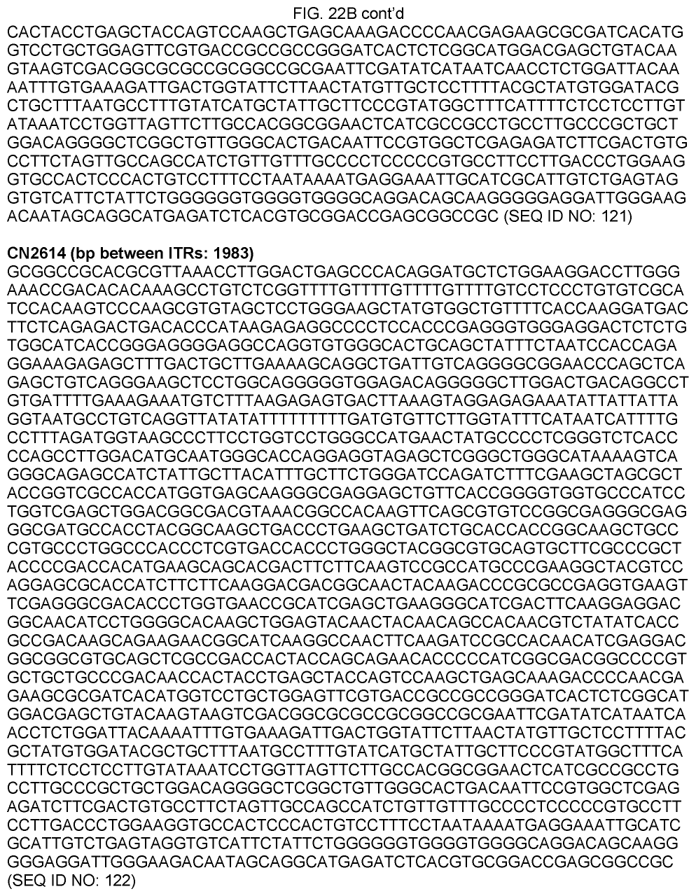

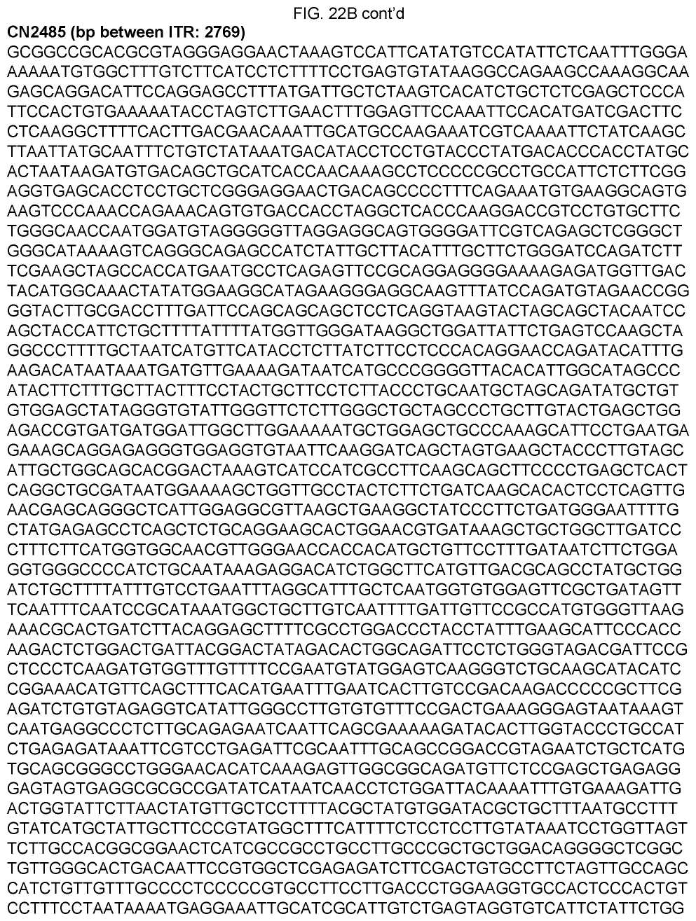

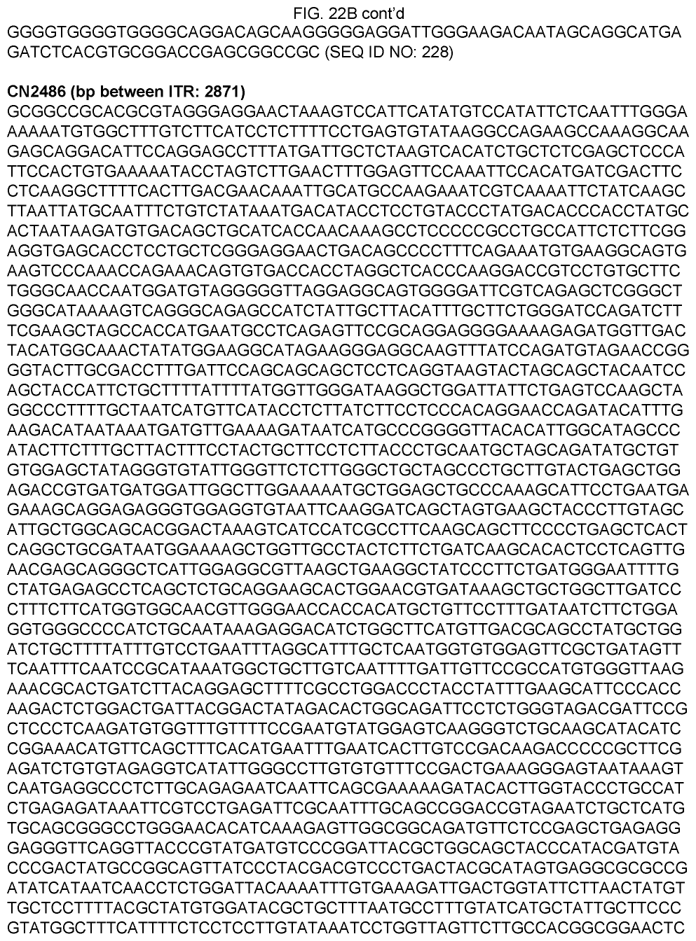

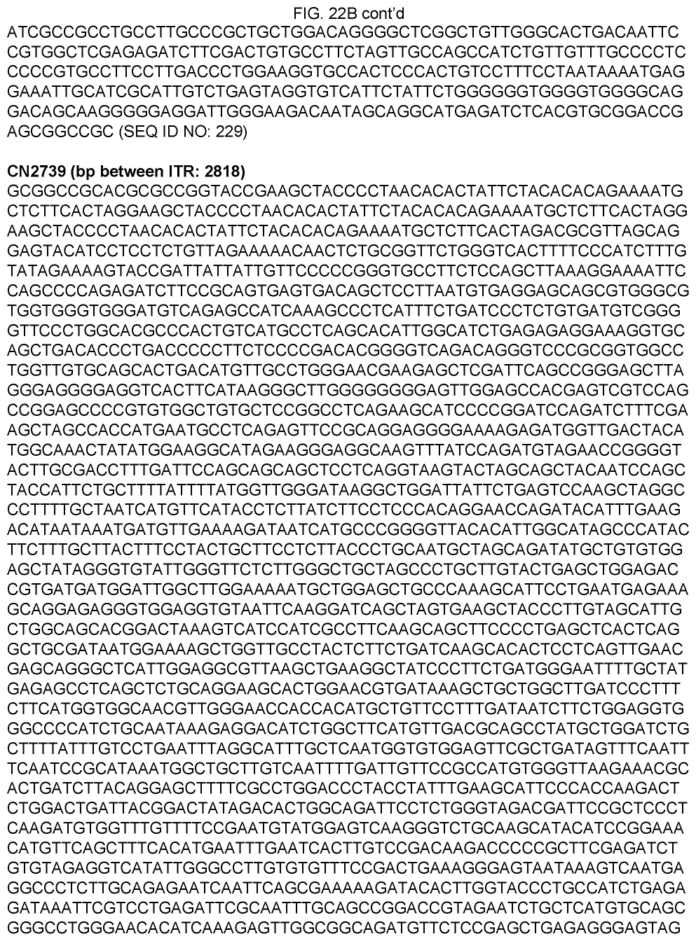

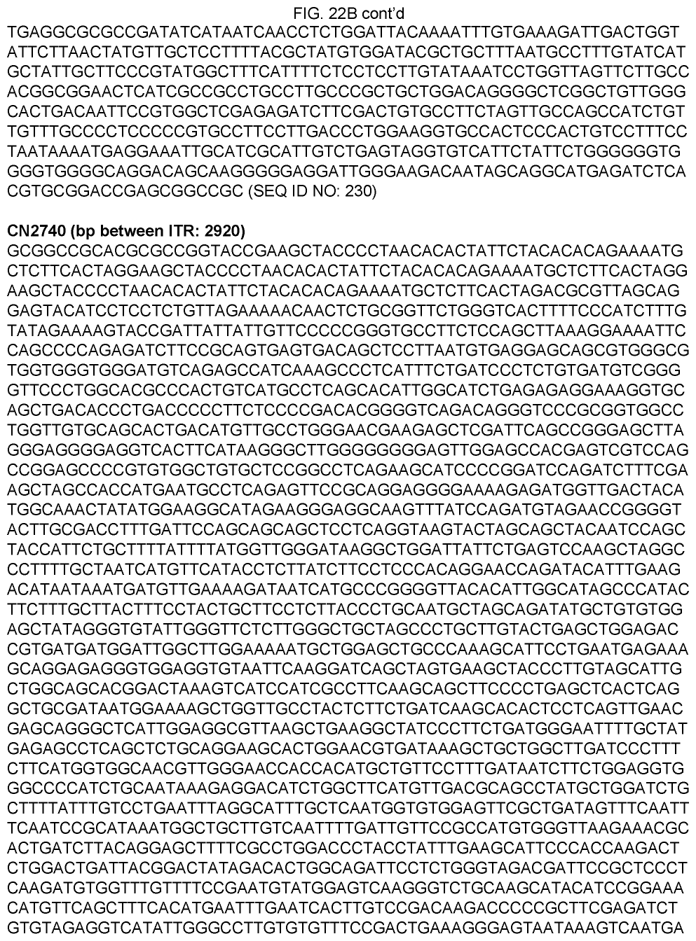

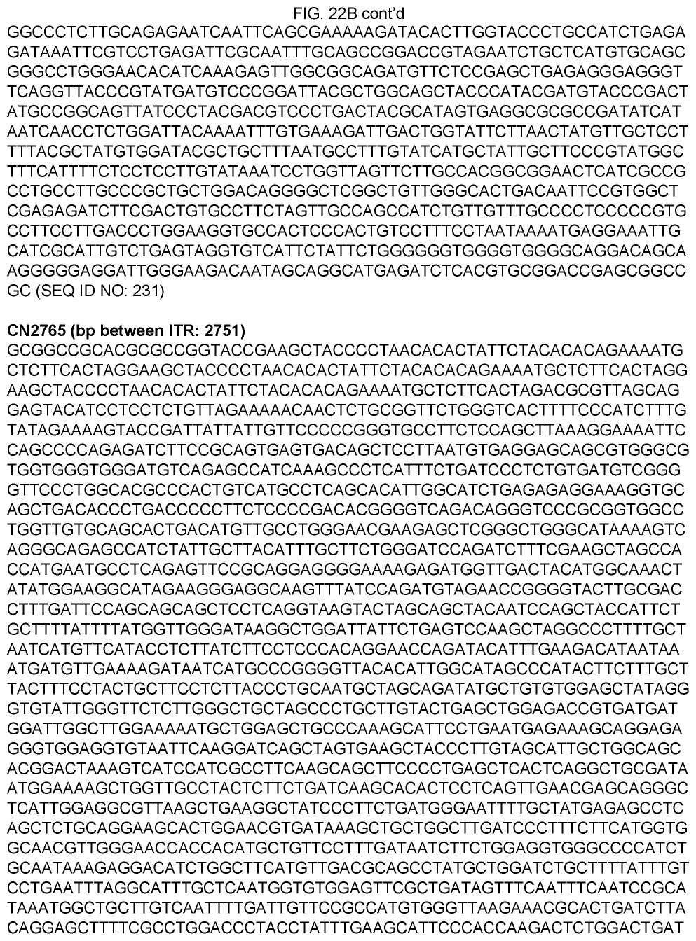

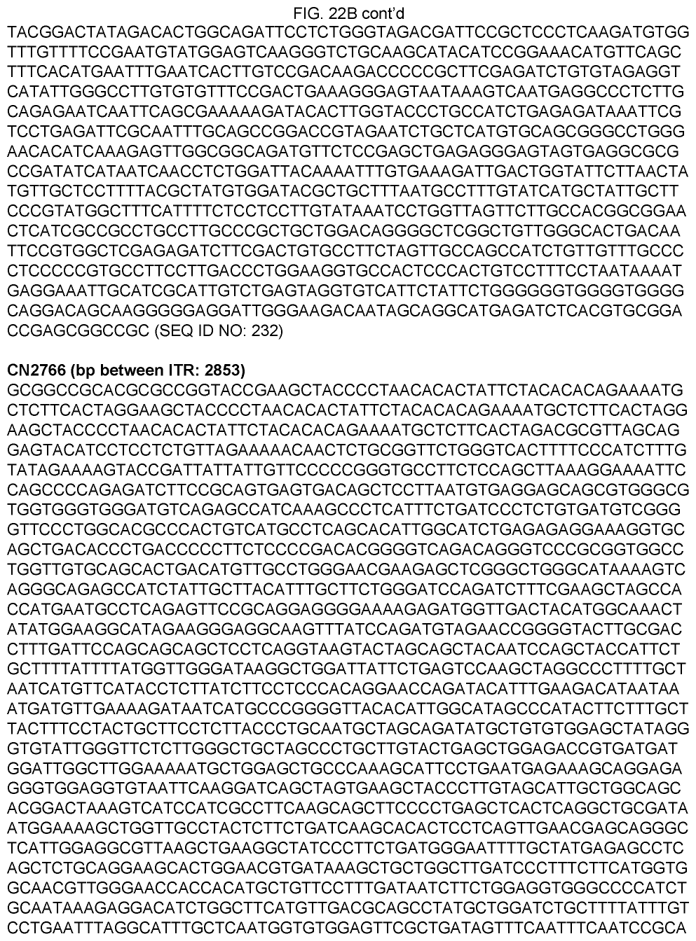

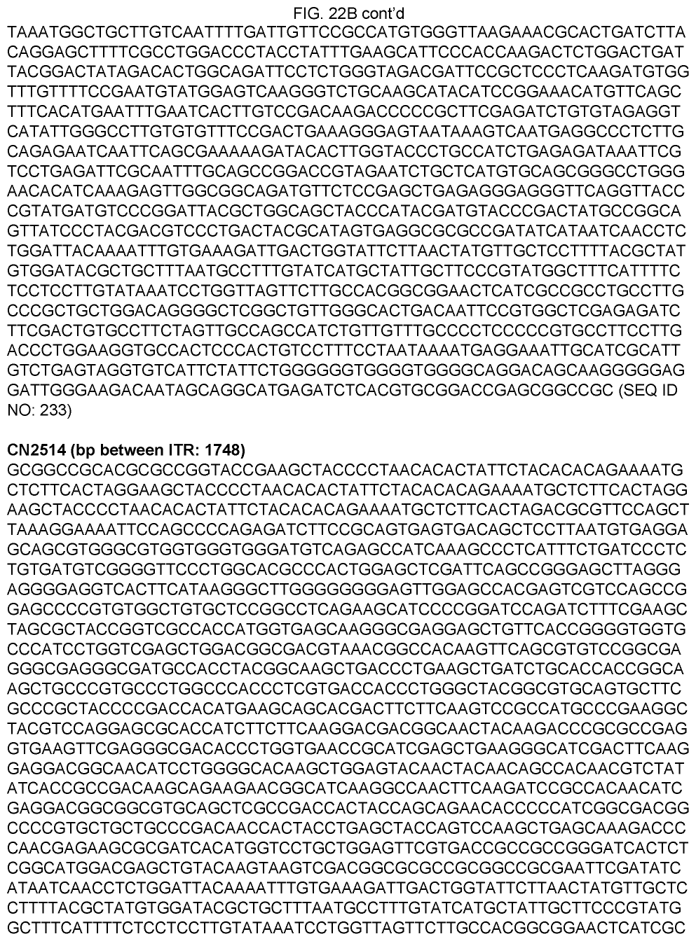

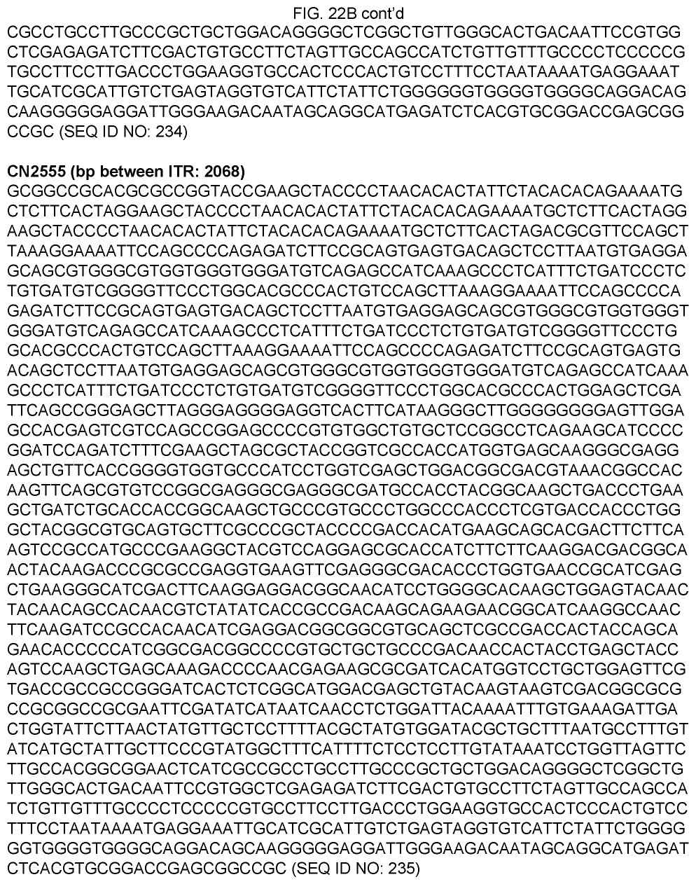

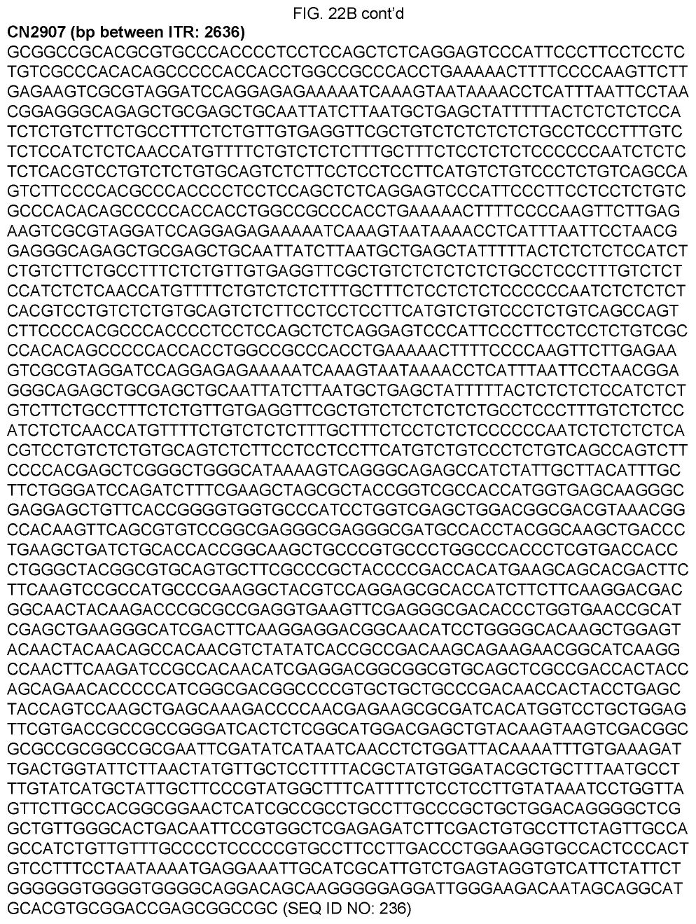

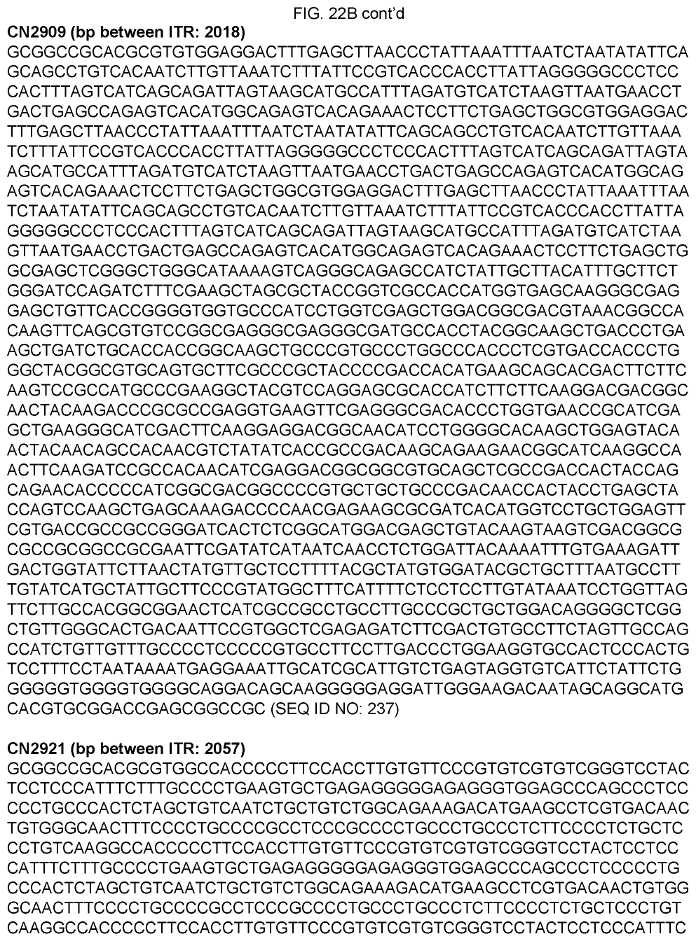

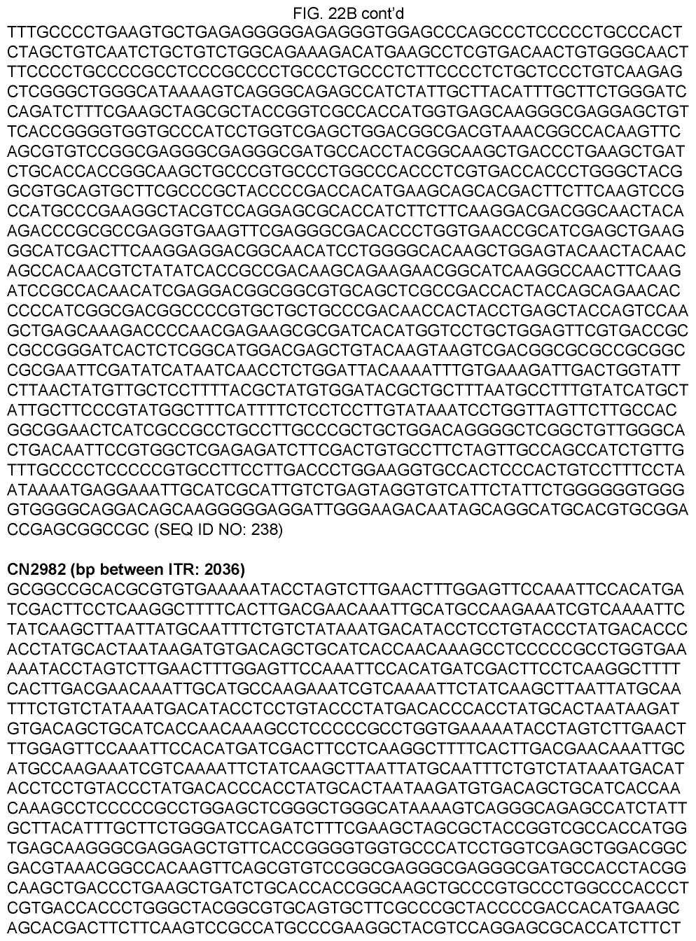

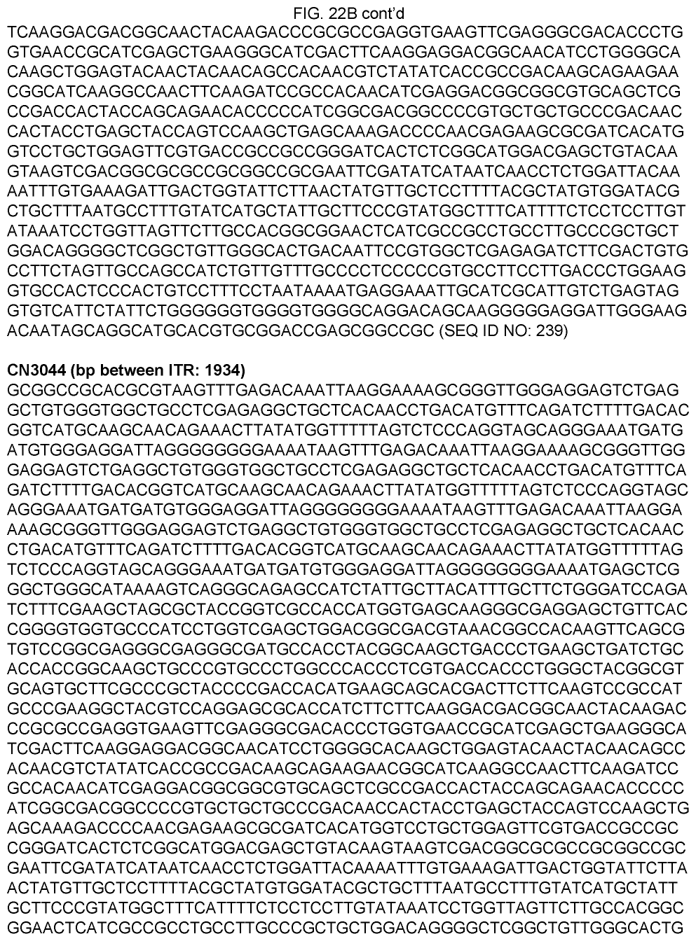

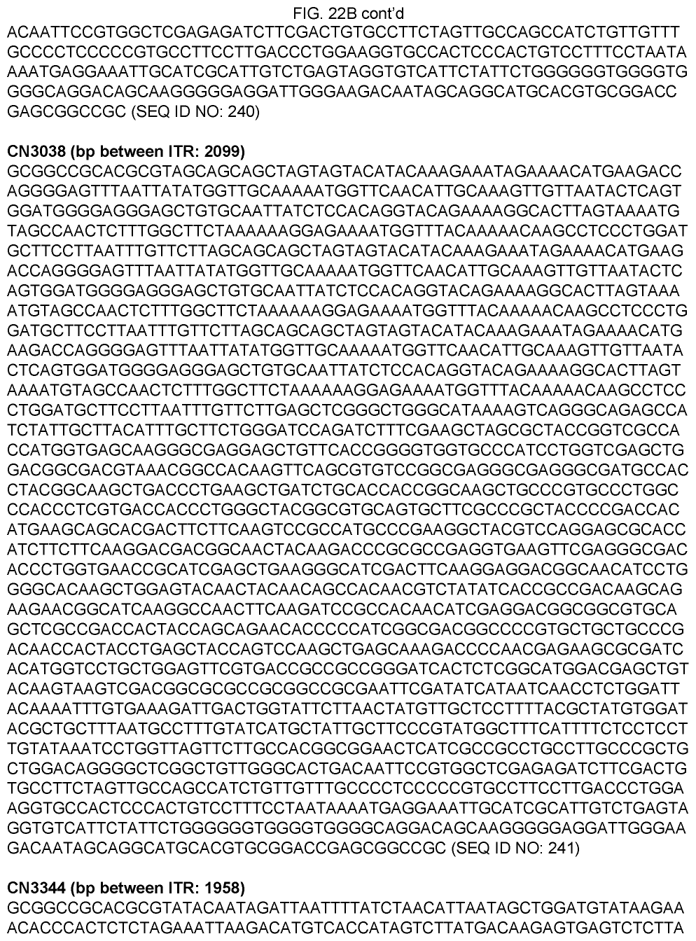

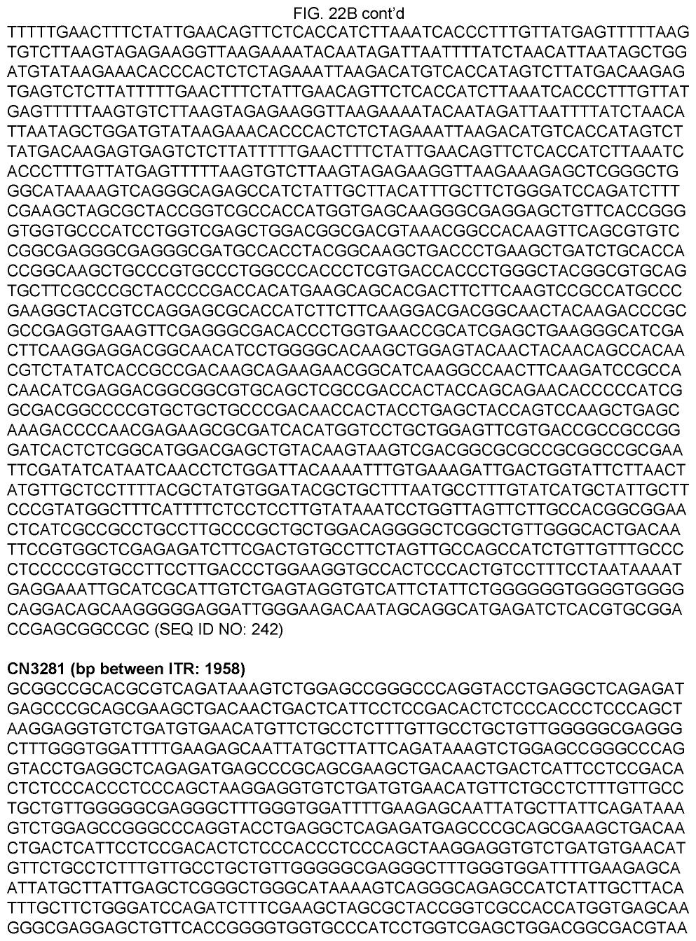

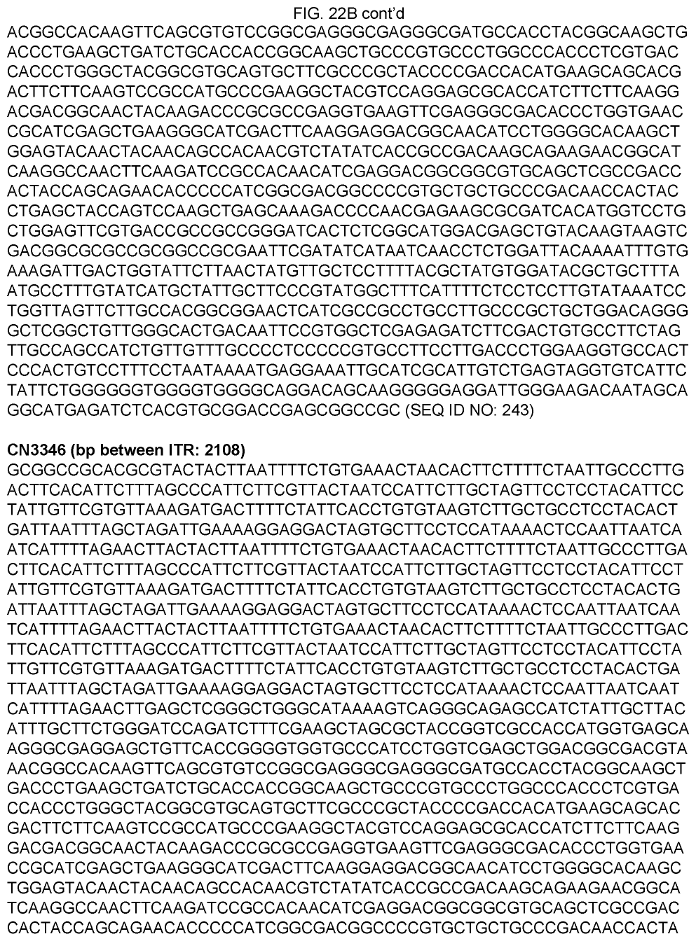

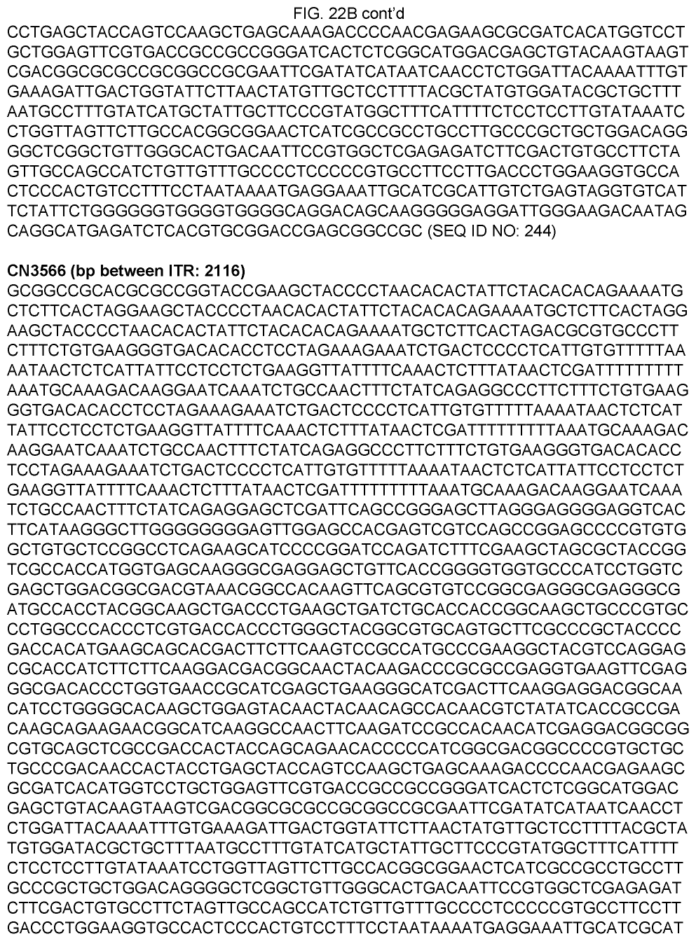

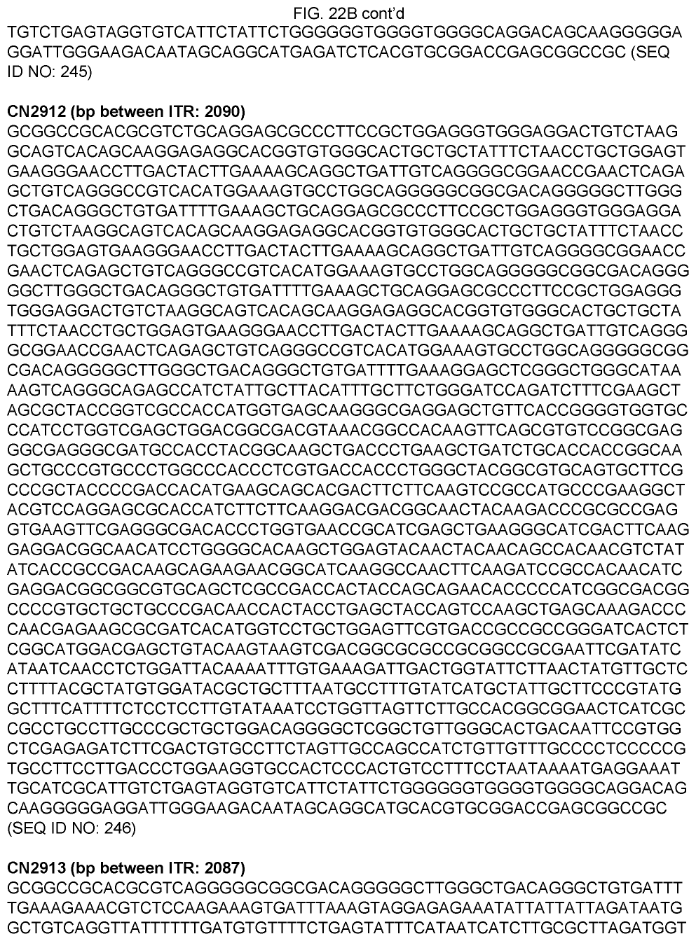

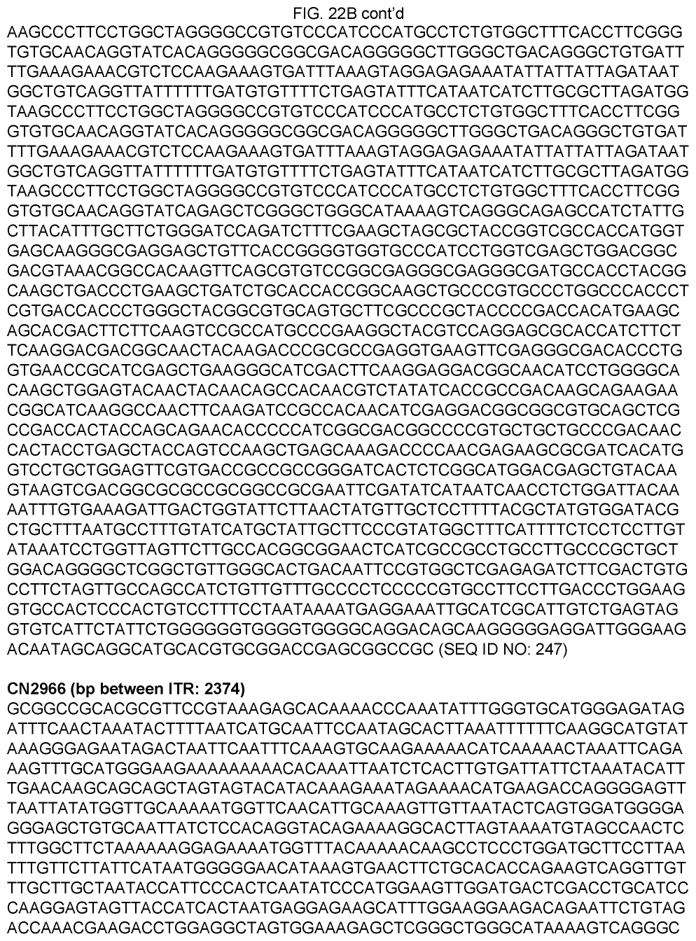

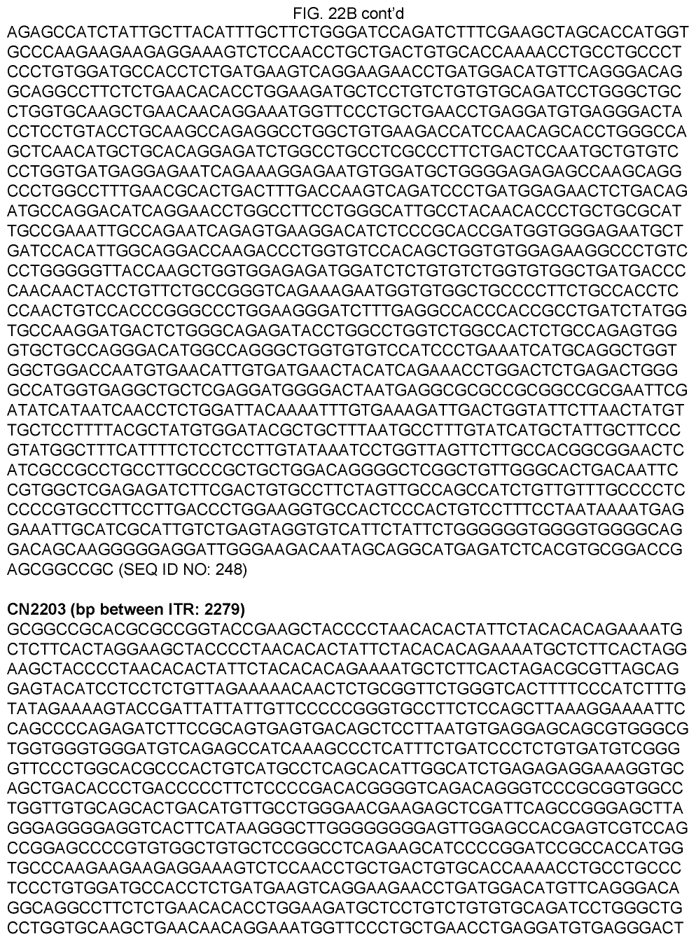

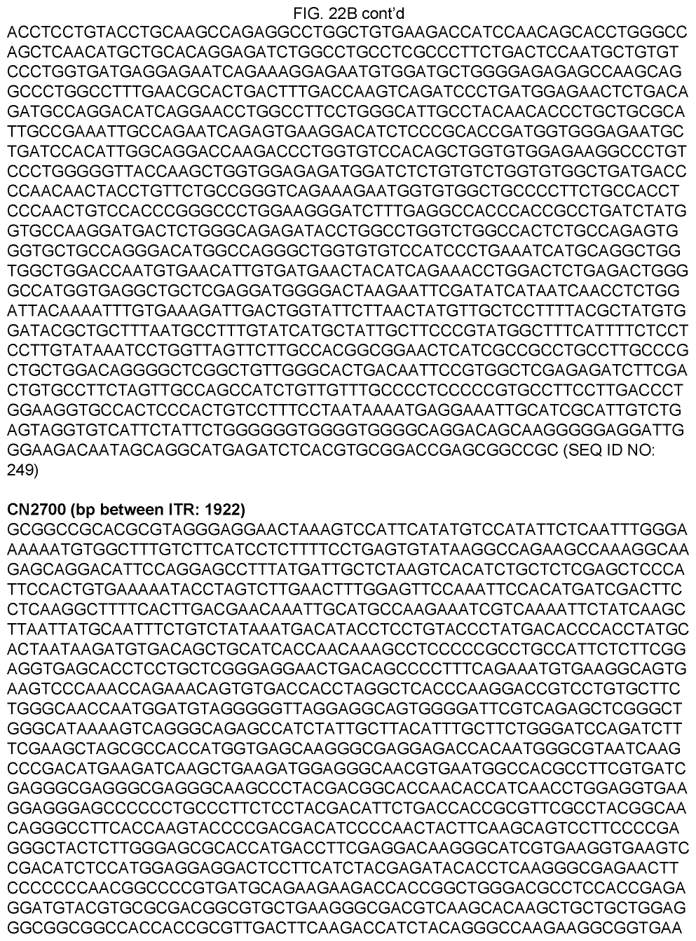

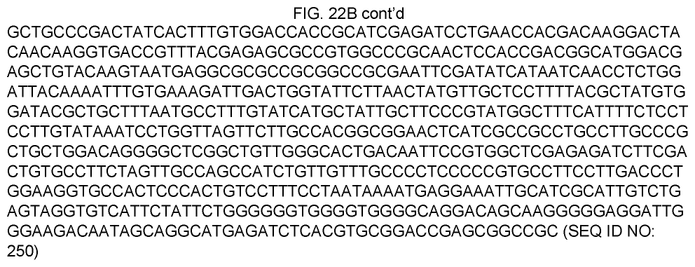

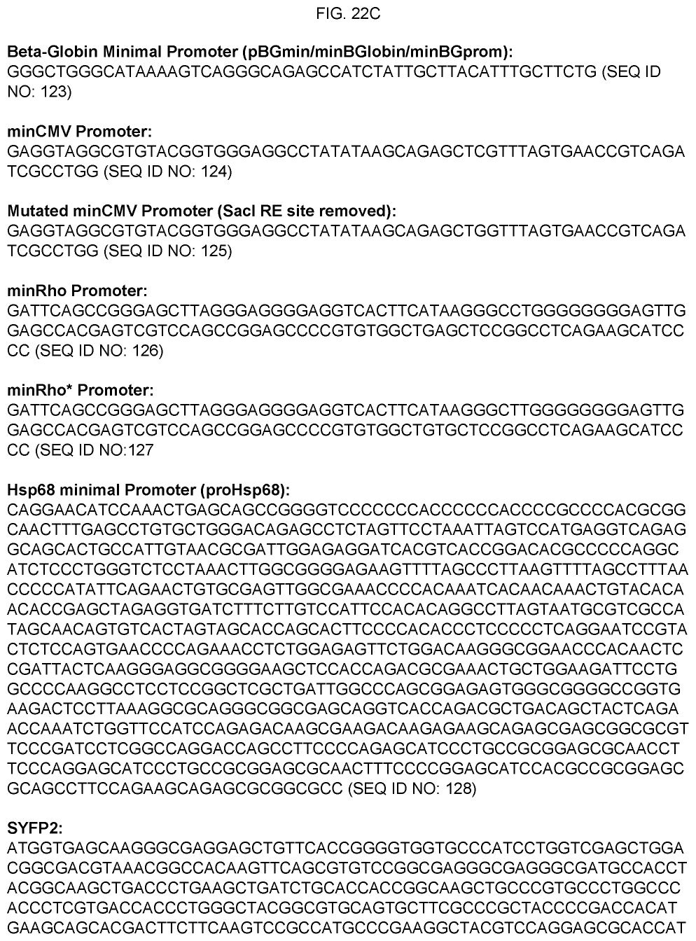

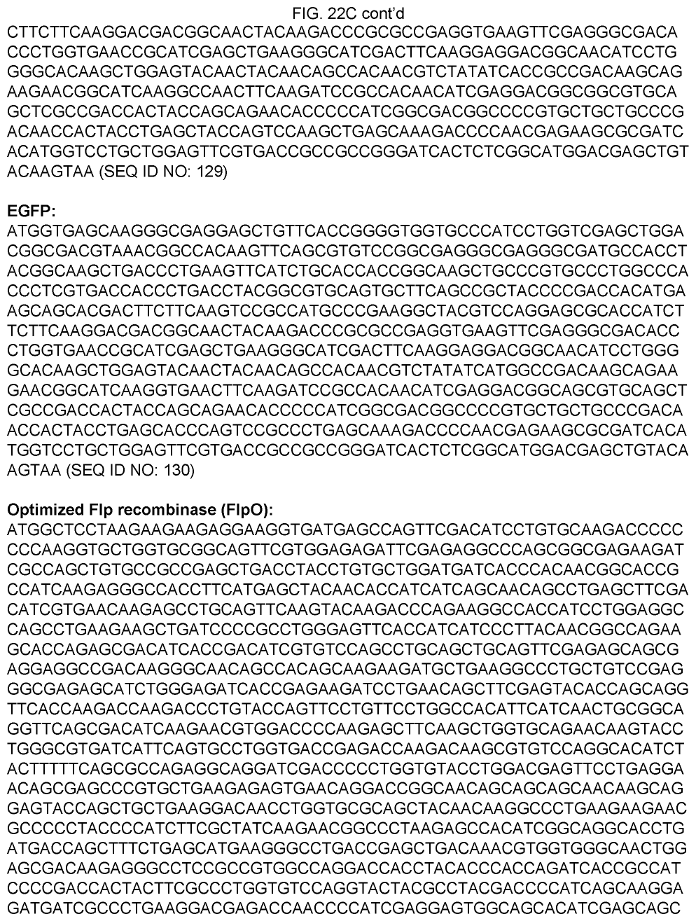

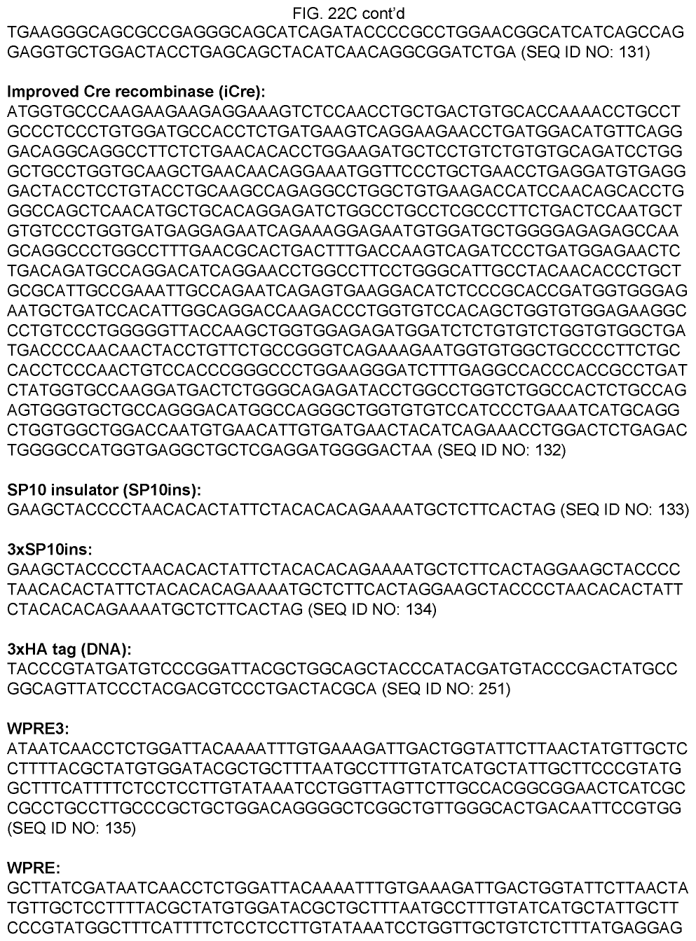

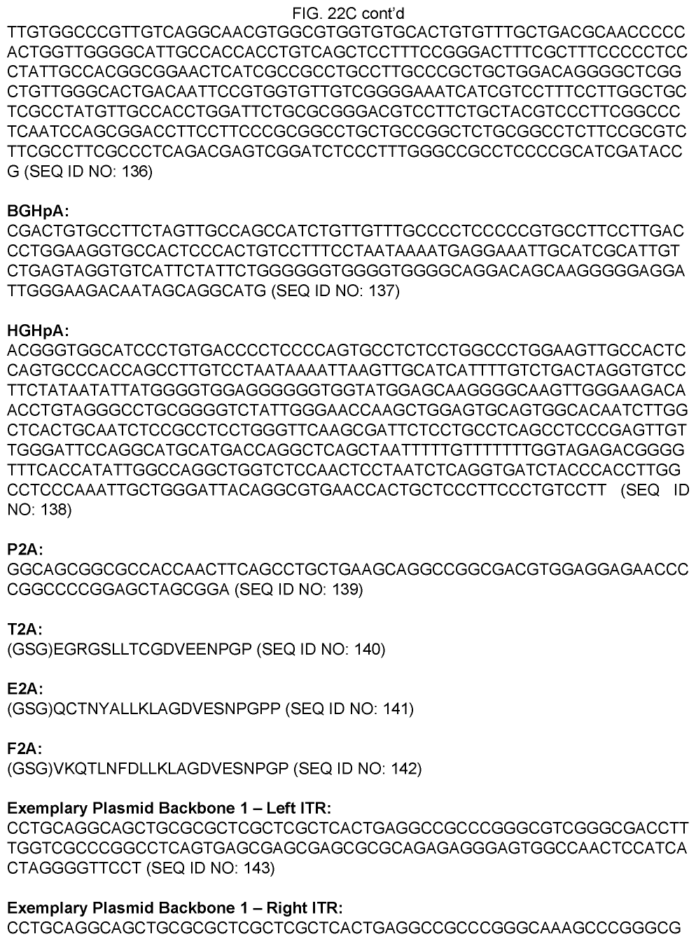

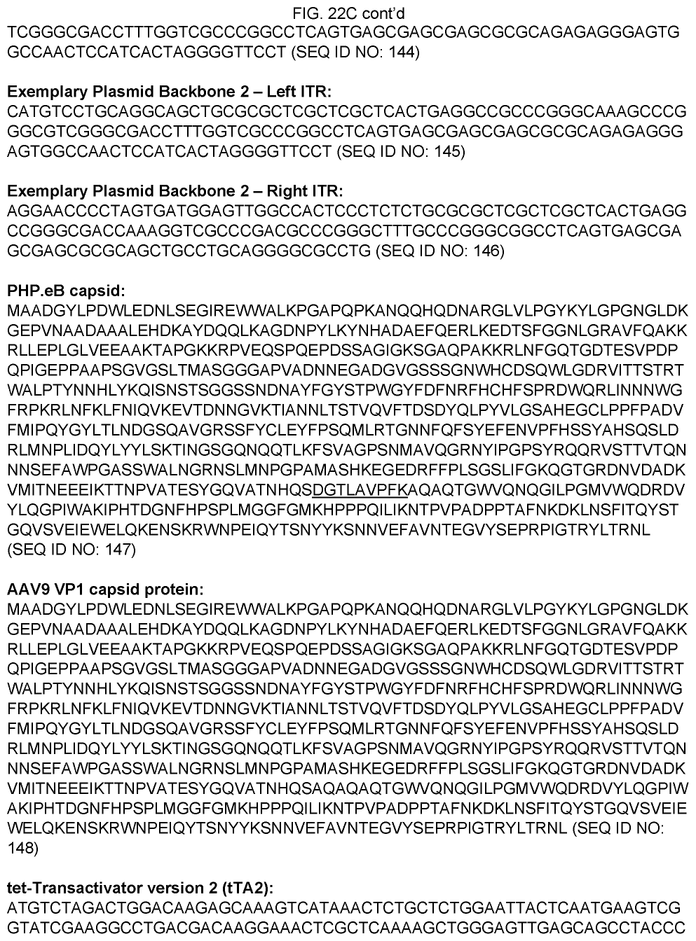

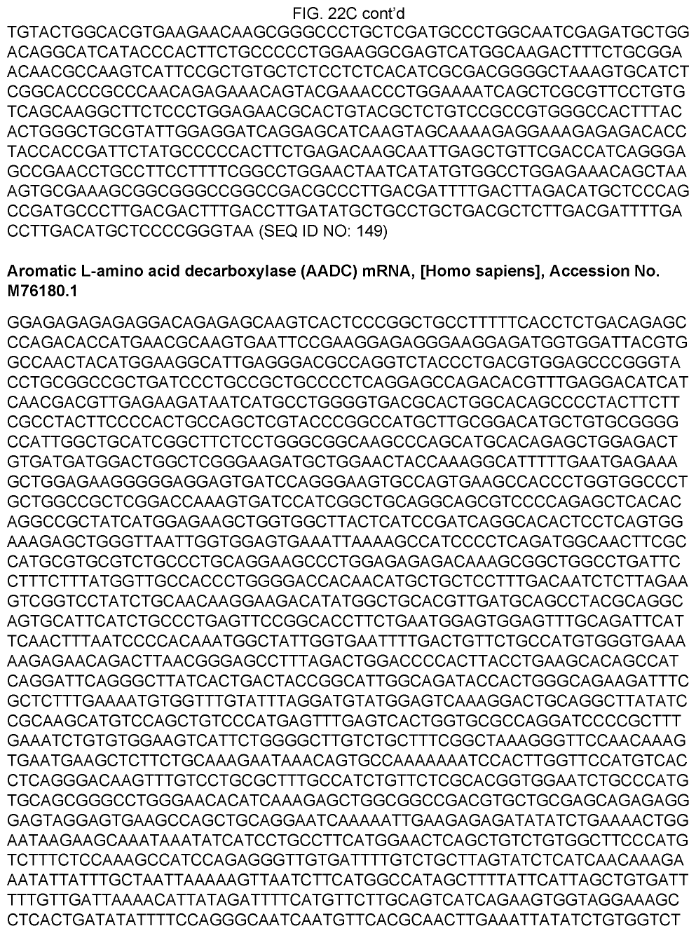

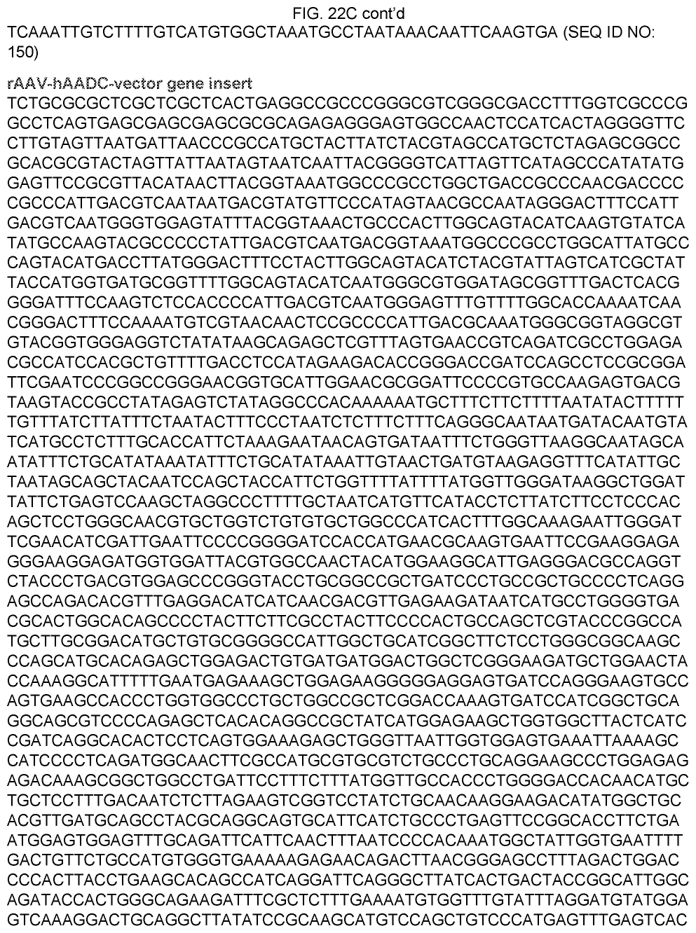

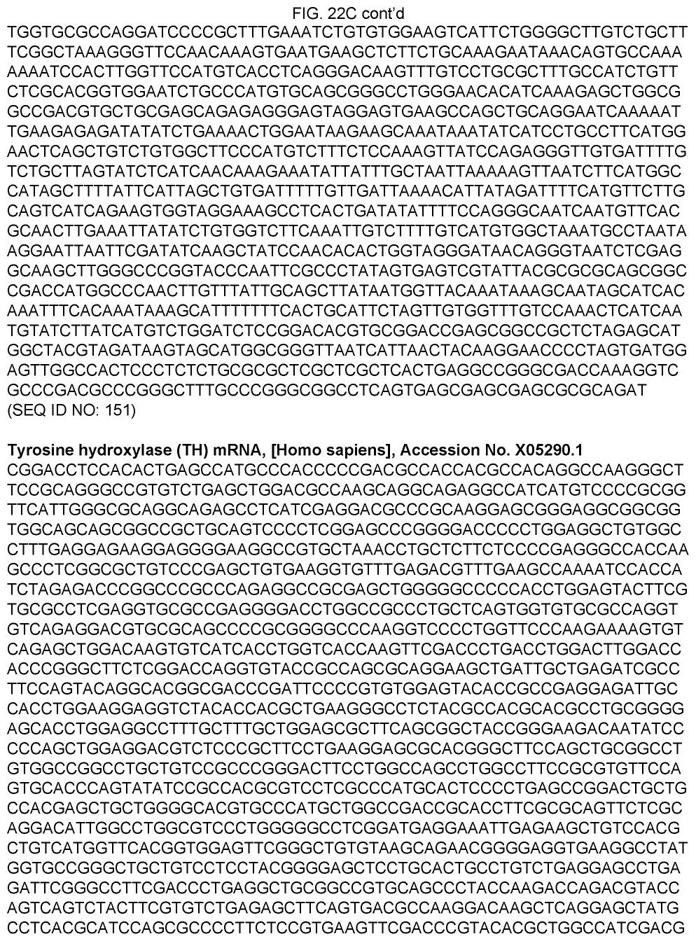

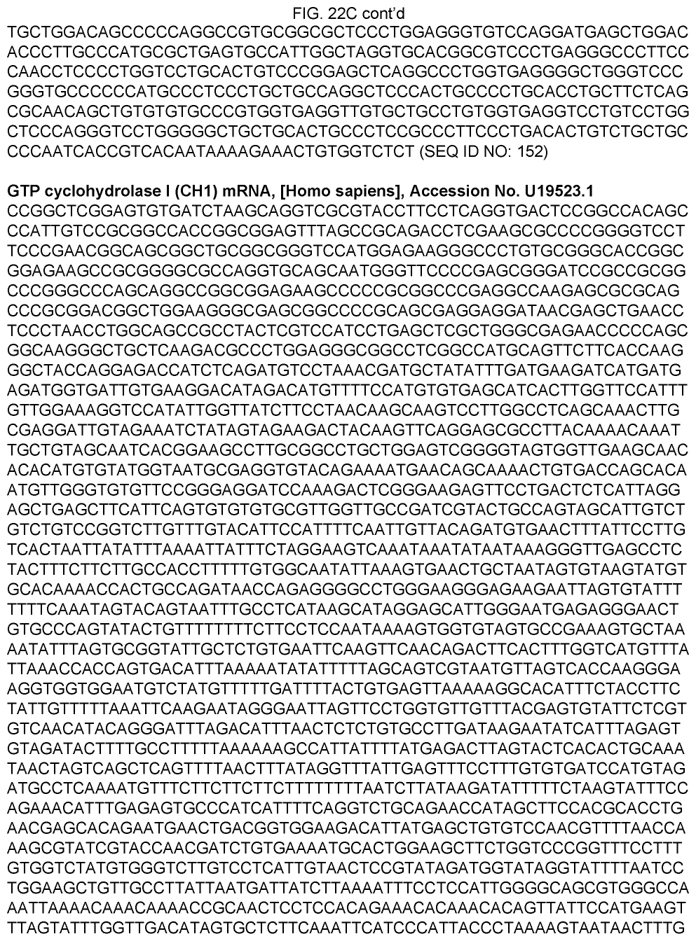

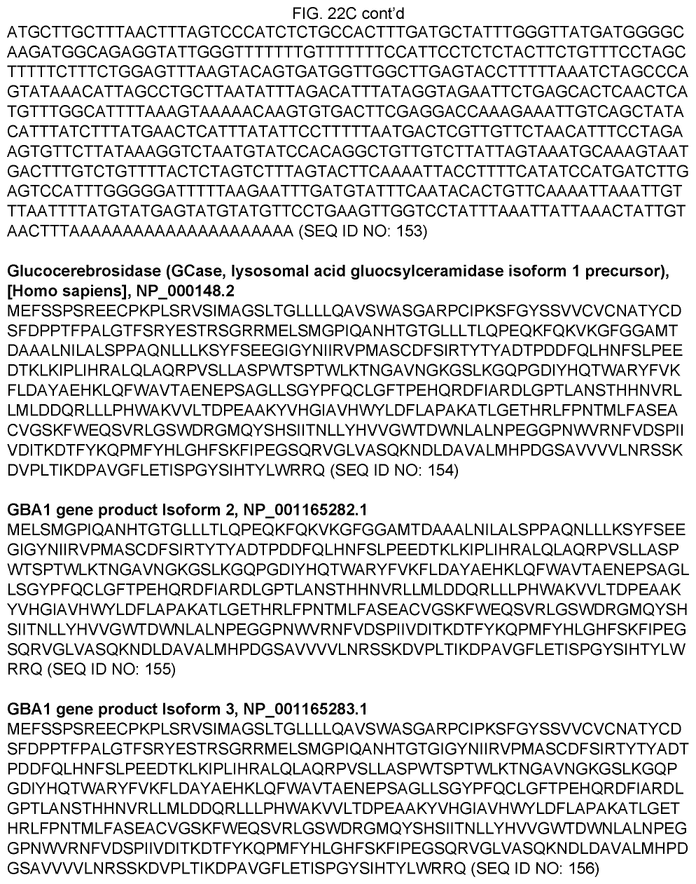

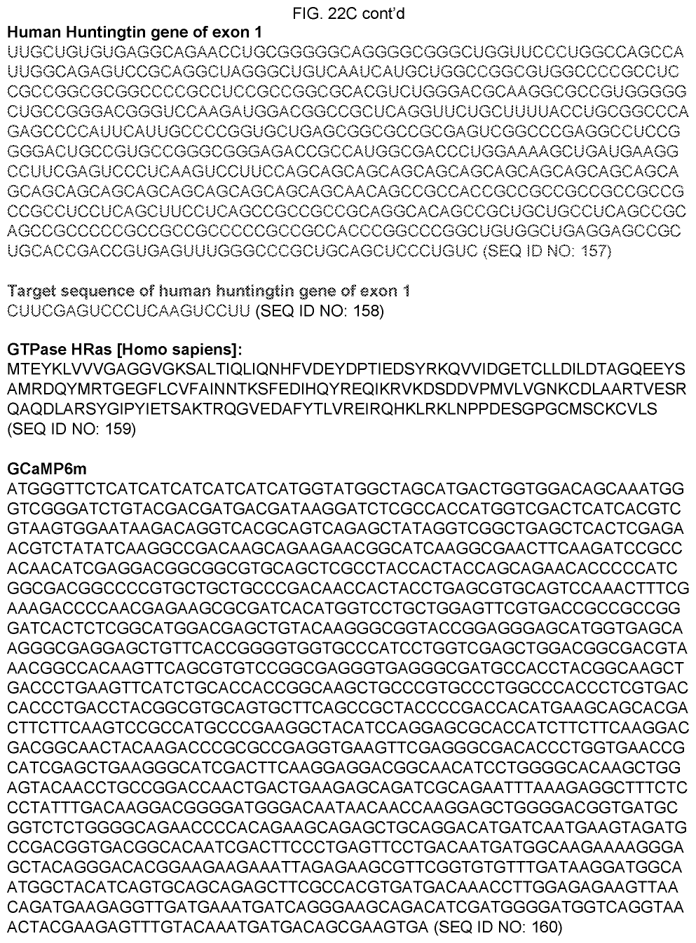

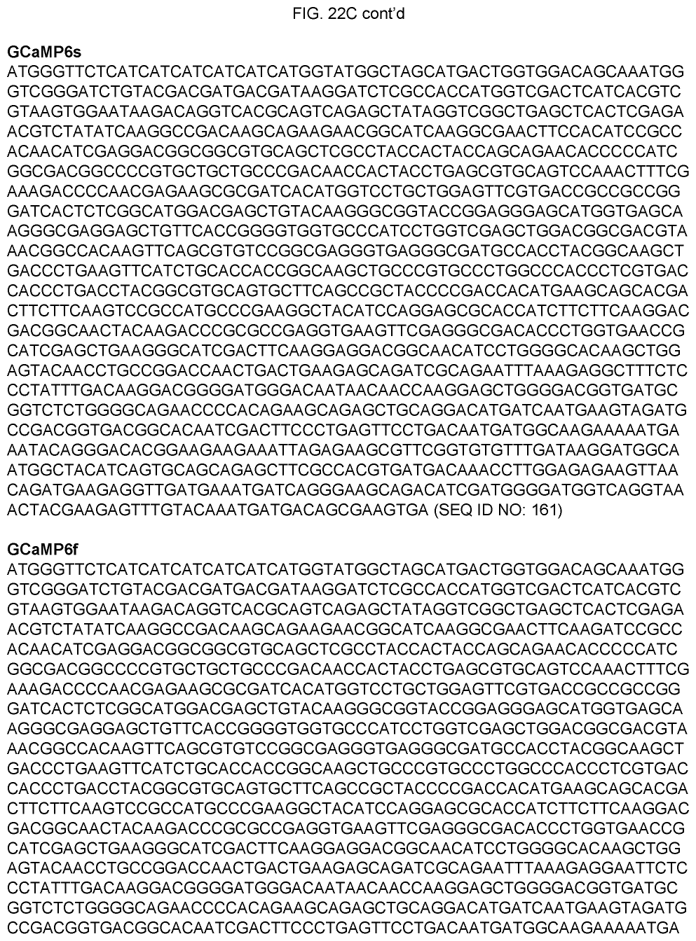

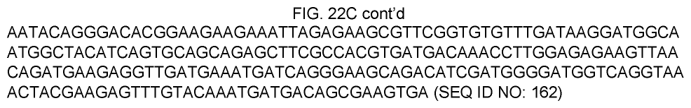

In particular embodiments, the nucleotide sequence encoding human aromatic L-amino acid decarboxylase (AADC) includes Accession No. M76180.1 shown as SEQ ID NO: 150 in FIG. 22C; the nucleotide sequence encoding human tyrosine hydroxylase (TH) includes Accession No. X05290.1 shown as SEQ ID NO: 152 in FIG. 22C; the nucleotide sequence encoding human GTP cyclohydrolase I (CH1) includes Accession No. U19523.1 shown as SEQ ID NO: 153 in FIG. 22C; and/or the amino acid sequence of human glucocerebrosidase includes Accession No. NP_000148.2 shown as SEQ ID NO: 154 in FIG. 22C. In particular embodiments, the amino acid sequence of the GBA1 gene as Isoform 1, Isoform 2, or Isoform 3 includes Accession Nos. NP_001005742.1, NP_001165282.1, or NP_001165283.1, respectively. In particular embodiments, the amino acid sequence of human lactase includes GenBank Accession No. EAX11622.1; the amino acid sequence of human lipase includes GenBank Accession No. AAA60129.1; the amino acid sequence of human helicase includes GenBank Accession No. AMD82207.1; the amino acid sequence of human amylase includes GenBank Accession No. AAA51724.1; and/or the amino acid sequence of human alpha-glucosidase includes GenBank Accession No. AB153718.1.

In particular embodiments, the amino acid sequence of human transcription factor SP1 includes UniProtKB/Swiss-Prot: P08047.3; the amino acid sequence of human transcription factor AP-1 includes NP_002219.1; the amino acid sequence of human heat shock factor protein 1 includes UniProtKB/Swiss-Prot: Q00613.1; the amino acid sequence of human CCAAT/enhancer-binding protein (C/EBP) beta isoform a includes NP_005185.2; and/or the amino acid sequence of human octamer-binding protein 1 (Oct-1) includes UniProtKB/Swiss-Prot: P14859.2.

In particular embodiments, the amino acid sequence of human transforming growth factor receptor beta 1 includes GenBank Accession No. CAF02096.2; the amino acid sequence of human platelet-derived growth factor receptor includes GenBank Accession No. AAA60049.1; the amino acid sequence of human epidermal growth factor receptor includes GenBank Accession No. CAA25240.1; the amino acid sequence of human vascular endothelial growth factor receptor includes GenBank Accession No. AAC16449.2; and/or the amino acid sequence of human interleukin 8 receptor alpha includes GenBank Accession No. AAB59436.1.

In particular embodiments, the amino acid sequence of human caveolin includes GenBank Accession No. CAA79476.1; the amino acid sequence of human dynamin includes GenBank Accession No. AAA88025.1; the amino acid sequence of human clathrin heavy chain 1 isoform 1 includes NP_004850.1; the amino acid sequence of human clathrin heavy chain 2 isoform 1 includes NP_009029.3; the amino acid sequence of human clathrin light chain A isoform a includes NP_001824.1; and/or the amino acid sequence of human clathrin light chain B isoform a includes NP_001825.1.

In particular embodiments, the amino acid sequence of human Ras-related protein Rab-4A isoform 1 includes NP_004569.2; the amino acid sequence of Ras-related protein Rab-11A includes UniProtKB/Swiss-Prot: P62491.3; the amino acid sequence of human platelet-derived growth factor includes GenBank Accession No. AAA60552.1; the amino acid sequence of human transforming growth factor-beta3 includes GenBank Accession No. AAA61161.1; the amino acid sequence of human nerve growth factor includes GenBank Accession No. CAA37703.1; the amino acid sequence of human epidermal growth factor (EGF) includes GenBank Accession No. CAA34902.2; and/or the amino acid sequence of human GTPase HRas can be found in FIG. 22C (SEQ ID NO: 159).

In particular embodiments, the amino acid sequence of human cocaine and amphetamine regulated transcript (Chain A) includes Protein Data Bank ID 1HY9_A; the amino acid sequence of Substance P includes positions 58-68 of Protachykinin-1; the amino acid sequence of human protachykinin-1 includes UniProtKB: P20366; the amino acid sequence of oxytocin includes positions 20-28 of oxytocin-neurophysin 1; the amino acid sequence of human oxytocin-neurophysin 1 includes UniProtKB: P01178; and/or the amino acid sequence of human somatostatin includes GenBank Accession No. AAH32625.1.

In particular embodiments, functional molecules include reporters of cell function and states such as calcium reporters. Intracellular calcium concentration is an important predictor of numerous cellular activities, which include neuronal activation, muscle cell contraction and second messenger signaling. A sensitive and convenient technique to monitor the intracellular calcium levels is through the genetically encoded calcium indicator (GECI). Among the GECIs, green fluorescent protein (GFP) based calcium sensors named GCaMPs are efficient and widely used tools. The GCaMPs are formed by fusion of M13 and calmodulin protein to N- and C-termini of circularly permutated GFP. Some GCaMPs yield distinct fluorescence emission spectra (Zhao et al., Science, 2011, 333(6051): 1888-1891). Exemplary GECIs with green fluorescence include GCaMP3, GCaMP5G, GCaMP6s, GCaMP6m, GCaMP6f, jGCaMP7s, jGCaMP7c, jGCaMP7b, and jGCaMP7f. Furthermore, GECIs with red fluorescence include jRGECO1a and jRGECO1b. AAV products containing GECIs are commercially available. For example, Vigene Biosciences provides AAV products including AAV8-CAG-GCaMP3 (Cat. No:BS4-CX3AAV8), AAV8-Syn-FLEX-GCaMP6s-WPRE (Cat. No:BS1-NXSAAV8), AAV8-Syn-FLEX-GCaMP6s-WPRE (Cat. No:BS1-NXSAAV8), AAV9-CAG-FLEX-GCaMP6m-WPRE (Cat. No:BS2-CXMAAV9), AAV9-Syn-FLEX-jGCaMP7s-WPRE (Cat. No:BS12-NXSAAV9), AAV9-CAG-FLEX-jGCaMP7f-WPRE (Cat. No:BS12-CXFAAV9), AAV9-Syn-FLEX-jGCaMP7b-WPRE (Cat. No:BS12-NXBAAV9), AAV9-Syn-FLEX-jGCaMP7c-WPRE (Cat. No:BS12-NXCAAV9), AAV9-Syn-FLEX-NES-jRGECO1a-WPRE (Cat. No:BS8-NXAAAV9), and AAV8-Syn-FLEX-NES-jRCaMP1b-WPRE (Cat. No:BS7-NXBAAV8).

In particular embodiments, the amino acid sequence of GCaMP6m includes SEQ ID NO: 160 in FIG. 22C; the amino acid sequence of GCaMP6s includes SEQ ID NO: 161 in FIG. 22C; and/or the amino acid sequence of GCaMP6f includes SEQ ID NO: 162 in FIG. 22C.

In particular embodiments calcium reporters include the genetically encoded calcium indicators GECI, NTnC; Myosin light chain kinase, GFP, Calmodulin chimera; Calcium indicator TN-XXL; BRET-based auto-luminescent calcium indicator; and/or Calcium indicator protein OeNL(Ca2+)-18u).

In particular embodiments, the amino acid sequence of myosin light chain kinase, Green fluorescent protein, Calmodulin chimera (Chain A) includes Protein Data Bank ID 3EKJ_A. In particular embodiments, the amino acid sequence of genetically-encoded green calcium indicator NTnC (chain A) includes PDB: 5MWC_The amino acid sequence of calcium indicator TN-XXL can include GenBank Accession No. ACF93133.1 and the amino acid sequence of BRET-based auto-luminescent calcium indicator can include GenBank Accession No. ADF42668.1. In particular embodiments, the amino acid sequence of calcium indicator protein OeNL(Ca2+)-18u includes GenBank Accession No. BBB18812.1.

In particular embodiments, functional molecules include modulators of neuronal activity like channelrhodopsins (e.g., channelrhodopsin-1, channelrhodopsin-2, and variants thereof). Channelrhodopsins are a subfamily of retinylidene proteins (rhodopsins) that function as light-gated ion channels. In addition to channelrhodopsin 1 (ChR1) and channelrhodopsin 2 (ChR2), several variants of channelrhodopsins have been developed. For example, Lin et al. (Biophys J, 2009, 96(5): 1803-14) describe making chimeras of the transmembrane domains of ChR1 and ChR2, combined with site-directed mutagenesis. Zhang et al. (Nat Neurosci, 2008, 11(6): 631-3) describe VChR1, which is a red-shifted channelrhodopsin variant. VChR1 has lower light sensitivity and poor membrane trafficking and expression. Other known channelrhodopsin variants include the ChR2 variant described in Nagel, et al., Proc Natl Acad Sci USA, 2003, 100(24): 13940-5), ChR2/H134R (Nagel, G., et al., Curr Biol, 2005, 15(24): 2279-84), and ChD/ChEF/ChIEF (Lin, J. Y., et al., Biophys J, 2009, 96(5): 1803-14), which are activated by blue light (470 nm) but show no sensitivity to orange/red light. Additional variants are described in Lin, Experimental Physiology, 2010, 96.1: 19-25 and Knopfel et al., The Journal of Neuroscience, 2010, 30(45): 14998-15004).

In particular embodiments, the amino acid sequence of channelopsin 1 [Mesostigma viride] includes UniProtKB: F8UV15; the amino acid sequence of channelopsin 1 [Chlamydomonas yellowstonensis] includes GenBank Accession No. AER58217.1; the amino acid sequence of channelrhodopsin-2 [Volvox carteri f. nagariensis] includes UniProtKB: B4Y105; and/or the amino acid sequence of channel rhodopsin 2 includes GenBank Accession No. AB064386.1.

In particular embodiments, functional molecules include DNA and RNA editing tools such CRISPR/CAS (e.g., guide RNA and a nuclease, such as Cas, Cas9 or cpf1). Functional molecules can also include engineered Cpf1s such as those described in US 2018/0030425, US 2016/0208243, WO/2017/184768 and Zetsche et al. (2015) Cell 163: 759-771; single gRNA (see e.g., Jinek et al. (2012) Science 337:816-821; Jinek et al. (2013) eLife 2:e00471; Segal (2013) eLife 2:e00563) or editase, guide RNA molecules or homologous recombination donor cassettes. In particular embodiments, functional molecules include RNA (e.g. microRNA) that suppresses or inhibits the expression of a pathogenic huntingtin (HTT) gene.

In particular embodiments, the nucleotide sequence encoding the human huntingtin gene of exon 1 includes the sequence set forth SEQ ID NO: 157 in FIG. 22C. An example of the HTT protein sequence includes NCBI Accession No. NP_002102.4 and the encoding sequence includes NCBI Accession No. NM_002111.7. A sequence set forth in SEQ ID NO: 158 corresponds to a target sequence of the human huntingtin gene of exon 1. RNA sequences targeting the target sequence of the human huntingtin gene of exon 1 can be found in U.S. Pat. No. 10,174,321; U.S. application Ser. No. 16/302,140; and PCT Application No. WO2018US52103. Additional RNAi molecules designed to target the nucleotide sequence that encodes the poly-glutamine repeat proteins are described in U.S. Pat. Nos. 9,169,483 and 9,181,544; International Patent Publication No. WO2015179525; and described elsewhere herein. The amino acid sequence of CRISPR-associated protein (Cas) is shown in GenBank Accession No. AKG27598.1; the amino acid sequence of Cas9 is shown in GenBank Accession No. AST09977.1; and the amino acid sequence of CRISPR-associated endonuclease Cpf1 is shown in UniProtKB/Swiss-Prot: U2UMQ6.1. In particular embodiments, the amino acid sequence of human ribonuclease 4 or ribonuclease L includes UniProtKB/Swiss-Prot: Q05823.2 and the amino acid sequence of human deoxyribonuclease II beta is shown in GenBank Accession No. AAF76893.1.

Exon 1 RNA sequence of the huntingtin (HTT) gene is set forth in SEQ ID NO: 157. The CAG repeat sequence is from nucleotides 367-429. Functional molecules can be used to disrupt or inhibit the expression of Exon 1 of HTT. In particular embodiments, double stranded RNAs are used to disrupt or inhibit the expression of Exon 1 of HTT. Exemplary double stranded RNAs include microRNA. In particular embodiments, doubles stranded RNAs target sequences of Exon H1 at the target sequences (Name, Nucleotide location) including H1, 185-205; H2, 186-206; H3, 189-209; H4, 191-211; H5, 194-214; H6, 196-216; H7, 250-270; H8, 261-281, H9, 310-330; H10, 311-331, H11, 339-359, H12, 345-365, H13, 454-474; H14, 459-479; H15, 477-497; H16, 486-506; H17, 492-512; H18, 498-518; H19, 549-569; H20, 557-577; H21, 558-578. In particular embodiments, 5′-CUUCGAGUCCCUCAAGUCCUU-3′ (SEQ ID NO: 158) is H12 which corresponds to a target sequence of the huntingtin gene of exon 1 (SEQ ID NO: 157) at nucleotides 345 to 365.

In particular embodiments, the double stranded RNA includes a first RNA sequence and a second RNA sequence attached via a linker or loop portion. Examples of loop portions can be found in U.S. Pat. No. 9,169,483. In particular embodiments, an RNA sequence has a sequence length of (i) at least 15 nucleotides; (ii) at least 19 nucleotides; or (iii) at least 19, at least 20, at least 21, at least 22, or at least 23 nucleotides. In particular embodiments, an RNA sequence has a sequence length of 15, 16, 17, 18, 19, 20, 21, 22, 23, 24, 25, 26, 27, 28, 29, or 30 nucleotides. Exemplary RNA sequences to include as a first RNA sequence can be selected from SEQ ID NOs: 163-167.

TABLE 1
First RNA Sequences

| SEQ ID | | |
|---|---|---|
| NO: | RNA Sequence | Length |
| 163 | 5′-AAGGACUUGAGGGACUCGA-3′ | 19 |
| 164 | 5′-AAGGACUUGAGGGACUCGAA-3′ | 20 |
| 165 | 5′-AAGGACUUGAGGGACUCGAAG-3′ | 21 |
| 166 | 5′-AAGGACUUGAGGGACUCGAAGG-3′ | 22 |
| 167 | 5′-AAGGACUUGAGGGACUCGAAGGC-3′ | 23 |

Exemplary RNA sequences to include as a second RNA sequence can be selected from SEQ ID NOs: 168-174.

TABLE 2
Second RNA Sequences

| SEQ ID | | |
|---|---|---|
| NO: | RNA Sequence | Length |
| 168 | 5′-UCGAGUCCCUCAAGUCCUU-3′ | 19 |
| 169 | 5′-UUCGAGUCCCUCAAGUCCUU-3′ | 20 |
| 170 | 5′-CUUCGAGUCCCUCAAGUCCUU-3′ | 21 |
| 171 | 5′-CCUUCGAGUCCCUCAAGUCCUU-3′ | 22 |
| 172 | 5′-GCCUUCGAGUCCCUCAAGUCCUU-3′ | 23 |
| 173 | 5′-CUUCGAGUCUCAAGUCCUU-3′ | 19 |
| 174 | 5′-ACGAGUCCCUCAAGUCCUC-3′ | 19 |

In some cases, the double stranded RNA includes a pre-miRNA or pri-miRNA scaffold. A pri-miRNA scaffold includes a pre-miRNA scaffold. The pre-miRNA scaffold includes the double stranded RNA, i.e. the first RNA sequence and the second RNA sequence. In particular embodiments, the sequence of pre-miRNAs are listed in Table 3 below.

TABLE 3
Pre-miRNA scaffolds with SEQ ID NO: 165.

| SEQ ID NO: | Name | Sequence |
|---|---|---|
| 175 | pre- | 5′- |
| | miR451a | CUUGGGAAUGGCAAGGAAGGACUU |
| | | GAGGGACUCGAAGACGAGUCCCUC |
| | | AAGUCCUCUCUUGCUAUACCCAG |
| | | A-3′ |
| 176 | Pre- | 5′- |
| | miR155 | UGCUGAAGGACUUGAGGGACUCG |
| | | AAGGUUUUGGCCACUGACUGACCU |
| | | UCGAGUCUCAAGUCCUUCAGGA-3′ |

In particular embodiments, double stranded RNA sequences include full sequences or part of the sequences of SEQ ID NOs: 177-195. Additional RNA sequences targeting the target sequence of human hungtingtin gene of exon 1 can be found in U.S. Pat. No. 10,174,321; U.S. application Ser. No. 16/302,140; U.S. Pat. Nos. 9,169,483 and 9,181,544; International Patent Publication No. WO2015179525; or International Patent Application No. WO2018US52103.

TABLE 4

| SEQ | |
|---|---|
| ID | |
| NO: | Sequence |
| 177 | AUGAAGGCCUUCGAGUCCCUC |
| 178 | GGCGACCCUGGAAAAGCUGAU |
| 179 | UGGCGACCCUGGAAAAGCUGA |
| 180 | AUGGCGACCCUGGAAAAGCUG |
| 181 | CGACCAUGCGAGCCAGCA |
| 182 | AGUCGCUGAUGACCGGGA |
| 183 | ACGUCGUAAACAAGAGGA |
| 184 | GUCGACCAUGCGAGCCAGCAC |
| 185 | AUAGUCGCUGAUGACCGGGAU |
| 186 | UUACGUCGUAAACAAGAGGAA |
| 187 | AAAACUCGAGUGAGCGCUGAAGGCCUUCGAGUCCCUCAUGAGGGAC |
| | UCGAAGGCCUUCAUCGCCUACUAGUAAAA |
| 188 | AAAACUCGAGUGAGCGCUGAAGGCCUUCGAGUCUUUUAUGAGGGAC |
| | UCGAAGGCCUUCAUCGCCUACUAGUAAAA |
| 189 | AAAACUCGAGUGAGCGCAUGAAGGCCUUCGAGUCCCUCGAGGGACU |
| | CGAAGGCCUUCAUCCGCCUACUAGUAAAA |
| 190 | AAAACUCGAGUGAGCGCAUGAAGGCCUUCGAGUCUUUUGAGGGACU |
| | CGAAGGCCUUCAUCCGCCUACUAGUAAAA |
| 191 | CUCGAGUGAGCGCUCCCGGUCAUCAGCGACUAUUCCGUAAAGCCAC |
| | AGAUGGGGAUAGUCGCUGAUGACCGGGAUCGCCUACUAG |
| 192 | CUCGAGUGAGCGAUGCUGGCUCGCAUGGUCGAUACUGUAAAGCCAC |
| | AGAUGGGUGUCGACCAUGCGAGCCAGCACCGCCUACUAGA |
| 193 | CUCGAGUGAGCGCUCCUCUUGUUUACGACGUGAUCUGUAAAGCCAC |
| | AGAUGGGAUUACGUCGUAAACAAGAGGAACGCCUACUAGU |
| 194 | GCGUUUAGUGAACCGUCAGAUGGUACCGUUUAAACUCGAGUGAGCG |
| | AUGCUGGCUCGCAUGGUCGAUACUGUAAAGCCACAGAUGGGUGUCG |
| | ACCAUGCGAGCCAGCACCGCCUACUAGAGCGGCCGCCACAGCGGGG |
| | AGAUCCAGACAUGAUAAGAUACAUU |
| 195 | GCGUUUAGUGAACCGUCAGAUGGUACCGUUUAAACUCGAGUGAGCG |
| | CUCCCGGUCAUCAGCGACUAUUCCGUAAAGCCACAGAUGGGGAUAG |
| | UCGCUGAUGACCGGGAUCGCCUACUAGAGCGGCCGCCACAGCGGGG |
| | AGAUCCAGACAUGAUAAGAUACAUU |

Additional effector elements include Ore, iCre, dgCre, FIpO, and tTA2. iCre refers to a codon-improved Ore. dgCre refers to an enhanced GFP/Cre recombinase fusion gene with an N terminal fusion of the first 159 amino acids of the Escherichia coli K-12 strain chromosomal dihydrofolate reductase gene (DHFR or foIA) harboring a G67S mutation and modified to also include the R12YNY1001 destabilizing domain mutation. FIpO refers to a codon-optimized form of FLPe that greatly increases protein expression and FRT recombination efficiency in mouse cells. Like the Cre/LoxP system, the FLP/FRT system has been widely used for gene expression (and generating conditional knockout mice, mediated by the FLP/FRT system). tTA2 refers to tetracycline transactivator.

Exemplary expressible elements are expression products that do not include effector elements, for example, a non-functioning or defective protein. In particular embodiments, expressible elements can provide methods to study the effects of their functioning counterparts.

In particular embodiments, expressible elements are non-functioning or defective based on an engineered mutation that renders them non-functioning. In these aspects, non-expressible elements are as similar in structure as possible to their functioning counterparts.

Exemplary self-cleaving peptides include the 2A peptides which lead to the production of two proteins from one mRNA. The 2A sequences are short (e.g., 20 amino acids), allowing more use in size-limited constructs. Particular examples include P2A, T2A, E2A, and F2A. In particular embodiments, the artificial expression constructs include an internal ribosome entry site (IRES) sequence. IRES allow ribosomes to initiate translation at a second internal site on a mRNA molecule, leading to production of two proteins from one mRNA.

Coding sequences encoding molecules (e.g., RNA, proteins) described herein can be obtained from publicly available databases and publications. Coding sequences can further include various sequence polymorphisms, mutations, and/or sequence variants wherein such alterations do not affect the function of the encoded molecule. The term “encode” or “encoding” refers to a property of sequences of nucleic acids, such as a vector, a plasmid, a gene, cDNA, mRNA, to serve as templates for synthesis of other molecules such as proteins.

The term “gene” may include not only coding sequences but also regulatory regions such as promoters, enhancers, insulators, and/or post-regulatory elements, such as termination regions. The term further can include all introns and other DNA sequences spliced from the mRNA transcript, along with variants resulting from alternative splice sites. The sequences can also include degenerate codons of a reference sequence or sequences that may be introduced to provide codon preference in a specific organism or cell type.

Promoters can include general promoters, tissue-specific promoters, cell-specific promoters, and/or promoters specific for the cytoplasm. Promoters may include strong promoters, weak promoters, constitutive expression promoters, and/or inducible promoters. Inducible promoters direct expression in response to certain conditions, signals or cellular events. For example, the promoter may be an inducible promoter that requires a particular ligand, small molecule, transcription factor or hormone protein in order to effect transcription from the promoter. Particular examples of promoters include minBglobin, CMV, minCMV, minCMV* (minCMV* is minCMV with a Sacl restriction site removed) minRho, minRho* (minRho* is minRho with a Sacl restriction site removed), SV40 immediately early promoter, the Hsp68 minimal promoter (proHSP68), and the Rous Sarcoma Virus (RSV) long-terminal repeat (LTR) promoter. Minimal promoters have no activity to drive gene expression on their own but can be activated to drive gene expression when linked to a proximal enhancer element.

In particular embodiments, expression constructs are provided within vectors. The term vector refers to a nucleic acid molecule capable of transferring or transporting another nucleic acid molecule, such as an expression construct. The transferred nucleic acid is generally linked to, e.g., inserted into, the vector nucleic acid molecule. A vector may include sequences that direct autonomous replication in a cell or may include sequences that permit integration into host cell DNA. Useful vectors include, for example, plasmids (e.g., DNA plasmids or RNA plasmids), transposons, cosmids, bacterial artificial chromosomes, and viral vectors.

Viral vector is widely used to refer to a nucleic acid molecule that includes virus-derived components that facilitate transfer and expression of non-native nucleic acid molecules within a cell. The term adeno-associated viral vector refers to a viral vector or plasmid containing structural and functional genetic elements, or portions thereof, that are primarily derived from AAV. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, that are primarily derived from a lentivirus, and so on. The term “hybrid vector” refers to a vector including structural and/or functional genetic elements from more than one virus type.

Adenovirus vectors refer to those constructs containing adenovirus sequences sufficient to (a) support packaging of an artificial expression construct and (b) to express a coding sequence that has been cloned therein in a sense or antisense orientation. A recombinant Adenovirus vector includes a genetically engineered form of an adenovirus. Knowledge of the genetic organization of adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range, and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection, and all the mRNAs issued from this promoter possess a 5-tripartite leader (TPL) sequence which makes them preferred mRNAs for translation.

Other than the requirement that an adenovirus vector be replication defective, or at least conditionally defective, the nature of the adenovirus vector is not believed to be crucial to the successful practice of particular embodiments disclosed herein. The adenovirus may be of any of the 42 different known serotypes or subgroups A-F. In particular embodiments, adenovirus type 5 of subgroup C is the preferred starting material in order to obtain a conditional replication-defective adenovirus vector for use in particular embodiments, since Adenovirus type 5 is a human adenovirus about which a great deal of biochemical and genetic information is known, and it has historically been used for most constructions employing adenovirus as a vector.

As indicated, the typical vector is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of a deleted E3 region in E3 replacement vectors or in the E4 region where a helper cell line or helper virus complements the E4 defect.

Adeno-Associated Virus (AAV) is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Various serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter.

The AAV DNA is 4.7 kilobases long. It contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three AAV viral promoters have been identified and named p5, p19, and p40, according to their map position. Transcription from p5 and p19 results in production of rep proteins, and transcription from p40 produces the capsid proteins.

AAVs stand out for use within the current disclosure because of their superb safety profile and because their capsids and genomes can be tailored to allow expression in targeted cell populations. scAAV refers to a self-complementary AAV. pAAV refers to a plasmid adeno-associated virus. rAAV refers to a recombinant adeno-associated virus.

Other viral vectors may also be employed. For example, vectors derived from viruses such as vaccinia virus, polioviruses and herpes viruses may be employed. They offer several attractive features for various mammalian cells.

Retroviruses are a common tool for gene delivery. “Retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and lentivirus.

“Lentivirus” refers to a group (or genus) of complex retroviruses. Illustrative lentiviruses include: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BlV); and simian immunodeficiency virus (SIV). In particular embodiments, HIV based vector backbones (i.e., HIV cis-acting sequence elements) can be used.

A safety enhancement for the use of some vectors can be provided by replacing the U3 region of the 5′ LTR with a heterologous promoter to drive transcription of the viral genome during production of viral particles. Examples of heterologous promoters which can be used for this purpose include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In particular embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include one or more chemical compounds or the physiological conditions such as temperature or pH, in which the host cells are cultured.

In particular embodiments, viral vectors include a TAR element. The term “TAR” refers to the “trans-activation response” genetic element located in the R region of lentiviral LTRs. This element interacts with the lentiviral trans-activator (tat) genetic element to enhance viral replication. However, this element is not required in embodiments wherein the U3 region of the 5′ LTR is replaced by a heterologous promoter.

The “R region” refers to the region within retroviral LTRs beginning at the start of the capping group (i.e., the start of transcription) and ending immediately prior to the start of the poly(A) tract. The R region is also defined as being flanked by the U3 and U5 regions. The R region plays a role during reverse transcription in permitting the transfer of nascent DNA from one end of the genome to the other.

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid. Examples include the woodchuck hepatitis virus posttranscriptional regulatory element (WPRE; Zufferey et al., 1999, J. Virol., 73:2886); the posttranscriptional regulatory element present in hepatitis B virus (HPRE) (Smith et al., Nucleic Acids Res. 26(21):4818-4827, 1998); and the like (Liu et al., 1995, Genes Dev., 9:1766). In particular embodiments, vectors include a posttranscriptional regulatory element such as a WPRE or HPRE. In particular embodiments, vectors lack or do not include a posttranscriptional regulatory element such as a WPRE or HPRE.

Elements directing the efficient termination and polyadenylation of a heterologous nucleic acid transcript can increase heterologous gene expression. Transcription termination signals are generally found downstream of the polyadenylation signal. In particular embodiments, vectors include a polyadenylation signal 3′ of a polynucleotide encoding a molecule (e.g., protein) to be expressed. The term “poly(A) site” or “poly(A) sequence” denotes a DNA sequence which directs both the termination and polyadenylation of the nascent RNA transcript by RNA polymerase II. Polyadenylation sequences can promote mRNA stability by addition of a poly(A) tail to the 3′ end of the coding sequence and thus, contribute to increased translational efficiency. Particular embodiments may utilize BGHpA or SV40 pA. In particular embodiments, a preferred embodiment of an expression construct includes a terminator element. These elements can serve to enhance transcript levels and to minimize read through from the construct into other plasmid sequences.

In particular embodiments, a viral vector further includes one or more insulator elements. Insulators elements may contribute to protecting viral vector-expressed sequences, e.g., effector elements or expressible elements, from integration site effects, which may be mediated by cis-acting elements present in genomic DNA and lead to deregulated expression of transferred sequences (i.e., position effect; see, e.g., Burgess-Beusse et al., PNAS., USA, 99:16433, 2002; and Zhan et al., Hum. Genet., 109:471, 2001). In particular embodiments, viral transfer vectors include one or more insulator elements at the 3′ LTR and upon integration of the provirus into the host genome, the provirus includes the one or more insulators at both the 5′ LTR and 3′ LTR, by virtue of duplicating the 3′ LTR. Suitable insulators for use in particular embodiments include the chicken p-globin insulator (see Chung et al., Cell 74:505, 1993; Chung et al., PNAS USA 94:575, 1997; and Bell et al., Cell 98:387, 1999), SP10 insulator (Abhyankar et al., JBC 282:36143, 2007), or other small CTCF recognition sequences that function as enhancer blocking insulators (Liu et al., Nature Biotechnology, 33:198, 2015).

Beyond the foregoing description, a wide range of suitable expression vector types will be known to a person of ordinary skill in the art. These can include commercially available expression vectors designed for general recombinant procedures, for example plasmids that contain one or more reporter genes and regulatory elements required for expression of the reporter gene in cells. Numerous vectors are commercially available, e.g., from Invitrogen, Stratagene, Clontech, etc., and are described in numerous associated guides. In particular embodiments, suitable expression vectors include any plasmid, cosmid or phage construct that is capable of supporting expression of encoded genes in mammalian cell, such as pUC or Bluescript plasmid series.

Particular embodiments of vectors disclosed herein include:

TABLE 5
Constructs and Construct Components

| Construct | |
|---|---|
| Name | Construct Components |
| CN2438 | rAAV-eHGT_608h-minBglobin-SYFP2-WPRE3-BGHpA |
| CN2439 | rAAV-eHGT_609h-minBglobin-SYFP2-WPRE3-BGHpA |
| CN2451 | rAAV-eHGT_621h-minBglobin-SYFP2-WPRE3-BGHpA |
| CN2463 | rAAV-eHGT_633h-minBglobin-SYFP2-WPRE3-BGHpA |
| CN2464 | rAAV-eHGT_634h-minBglobin-SYFP2-WPRE3-BGHpA |
| CN2465 | rAAV-eHGT_635h-minBglobin-SYFP2-WPRE3-BGHpA |
| CN2466 | rAAV-eHGT_636h-minBglobin-SYFP2-WPRE3-BGHpA |
| CN2013 | rAAV-3xSP10ins-eHGT_351h-minRho*-SYFP2-WPRE3-BGHpA |
| CN2025 | rAAV-3xSP10ins-eHGT_367h-minRho*-SYFP2-WPRE3-BGHpA |
| CN2229 | rAAV-eHGT_441h-minBglobin-SYFP2-WPRE3-BGHpA |
| CN2442 | rAAV-eHGT_612h-minBglobin-SYFP2-WPRE3-BGHpA |
| CN2443 | rAAV-eHGT_613h-minBglobin-SYFP2-WPRE3-BGHpA |
| CN2444 | rAAV-eHGT_614h-minBglobin-SYFP2-WPRE3-BGHpA |
| CN2447 | rAAV-eHGT_617h-minBglobin-SYFP2-WPRE3-BGHpA |
| CN2448 | rAAV-eHGT_618h-minBglobin-SYFP2-WPRE3-BGHpA |
| CN2449 | rAAV-eHGT_619h-minBglobin-SYFP2-WPRE3-BGHpA |
| CN2450 | rAAV-eHGT_620h-minBglobin-SYFP2-WPRE3-BGHpA |
| CN2467 | rAAV-eHGT_442h-minBglobin-SYFP2-WPRE3-BGHpA |
| CN2421 | rAAV-eHGT_444h-minBglobin-SYFP2-WPRE3-BGHpA |
| CN2231 | rAAV-eHGT_445h-minBglobin-SYFP2-WPRE3-BGHpA |
| CN2236 | rAAV-eHGT_450h-minBglobin-SYFP2-WPRE3-BGHpA |
| CN2237 | rAAV-eHGT_452h-minBglobin-SYFP2-WPRE3-BGHpA |
| CN2440 | rAAV-eHGT_610h-minBglobin-SYFP2-WPRE3-BGHpA |
| CN2441 | rAAV-eHGT_611h-minBglobin-SYFP2-WPRE3-BGHpA |
| CN2445 | rAAV-eHGT_615h-minBglobin-SYFP2-WPRE3-BGHpA |
| CN2446 | rAAV-eHGT_616h-minBglobin-SYFP2-WPRE3-BGHpA |
| CN2457 | rAAV-eHGT_627h-minBglobin-SYFP2-WPRE3-BGHpA |
| CN2458 | rAAV-eHGT_628h-minBglobin-SYFP2-WPRE3-BGHpA |
| CN2459 | rAAV-eHGT_629h-minBglobin-SYFP2-WPRE3-BGHpA |
| CN2232 | rAAV-eHGT_446h-minBglobin-SYFP2-WPRE3-BGHpA |
| CN2233 | rAAV-eHGT_447h-minBglobin-SYFP2-WPRE3-BGHpA |
| CN2452 | rAAV-eHGT_622h-minBglobin-SYFP2-WPRE3-BGHpA |
| CN2453 | rAAV-eHGT_623h-minBglobin-SYFP2-WPRE3-BGHpA |
| CN2454 | rAAV-eHGT_624h-minBglobin-SYFP2-WPRE3-BGHpA |
| CN2455 | rAAV-eHGT_625h-minBglobin-SYFP2-WPRE3-BGHpA |
| CN2460 | rAAV-eHGT_630h-minBglobin-SYFP2-WPRE3-BGHpA |
| CN2461 | rAAV-eHGT_631h-minBglobin-SYFP2-WPRE3-BGHpA |
| CN2628 | rAAV-eHGT_735m-minBglobin-SYFP2-WPRE3-BGHpA |
| CN2641 | rAAV-eHGT_736m-minBglobin-SYFP2-WPRE3-BGHpA |
| CN2642 | rAAV-eHGT_737m-minBglobin-SYFP2-WPRE3-BGHpA |
| CN2643 | rAAV-eHGT_738m-minBglobin-SYFP2-WPRE3-BGHpA |
| CN2629 | rAAV-eHGT_739m-minBglobin-SYFP2-WPRE3-BGHpA |
| CN2630 | rAAV-eHGT_740m-minBglobin-SYFP2-WPRE3-BGHpA |
| CN2745 | rAAV-eHGT_741m-minBglobin-SYFP2-WPRE3-BGHpA |
| CN2746 | rAAV-eHGT_742m-minBglobin-SYFP2-WPRE3-BGHpA |
| CN2631 | rAAV-eHGT_743m-minBglobin-SYFP2-WPRE3-BGHpA |
| CN2747 | rAAV-eHGT_744m-minBglobin-SYFP2-WPRE3-BGHpA |
| CN2632 | rAAV-eHGT_746m-minBglobin-SYFP2-WPRE3-BGHpA |
| CN2644 | rAAV-eHGT_747m-minBglobin-SYFP2-WPRE3-BGHpA |
| CN2748 | rAAV-eHGT_748m-minBglobin-SYFP2-WPRE3-BGHpA |
| CN2633 | rAAV-eHGT_749m-minBglobin-SYFP2-WPRE3-BGHpA |
| CN2634 | rAAV-eHGT_750m-minBglobin-SYFP2-WPRE3-BGHpA |
| CN2635 | rAAV-eHGT_751m-minBglobin-SYFP2-WPRE3-BGHpA |
| CN2609 | rAAV-eHGT_779m-minBglobin-SYFP2-WPRE3-BGHpA |
| CN2610 | rAAV-eHGT_780m-minBglobin-SYFP2-WPRE3-BGHpA |
| CN2749 | rAAV-eHGT_781m-minBglobin-SYFP2-WPRE3-BGHpA |
| CN2626 | rAAV-eHGT_782m-minBglobin-SYFP2-WPRE3-BGHpA |
| CN2611 | rAAV-eHGT_783m-minBglobin-SYFP2-WPRE3-BGHpA |
| CN2750 | rAAV-eHGT_784m-minBglobin-SYFP2-WPRE3-BGHpA |
| CN2614 | rAAV-eHGT_785m-minBglobin-SYFP2-WPRE3-BGHpA |
| CN2485 | rAAV-eHGT_452h-minBglobin-hAADC-Intron-WPRE3-BGHpA |
| CN2486 | rAAV-eHGT_452h-minBglobin-hAADC-Intron-3xHA-WPRE3-BGHpA |
| CN2739 | rAAV-3xSP10ins-eHGT_367h-minRho-hAADC-Intron-WPRE3-BGHpA |
| CN2740 | rAAV-3xSP10ins-eHGT_367h-minRho-hAADC-Intron-3xHA-WPRE3-BGHpA |
| CN2765 | pAAV-3xSP10ins-eHGT_367h-minBglobin-hAADC-Intron-WPRE3-BGHpA |
| CN2766 | pAAV-3xSP10ins-eHGT_367h-minBglobin-hAADC-Intron-3xHA-WPRE3- |
| | BGHPA |
| CN2514 | rAAV-3xSP10ins-core2_eHGT_367h-minRho*-SYFP2-WPRE3-BGHpA |
| CN2555 | rAAV-3xSP10ins-3xcore2_eHGT_367h-minRho*-SYFP2-WPRE3-BGHpA |
| CN2907 | rAAV-3xcore_eHGT_441h-minBglobin-SYFP2-WPRE3-BGHpA |
| CN2909 | rAAV-3xcore2_eHGT_445h-minBglobin-SYFP2-WPRE3-BGHpA |
| CN2921 | rAAV-3xcore2_eHGT_444h-minBglobin-SYFP2-WPRE3-BGHpA |
| CN2982 | rAAV-3xcore2_eHGT_452h-minBglobin-SYFP2-WPRE3-BGHpA |
| CN3044 | rAAV-3xcore2_eHGT_779m-minBglobin-SYFP2-WPRE3-BGHpA |
| CN3038 | rAAV-3xcore2_eHGT_743m-minBglobin-SYFP2-WPRE3-BGHpA |
| CN3344 | rAAV-3xCore-eHGT_621h-minBglobin-SYFP2-WPRE3-BGHpA |
| CN3281 | rAAV-3xCore2_eHGT_780m-minBglobin-SYFP2-WPRE3-BGHpA |
| CN3346 | rAAV-3xCore-eHGT_447h-minBglobin-SYFP2-WPRE3-BGHpA |
| CN3566 | rAAV-3xSP10ins_3xcore2_eHGT_351h-minBglobin-SYFP2-WPRE3-BGHpA |
| CN2912 | rAAV- 3xcore2_eHGT_450h-minBglobin-SYFP2-WPRE3-BGHpA |
| CN2913 | rAAV-3xcore3_eHGT_450h-minBglobin-SYFP2-WPRE3-BGHpA |
| CN2966 | rAAV-eHGT_743m-minBglobin-iCre-WPRE3-BGHpA |
| CN2203 | rAAV-3xSP10ins-eHGT_367h-minRho*-iCre-WPRE3-BGHpA |
| CN2700 | rAAV-eHGT_452h-minBglobin-mTFP1-WPRE3-BGHpA |

Subcomponent sequences within the larger vector sequences can be readily identified by one of ordinary skill in the art and based on the contents of the current disclosure (see FIG. 22C). Nucleotides between identifiable and enumerated subcomponents reflect restriction enzyme recognition sites used in assembly (cloning) of the constructs, and in some cases, additional nucleotides do not convey any identifiable function. These segments of complete vector sequences can be adjusted based on use of different cloning strategies and/or vectors. In general, short 6-nucleotide palindromic sequences reflect vector construction artifacts that are not important to vector function.

In particular embodiments vectors (e.g., AAV) with capsids that cross the blood-brain barrier (BBB) are selected. In particular embodiments, vectors are modified to include capsids that cross the BBB. Examples of AAV with viral capsids that cross the blood brain barrier include AAV9 (Gombash et al., Front Mol Neurosci. 2014; 7:81), AAVrh.10 (Yang, et al., Mol Ther. 2014; 22(7): 1299-1309), AAV1R6, AAV1R7 (Albright et al., Mol Ther. 2018; 26(2): 510), rAAVrh.8 (Yang, et al., supra), AAV-BR1 (Marchio et al., EMBO Mol Med. 2016; 8(6): 592), AAV-PHP.S (Chan et al., Nat Neurosci. 2017; 20(8): 1172), AAV-PHP.B (Deverman et al., Nat Biotechnol. 2016; 34(2): 204), AAV-PPS (Chen et al., Nat Med. 2009; 15: 1215), and PHP.eB. In particular embodiments, the PHP.eB capsid differs from AAV9 such that, using AAV9 as a reference, amino acids starting at residue 586: S-AQ-A (SEQ ID NO: 196) are changed to S-DGTLAVPFK-A (SEQ ID NO: 197). In particular embodiments, PHP.eb refers to the associated sequence provided in FIG. 22C.

AAV9 is a naturally occurring AAV serotype that, unlike many other naturally occurring serotypes, can cross the BBB following intravenous injection. It transduces large sections of the central nervous system (CNS), thus permitting minimally invasive treatments (Naso et al., BioDrugs. 2017; 31(4): 317), for example, as described in relation to clinical trials for the treatment of spinal muscular atrophy (SMA) syndrome by AveXis (AVXS-101, NCT03505099) and the treatment of CLN3 gene-Related Neuronal Ceroid-Lipofuscinosis (NCT03770572).

AAVrh.10, was originally isolated from rhesus macaques and shows low seropositivity in humans when compared with other common serotypes used for gene delivery applications (Selot et al., Front Pharmacol. 2017; 8: 441) and has been evaluated in clinical trials LYS-SAF302, LYSOGENE, and NCT03612869.

AAV1 R6 and AAV1 R7, two variants isolated from a library of chimeric AAV vectors (AAV1 capsid domains swapped into AAVrh.10 (also referred to as Rh10)), retain the ability to cross the BBB and transduce the CNS while showing significantly reduced hepatic and vascular endothelial transduction.

rAAVrh.8, also isolated from rhesus macaques, shows a global transduction of glial and neuronal cell types in regions of clinical importance following peripheral administration and also displays reduced peripheral tissue tropism compared to other vectors.

AAV-BR1 is an AAV2 variant displaying the NRGTEWD (SEQ ID NO: 198) epitope that was isolated during in vivo screening of a random AAV display peptide library. It shows high specificity accompanied by high transgene expression in the brain with minimal off-target affinity (including for the liver) (K6rbelin et al., EMBO Mol Med. 2016; 8(6): 609).

AAV-PHP.S (Addgene, Watertown, MA) is a variant of AAV9 generated with the CREATE method that encodes the 7-mer sequence QAVRTSL (SEQ ID NO: 199), transduces neurons in the enteric nervous system, and strongly transduces peripheral sensory afferents entering the spinal cord and brain stem.

AAV-PHP.B (Addgene, Watertown, MA) is a variant of AAV9 generated with the CREATE method that encodes the 7-mer sequence TLAVPFK (SEQ ID NO: 200). It transfers genes throughout the CNS with higher efficiency than AAV9 and transduces the majority of astrocytes and neurons across multiple CNS regions.

AAV-PPS, an AAV2 variant crated by insertion of the DSPAHPS (SEQ ID NO: 201) epitope into the capsid of AAV2, shows a dramatically improved brain tropism relative to AAV2.

Additional capsids include PHP.V1, AAV1, AAV5, AAV8, AAV11, Hull, AAV2-retro, AAV9-retro, CAP. B10, and CAP.B22. For additional information regarding capsids that cross the blood brain barrier, see Chan et al., Nat. Neurosci. 2017 Aug: 20(8): 1172-1179.

(ii) Compositions for Administration. Artificial expression constructs and vectors of the present disclosure (referred to herein as physiologically active components) can be formulated with a carrier that is suitable for administration to a cell, tissue slice, animal (e.g., mouse, non-human primate), or human. Physiologically active components within compositions described herein can be prepared in neutral forms, as freebases, or as pharmacologically acceptable salts.

Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Carriers of physiologically active components can include solvents, dispersion media, vehicles, coatings, diluents, isotonic and absorption delaying agents, buffers, solutions, suspensions, colloids, and the like. The use of such carriers for physiologically active components is well known in the art. Except insofar as any conventional media or agent is incompatible with the physiologically active components, it can be used with compositions as described herein.

The phrase “pharmaceutically-acceptable carriers” refer to carriers that do not produce an allergic or similar untoward reaction when administered to a human, and in particular embodiments, when administered intravenously (e.g. at the retro-orbital plexus).

In particular embodiments, compositions can be formulated for intravenous, intraparenchymal, intraocular, intravitreal, parenteral, subcutaneous, intracerebro-ventricular, intramuscular, intrathecal, intraspinal, intraperitoneal, oral or nasal inhalation, or by direct injection in or application to one or more cells, tissues, or organs.

Compositions may include liposomes, lipids, lipid complexes, microspheres, microparticles, nanospheres, and/or nanoparticles.

The formation and use of liposomes is generally known to those of skill in the art.

Liposomes have been developed with improved serum stability and circulation half-times (see, for instance, U.S. Pat. No. 5,741,516). Further, various methods of liposome and liposome like preparations as potential drug carriers have been described (see, for instance U.S. Pat. Nos. 5,567,434; 5,552,157; 5,565,213; 5,738,868; and 5,795,587).

The disclosure also provides for pharmaceutically acceptable nanocapsule formulations of the physiologically active components. Nanocapsules can generally entrap compounds in a stable and reproducible way (Quintanar-Guerrero et al., Drug Dev Ind Pharm 24(12):1113-1128, 1998; Quintanar-Guerrero et al., Pharm Res. 15(7):1056-1062, 1998; Quintanar-Guerrero et al., J. Microencapsul. 15(1):107-119, 1998; Douglas et al., Crit Rev Ther Drug Carrier Syst 3(3):233-261, 1987). To avoid side effects due to intracellular polymeric overloading, such ultrafine particles can be designed using polymers able to be degraded in vivo. Biodegradable polyalkyl-cyanoacrylate nanoparticles that meet these requirements are contemplated for use in the present disclosure. Such particles can be easily made, as described in Couvreur et al., J Pharm Sci 69(2):199-202, 1980; Couvreur et al., Crit Rev Ther Drug Carrier Syst. 5(1)1-20, 1988; zur Muhlen et al., Eur J Pharm Biopharm, 45(2):149-155, 1998; Zambaux et al., J Control Release 50(1-3):31-40, 1998; and U.S. Pat. No. 5,145,684.

Injectable compositions can include sterile aqueous solutions or dispersions and sterile powders for the extemporaneous preparation of sterile injectable solutions or dispersions (U.S. Pat. No. 5,466,468). For delivery via injection, the form is sterile and fluid to the extent that it can be delivered by syringe. In particular embodiments, it is stable under the conditions of manufacture and storage, and optionally contains one or more preservative compounds against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion, and/or by the use of surfactants. The prevention of the action of microorganisms can be brought about by various antibacterial and/or antifungal agents, for example, parabens, chlorobutanol, phenol, sorbic acid, thimerosal, and the like. In various embodiments, the preparation will include an isotonic agent(s), for example, sugar(s) or sodium chloride. Prolonged absorption of the injectable compositions can be accomplished by including in the compositions of agents that delay absorption, for example, aluminum monostearate and gelatin. Injectable compositions can be suitably buffered, if necessary, and the liquid diluent first rendered isotonic with sufficient saline or glucose.

Dispersions may also be prepared in glycerol, liquid polyethylene glycols, and mixtures thereof and in oils. As indicated, under ordinary conditions of storage and use, these preparations can contain a preservative to prevent the growth of microorganisms.

Sterile compositions can be prepared by incorporating the physiologically active component in an appropriate amount of a solvent with other optional ingredients (e.g., as enumerated above), followed by filtered sterilization. Generally, dispersions are prepared by incorporating the various sterilized physiologically active components into a sterile vehicle that contains the basic dispersion medium and the required other ingredients (e.g., from those enumerated above). In the case of sterile powders for the preparation of sterile injectable solutions, preferred methods of preparation can be vacuum-drying and freeze-drying techniques which yield a powder of the physiologically active components plus any additional desired ingredient from a previously sterile-filtered solution thereof.

Oral compositions may be in liquid form, for example, as solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The compositions may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets may be coated by methods well-known in the art.

Inhalable compositions can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions can also include microchip devices (U.S. Pat. No. 5,797,898), ophthalmic formulations (Bourlais et al., Prog Retin Eye Res, 17(1):33-58, 1998), transdermal matrices (U.S. Pat. Nos. 5,770,219 and 5,783,208) and feedback-controlled delivery (U.S. Pat. No. 5,697,899).

Supplementary active ingredients can also be incorporated into the compositions.

Typically, compositions can include at least 0.1% of the physiologically active components or more, although the percentage of the physiologically active components may, of course, be varied and may conveniently be between 1 or 2% and 70% or 80% or more or 0.5-99% of the weight or volume of the total composition. Naturally, the amount of physiologically active components in each physiologically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of compositions and dosages may be desirable.

In particular embodiments, for administration to humans, compositions should meet sterility, pyrogenicity, and the general safety and purity standards as required by United States Food and Drug Administration (FDA) or other applicable regulatory agencies in other countries.

(iii) Cell Lines Including Artificial Expression Constructs. The present disclosure includes cells including an artificial expression construct described herein. A cell that has been transformed with an artificial expression construct can be used for many purposes, including in neuroanatomical studies, assessments of functioning and/or non-functioning proteins, and drug screens that assess the regulatory properties of enhancers.

A variety of host cell lines can be used, but in particular embodiments, the cell is a mammalian cell. In particular embodiments, the artificial expression construct includes eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, or 3×core2_eHGT_743m and/or CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, or CN2700, and the cell line is a human, primate, or murine cell. Cell lines which can be utilized for transgenesis in the present disclosure also include primary cell lines derived from living tissue such as rat or mouse brains and organotypic cell cultures, including brain slices from animals such as rats or mice. The PC12 cell line (available from the American Type Culture Collection, ATCC, Manassas, VA) has been shown to express a number of neuronal marker proteins in response to Neuronal Growth Factor (NGF). The PC12 cell line is considered to be a neuronal cell line and is applicable for use with this disclosure. JAR cells (available from ATCC) are a platelet derived cell-line that express some neuronal genes, such as the serotonin transporter gene, and may be used with embodiments described herein.

WO 91/13150 describes a variety of cell lines, including neuronal cell lines, and methods of producing them. Similarly, WO 97/39117 describes a neuronal cell line and methods of producing such cell lines. The neuronal cell lines disclosed in these patent applications are applicable for use in the present disclosure.

In particular embodiments, “neuronal” describes something that is of, related to, or includes, neuronal cells. Neuronal cells are defined by the presence of an axon and dendrites. The term “neuronal-specific” refers to something that is found, or an activity that occurs, in neuronal cells or cells derived from neuronal cells, but is not found in or occur in, or is not found substantially in or occur substantially in, non-neuronal cells or cells not derived from neuronal cells, for example glial cells such as astrocytes or oligodendrocytes.

In particular embodiments, non-neuronal cell lines may be used, including mouse embryonic stem cells. Cultured mouse embryonic stem cells can be used to analyze expression of genetic constructs using transient transfection with plasmid constructs. Mouse embryonic stem cells are pluripotent and undifferentiated. These cells can be maintained in this undifferentiated state by Leukemia Inhibitory Factor (LIF). Withdrawal of LIF induces differentiation of the embryonic stem cells. In culture, the stem cells form a variety of differentiated cell types. Differentiation is caused by the expression of tissue specific transcription factors, allowing the function of an enhancer sequence to be evaluated. (See for example Fiskerstrand et al., FEBS Lett 458: 171-174, 1999).

Methods to differentiate stem cells into neuronal cells include replacing a stem cell culture media with a media including basic fibroblast growth factor (bFGF) heparin, an N2 supplement (e.g., transferrin, insulin, progesterone, putrescine, and selenite), laminin and polyornithine. A process to produce myelinating oligodendrocytes from stem cells is described in Hu, et al., 2009, Nat. Protoc. 4:1614-22. Bibel, et al., 2007, Nat. Protoc. 2:1034-43 describes a protocol to produce glutamatergic neurons from stem cells while Chatzi, et al., 2009, Exp. Neurol. 217:407-16 describes a procedure to produce GABAergic neurons. This procedure includes exposing stem cells to all-trans-RA for three days. After subsequent culture in serum-free neuronal induction medium including Neurobasal medium supplemented with B27, bFGF and EGF, 95% GABA neurons develop

U.S. Publication No. 2012/0329714 describes use of prolactin to increase neural stem cell numbers while U.S. Publication No. 2012/0308530 describes a culture surface with amino groups that promotes neuronal differentiation into neurons, astrocytes and oligodendrocytes. Thus, the fate of neural stem cells can be controlled by a variety of extracellular factors. Commonly used factors include brain derived growth factor (BDNF; Shetty and Turner, 1998, J. Neurobiol. 35:395-425); fibroblast growth factor (bFGF; U.S. Pat. No. 5,766,948; FGF-1, FGF-2); Neurotrophin-3 (NT-3) and Neurotrophin-4 (NT-4); Caldwell, et al., 2001, Nat. Biotechnol. 1; 19:475-9); ciliary neurotrophic factor (CNTF); BMP-2 (U.S. Pat. Nos. 5,948,428 and 6,001,654); isobutyl 3-methylxanthine; leukemia inhibitory growth factor (LIF; U.S. Pat. No. 6,103,530); somatostatin; amphiregulin; neurotrophins (e.g., cyclic adenosine monophosphate; epidermal growth factor (EGF); dexamethasone (glucocorticoid hormone); forskolin; GDNF family receptor ligands; potassium; retinoic acid (U.S. Pat. No. 6,395,546); tetanus toxin; and transforming growth factor-α and TGF-β (U.S. Pat. Nos. 5,851,832 and 5,753,506).

In particular embodiments, yeast one-hybrid systems may also be used to identify compounds that inhibit specific protein/DNA interactions, such as transcription factors for eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, or 3×core2_eHGT_743m.

Transgenic animals are described below. Cell lines may also be derived from such transgenic animals. For example, primary tissue culture from transgenic mice (e.g., also as described below) can provide cell lines with the artificial expression construct already integrated into the genome. (for an example see MacKenzie & Quinn, Proc Natl Acad Sci USA 96: 15251-15255, 1999).

(iv) Transgenic Animals. Another aspect of the disclosure includes transgenic animals, the genome of which contains an artificial expression construct including eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, and 3×core2_eHGT_743m operatively linked to a heterologous coding sequence. In particular embodiments, the genome of a transgenic animal includes CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, and CN2700. In particular embodiments, when a non-integrating vector is utilized, a transgenic animal includes an artificial expression construct including eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, or 3×core2_eHGT_743m and/or CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, or CN2700 within one or more of its cells.

Detailed methods for producing transgenic animals are described in U.S. Pat. No. 4,736,866. Transgenic animals may be of any nonhuman species, but preferably include nonhuman primates (NHPs), sheep, horses, cattle, pigs, goats, dogs, cats, rabbits, chickens, and rodents such as guinea pigs, hamsters, gerbils, rats, mice, and ferrets.

In particular embodiments, construction of a transgenic animal results in an organism that has an engineered construct present in all cells in the same genomic integration site. Thus, cell lines derived from such transgenic animals will be consistent in as much as the engineered construct will be in the same genomic integration site in all cells and hence will suffer the same position effect variegation. In contrast, introducing genes into cell lines or primary cell cultures can give rise to heterologous expression of the construct. A disadvantage of this approach is that the expression of the introduced DNA may be affected by the specific genetic background of the host animal.

Transgenic animals can be used to model movements disorders. For example, an animal model of Parkinson's disease includes daily delivery of conduritol-b-epoxide (CBE), an inhibitor of GCase to mice. A genetic model of Parkinson's disease is one in which mice carry a homozygous GBA1 mutation and are partially deficient in saposins (4L/PS-NA). An example of a non-human primate model of Parkinson's disease is one in which adult Macaca fascicularis are administered the neurotoxin 1-methyl-4-phenyl-1,2,3,6-tetrahydropyridine (MPTP) until they reach a severe and stable bilateral Parkinsonian syndrome, including akinesia, flexed posture, balance impairment and tremor.

In particular embodiments, transgenic animals can be used in animal models of Huntington's disease. In particular embodiments, the animal model is a LV-171-82Q Huntington's disease (HD) rat model. This model is based on the striatal overexpression of the first 171 amino acids of the HTT mutant fragment with 82 CAG repeats linked to a fragment of exon 67 containing the SNP C/T. In particular embodiments, the animal model is a Hu128/21 HD mouse or a YAC128 HD mouse.

As indicated above in relation to cell lines, the artificial expression constructs of this disclosure can be used to genetically modify mouse embryonic stem cells using techniques known in the art. Typically, the artificial expression construct is introduced into cultured murine embryonic stem cells. Transformed ES cells are then injected into a blastocyst from a host mother and the host embryo re-implanted into the mother. This results in a chimeric mouse whose tissues are composed of cells derived from both the embryonic stem cells present in the cultured cell line and the embryonic stem cells present in the host embryo. Usually the mice from which the cultured ES cells used for transgenesis are derived are chosen to have a different coat color from the host mouse into whose embryos the transformed cells are to be injected. Chimeric mice will then have a variegated coat color. As long as the germ-line tissue is derived, at least in part, from the genetically modified cells, then the chimeric mice crossed with an appropriate strain can produce offspring that will carry the transgene.

In addition to the methods of delivery described above, the following techniques are also contemplated as alternative methods of delivering artificial expression constructs to target cells or targeted tissues and organs of an animal, and in particular, to cells, organs, or tissues of a vertebrate mammal: sonophoresis (e.g., ultrasound, as described in U.S. Pat. No. 5,656,016); intraosseous injection (U.S. Pat. No. 5,779,708); microchip devices (U.S. Pat. No. 5,797,898); ophthalmic formulations (Bourlais et al., Prog Retin Eye Res, 17(1):33-58, 1998); transdermal matrices (U.S. Pat. Nos. 5,770,219 and 5,783,208); feedback-controlled delivery (U.S. Pat. No. 5,697,899), and any other delivery method available and/or described elsewhere in the disclosure.

(v) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions for administration disclosed herein. Within the current disclosure, a subject can also include an isolated cell, a network of cells, or a tissue slice.

Treating subjects includes delivering therapeutically effective amounts. Therapeutically effective amounts include those that provide effective amounts, prophylactic treatments and/or therapeutic treatments.

In particular embodiments, the disclosure includes the use of the artificial expression constructs described herein to modulate expression of a heterologous gene which is either partially or wholly encoded in a location downstream to that enhancer in an engineered sequence. Thus, there are provided herein methods of use of the disclosed artificial expression constructs in the research, study, and potential development of medicaments for preventing, treating or ameliorating the symptoms of a disease, dysfunction, or disorder.

Particular embodiments include methods of administering to a subject an artificial expression construct including the enhancer eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, and 3×core2_eHGT_743m operatively linked to a heterologous coding sequence to drive expression of a gene in a targeted cell type.

In particular embodiments, the targeted cell type includes cells in the striatum including striatal interneuron-cholinergic, striatal medium spiny neuron-direct pathway, striatal medium spiny neuron-indirect pathway, striatal medium spiny neuron-pan, or Drd3+ medium spiny neurons.

In addition to targeting the striatum, some enhancers target additional brain regions and cell types.

In particular embodiments, 3×core2_eHGT_367h additionally targets parafascicular nucleus in the thalamus; L2/3 IT neurons and L6 neurons in the cortex; and superior colliculus, CA1, and hilus neurons in the hippocampus.

In particular embodiments, core2_eHGT_367h additionally targets superior colliculus, CA1, and hilus neurons in the hippocampus; with scattered sparse expression in the cortex.

In particular embodiments, eHGT_743m additionally targets lateral septal nucleus putative cholinergic interneurons.

In particular embodiments, eHGT_779m additionally targets cells in the caudodorsal part of lateral septum and superior colliculus.

In particular embodiments, eHGT_780m additionally targets central amygdala nucleus, accessory olfactory nucleus (AON), upper band of L2/3 pyramidal neurons in Piriform area, piriform-amygdalar area, entorhinal, perirhinal, frontal areas especially motor cortex areas, and cells around the glomeruli in the olfactory bulb (OB).

In particular embodiments, eHGT_785m additionally targets central amygdala (CEA); cells around the glomeruli in OB; and L3 in caudal entorhinal cortex.

In particular embodiments, eHGT_452h additionally targets TTd (Taenia tecta, dorsal) putative excitatory neurons.

In particular embodiments, eHGT_444h additionally targets cortex, amygdala, and glomeruli in olfactory bulb.

In particular embodiments, eHGT_621h additionally targets cells around the glomeruli in OB; paraventricular hypothalamic nucleus; and lateral, medial and main intercalated (ITC) nucleus of the amygdala.

In particular embodiments, eHGT_619h additionally targets CA2 in the hippocampus.

In particular embodiments, eHGT_617h additionally targets CA3 and hilus neurons in the hippocampus; cells around the glomeruli in OB; molecular layer of piriform area; paraventricular nucleus of the thalamus; dorsal tip of interpeduncular nucleus; and dorsal tegmental nucleus.

In particular embodiments, eHGT_612h additionally targets posterior basolateral amygdala (BLA) neurons, CA2, CA3, and caudal CA1/Subiculum pyramidal neurons; superior colliculus cells; paraventricular nucleus of the thalamus; AON cells; and scattered cells in OB.

In particular embodiments, eHGT_610h additionally targets cells in cortical amygdalar area (COAp), piriform area L2, CEA, BLA, L5 neurons in retrosplenial (RSP) cortex, AON, and OB.

In particular embodiments, eHGT_742m additionally targets L2/3 neocortex.

Particular embodiments include methods of administering to a subject an artificial expression construct including CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, and CN2700 to drive expression of a gene in a targeted cell type.

Particular embodiments include methods of administering to a subject an artificial expression construct including CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2514, CN2555, CN2907, CN2909, CN2921, CN2982, CN3044, CN3038, CN3344, CN3281, CN3346, CN3566, CN2912, CN2913, CN2966, CN2203, and/or CN2700 wherein the gene encoding sequence is replaced with or supplemented with a sequence encoding a therapeutic gene product to drive expression of a gene in a targeted cell type.

A therapeutic gene product refers to a biochemical material resulting from the expression of a gene that produces an intended physiological effect meant to alleviate a condition. In particular embodiments, a therapeutic gene product includes AADC (e.g. hAADC), GCase, survival motor neuron 1 (SMN1), nerve growth factor (NFG), glutamic acid decarboxylase (GAD), glial derived neurotrophic factor (GDNF; e.g. neurturin (NTN)), tyrosine hydroxylase (TH), guanosine triphosphate cyclohydrolase (GCH), brain-derived neurotrophic factor (BDNF), or RNA suppressing or inhibiting the expression of a pathogenic hungtingtin (HTT) gene.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of a movement disorder's development, progression, and/or resolution.

A “prophylactic treatment” includes a treatment administered to a subject who does not display signs or symptoms of a movement disorder or displays only early signs or symptoms of a movement disorder such that treatment is administered for the purpose of diminishing or decreasing the risk of developing the movement disorder further. Thus, a prophylactic treatment functions as a preventative treatment against a movement disorder. In particular embodiments, prophylactic treatments reduce, delay, or prevent the worsening of a movement disorder.

A “therapeutic treatment” includes a treatment administered to a subject who displays symptoms or signs of a movement disorder and is administered to the subject for the purpose of diminishing or eliminating those signs or symptoms of the movement disorder. The therapeutic treatment can reduce, control, or eliminate the presence or activity of the movement disorder and/or reduce control or eliminate side effects of the movement disorder.

Function as an effective amount, prophylactic treatment or therapeutic treatment are not mutually exclusive, and in particular embodiments, administered dosages may accomplish more than one treatment type.

Exemplary movements disorders that can be treated include Parkinson's disease, Huntington's disease, ataxia, corticobasal ganglionic degeneration (CBGD), dyskinesia, dystonia, tremors, hereditary spastic paraplegia, multiple system atrophy, myoclonus, progressive supranuclear palsy, restless legs syndrome, Rett syndrome, spasticity, Sydenham's chorea, other choreas, athetosis, ballism, stereotypy, tardive dyskinesia/dystonia, tics, Tourette's syndrome, olivopontocerebellar atrophy (OPCA), diffuse Lewy body disease, hemibalismus, hemi-facial spasm, Wilson's disease, stiff man syndrome, akinetic mutism, psychomotor retardation, painful legs, moving toes syndrome, a gait disorder, a drug-induced movement disorder, or other movement disorders described herein.

In particular embodiments, methods to determine the efficacy of the treatments using constructs disclosed herein will be measured before treatment, during the first year after treatment, and at other times. In particular embodiments, efficacy of the treatments using constructs disclosed herein will be determined to be effective if the evaluated measurements can be maintained at a normal, non-movement disorder level, reduced to a non-movement disorder level, or reduced such that it is still elevated compared to a non-movement disorder individual, but is still less than the level which would be expected in an individual without treatment.

Therapeutically effective amounts disclosed herein can improve motor control and/or reduce tremors.

Therapeutically effective amounts can be assessed using developmental tests for cognitive and motor function, MRI and CT assessments, FDOPA Positron Emission Tomography (PET) putamen-specific radioactivity uptake values, CSF neurotransmitter metabolite values, and neurological evaluation.

In particular embodiments, additional methods for determining the efficacy of Parkinson's disease treatments include measuring metabolic activity of the subthalamic nucleus (STN), measuring the firing rate of internal globus pallidus (GPi) neurons and the proportion of spikes per burst and the number of burst events in the neuronal firing pattern.

For Huntington's disease, therapeutically effective amounts can additionally be assessed using standard evaluations for this disease including the Unified Huntington's Disease Ratings Scale (UHDRS) or the Prognostic Index for Huntington's Disease. The Prognostic Index for Huntington's Disease predicts the probability of a motor diagnosis by calculating the total motor score (TMS) from the Unified Huntington's Disease Rating Scale (UHDRS), the Symbol Digit Modality Test (SDMT), base-line age, and cytosine-adenine-guanine (CAG) expansion. Potential biomarkers in brain imaging for premanifest and early progression of Huntington's disease include striatal volume, subcortical white-matter volume, cortical thickness, whole brain and ventricular volumes, functional imaging (e.g., functional MRI), PET (e.g., with fluorodeoxy glucose), and magnetic resonance spectroscopy (e.g., lactate). Potential biomarkers for quantitative clinical tools for premanifest and early progression of Huntington's disease include quantitative motor assessments, motor physiological assessments (e.g., transcranial magnetic stimulation), and quantitative eye movement measurements.

Numerous movement disorders affect infants and children and a number of motor and developmental tests can be utilized to assess therapeutically effective amounts within this context. Examples include the Peabody Developmental Motor Scale (PDMS-II), Alberta Infant Motor Scale (AIMS), Bayley Scales of Infant and Toddler Development®-Third Edition (Bayley-III), or the Comprehensive Developmental Inventory for Infants and Toddlers (CDIIT).

PDMS-II is a skill-based measure of gross and fine motor development for infants and children from birth through 5 years of age. This tool separates motor development into gross and fine motor skills. Through a combination of the composite scores for the gross and fine motor skills, the examiner has a reliable estimate of the child's motor skills. It consists of 4 gross motor and 2 fine motor subtests, as follows: Reflexes (gross motor); Stationary (gross motor); Locomotion (gross motor); Object Manipulation (gross motor); Grasping (fine motor); and Visual-Motor Integration (fine motor).

Scoring the PDMS-II relies on raw scores, percentiles, standard scores, and age equivalents for the subtests, and quotients for the composites. Raw scores are total points accumulated by a child on a subtest. Developmental ages are often used to convey information to parents of young children. Age equivalents for PDMS-II are called “motor ages” which convey to parents that their child is “passing” on items that a child of a certain chronological age would typically pass. Age equivalents for PDMS-II subtests are generated from Table C.1 in the PDMS-II manual or by PDMS-II software scoring and report systems.

AIMS is a 58-item observational measure of infant motor performance for use from birth through the age of independent walking (18 months). It assesses the sequential development of motor milestones in terms of progressive development and integration of antigravity muscle control. The test assesses infant movement in 4 positions: prone, supine, sitting, and standing.

The AIMS total score is calculated by summing the scores for the 58 items with a range of scores between 0 and 58. Higher scores indicate more mature motor development. The infant's score can then be converted to a percentile and compared with age-equivalent peers from the normative sample.

Bayley-III offers a standardized assessment of cognitive and motor development for children between 1 and 42 months of age. The assessment measures cognitive, communication, physical, social/emotional, and adaptive areas of development to identify children with developmental delays. The test consists of 5 scales of development: Cognitive Scale, Language Scale, Motor Scale, Social Emotional Scale, and Adaptive Behavior Scale. It is possible to present results for developmental age corresponding to each subscale vs chronological age.

The diagnostic test of the CDIIT is one of the child developmental tests covering 5 developmental subtests used for children aged 3 to 72 months.

The amount of expression constructs and time of administration of such compositions will be within the purview of the skilled artisan having benefit of the present teachings. It is likely, however, that the administration of effective amounts of the disclosed compositions may be achieved by a single administration, such as for example, a single injection of sufficient numbers of infectious particles to provide an effect in the subject. Alternatively, in some circumstances, it may be desirable to provide multiple, or successive administrations of the artificial expression construct compositions or other genetic constructs, either over a relatively short, or a relatively prolonged period of time, as may be determined by the individual overseeing the administration of such compositions. For example, the number of infectious particles administered to a mammal may be 10⁵, 10⁶, 10⁷, 10⁸, 10⁹, 10¹⁰, 10¹¹, 10¹², 10¹³, 10¹⁴, 10¹⁵, 10¹⁶, or even higher, viral genomes/ml given either as a single dose or divided into two or more administrations as may be required to achieve an intended effect. In fact, in certain embodiments, it may be desirable to administer two or more different expression constructs in combination to achieve a desired effect. In particular embodiments, a patient receiving intravenous, intraparenchymal, intraspinal, retro-orbital, or intrathecal administration can be infused with from 10⁵ to 10²² copies of the artificial expression construct. In particular embodiments, dosages for any one subject depends upon many factors, including the subject's size, surface area, age, the particular compound to be administered, sex, time and route of administration, general health, and other drugs being administered concurrently.

In certain circumstances it will be desirable to deliver the artificial expression construct in suitably formulated compositions disclosed herein either by pipette, retro-orbital injection, subcutaneously, intraocularly, intracisternally, intravitreally, parenterally, subcutaneously, intravenously, intraparenchymally, intracerebro-ventricularly, intramuscularly, intrathecally, intraspinally, intraperitoneally, by oral or nasal inhalation, or by direct application or injection to one or more cells, tissues, or organs. The methods of administration may also include those modalities as described in U.S. Pat. Nos. 5,543,158; 5,641,515 and 5,399,363.

(vi) Kits and Commercial Packages. Kits and commercial packages contain an artificial expression construct described herein. The artificial expression construct can be isolated. In particular embodiments, the components of an expression product can be isolated from each other. In particular embodiments, the expression product can be within a vector, within a viral vector, within a cell, within a tissue slice or sample, and/or within a transgenic animal. Such kits may further include one or more reagents, restriction enzymes, peptides, therapeutics, pharmaceutical compounds, or means for delivery of the compositions such as syringes, injectables, and the like.

Embodiments of a kit or commercial package will also contain instructions regarding use of the included components, for example, in basic research, electrophysiological research, neuroanatomical research, and/or the research and/or treatment of a disorder, disease or condition.

The Exemplary Embodiments and Experimental Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

(vii) Exemplary Embodiments.
1. A core of the eHGT_351h, eHGT_367h, eHGT_441h, eHGT_444h, eHGT_445h, eHGT_447h, eHGT_450h, eHGT_452h, eHGT_621h, eHGT_743m, eHGT_779m, or eHGT_780m enhancer.
2. The core of embodiment 1, wherein the core has the sequence as set forth in SEQ ID NO: 202; SEQ ID NO: 204; SEQ ID NO: 206; SEQ ID NO: 208; SEQ ID NO: 210; SEQ ID NO: 212; SEQ ID NO: 214; SEQ ID NO: 216; SEQ ID NO: 218; SEQ ID NO: 220; SEQ ID NO: 222; SEQ ID NO: 224; or SEQ ID NO: 226.
3. The core of embodiments 1 or 2, wherein the core is concatenated into a concatemer including 2, 3, 4, 5, 6, 7, 8, 9, or 10 copies of the enhancer core.
4. The core of embodiment 3, wherein the concatenated core includes 2, 3, 4, 5, 6, 7, 8, 9, or 10 copies of SEQ ID NO: 202; SEQ ID NO: 204; SEQ ID NO: 206; SEQ ID NO: 208; SEQ ID NO: 210; SEQ ID NO: 212; SEQ ID NO: 214; SEQ ID NO: 216; SEQ ID NO: 218; SEQ ID NO: 220; SEQ ID NO: 222; SEQ ID NO: 224; and/or SEQ ID NO: 226.
5. The core of any of embodiments 3 or 4, wherein the concatenated core includes 3 copies of SEQ ID NO: 206.
6. The core of any of embodiments 3 or 4, wherein the concatenated core includes 3 copies of SEQ ID NO: 202.
7. The core of any of embodiments 3 or 4, wherein the concatenated core includes 3 copies of SEQ ID NO: 204.
8. The core of any of embodiments 3 or 4, wherein the concatenated core includes 3 copies of SEQ ID NO: 208.
9. The core of any of embodiments 3 or 4, wherein the concatenated core includes 3 copies of SEQ ID NO: 210.
10. The core of any of embodiments 3 or 4, wherein the concatenated core includes 3 copies of SEQ ID NO: 212.
11. The core of any of embodiments 3 or 4, wherein the concatenated core includes 3 copies of SEQ ID NO: 214.
12. The core of any of embodiments 3 or 4, wherein the concatenated core includes 3 copies of SEQ ID NO: 216.
13. The core of any of embodiments 3 or 4, wherein the concatenated core includes 3 copies of SEQ ID NO: 218.
14. The core of any of embodiments 3 or 4, wherein the concatenated core includes 3 copies of SEQ ID NO: 220.
15. The core of any of embodiments 3 or 4, wherein the concatenated core includes 3 copies of SEQ ID NO: 222.
16. The core of any of embodiments 3 or 4, wherein the concatenated core includes 3 copies of SEQ ID NO: 224.
17. The core of any of embodiments 3 or 4, wherein the concatenated core includes 3 copies of SEQ ID NO: 226.
18. The core of embodiment 5, wherein the concatenated core includes SEQ ID NO: 207.
19. The core of embodiment 6, wherein the concatenated core includes SEQ ID NO: 203.
20. The core of embodiment 7, wherein the concatenated core includes SEQ ID NO: 205.
21. The core of embodiment 8, wherein the concatenated core includes SEQ ID NO: 209.
22. The core of embodiment 9, wherein the concatenated core includes SEQ ID NO: 211.
23. The core of embodiment 10, wherein the concatenated core includes SEQ ID NO: 213.
24. The core of embodiment 11, wherein the concatenated core includes SEQ ID NO: 215.
25. The core of embodiment 12, wherein the concatenated core includes SEQ ID NO: 217. 26. The core of embodiment 13, wherein the concatenated core includes SEQ ID NO: 219.
27. The core of embodiment 14, wherein the concatenated core includes SEQ ID NO: 221.
28. The core of embodiment 15, wherein the concatenated core includes SEQ ID NO: 223.
29. The core of embodiment 16, wherein the concatenated core includes SEQ ID NO: 225.
30. The core of embodiment 17, wherein the concatenated core includes SEQ ID NO: 227.
31. An artificial expression construct including (i) an enhancer selected from eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, core2_eHGT_367h, core_eHGT_441h, Core-eHGT_621h, Core-eHGT_447h, core2_eHGT_351h, core2_eHGT_445h, core2_eHGT_444h, core2_eHGT_452h, core2_eHGT_450h, core3_eHGT_450h, core2_eHGT_779m, Core2_eHGT_780m, and core2_eHGT_743m; (ii) a promoter; and (iii) a heterologous encoding sequence.
32. The artificial expression construct of claim 4, wherein the enhancer is concatenated including 3×core2_eHGT_743m, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, and 3×Core2_eHGT_780m.
33. The artificial expression construct of embodiments 31 or 32, wherein the heterologous encoding sequence encodes an effector element or an expressible element.
34. The artificial expression construct of embodiment 33, wherein the effector element includes a reporter protein or a functional molecule.
35. The artificial expression construct of embodiment 34, wherein the reporter protein includes a fluorescent protein.
36. The artificial expression construct of embodiment 34 or 35, wherein the functional molecule includes a functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, microRNA, homologous recombination donor cassette, or a designer receptor exclusively activated by designer drug (DREADD).
37. The artificial expression construct of embodiment 36, wherein the functional molecule includes aromatic L-amino acid decarboxylase (AADC), tyrosine hydroxylase (TH), GTP cyclohydrolase I (CH1), tetrahydrobiopterin (BH4) and/or glucocerebrosidase (GCase).
38. The artificial expression construct of embodiment 36, wherein the RNA suppresses or inhibits the expression of a pathogenic huntingtin (HTT) gene.
39. The artificial expression construct of embodiment 38, wherein the RNA sequence includes SEQ ID NOs: 163-195.
40. The artificial expression construct of embodiment 33, wherein the expressible element includes a non-functional molecule.
41. The artificial expression construct of embodiment 40, wherein the non-functional molecule includes a non-functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, microRNA, homologous recombination donor cassette, or a DREADD.
42. The artificial expression construct of any of embodiments 31-41, wherein the artificial expression construct is associated with a capsid that crosses the blood brain barrier.
43. The artificial expression construct of embodiment 42, wherein the capsid includes PHP.eB, AAV-BR1, AAV-PHP.S, AAV-PHP.B, AAV-PPS, PHP.V1, AAV1, AAV2, AAV5, AAV8, AAV9, AAV11, Rh10, Hu11, AAV2-retro, AAV9-retro, CAP.B10, or CAP.B22.
44. The artificial expression construct of any of embodiments 31-43, wherein the artificial expression construct includes or encodes a skipping element.
45. The artificial expression construct of embodiment 44, wherein the skipping element includes a 2A peptide and/or an internal ribosome entry site (IRES).
46. The artificial expression construct of embodiment 45, wherein the 2A peptide is selected from T2A, P2A, E2A, or F2A.
47. The artificial expression construct of any of embodiments 31-46, wherein the artificial expression construct includes or encodes a set of features selected from eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, 3×core2_eHGT_743m, AAV, scAAV, rAAV, pAAV, minBglobin, CMV, minCMV, minRho, minRho*, AADC, TH, CH1, BH4 GCase, Intron, 3×HA, an RNA that suppresses or inhibits the expression of a pathogenic HTT gene, a gene whose expression treats a movement disorder, fluorescent protein (e.g. EGFP, SYFP2, GFP, mTFP1), Cre, iCre, dgCre, FlpO, tTA2, SP10ins, WPRE3, hGHpA, and/or BGHpA.
48. The artificial expression construct of any of embodiments 31-47, wherein the artificial expression construct includes or encodes a set of features selected from:
   3×core2_eHGT_743m-minBglobin-[gene encoding functional molecule]-WPRE3-BGHpA;
   3×SP10ins-core2_eHGT_367h-minRho*-[gene encoding functional molecule]-WPRE3-BGHpA;
   eHGT_608h-minBglobin-[gene encoding functional molecule]-WPRE3-BGHpA;
   eHGT_609h-minBglobin-[gene encoding functional molecule]-WPRE3-BGHpA;
   eHGT_621h-minBglobin-[gene encoding functional molecule]-WPRE3-BGHpA;
   eHGT_633h-minBglobin-[gene encoding functional molecule]-WPRE3-BGHpA;
   eHGT_634h-minBglobin-[gene encoding functional molecule]-WPRE3-BGHpA;
   eHGT_635h-minBglobin-[gene encoding functional molecule]-WPRE3-BGHpA;
   eHGT_636h-minBglobin-[gene encoding functional molecule]-WPRE3-BGHpA;
   3×SP10ins-eHGT_351h-minRho*-[gene encoding functional molecule]-WPRE3-BGHpA;
   3×SP10ins-eHGT_367h-minRho*-[gene encoding functional molecule]-WPRE3-BGHpA;
   eHGT_441h-minBglobin-[gene encoding functional molecule]-WPRE3-BGHpA;
   eHGT_612h-minBglobin-[gene encoding functional molecule]-WPRE3-BGHpA;
   eHGT_613h-minBglobin-[gene encoding functional molecule]-WPRE3-BGHpA;
   eHGT_614h-minBglobin-[gene encoding functional molecule]-WPRE3-BGHpA;
   eHGT_617h-minBglobin-[gene encoding functional molecule]-WPRE3-BGHpA;
   eHGT_618h-minBglobin-[gene encoding functional molecule]-WPRE3-BGHpA;
   eHGT_619h-minBglobin-[gene encoding functional molecule]-WPRE3-BGHpA;
   eHGT_620h-minBglobin-[gene encoding functional molecule]-WPRE3-BGHpA;
   eHGT_442h-minBglobin-[gene encoding functional molecule]-WPRE3-BGHpA;
   eHGT_444h-minBglobin-[gene encoding functional molecule]-WPRE3-BGHpA;
   eHGT_445h-minBglobin-[gene encoding functional molecule]-WPRE3-BGHpA;
   eHGT_450h-minBglobin-[gene encoding functional molecule]-WPRE3-BGHpA;
   eHGT_452h-minBglobin-[gene encoding functional molecule]-WPRE3-BGHpA;
   eHGT_610h-minBglobin-[gene encoding functional molecule]-WPRE3-BGHpA;
   eHGT_611h-minBglobin-[gene encoding functional molecule]-WPRE3-BGHpA;
   eHGT_615h-minBglobin-[gene encoding functional molecule]-WPRE3-BGHpA;
   eHGT_616h-minBglobin-[gene encoding functional molecule]-WPRE3-BGHpA;
   eHGT_627h-minBglobin-[gene encoding functional molecule]-WPRE3-BGHpA;
   eHGT_628h-minBglobin-[gene encoding functional molecule]-WPRE3-BGHpA;
   eHGT_629h-minBglobin-[gene encoding functional molecule]-WPRE3-BGHpA;
   eHGT_446h-minBglobin-[gene encoding functional molecule]-WPRE3-BGHpA;
   eHGT_447h-minBglobin-[gene encoding functional molecule]-WPRE3-BGHpA;
   eHGT_622h-minBglobin-[gene encoding functional molecule]-WPRE3-BGHpA;
   eHGT_623h-minBglobin-[gene encoding functional molecule]-WPRE3-BGHpA;
   eHGT_624h-minBglobin-[gene encoding functional molecule]-WPRE3-BGHpA;
   eHGT_625h-minBglobin-[gene encoding functional molecule]-WPRE3-BGHpA;
   eHGT_630h-minBglobin-[gene encoding functional molecule]-WPRE3-BGHpA;
   eHGT_631h-minBglobin-[gene encoding functional molecule]-WPRE3-BGHpA;
   eHGT_735m-minBglobin-[gene encoding functional molecule]-WPRE3-BGHpA;
   eHGT_736m-minBglobin-[gene encoding functional molecule]-WPRE3-BGHpA;
   eHGT_737m-minBglobin-[gene encoding functional molecule]-WPRE3-BGHpA;
   eHGT_738m-minBglobin-[gene encoding functional molecule]-WPRE3-BGHpA;
   eHGT_739m-minBglobin-[gene encoding functional molecule]-WPRE3-BGHpA;
   eHGT_740m-minBglobin-[gene encoding functional molecule]-WPRE3-BGHpA;
   eHGT_741m-minBglobin-[gene encoding functional molecule]-WPRE3-BGHpA;
   eHGT_742m-minBglobin-[gene encoding functional molecule]-WPRE3-BGHpA;
   eHGT_743m-minBglobin-[gene encoding functional molecule]-WPRE3-BGHpA;
   eHGT_744m-minBglobin-[gene encoding functional molecule]-WPRE3-BGHpA;
   eHGT_746m-minBglobin-[gene encoding functional molecule]-WPRE3-BGHpA;
   eHGT_747m-minBglobin-[gene encoding functional molecule]-WPRE3-BGHpA;
   eHGT_748m-minBglobin-[gene encoding functional molecule]-WPRE3-BGHpA;
   eHGT_749m-minBglobin-[gene encoding functional molecule]-WPRE3-BGHpA;
   eHGT_750m-minBglobin-[gene encoding functional molecule]-WPRE3-BGHpA;
   eHGT_751m-minBglobin-[gene encoding functional molecule]-WPRE3-BGHpA;
   eHGT_779m-minBglobin-[gene encoding functional molecule]-WPRE3-BGHpA;
   eHGT_780m-minBglobin-[gene encoding functional molecule]-WPRE3-BGHpA;
   eHGT_781m-minBglobin-[gene encoding functional molecule]-WPRE3-BGHpA;
   eHGT_782m-minBglobin-[gene encoding functional molecule]-WPRE3-BGHpA;
   eHGT_783m-minBglobin-[gene encoding functional molecule]-WPRE3-BGHpA;
   eHGT_784m-minBglobin-[gene encoding functional molecule]-WPRE3-BGHpA;
   eHGT_785m-minBglobin-[gene encoding functional molecule]-WPRE3-BGHpA;
   eHGT_452h-minBglobin-[gene encoding functional molecule]A-WPRE3-BGHpA;
   3×SP10ins-eHGT_367h-minRho-[gene encoding functional molecule]-WPRE3-BGHpA;
   3×SP10ins-eHGT_367h-minBglobin-[gene encoding functional molecule]-WPRE3-BGHpA;
   3×SP10ins-3×core2_eHGT_367h-minRho*-[gene encoding functional molecule]-WPRE3-BGHpA;
   3×core_eHGT_441h-minBglobin-[gene encoding functional molecule]-WPRE3-BGHpA;
   3×core2_eHGT_445h-minBglobin-[gene encoding functional molecule]-WPRE3-BGHpA;
   3×core2_eHGT_444h-minBglobin-[gene encoding functional molecule]-WPRE3-BGHpA;
   3×core2_eHGT_452h-minBglobin-[gene encoding functional molecule]-WPRE3-BGHpA;
   3×core2_eHGT_779m-minBglobin-[gene encoding functional molecule]-WPRE3-BGHpA;
   3×Core-eHGT_621h-minBglobin-[gene encoding functional molecule]-WPRE3-BGHpA;
   3×Core2_eHGT_780m-minBglobin-[gene encoding functional molecule]-WPRE3-BGHpA;
   3×Core-eHGT_447h-minBglobin-[gene encoding functional molecule]-WPRE3-BGHpA;
   3×SP10ins_3×core2_eHGT_351h-minBglobin-[gene encoding functional molecule]-WPRE3-BGHpA;
3×core2_eHGT_450h-minBglobin-[gene encoding functional molecule]-WPRE3-BGHpA;
3×core3_eHGT_450h-minBglobin-[gene encoding functional molecule]-WPRE3-BGHpA;
3×core2_eHGT_743m-[minimal promoter]-[gene encoding functional molecule]-WPRE3-BGHpA;
3×SP10ins-core2_eHGT_367h-[minimal promoter]-[gene encoding functional molecule]-WPRE3-BGHpA;
eHGT_608h-[minimal promoter]-[gene encoding functional molecule]-WPRE3-BGHpA;
eHGT_609h-[minimal promoter]-[gene encoding functional molecule]-WPRE3-BGHpA;
eHGT_621h-[minimal promoter]-[gene encoding functional molecule]-WPRE3-BGHpA;
eHGT_633h-[minimal promoter]-[gene encoding functional molecule]-WPRE3-BGHpA;
eHGT_634h-[minimal promoter]-[gene encoding functional molecule]-WPRE3-BGHpA;
eHGT_635h-[minimal promoter]-[gene encoding functional molecule]-WPRE3-BGHpA;
eHGT_636h-[minimal promoter]-[gene encoding functional molecule]-WPRE3-BGHpA;
3×SP10ins-eHGT_351h-[minimal promoter]-[gene encoding functional molecule]-WPRE3-BGHpA;
3×SP10ins-eHGT_367h-[minimal promoter]-[gene encoding functional molecule]-WPRE3-BGHpA;
eHGT_441h-[minimal promoter]-[gene encoding functional molecule]-WPRE3-BGHpA;
eHGT_612h-[minimal promoter]-[gene encoding functional molecule]-WPRE3-BGHpA;
eHGT_613h-[minimal promoter]-[gene encoding functional molecule]-WPRE3-BGHpA;
eHGT_614h-[minimal promoter]-[gene encoding functional molecule]-WPRE3-BGHpA;
eHGT_617h-[minimal promoter]-[gene encoding functional molecule]-WPRE3-BGHpA;
eHGT_618h-[minimal promoter]-[gene encoding functional molecule]-WPRE3-BGHpA;
eHGT_619h-[minimal promoter]-[gene encoding functional molecule]-WPRE3-BGHpA;
eHGT_620h-[minimal promoter]-[gene encoding functional molecule]-WPRE3-BGHpA;
eHGT_442h-[minimal promoter]-[gene encoding functional molecule]-WPRE3-BGHpA;
eHGT_444h-[minimal promoter]-[gene encoding functional molecule]-WPRE3-BGHpA;
eHGT_445h-[minimal promoter]-[gene encoding functional molecule]-WPRE3-BGHpA;
eHGT_450h-[minimal promoter]-[gene encoding functional molecule]-WPRE3-BGHpA;
eHGT_452h-[minimal promoter]-[gene encoding functional molecule]-WPRE3-BGHpA;
eHGT_610h-[minimal promoter]-[gene encoding functional molecule]-WPRE3-BGHpA;
eHGT_611h-[minimal promoter]-[gene encoding functional molecule]-WPRE3-BGHpA;
eHGT_615h-[minimal promoter]-[gene encoding functional molecule]-WPRE3-BGHpA;
eHGT_616h-[minimal promoter]-[gene encoding functional molecule]-WPRE3-BGHpA;
eHGT_627h-[minimal promoter]-[gene encoding functional molecule]-WPRE3-BGHpA;
eHGT_628h-[minimal promoter]-[gene encoding functional molecule]-WPRE3-BGHpA;
eHGT_629h-[minimal promoter]-[gene encoding functional molecule]-WPRE3-BGHpA;
eHGT_446h-[minimal promoter]-[gene encoding functional molecule]-WPRE3-BGHpA;
eHGT_447h-[minimal promoter]-[gene encoding functional molecule]-WPRE3-BGHpA;
eHGT_622h-[minimal promoter]-[gene encoding functional molecule]-WPRE3-BGHpA;
eHGT_623h-[minimal promoter]-[gene encoding functional molecule]-WPRE3-BGHpA;
eHGT_624h-[minimal promoter]-[gene encoding functional molecule]-WPRE3-BGHpA;
eHGT_625h-[minimal promoter]-[gene encoding functional molecule]-WPRE3-BGHpA;
eHGT_630h-[minimal promoter]-[gene encoding functional molecule]-WPRE3-BGHpA;
eHGT_631h-[minimal promoter]-[gene encoding functional molecule]-WPRE3-BGHpA;
eHGT_735m-[minimal promoter]-[gene encoding functional molecule]-WPRE3-BGHpA;
eHGT_736m-[minimal promoter]-[gene encoding functional molecule]-WPRE3-BGHpA;
eHGT_737m-[minimal promoter]-[gene encoding functional molecule]-WPRE3-BGHpA;
eHGT_738m-[minimal promoter]-[gene encoding functional molecule]-WPRE3-BGHpA;
eHGT_739m-[minimal promoter]-[gene encoding functional molecule]-WPRE3-BGHpA;
eHGT_740m-[minimal promoter]-[gene encoding functional molecule]-WPRE3-BGHpA;
eHGT_741m-[minimal promoter]-[gene encoding functional molecule]-WPRE3-BGHpA;
eHGT_742m-[minimal promoter]-[gene encoding functional molecule]-WPRE3-BGHpA;
eHGT_743m-[minimal promoter]-[gene encoding functional molecule]-WPRE3-BGHpA;
eHGT_744m-[minimal promoter]-[gene encoding functional molecule]-WPRE3-BGHpA;
eHGT_746m-[minimal promoter]-[gene encoding functional molecule]-WPRE3-BGHpA;
eHGT_747m-[minimal promoter]-[gene encoding functional molecule]-WPRE3-BGHpA;
eHGT_748m-[minimal promoter]-[gene encoding functional molecule]-WPRE3-BGHpA;
eHGT_749m-[minimal promoter]-[gene encoding functional molecule]-WPRE3-BGHpA;
eHGT_750m-[minimal promoter]-[gene encoding functional molecule]-WPRE3-BGHpA;
eHGT_751m-[minimal promoter]-[gene encoding functional molecule]-WPRE3-BGHpA;
eHGT_779m-[minimal promoter]-[gene encoding functional molecule]-WPRE3-BGHpA;
eHGT_780m-[minimal promoter]-[gene encoding functional molecule]-WPRE3-BGHpA;
eHGT_781m-[minimal promoter]-[gene encoding functional molecule]-WPRE3-BGHpA;
eHGT_782m-[minimal promoter]-[gene encoding functional molecule]-WPRE3-BGHpA;
eHGT_783m-[minimal promoter]-[gene encoding functional molecule]-WPRE3-BGHpA;
eHGT_784m-[minimal promoter]-[gene encoding functional molecule]-WPRE3-BGHpA;
eHGT_785m-[minimal promoter]-[gene encoding functional molecule]-WPRE3-BGHpA;
eHGT_452h-[minimal promoter]-[gene encoding functional molecule]A-WPRE3-BGHpA;
3×SP10ins-3×core2_eHGT_367h-[minimal promoter]-[gene encoding functional molecule]-WPRE3-BGHpA;
3×core_eHGT_441h-[minimal promoter]-[gene encoding functional molecule]-WPRE3-BGHpA;
3×core2_eHGT_445h-[minimal promoter]-[gene encoding functional molecule]-WPRE3-BGHpA;
3×core2_eHGT_444h-[minimal promoter]-[gene encoding functional molecule]-WPRE3-BGHpA;
3×core2_eHGT_452h-[minimal promoter]-[gene encoding functional molecule]-WPRE3-BGHpA;
3×core2_eHGT_779m-[minimal promoter]-[gene encoding functional molecule]-WPRE3-BGHpA;
3×Core-eHGT_621h-[minimal promoter]-[gene encoding functional molecule]-WPRE3-BGHpA;
3×Core2_eHGT_780m-[minimal promoter]-[gene encoding functional molecule]-WPRE3-BGHpA;
3×Core-eHGT_447h-[minimal promoter]-[gene encoding functional molecule]-WPRE3-BGHpA;
3×SP10ins_3×core2_eHGT_351h-[minimal promoter]-[gene encoding functional molecule]-WPRE3-BGHpA;
3×core2_eHGT_450h-[minimal promoter]-[gene encoding functional molecule]-WPRE3-BGHpA; or
3×core3_eHGT_450h-[minimal promoter]-[gene encoding functional molecule]-WPRE3-BGHpA
   Wherein the functional molecule in each of the foregoing embodiments can be a therapeutic gene product.
49. The artificial expression construct of embodiment 48, wherein the functional molecule is a therapeutic gene product that treats a movement disorder.
50. A vector including an artificial expression construct of any of embodiments 31-49.
51. The vector of embodiment 50, wherein the vector includes a viral vector.
52. The vector of embodiment 51, wherein the viral vector includes a recombinant adeno-associated viral (AAV) vector or a plasmid (AAV).
53. An adeno-associated viral (AAV) vector including at least one heterologous encoding sequence, wherein the heterologous encoding sequence is under control of a promoter and an enhancer selected from eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core2_eHGT_367h, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, and 3×core2_eHGT_743m.
54. A transgenic cell including an artificial expression construct or vector of any of the preceding embodiments.
55. The transgenic cell of embodiment 54, wherein the transgenic cell is a striatal neuron.
56. The transgenic cell of embodiments 54 or 55, wherein the transgenic cell is a striatal interneuron-cholinergic cell, a striatal medium spiny neuron-direct pathway cell, a striatal medium spiny neuron-indirect pathway cell, a striatal medium spiny neuron-pan cell, or a Drd3+ medium spiny cell.
57. The transgenic cell of embodiments 54, wherein the transgenic cell includes cells in the thalamus, cortex, hippocampus, olfactory bulb, Taenia tecta dorsal, hypothalamus, or amygdala.
58. The transgenic cell of embodiments 57, wherein the cells in the thalamus include cells within the paraventricular nucleus or the parafascicular nucleus.
59. The transgenic cell of embodiments 57, wherein the cells in the cortex include L2/3 IT neurons or L6 neurons.
60. The transgenic cell of embodiments 57, wherein the cells in the hippocampus include superior colliculus, CA1, CA2, CA3, hilus, or lateral pathway axons.
61. The transgenic cell of embodiments 57, wherein the cells in the olfactory bulb include periglomerular cells or glomeruli cells.
62. The transgenic cell of embodiments 57, wherein the cells in the Taenia tecta dorsal include putative excitatory neurons.
63. The transgenic cell of embodiments 57, wherein the cells in the hypothalamus include cells within the paraventricular hypothalamic nucleus.
64. The transgenic cell of embodiments 57, wherein the cells in the amygdala include cells within the lateral, medial and main intercalated (ITC) nucleus, central amygdala (CEA) nucleus, or cortical amygdalar area (COAp).
65. The transgenic cell of embodiment 54, wherein the transgenic cell is (or is found within) a putative cholinergic interneuron in the lateral septal nucleus, caudodorsal part of the lateral septum, accessory olfactory nucleus (AON), upper band of L2/3 pyramidal neurons in Pir, piriform-amygdalar area, entorhinal, perirhinal, frontal areas especially motor cortex areas, L3 in caudal entorhinal cortex, molecular layer of piriform area, dorsal tip of the interpeduncular nucleus, dorsal tegmental nucleus, axons in the supragenual nucleus, posterior of the basolateral amygdala (BLA), CA2, CA3, caudal CA1/Subiculum pyramidal neurons, substantia innominata axon, piriform area L2, CEA dense, BLA sparse, L5, or retrosplenial (RSP) cells.
66. A non-human transgenic animal including an artificial expression construct, vector, or transgenic cell of any of the preceding embodiments.
67. The non-human transgenic animal of embodiment 66, wherein the non-human transgenic animal is a mouse or a non-human primate.
68. An administrable composition including an artificial expression construct, vector, ortransgenic cell of any of the preceding embodiments.
69. A kit including an artificial expression construct, vector, transgenic cell, transgenic animal, and/or administrable compositions of any of the preceding embodiments.
70. A method for expressing a heterologous gene within striatal neurons in vivo or in vitro, the method including administering the administrable composition of embodiment 58 in a sufficient dosage and for a sufficient time to a sample or subject including the striatal neurons thereby expressing the gene within the striatal neurons.
71. The method of embodiment 70, wherein the heterologous gene encodes an effector element or an expressible element.
72. The method of embodiment 71, wherein the effector element includes a reporter protein or a functional molecule.
73. The method of embodiment 72, wherein the reporter protein includes a fluorescent protein.
74. The method of embodiment 72 or 73, wherein the functional molecule includes a functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, microRNA, homologous recombination donor cassette, DREADD, and/or a therapeutic gene product.
75. The method of embodiment 74, wherein the functional molecule includes aromatic L-amino acid decarboxylase (AADC), improved Cre (iCre), monomeric teal fluorescent protein 1 (mTFP1), tyrosine hydroxylase (TH), GTP cyclohydrolase I (CH1), tetrahydrobiopterin (BH4) and/or glucocerebrosidase (GCase).
76. The method of embodiment 74, wherein the RNA suppresses or inhibits the expression of a pathogenic huntingtin (HTT) gene.
77. The method of embodiment 76, wherein the RNA sequence includes SEQ ID NOs: 163-195.
78. The method of embodiment 71, wherein the expressible element includes a non-functional molecule.
79. The method of embodiment 78, wherein the non-functional molecule includes a non-functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, microRNA, homologous recombination donor cassette, or DREADD.
80. The method of any of embodiments 70-79, wherein the administering includes pipetting.
81. The method of embodiment 80, wherein the pipetting is to a brain slice.
82. The method of embodiment 81, wherein the brain slice includes a striatal interneuron-cholinergic cell, a striatal medium spiny neuron-direct pathway cell, a striatal medium spiny neuron-indirect pathway cell, a striatal medium spiny neuron-pan cell, or a Drd3+ medium spiny cells.
83. The method of embodiment 81 or 82, wherein the brain slice is murine, human, or non-human primate.
84. The method of any of embodiments 70-79, wherein the administering includes administering to a living subject.
85. The method of embodiment 84, wherein the living subject is a human, non-human primate, or a mouse.
86. The method of embodiment 84 or 85, wherein the administering provides a therapeutically effective amount.
87. The method of embodiment 86, wherein the therapeutically effective amount treats a movement disorder.
88. The method of embodiment 86, wherein the therapeutically effective amount provides an effective amount, a prophylactic treatment and/or a therapeutic treatment against a movement disorder.
89. The method of embodiment 87 or 88, wherein the movement disorder includes Parkinson's disease, Huntington's disease, ataxia, corticobasal ganglionic degeneration (CBGD), dyskinesia, dystonia, tremors, hereditary spastic paraplegia, multiple system atrophy, myoclonus, progressive supranuclear palsy, restless legs syndrome, Rett syndrome, spasticity, Sydenham's chorea, other choreas, athetosis, ballism, stereotypy, tardive dyskinesia/dystonia, tics, Tourette's syndrome, olivopontocerebellar atrophy (OPCA), diffuse Lewy body disease, hemibalismus, hemi-facial spasm, Wilson's disease, stiff man syndrome, akinetic mutism, psychomotor retardation, painful legs, moving toes syndrome, a gait disorder, or a drug-induced movement disorder.
90. The method of any of embodiments 84-89, wherein the administering to a living subject is through injection.
91. The method of embodiment 90, wherein the injection includes intravenous injection, intraparenchymal injection into brain tissue, intracerebroventricular (ICV) injection, intra-cisterna magna (ICM) injection, or intrathecal injection.
92. An artificial expression construct including CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2514, CN2555, CN2907, CN2909, CN2921, CN2982, CN3044, CN3038, CN3344, CN3281, CN3346, CN3566, CN2912, CN2913, CN2966, CN2203, or CN2700.

(viii) Closing Paragraphs. Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The term concatenate is broadly used to describe linking together into a chain or series. It is used to describe the linking together of nucleotide or amino acid sequences into a single nucleotide or amino acid sequence, respectively. The term “concatamerize” should be interpreted to recite: “concatenate.” In particular embodiments, enhancer are concatenated into 2, 3, 4, 5, 6, 7, 9, or 10 copies within a single artificial expression construct.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in targeted expression in the targeted cell population as determined by scRNA-Seq and the targeted cell population and enhancer pairings:
striatal medium spiny neuron-pan: eHGT_608h, eHGT_609h, eHGT_633h, eHGT_634h, eHGT_635h, eHGT_636h, eHGT_351h, eHGT_367h, eHGT_450h, eHGT_447h, eHGT_744m, eHGT_782m, eHGT_785m, eHGT_441h, core2_eHGT_367h, 3×core2_eHGT_367h, 3×core_eHGT_441h, 3×Core2_eHGT_444h, 3×Core-eHGT_447h, and 3×core2_eHGT_351h;
striatal medium spiny neuron-indirect pathway: eHGT_612h, eHGT_613h, eHGT_614h, eHGT_617h, eHGT_618h, eHGT_619h, eHGT_620h, eHGT_442h, eHGT_444h, eHGT_445h, eHGT_452h, eHGT_784m, 3×core2_eHGT_445h, 3×core2_eHGT_444h, 3×core2_eHGT_452h, 3×core2_eHGT_450h, and 3×core3_eHGT_450h;
striatal medium spiny neuron-direct pathway: eHGT_610h, eHGT_611h, eHGT_615h, eHGT_616h, eHGT_627h, eHGT_628h, eHGT_629h, eHGT_446h, eHGT_779m, eHGT_780m, eHGT_781m, eHGT_783m, 3×core2_eHGT_779m and 3×Core2_eHGT_780m;
striatal interneuron-cholinergic: eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_743m, eHGT_742m, HGT_746m, eHGT_747m, eHGT_748m, eHGT_749m, eHGT_750m, eHGT_751m, and 3×core2_eHGT_743m;
Drd3+ medium spiny neurons in olfactory tubercle: eHGT_621h and 3×core-eHGT_621h.

In particular embodiments, artificial means not naturally occurring.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. An artificial expression construct comprising (i) an enhancer selected from 3×core2_eHGT_743m or core2_eHGT_367h; (ii) a promoter; and (iii) a heterologous encoding sequence.

2. The artificial expression construct of claim 1, wherein the enhancer comprises 3×core2_eHGT_743m, the promoter is minBglobin, and the heterologous encoding sequence encodes human aromatic L-amino acid decarboxylase.

3. The artificial expression construct of claim 1, wherein the enhancer comprises core2_eHGT_367h, the promoter is minRho*, and the heterologous encoding sequence encodes human aromatic L-amino acid decarboxylase.

4. A core of the eHGT_351h, eHGT_367h, eHGT_441h, eHGT_444h, eHGT_445h, eHGT_447h, eHGT_450h, eHGT_452h, eHGT_621h, eHGT_743m, eHGT_779m, or eHGT_780m enhancer.

5. The core of claim 4, wherein the core has the sequence as set forth in SEQ ID NO: 202; SEQ ID NO: 204; SEQ ID NO: 206; SEQ ID NO: 208; SEQ ID NO: 210; SEQ ID NO: 212; SEQ ID NO: 214; SEQ ID NO: 216; SEQ ID NO: 218; SEQ ID NO: 220; SEQ ID NO: 222; SEQ ID NO: 224; or SEQ ID NO: 226.

6. The core of claim 4, wherein the core is concatenated into a concatemer comprising 2, 3, 4, 5, 6, 7, 8, 9, or 10 copies of the enhancer core.

7. The core of claim 6, wherein concatenated core comprises 2, 3, 4, 5, 6, 7, 8, 9, or 10 copies of SEQ ID NO: 202; SEQ ID NO: 204; SEQ ID NO: 206; SEQ ID NO: 208; SEQ ID NO: 210; SEQ ID NO: 212; SEQ ID NO: 214; SEQ ID NO: 216; SEQ ID NO: 218; SEQ ID NO: 220; SEQ ID NO: 222; SEQ ID NO: 224; and/or SEQ ID NO: 226.

8. The core of claim 6, wherein the concatenated core comprises 3 copies of SEQ ID NO: 206.

9. The core of claim 6, wherein the concatenated core comprises 3 copies of SEQ ID NO: 202.

10. The core of claim 6, wherein the concatenated core comprises 3 copies of SEQ ID NO: 204.

11. The core of claim 6, wherein the concatenated core comprises 3 copies of SEQ ID NO: 208.

12. The core of claim 6, wherein the concatenated core comprises 3 copies of SEQ ID NO: 210.

13. The core of claim 6, wherein the concatenated core comprises 3 copies of SEQ ID NO: 212.

14. The core of claim 6, wherein the concatenated core comprises 3 copies of SEQ ID NO: 214.

15. The core of claim 6, wherein the concatenated core comprises 3 copies of SEQ ID NO: 216.

16. The core of claim 6, wherein the concatenated core comprises 3 copies of SEQ ID NO: 218.

17. The core of claim 6, wherein the concatenated core comprises 3 copies of SEQ ID NO: 220.

18. The core of claim 6, wherein the concatenated core comprises 3 copies of SEQ ID NO: 222.

19. The core of claim 6, wherein the concatenated core comprises 3 copies of SEQ ID NO: 224.

20. The core of claim 6, wherein the concatenated core comprises 3 copies of SEQ ID NO: 226.

21. The core of claim 8, wherein the concatenated core comprises SEQ ID NO: 207.

22. The core of claim 9, wherein the concatenated core comprises SEQ ID NO: 203.

23. The core of claim 10, wherein the concatenated core comprises SEQ ID NO: 205.

24. The core of claim 11, wherein the concatenated core comprises SEQ ID NO: 209.

25. The core of claim 12, wherein the concatenated core comprises SEQ ID NO: 211.

26. The core of claim 13, wherein the concatenated core comprises SEQ ID NO: 213.

27. The core of claim 14, wherein the concatenated core comprises SEQ ID NO: 215.

28. The core of claim 15, wherein the concatenated core comprises SEQ ID NO: 217.

29. The core of claim 16, wherein the concatenated core comprises SEQ ID NO: 219.

30. The core of claim 17, wherein the concatenated core comprises SEQ ID NO: 221.

31. The core of claim 18, wherein the concatenated core comprises SEQ ID NO: 223.

32. The core of claim 19, wherein the concatenated core comprises SEQ ID NO: 225.

33. The core of claim 20, wherein the concatenated core comprises SEQ ID NO: 227.

34. An artificial expression construct comprising (i) an enhancer selected from core2_eHGT_743m, core2_eHGT_367h, eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, core_eHGT_441h, Core-eHGT_621h, Core-eHGT_447h, core2_eHGT_351h, core2_eHGT_445h, core2_eHGT_444h, core2_eHGT_452h, core2_eHGT_450h, core3_eHGT_450h, core2_eHGT_779m, and Core2_eHGT_780m; (ii) a promoter; and (iii) a heterologous encoding sequence.

35. The artificial expression construct of claim 4, wherein the enhancer is concatenated comprising 3×core2_eHGT_743m, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, and 3×Core2_eHGT_780m

36. The artificial expression construct of claim 34, wherein the heterologous encoding sequence encodes an effector element or an expressible element.

37. The artificial expression construct of claim 36, wherein the effector element comprises a reporter protein or a functional molecule.

38. The artificial expression construct of claim 37, wherein the reporter protein comprises a fluorescent protein.

39. The artificial expression construct of claim 37, wherein the functional molecule comprises a functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, microRNA, homologous recombination donor cassette, or a designer receptor exclusively activated by designer drug (DREADD).

40. The artificial expression construct of claim 39, wherein the functional molecule comprises aromatic L-amino acid decarboxylase (AADC), improved Cre (iCre), monomeric teal fluorescent protein 1 (mTFP1), tyrosine hydroxylase (TH), GTP cyclohydrolase I (CH1), tetrahydrobiopterin (BH4) and/or glucocerebrosidase (GCase).

41. The artificial expression construct of claim 39, wherein the mRNA suppresses or inhibits the expression of a pathogenic huntingtin (HTT) gene.

42. The artificial expression construct of claim 41, wherein the RNA sequence comprises SEQ ID NOs: 163-195.

43. The artificial expression construct of claim 36, wherein the expressible element comprises a non-functional molecule.

44. The artificial expression construct of claim 43, wherein the non-functional molecule comprises a non-functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, homologous recombination donor cassette, or a DREADD.

45. The artificial expression construct of claim 34, wherein the artificial expression construct is associated with a capsid that crosses the blood brain barrier.

46. The artificial expression construct of claim 45, wherein the capsid comprises PHP.eB, AAV-BR1, AAV-PHP.S, AAV-PHP.B, AAV-PPS, PHP.V1, AAV1, AAV2, AAV5, AAV8, AAV9, AAV11, Rh10, Hu11, AAV2-retro, AAV9-retro, CAP.B10, or CAP.B22.

47. The artificial expression construct of claim 34, wherein the artificial expression construct comprises or encodes a skipping element.

48. The artificial expression construct of claim 47, wherein the skipping element comprises a 2A peptide and/or an internal ribosome entry site (IRES).

49. The artificial expression construct of claim 48, wherein the 2A peptide is selected from T2A, P2A, E2A, or F2A.

50. The artificial expression construct of claim 34, wherein the artificial expression construct comprises or encodes a set of features selected from 3×core2_eHGT_743m, core2_eHGT_367h, eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, 3×Core2_eHGT_780m, AAV, scAAV, rAAV, pAAV, minBglobin, CMV, minCMV, minRho, minRho*, AADC, TH, CH1, BH4 GCase, Intron, 3×HA, an RNA that suppresses or inhibits the expression of a pathogenic HTT gene, a gene whose expression treats a movement disorder, fluorescent protein, Cre, iCre, dgCre, FlpO, tTA2, SP10ins, WPRE3, hGHpA, and/or BGHpA.

51. The artificial expression construct of claim 34, wherein the artificial expression construct comprises or encodes a set of features selected from:
3×core2_eHGT_743m-minBglobin-[gene encoding functional molecule]-WPRE3-BGHpA;
3×SP10ins-core2_eHGT_367h-minRho*-[gene encoding functional molecule]-WPRE3-BGHpA;
eHGT_608h-minBglobin-[gene encoding functional molecule]-WPRE3-BGHpA;
eHGT_609h-minBglobin-[gene encoding functional molecule]-WPRE3-BGHpA;
eHGT_621h-minBglobin-[gene encoding functional molecule]-WPRE3-BGHpA;
eHGT_633h-minBglobin-[gene encoding functional molecule]-WPRE3-BGHpA;
eHGT_634h-minBglobin-[gene encoding functional molecule]-WPRE3-BGHpA;
eHGT_635h-minBglobin-[gene encoding functional molecule]-WPRE3-BGHpA;
eHGT_636h-minBglobin-[gene encoding functional molecule]-WPRE3-BGHpA;
3×SP10ins-eHGT_351h-minRho*-[gene encoding functional molecule]-WPRE3-BGHpA;
3×SP10ins-eHGT_367h-minRho*-[gene encoding functional molecule]-WPRE3-BGHpA;
eHGT_441h-minBglobin-[gene encoding functional molecule]-WPRE3-BGHpA;
eHGT_612h-minBglobin-[gene encoding functional molecule]-WPRE3-BGHpA;
eHGT_613h-minBglobin-[gene encoding functional molecule]-WPRE3-BGHpA;
eHGT_614h-minBglobin-[gene encoding functional molecule]-WPRE3-BGHpA;
eHGT_617h-minBglobin-[gene encoding functional molecule]-WPRE3-BGHpA;
eHGT_618h-minBglobin-[gene encoding functional molecule]-WPRE3-BGHpA;
eHGT_619h-minBglobin-[gene encoding functional molecule]-WPRE3-BGHpA;
eHGT_620h-minBglobin-[gene encoding functional molecule]-WPRE3-BGHpA;
eHGT_442h-minBglobin-[gene encoding functional molecule]-WPRE3-BGHpA;
eHGT_444h-minBglobin-[gene encoding functional molecule]-WPRE3-BGHpA;
eHGT_445h-minBglobin-[gene encoding functional molecule]-WPRE3-BGHpA;
eHGT_450h-minBglobin-[gene encoding functional molecule]-WPRE3-BGHpA;
eHGT_452h-minBglobin-[gene encoding functional molecule]-WPRE3-BGHpA;
eHGT_610h-minBglobin-[gene encoding functional molecule]-WPRE3-BGHpA;
eHGT_611h-minBglobin-[gene encoding functional molecule]-WPRE3-BGHpA;
eHGT_615h-minBglobin-[gene encoding functional molecule]-WPRE3-BGHpA;
eHGT_616h-minBglobin-[gene encoding functional molecule]-WPRE3-BGHpA;
eHGT_627h-minBglobin-[gene encoding functional molecule]-WPRE3-BGHpA;
eHGT_628h-minBglobin-[gene encoding functional molecule]-WPRE3-BGHpA;
eHGT_629h-minBglobin-[gene encoding functional molecule]-WPRE3-BGHpA;
eHGT_446h-minBglobin-[gene encoding functional molecule]-WPRE3-BGHpA;
eHGT_447h-minBglobin-[gene encoding functional molecule]-WPRE3-BGHpA;
eHGT_622h-minBglobin-[gene encoding functional molecule]-WPRE3-BGHpA;
eHGT_623h-minBglobin-[gene encoding functional molecule]-WPRE3-BGHpA;
eHGT_624h-minBglobin-[gene encoding functional molecule]-WPRE3-BGHpA;
eHGT_625h-minBglobin-[gene encoding functional molecule]-WPRE3-BGHpA;
eHGT_630h-minBglobin-[gene encoding functional molecule]-WPRE3-BGHpA;
eHGT_631h-minBglobin-[gene encoding functional molecule]-WPRE3-BGHpA;
eHGT_735m-minBglobin-[gene encoding functional molecule]-WPRE3-BGHpA;
eHGT_736m-minBglobin-[gene encoding functional molecule]-WPRE3-BGHpA;
eHGT_737m-minBglobin-[gene encoding functional molecule]-WPRE3-BGHpA;
eHGT_738m-minBglobin-[gene encoding functional molecule]-WPRE3-BGHpA;
eHGT_739m-minBglobin-[gene encoding functional molecule]-WPRE3-BGHpA;
eHGT_740m-minBglobin-[gene encoding functional molecule]-WPRE3-BGHpA;
eHGT_741m-minBglobin-[gene encoding functional molecule]-WPRE3-BGHpA;
eHGT_742m-minBglobin-[gene encoding functional molecule]-WPRE3-BGHpA;
eHGT_743m-minBglobin-[gene encoding functional molecule]-WPRE3-BGHpA;
eHGT_744m-minBglobin-[gene encoding functional molecule]-WPRE3-BGHpA;
eHGT_746m-minBglobin-[gene encoding functional molecule]-WPRE3-BGHpA;
eHGT_747m-minBglobin-[gene encoding functional molecule]-WPRE3-BGHpA;
eHGT_748m-minBglobin-[gene encoding functional molecule]-WPRE3-BGHpA;
eHGT_749m-minBglobin-[gene encoding functional molecule]-WPRE3-BGHpA;
eHGT_750m-minBglobin-[gene encoding functional molecule]-WPRE3-BGHpA;
eHGT_751m-minBglobin-[gene encoding functional molecule]-WPRE3-BGHpA;
eHGT_779m-minBglobin-[gene encoding functional molecule]-WPRE3-BGHpA;
eHGT_780m-minBglobin-[gene encoding functional molecule]-WPRE3-BGHpA;
eHGT_781m-minBglobin-[gene encoding functional molecule]-WPRE3-BGHpA;
eHGT_782m-minBglobin-[gene encoding functional molecule]-WPRE3-BGHpA;
eHGT_783m-minBglobin-[gene encoding functional molecule]-WPRE3-BGHpA;
eHGT_784m-minBglobin-[gene encoding functional molecule]-WPRE3-BGHpA;
eHGT_785m-minBglobin-[gene encoding functional molecule]-WPRE3-BGHpA;
eHGT_452h-minBglobin-[gene encoding functional molecule]A-WPRE3-BGHpA;
3×SP10ins-eHGT_367h-minRho-[gene encoding functional molecule]-WPRE3-BGHpA;
3×SP10ins-eHGT_367h-minBglobin-[gene encoding functional molecule]-WPRE3-BGHpA;
3×SP10ins-3×core2_eHGT_367h-minRho*-[gene encoding functional molecule]-WPRE3-BGHpA;
3×core_eHGT_441h-minBglobin-[gene encoding functional molecule]-WPRE3-BGHpA;
3×core2_eHGT_445h-minBglobin-[gene encoding functional molecule]-WPRE3-BGHpA;
3×core2_eHGT_444h-minBglobin-[gene encoding functional molecule]-WPRE3-BGHpA;
3×core2_eHGT_452h-minBglobin-[gene encoding functional molecule]-WPRE3-BGHpA;
3×core2_eHGT_779m-minBglobin-[gene encoding functional molecule]-WPRE3-BGHpA;
3×Core-eHGT_621h-minBglobin-[gene encoding functional molecule]-WPRE3-BGHpA;
3×Core2_eHGT_780m-minBglobin-[gene encoding functional molecule]-WPRE3-BGHpA;
3×Core-eHGT_447h-minBglobin-[gene encoding functional molecule]-WPRE3-BGHpA;
3×SP10ins_3×core2_eHGT_351h-minBglobin-[gene encoding functional molecule]-WPRE3-BGHpA;
3×core2_eHGT_450h-minBglobin-[gene encoding functional molecule]-WPRE3-BGHpA;
3×core3_eHGT_450h-minBglobin-[gene encoding functional molecule]-WPRE3-BGHpA;
3×core2_eHGT_743m-[minimal promoter]-[gene encoding functional molecule]-WPRE3-BGHpA;
3×SP10ins-core2_eHGT_367h-[minimal promoter]-[gene encoding functional molecule]-WPRE3-BGHpA;
eHGT_608h-[minimal promoter]-[gene encoding functional molecule]-WPRE3-BGHpA;
eHGT_609h-[minimal promoter]-[gene encoding functional molecule]-WPRE3-BGHpA;
eHGT_621h-[minimal promoter]-[gene encoding functional molecule]-WPRE3-BGHpA;
eHGT_633h-[minimal promoter]-[gene encoding functional molecule]-WPRE3-BGHpA;
eHGT_634h-[minimal promoter]-[gene encoding functional molecule]-WPRE3-BGHpA;
eHGT_635h-[minimal promoter]-[gene encoding functional molecule]-WPRE3-BGHpA;
eHGT_636h-[minimal promoter]-[gene encoding functional molecule]-WPRE3-BGHpA;
3×SP10ins-eHGT_351h-[minimal promoter]-[gene encoding functional molecule]-WPRE3-BGHpA;
3×SP10ins-eHGT_367h-[minimal promoter]-[gene encoding functional molecule]-WPRE3-BGHpA;
eHGT_441h-[minimal promoter]-[gene encoding functional molecule]-WPRE3-BGHpA;
eHGT_612h-[minimal promoter]-[gene encoding functional molecule]-WPRE3-BGHpA;
eHGT_613h-[minimal promoter]-[gene encoding functional molecule]-WPRE3-BGHpA;
eHGT_614h-[minimal promoter]-[gene encoding functional molecule]-WPRE3-BGHpA;
eHGT_617h-[minimal promoter]-[gene encoding functional molecule]-WPRE3-BGHpA;
eHGT_618h-[minimal promoter]-[gene encoding functional molecule]-WPRE3-BGHpA;
eHGT_619h-[minimal promoter]-[gene encoding functional molecule]-WPRE3-BGHpA;
eHGT_620h-[minimal promoter]-[gene encoding functional molecule]-WPRE3-BGHpA;
eHGT_442h-[minimal promoter]-[gene encoding functional molecule]-WPRE3-BGHpA;
eHGT_444h-[minimal promoter]-[gene encoding functional molecule]-WPRE3-BGHpA;
eHGT_445h-[minimal promoter]-[gene encoding functional molecule]-WPRE3-BGHpA;
eHGT_450h-[minimal promoter]-[gene encoding functional molecule]-WPRE3-BGHpA;
eHGT_452h-[minimal promoter]-[gene encoding functional molecule]-WPRE3-BGHpA;
eHGT_610h-[minimal promoter]-[gene encoding functional molecule]-WPRE3-BGHpA;
eHGT_611h-[minimal promoter]-[gene encoding functional molecule]-WPRE3-BGHpA;
eHGT_615h-[minimal promoter]-[gene encoding functional molecule]-WPRE3-BGHpA;
eHGT_616h-[minimal promoter]-[gene encoding functional molecule]-WPRE3-BGHpA;
eHGT_627h-[minimal promoter]-[gene encoding functional molecule]-WPRE3-BGHpA;
eHGT_628h-[minimal promoter]-[gene encoding functional molecule]-WPRE3-BGHpA;
eHGT_629h-[minimal promoter]-[gene encoding functional molecule]-WPRE3-BGHpA;
eHGT_446h-[minimal promoter]-[gene encoding functional molecule]-WPRE3-BGHpA;
eHGT_447h-[minimal promoter]-[gene encoding functional molecule]-WPRE3-BGHpA;
eHGT_622h-[minimal promoter]-[gene encoding functional molecule]-WPRE3-BGHpA;
eHGT_623h-[minimal promoter]-[gene encoding functional molecule]-WPRE3-BGHpA;
eHGT_624h-[minimal promoter]-[gene encoding functional molecule]-WPRE3-BGHpA;
eHGT_625h-[minimal promoter]-[gene encoding functional molecule]-WPRE3-BGHpA;
eHGT_630h-[minimal promoter]-[gene encoding functional molecule]-WPRE3-BGHpA;
eHGT_631h-[minimal promoter]-[gene encoding functional molecule]-WPRE3-BGHpA;
eHGT_735m-[minimal promoter]-[gene encoding functional molecule]-WPRE3-BGHpA;
eHGT_736m-[minimal promoter]-[gene encoding functional molecule]-WPRE3-BGHpA;
eHGT_737m-[minimal promoter]-[gene encoding functional molecule]-WPRE3-BGHpA;
eHGT_738m-[minimal promoter]-[gene encoding functional molecule]-WPRE3-BGHpA;
eHGT_739m-[minimal promoter]-[gene encoding functional molecule]-WPRE3-BGHpA;
eHGT_740m-[minimal promoter]-[gene encoding functional molecule]-WPRE3-BGHpA;
eHGT_741m-[minimal promoter]-[gene encoding functional molecule]-WPRE3-BGHpA;
eHGT_742m-[minimal promoter]-[gene encoding functional molecule]-WPRE3-BGHpA;
eHGT_743m-[minimal promoter]-[gene encoding functional molecule]-WPRE3-BGHpA;
eHGT_744m-[minimal promoter]-[gene encoding functional molecule]-WPRE3-BGHpA;
eHGT_746m-[minimal promoter]-[gene encoding functional molecule]-WPRE3-BGHpA;
eHGT_747m-[minimal promoter]-[gene encoding functional molecule]-WPRE3-BGHpA;
eHGT_748m-[minimal promoter]-[gene encoding functional molecule]-WPRE3-BGHpA;
eHGT_749m-[minimal promoter]-[gene encoding functional molecule]-WPRE3-BGHpA;
eHGT_750m-[minimal promoter]-[gene encoding functional molecule]-WPRE3-BGHpA;
eHGT_751m-[minimal promoter]-[gene encoding functional molecule]-WPRE3-BGHpA;
eHGT_779m-[minimal promoter]-[gene encoding functional molecule]-WPRE3-BGHpA;
eHGT_780m-[minimal promoter]-[gene encoding functional molecule]-WPRE3-BGHpA;
eHGT_781m-[minimal promoter]-[gene encoding functional molecule]-WPRE3-BGHpA;
eHGT_782m-[minimal promoter]-[gene encoding functional molecule]-WPRE3-BGHpA;
eHGT_783m-[minimal promoter]-[gene encoding functional molecule]-WPRE3-BGHpA;
eHGT_784m-[minimal promoter]-[gene encoding functional molecule]-WPRE3-BGHpA;
eHGT_785m-[minimal promoter]-[gene encoding functional molecule]-WPRE3-BGHpA;
eHGT_452h-[minimal promoter]-[gene encoding functional molecule]A-WPRE3-BGHpA;
3×SP10ins-3×core2_eHGT_367h-[minimal promoter]-[gene encoding functional molecule]-WPRE3-BGHpA;
3×core_eHGT_441h-[minimal promoter]-[gene encoding functional molecule]-WPRE3-BGHpA;
3×core2_eHGT_445h-[minimal promoter]-[gene encoding functional molecule]-WPRE3-BGHpA;
3×core2_eHGT_444h-[minimal promoter]-[gene encoding functional molecule]-WPRE3-BGHpA;
3×core2_eHGT_452h-[minimal promoter]-[gene encoding functional molecule]-WPRE3-BGHpA;
3×core2_eHGT_779m-[minimal promoter]-[gene encoding functional molecule]-WPRE3-BGHpA;
3×Core-eHGT_621h-[minimal promoter]-[gene encoding functional molecule]-WPRE3-BGHpA;
3×Core2_eHGT_780m-[minimal promoter]-[gene encoding functional molecule]-WPRE3-BGHpA;
3×Core-eHGT_447h-[minimal promoter]-[gene encoding functional molecule]-WPRE3-BGHpA;
3×SP10ins_3×core2_eHGT_351h-[minimal promoter]-[gene encoding functional molecule]-WPRE3-BGHpA;
3×core2_eHGT_450h-[minimal promoter]-[gene encoding functional molecule]-WPRE3-BGHpA; or
3×core3_eHGT_450h-[minimal promoter]-[gene encoding functional molecule]-WPRE3-BGHpA. wherein the functional molecule optionally comprises a therapeutic gene product.

52. The artificial expression construct of claim 51, wherein the functional molecule is a therapeutic gene product that treats a movement disorder.

53. A vector comprising an artificial expression construct of any of claim 34.

54. The vector of claim 53, wherein the vector comprises a viral vector.

55. The vector of claim 54, wherein the viral vector comprises a recombinant adeno-associated viral (AAV) vector.

56. An adeno-associated viral (AAV) vector comprising at least one heterologous encoding sequence, wherein the heterologous encoding sequence is under control of a promoter and an enhancer selected from 3×core2_eHGT_743m, core2_eHGT_367h, eHGT_608h, eHGT_609h, eHGT_621h, eHGT_633h, eHGT_634h, eHGT_635h, eHGT_636h, eHGT_351h, eHGT_367h, eHGT_441h, eHGT_612h, eHGT_613h, eHGT_614h, eHGT_617h, eHGT_618h, eHGT_619h, eHGT_620h, eHGT_442h, eHGT_444h, eHGT_445h, eHGT_450h, eHGT_452h, eHGT_610h, eHGT_611h, eHGT_615h, eHGT_616h, eHGT_627h, eHGT_628h, eHGT_629h, eHGT_446h, eHGT_447h, eHGT_622h, eHGT_623h, eHGT_624h, eHGT_625h, eHGT_630h, eHGT_631h, eHGT_735m, eHGT_736m, eHGT_737m, eHGT_738m, eHGT_739m, eHGT_740m, eHGT_741m, eHGT_742m, eHGT_743m, eHGT_744m, eHGT_746m, eHGT_747m, eHGT_748m, eHGT_749m, eHGT_750m, eHGT_751m, eHGT_779m, eHGT_780m, eHGT_781m, eHGT_782m, eHGT_783m, eHGT_784m, eHGT_785m, 3×core2_eHGT_367h, 3×core_eHGT_441h, 3×Core-eHGT_621h, 3×Core-eHGT_447h, 3×core2_eHGT_351h, 3×core2_eHGT_445h, 3×core2_eHGT_444h, 3×core2_eHGT_452h, 3×core2_eHGT_450h, 3×core3_eHGT_450h, 3×core2_eHGT_779m, and 3×Core2_eHGT_780m.

57. A transgenic cell comprising an artificial expression construct of claim 34.

58. The transgenic cell of claim 57, wherein the transgenic cell is a striatal neuron.

59. The transgenic cell of claim 58, wherein the striatal neuron is a striatal interneuron-cholinergic cell, a striatal medium spiny neuron-direct pathway cell, a striatal medium spiny neuron-indirect pathway cell, a striatal medium spiny neuron-pan cell, or a Drd3+ medium spiny cells.

60. A non-human transgenic animal comprising an artificial expression construct of claim 34.

61. The non-human transgenic animal of claim 60, wherein the non-human transgenic animal is a mouse or a non-human primate.

62. An administrable composition comprising an artificial expression construct of claim 34.

63. A kit comprising an artificial expression construct of claim 34.

64. A method for expressing a heterologous gene within striatal neurons in vivo or in vitro, the method comprising administering the administrable composition of claim 62 in a sufficient dosage and for a sufficient time to a sample or subject comprising the striatal neurons thereby expressing the gene within the striatal neurons.

65. The method of claim 64, wherein the heterologous gene encodes an effector element or an expressible element.

66. The method of claim 65, wherein the effector element comprises a reporter protein or a functional molecule.

67. The method of claim 66, wherein the reporter protein comprises a fluorescent protein.

68. The method of claim 66, wherein the functional molecule comprises a functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, homologous recombination donor cassette, or a DREADD.

69. The method of claim 68, wherein the functional molecule comprises aromatic L-amino acid decarboxylase (AADC), improved Cre (iCre), monomeric teal fluorescent protein 1 (mTFP1), tyrosine hydroxylase (TH), GTP cyclohydrolase I (CH1), tetrahydrobiopterin (BH4) and/or glucocerebrosidase (GCase).

70. The method of claim 68, wherein the RNA suppresses or inhibits the expression of a pathogenic huntingtin (HTT) gene.

71. The method of claim 70, wherein the RNA sequence comprises SEQ ID NOs: 163-195.

72. The method of claim 65, wherein the expressible element comprises a non-functional molecule.

73. The method of claim 72, wherein the non-functional molecule comprises a non-functional ion transporter, enzyme, transcription factor, receptor, membrane protein, cellular trafficking protein, signaling molecule, neurotransmitter, calcium reporter, channelrhodopsin, CRISPR/CAS molecule, editase, guide RNA molecule, RNA, homologous recombination donor cassette, or DREADD.

74. The method of claim 64, wherein the administering comprises pipetting.

75. The method of claim 74, wherein the pipetting is to a brain slice.

76. The method of claim 75, wherein the brain slice comprises a striatal interneuron-cholinergic cell, a striatal medium spiny neuron-direct pathway cell, a striatal medium spiny neuron-indirect pathway cell, a striatal medium spiny neuron-pan cell, or a Drd3+ medium spiny cells.

77. The method of claim 75, wherein the brain slice is murine, human, or non-human primate.

78. The method of claim 64, wherein the administering comprises administering to a living subject.

79. The method of claim 78, wherein the living subject is a human, non-human primate, or a mouse.

80. The method of claim 78, wherein the administering provides a therapeutically effective amount.

81. The method of claim 80, wherein the therapeutically effective amount treats a movement disorder.

82. The method of claim 80, wherein the therapeutically effective amount provides an effective amount, a prophylactic treatment and/or a therapeutic treatment against a movement disorder.

83. The method of claim 81 or 82, wherein the movement disorder comprises Parkinson's disease, Huntington's disease, ataxia, corticobasal ganglionic degeneration (CBGD), dyskinesia, dystonia, tremors, hereditary spastic paraplegia, multiple system atrophy, myoclonus, progressive supranuclear palsy, restless legs syndrome, Rett syndrome, spasticity, Sydenham's chorea, other choreas, athetosis, ballism, stereotypy, tardive dyskinesia/dystonia, tics, Tourette's syndrome, olivopontocerebellar atrophy (OPCA), diffuse Lewy body disease, hemibalismus, hemi-facial spasm, Wilson's disease, stiff man syndrome, akinetic mutism, psychomotor retardation, painful legs, moving toes syndrome, a gait disorder, or a drug-induced movement disorder.

84. The method of claim 78, wherein the administering to a living subject is through injection.

85. The method of claim 84, wherein the injection comprises intravenous injection, intraparenchymal injection into brain tissue, intracerebroventricular (ICV) injection, intra-cisterna magna (ICM) injection, or intrathecal injection.

86. An artificial expression construct comprising CN3038, CN2514, CN2438, CN2439, CN2451, CN2463, CN2464, CN2465, CN2466, CN2013, CN2025, CN2229, CN2442, CN2443, CN2444, CN2447, CN2448, CN2449, CN2450, CN2467, CN2421, CN2231, CN2236, CN2237, CN2440, CN2441, CN2445, CN2446, CN2457, CN2458, CN2459, CN2232, CN2233, CN2452, CN2453, CN2454, CN2455, CN2460, CN2461, CN2628, CN2641, CN2642, CN2643, CN2629, CN2630, CN2745, CN2746, CN2631, CN2747, CN2632, CN2644, CN2748, CN2633, CN2634, CN2635, CN2609, CN2610, CN2749, CN2626, CN2611, CN2750, CN2614, CN2485, CN2486, CN2739, CN2740, CN2765, CN2766, CN2555, CN2907, CN2909, CN2921, CN2982, CN3044, CN3344, CN3281, CN3346, CN3566, CN2912, CN2913, CN2966, CN2203, and CN2700.
